SEQUENCE LISTING (94 sequences)

SEQ ID NO: 1 - DNA, 2512 bp, Artificial Sequence
  HRP isoenzyme C1B
ttaattagttttctttttatctttaaaaatatgcattccccttcttctacttcgtttacttggatcttaatcacattgggatgtcttgcgttttatgcgtctttgtccgatgctcagcttacccctaccttctacgacacttcatgtcctaatgtctcaaacatcgtacgagacatcattattaatgagctacgatcggaccctcgtatcaccgcgagtatccttcgtcttcacttccacgactgctttgttaatgtaagataatactttttcatatttctattgcgttatgaattattgtgcgttttatcctttagatattgataaatcacctcaagtcaaaatttaataaaacattaaataaaatatgatacaaagagtcaatttgttttgtaggaatataatagaaatttcaacatgtttttaaatatgggtccaaaatgttgaaaacgacatttcttatgaaaaagagtgagtatgtattaaatcataatttgctataattatcgggttgtgaaagtagttctattcattttgacatgtagatggttgcatagtacgttttgtctacaacatttttttcttgattcattttacaaaattacaagttcacttgcctccgaaaaatatgtatagccttactatgacataattacataatttacattcaataataatttttattttttatataataatttttatttttatttatataaaaaagaaagatattattgtttgtggtgtcagttgggtgaaatcgtatcctaaataaaagtcactcgagtaacggttctgatccagattaaaaaatcagtaccaaatttcacatggttagatgtcgtgtgttagattttgctgttgaataattaaatacttaactctcgtcgacaatgataatgctaaaatatttatgaaatcggattcacgcccgtgttacagtattaagagcatggtgccgtaccaaacacgatacgaatttaatggtgacaaaaaatctctgttaaattgttaccggtggtaaagagttagctatggatgtaatacatgtactaataatttgttaattaatattttggatgtttgaaagggttgtgacgcatcgatattgttagacaacacaacatcatttctaacagagaaagatgcgcttggaaacgcaaactcggctagaggatttcctacagttgacagaatcaaggccgcggtggagagggcatgcccaagaacagtttcatgcgcagatgtgcttaccattgcagctcaacaatctgttaatttggtatgctccattcattacaaacattgttttttaattttaacatattttttagttgtttgagagcgtcacaatctatatttagtatcaacaactgttcgactatatgaggtattcatggattaatcgagaaatattcaaaacgcgtggtcccgttaagaaaatttgacaagtttaatatcattggaaaaattaggtctcatcacaaggttttaccttggtaggcaaagctactttataattaaccaaaaaggataattttcattttttccaaaaatagtggagataaaacaaactccactttgagtatatcatcaatcaatttatactacatgtttatctttttctctttacatattgaaacttccgagtgacaaattaatctcacaaaaataattattttgaatataatggttactattatattataggcaggaggtccttcttggagggttcctttgggaagaagagacagcttacaagcatttttagatcttgctaatgcaaatcttccagctccattctttacacttccacaacttaaggatgcctttgcaaaagttggcctcgaccgtccttctgatctcgttgctctctccggtagttaacaaaagaaaattaaacaccatttgatataagttcaattagatatttcattattgatcttattatatggtctttcttttgttaggtggtcacacatttggaaaaaatcagtgtagatttattatggacagattatacaacttcagcaacaccggactacccgaccctaccctcaacactacttaccttcaaactcttcgtcaacaatgtcccctaaatggaaaccaaagtgtattggtggatttcgatctgcgtacgccaacggttttcgataacaaatactatgtgaatcttaaagagcaaaaaggtctcattcagagtgaccaagagttgttctctagccccaatgccactgacacaatccccttggtgaggtcatttgctgatggcacacaaaaattcttcaatgcgtttgtggaggctatgaataggatgggaaatattacacctcttacaggaactcaaggagaaatcaggttgaattgtagggtggtgaactccaactctctactccatgatatagtggaggtcgttgactttgttagctctatgtgagaaaagttgagtcaatatctggctaccagagtacacgttaagataaataaagcgctctcaagatgttacttg

SEQ ID NO: 2 - DNA, 2625 bp, Artificial Sequence
  HRP isoenzyme C1C
ttttttttttctcttaaaaaatgcattccccttcttctacttcgtttacttgggcaaccttaatcacattgggatgtcttatgcttcatgcatctttttccaatgctcaacttacccctaccttctacgacaattcatgtcctaacgtctcaaacatagtacgggacatcattatcaatgagttacgatcggaccctcgtatcgccgcgagcatccttcgtcttcacttccacgactgctttgttaatgtaagatattacttttcatatttctattgcgttgtgaattattgtgttttatctttttttgaagatatgtgttttatcttttagatattgatatatcacacctctagtcaaaatttaataaaacataaaataaaatataatacaaagaggagtataattaataaaaaatttaacatgtttttaatctggctcaaaaatgtcgaaacgagatttcttatgaaaaagagtaagtatgtattaaatcataatttgctacaattattgggtggtaaaagtaaggaattctattcattttgaaatttagctggttgcatagctttattctacaacatttttactggattcattttataaagttataatttcaccttttttttttgttaaatagttataatttcacttgccttcggaaaatatgtatagccttactataacataattacattcaataataatttttattttatttatataaaaagattatattattttttgttttgttcagttagagtgaaccgtatcctaaataaaagtcactcgagtaacgggtctgatccagacaaaaaaaaaaaattcaataccaaatttcacatggttagatgtcgtatgtttaattttgctggtgaataattaatacttaactctcgtcgacaatgataatgccaaaacatttatgaaatcggattcatgaattgtccgtgttacagtattaagagcatggtgccgtaccccaaacacgatatgaatttaatggatgacaaaaaaaaaattcattgttaaattgttaccggtggtaaagatttagctatggatgtaatacatttactaataatttgttaattaatattttggatttttgatagggttgtgacgcatcgatcttgttagacaacacaacatcatttcgaacagagaaagatgcgtttggaaacgcaaactcggctcgaggatttcctgtggttgacagaatcaaggccgcggtggagagggcatgcccaagaactgtttcatgcgcagatgtgcttaccattgcagctcaacaatctgttaatttggtatgttccaataacttctgtcattacaaacattgtttttaatttttttttttttttgttaaaatcattgttttaaattttaatatatcaattcctcacttttacatatatcaaacgtacaacgatagcctaagtttgaaaagaaaagtattcaaaacgtcacaatctatatatttagcatcaacaacttttcaactatatatatgaggtatgtatggattaatctagaaatattcaaaacgcgtggtcgcgttatttgacaagtttaataatatcattgaaaaagtaggtatcatcacaagaatttaccttgtaataggcaaagcctacctttatagttaaccaaaaagaataattttcatttttttccaaaaattagtggagataaaacaaagctccactactttgagtatatattatcaatcaatttatactacttgtttatctttttctctttacatattgaaacttccgagtgacaaaattaatctcaaaaaaaataattattttgaatataatggttattatataggcaggaggtccttcttggagggttcctttgggaagaagagacagccgacaagcatttttagatctcgctaatacgaatcttccagctccatccttcacacttccacaacttaaggctgcttttgcaaatgttggcctcaaccgtccttctgatctcgttgctctctctggtaattaacaaaagaaaactaaacaccatttggtatagtttaatgagcgatttcattattaatcttattatggtctttcttttgttaggtggtcacacatttggtaaaaatcaatgtcgatttattatggacagattatacaacttcagcaacaccggactacccgaccctaccctcaacactacttaccttcaaactcttcgtcaacaatgtccccgtaatggtaaccaaagcgtcttggtggatttcgatctgcgtacgccaacagttttcgataacaaatactatgtgaatcttaaagagcaaaaaggtctcatccagagtgaccaagagttgttctctagccccaatgccactgacacaatccccttggtgagatcatatgctgatggcacacaaacattcttcaatgcctttgtggaggccatgaataggatgggaaacattacacctcttacaggaactcaaggagaaatcaggttgaactgtagggtggtgaactccaactctctactccatgatatagtggaggtcgttgactttgttagctctatgtgagaaaagttgactcaatatctggctaccagagtatacgttaagataaataaagcgctctcaaga

SEQ ID NO: 3 - DNA, 2631 bp, Artificial Sequence
  HRP isoenzyme C2
aaacctaaccaaagaattttatcttagagagcaaagaaaatgcattcctcttccagtttgataaaattgggatttcttcttcttcttcttaatgtatcattgtctcacgctcaactaagcccttcattttacgataaaacatgtccacaagtctttgacattgcaaccaataccattaaaactgcgctgagatcagaccctcgcatcgctgcgagcatccttcgtcttcatttccacgactgctttgttaatgtaagataccactagattcaatattttcacgtatatattaattaaggccaaatgactttttattaacgctagtaaaaaacctgtttaagtacttcaactgtaaactaccatcttaaatctagctaatctatatgggttacaatatttttgtacacaagattatattagatattatataaaattaaaattattcatatggtgtctaatacttgtcgccgttcaccaatatatccataaattattgtcctgtcgcagaaaaaaatacaaaagagttgaaatatactaaaaatataatcttactagagatgagtatttatcatagatttgttgtcgttttcggatatgattgattaagaaattttattatatagaatgtcttttggttccctgatatatcacaatcacaagcttgatattaggatatcaaaaagtgatagacattttgatttgtggttcgactcaaattgttttttttgcttaaaatcgactcaaattgttcgtttctcggtttattcatttcaaatagaatatactcaataaagtctcaaactgtataaaagtttgtgttagttttggttattgaaatgttgggtttcttttggttcagattaaaacaaaagaaaacaccggagttaaataaaaatagaaataacaaaataaaattattatatcaacaaaaaatatgtcttttagttaaattattttataagaacttgattcgacttattttgactaattttggttcaatttggaggggcagtatttataaatggaccaatcgcttcttaagcaaattaatatgatttaaatagtacaacgtagattgctttcttatcttaactctcaagtttcagattctgtcgtcaaagtatagtcaaagatgattagttaataattagcgaactcatggctgtttcaattaactcggcaaaaacaaaaacaagtattggacttttgatggatttttatttgggagatcaaatcagtttttttgtccaccatcgaaatttgaactctaaattttcaaaatctctctctttttttttttttttttgttaaacctctttataatttttttttctcacagcaaatcattatttactgattagatagctcaggaataggatgggaaacacaaattcttgcactgtttacggatgttaagatgttttgatttaactatgaatatggtagttgactaaatgtgaagaactatatttaattttgaaacagggatgtgatgcatcgatattgctagacaacactacatcatttaggactgagaaagatgcgtttgggaacgcaagatcagctcgaggcttcgacgtaatcgacacaatgaaggctgccgtggagaaagcatgtcctaaaaccgtttcatgtgctgatttgctcgccattgcagctcaaaaatctgtcgttttggtattcttcttttacaatgctatgcatctatacatctttatttttctttcctttttcatttaaatggttttcatatcatcttattgattttaatttttaggagaaacaagtatttaaaccatgcaaatataattgtttacttgaaaatgaaaataaaataggcgggaggtccttcatggaaggttccaagtggaagaagagacagcttaagagggttcatggatctcgctaatgataaccttccaggtccatcctctacacttcaagtacttaaggacaaattcagaaatgtcggactcgaccgtccttctgatctcgttgctctttctggtacattatagttaaaaacattttcattttcatatataacctatagttgtagtcattaataaactctaaattattatttgctttggtttattaattcttttgatatttttctttggttaggtggtcacacctttggcaaaaaccaatgtcagttcataatggatcggctttacaacttcagtaactccggtaaacccgacccaacccttgataaatcgtacctcagcacgctaagaaaacaatgcccacgtaatggaaacctgagtgtattggtagattttgatttacgtacaccgacaatctttgacaacaaatactatgtgaatctcaaagagaacaaaggtcttatccagagtgaccaagagttattctctagccctgatgcttctgacactatccctttggtccgagcatacgctgatggtcaaggaaagttttttgatgcattcgtggaggcaatgataaggatgggaaatctttcaccttcaactgggaaacaaggagaaattagattgaattgtagagtggtgaattctaaacctaaaatcatggatgtggttgatactaatgactttgccagctccatctgaagaaatgactttctcctaataataaatgatcaat

SEQ ID NO: 4 - DNA, 1422 bp, Artificial Sequence
  HRP isoenzyme A2A
atcatacctctaaaatcattattttgtaaaacctaattaatggctgtaacaaatctatctactacttgtgatggtttgtttatcatcagccttcttgttatcgtttcttcattgtttggaacatcatctgcgcagctaaatgcaacgttttactccgggacttgccctaacgcatctgccatcgtacgcagcactattcagcaagcttttcaatccgatacaagaatcggagccagcctcatccgccttcattttcacgactgctttgttaatgtatactaatcttcccaatgcagctctttacataaaggcttcttgatatttttcgctctaaaccgctactttgcttctttattttttcaaagggttgtgatgcgtcgatcttgcttgacgacagtggaagcatccagagcgagaagaacgctggtccgaatgcaaactcagctagaggattcaatgttgtcgataacatcaagactgcccttgaaaacacttgccctggtgttgtctcttgctctgacattttagcccttgcctcagaggcttccgtgtctttggtaattagtaattacactttctttgtgaacatatgaaacaaaacataactaaaaatttcaaatttgctcttaatttcttgttatatatatataatttgtcttataaattatgttttagtaataatatagatacgtatatgttcatatatatgtttgatcatcttcagacaggagggccatcatggactgtattattaggaagacgagatagtctcaccgcaaacctcgccggggcaaattcggctattccttctcccttcgaaggccttagcaatatcacatctaaattttcggctgtcgggctaaacacgaacgatctagtagccttatctggtaagttcatctacatgtttagttacttgcggttcaagttaattcaaaaccctgacgtcatctcttgtctacgtaggtgcgcatacgttcgggcgtgctcgatgtggagtgttcaacaacagactatttaacttcagcgggacaggaaatccagacccgactctaaactcaacgctactgagcagtcttcaacagctatgtcctcaaaacggcagcgcatcaaccatcaccaatctcgatctgagcacacctgatgcgttcgataacaattacttcgccaaccttcagagcaacaatgggcttcttcagtcagaccaagagctgttctctaccacgggttcagccacaatagcggttgttacctcctttgcaagtaaccagactctgttttttcaggcctttgctcagtccatgatcaacatggggaatattagtcccttgacagggagtaatggagagattagattagactgtaagaaggttaatggaagttgatttccataaagctcttgtttttcaaaaaacacataat

SEQ ID NO: 5 - DNA, 1847 bp, Artificial Sequence
  HRP isoenzyme E5
catagtctatcatcctcctaaaaattaaagagaaatggtggtttctcctttcttttcttgcagtgctatgggagccctaatattgggttgccttctgcttcaagcatctaatgctcagttgaggcctgacttctactctaggacttgcccatctgttttcaatattattaagaatgtcatcgtcgatgaactgcagactgatcctcgcattgccgctagtatccttcgccttcactttcatgactgctttgttcgtgtaagtacttaacttatgtttttattattaataaaaaaaaaccatgacgaattcatattggacgaaactactttttataccatttaatttacttataggttagaggccaaaaggcatatttgaatcaacaagtacaatcgtcatataacgtataatatctatggtttttgtagggttgtgatgcatcgatcctgcttgacacttccaaatcgttccgaaccgaaaaagatgctgctccaaacgtaaattcggctcgagggttcaatgtcatagatagaatgaaaacagcacttgagagagcttgtcctagaacagtgtcttgcgcagatattctcaccatcgcctctcaaatatcagtgcttttggtatgtacatatacctatatatgacttataatatcgggtgaaattaataaaaatatgttatgaaattttgacgtcaatgctttatatgttatagtcgggaggtccatcttgggcagttccgttggggaggagagacagcgtagaagctttctttgacctagctaatacagctcttccctctccatttttcactcttgctcaacttaaaaaagcttttgctgacgttggtttaaaccgcccctcagatctagtcgctctttctggtaaactatatatattcatgttgtttataatataaagtgttttatataaaatgatagctaaccacacccactccgcgatcgatgaggtctagaatttacacactaatttataagttatagaattacaaaattttattattttgtatttattaatatgatttgcttttggctttgaaatacatgaccaagtataaaatgaaaaacaaaatggataatataatcaaataataatactatttcagacaaaacatcatctagcacgtgattttgaattataagataagatggtaaaacgacaaaacatgactattatttttttaccttttttttttttaattgtcaaactgacacatacaatgtatctaaaccttattgtcaaaatggtaggtggtcacacatttggaagagcacgatgcctatttgtgacagctcgtctctacaacttcaacggtacaaacagaccagacccaactctgaacccatcttacctcgccgacctccgtcgattgtgccctcgaaacggaaacggcaccgttctggtcaacttcgatgtcatgactccgaatactttcgataatcaattctacactaatcttagaaatgggaaaggtctgattcagagtgaccaagagctcttctcgactccaggagccgacacgatcccactagtaaacctatacagcagcaacacgttatcgttcttcggagcattcgctgatgcaatgattaggatgggaaatcttagacctttgactggaactcaaggcgagataagacagaattgtagggttgtgaattcgcgaattaggggtatggagaacgatgatggagttgtgagttctatgtgattatgttgggaatatatatcatatatggttatgtatcaaatcataaaatgtgtgggaacatgcatgtcgactaaataaaagttctaacgagttgtgaaatgactatgaaattttg

SEQ ID NO: 6 - DNA, 2901 bp, Artificial Sequence
  HRP isoenzyme 01805
cttaaaccaataaaagataagtttcctcttaccaaaaatgcatttctctacttcttcttcttccttgtctacttggacaaccctaataacattggggtgtcttatgcttcattcatttaagtccagtgctcaactaacccctaccttttacgacagtacctgccccagcgtctttagcatcgtacgggacaccatcgtgaacgagctaagatcagatcctcgaattgctgcaagtatccttcgtcttcacttccacgactgcttcgttaatgtaagatattatttttcagtttaattgatgattttgtggatacgctagtatcttttataattgaataaaagaattattttatctaattttcacacatataattgatatatttggtactaatataagttcttcaaaaacaatttcaacgatttgtgttgtcaacttagttgcaattcggatcgacttgtagttaacgtatttgttttctgaaataagttttttgggtggggataatggcgccactcaagaaaaaccagttatttctttgtaagttttaattgttgagaaagacaacaaaaaaaaacgcgtggatacggcacttacatttattaaactaaaggtttgtcagaattaattaattatagtaatagttgtttctgtggtattagtttttttgtttgttaaagcgtttctgtggtattagttaccatgacatgttatcatcaatacaaaagttcacgtagttagatgtaatatttttggtttttactgtcgagtatttttcttcttgtcgtcgactcatgacataaaataatgattccaaaacatctattaaactttgacctcccattcggcgaacatgaattattcgtacaataatttaataaaagcatggacccgtacaaagcacgttatgaaaatttaaatcagtccctttttttagcataaaaataattttttttgtgaaatcattgaaaagtctcaagcttatcctcaaccccaaataacctacagatatgaaaaagaaacagaaatgtacgtcaaaacctactatataatttaatttattatttttgggattttcaactctcgaatctgatgtagttgttagcatactttatttgaacttttaatgtttttattcgaaaacatagtatatacccagacccagatattatagccaaataatgtagaccaacaactttttgaattttgtgtgaaaagattttagatttatatgtaaactaatctgtttataattatgtatatattatcatgccttgttgaagaggaacaaatggtttcaaggaatgaaaatgcaatctaacaatgtttataggtagtaattaaagatttgattatgatttcaacttatttctttgcaaaagggctgtgatgcatcgattttgttagacaacacaacatcatttcgaacagagaaagatgcagctccaaacgcaaactcagctcgaggatttccagtgattgatacaatgaaagctgcagtggaaagagcatgtccaagaaccgtatcatgcgcagatttgcttaccatcgcagctcaacaatctgtgaatttggtatgcaacattaatttatctcttttttttagttttaattttatctattttttctccacttcttgcaaacatggtctcaaattccttgttgaagccgtaactgaacccgttaaacatgactacatagcattgaaaaatgagatggactgttgcatgttttataatttagatttaatattaccattcttaaatattaattaccatacttgtcttgaaaattcattgtaagaaataatcaaaaacacatggtaataatattgtcttgtttttgtaaaagggttgggttttctaataatataaagtctcattacaataataaacttgattagataaaaactcttttctgaattaactaaagtgaatataagcaatatttgtaaaagaactgggaaaataacagtacaattgagaacctaattttgggatttatattttatatatcctttttaataattatgactttgacgtggtaagaattattttcatctataaaatcattattttaagatatgggtattttaggcaggaggtccttcttggagggttcctttggggagaagagacagcgtacaagcattttttgatcttgccaatacaaatcttcccgctccattcttcacgcttccacaacttaaggccagctttagtaatgttggacttgaccgtccagaagatctcgttgcactctctggtaattatgaggagtaatagtaacaaccaaaacttttatttgatctaattagtttataaaatattattaatttatcatcttttgattaggtggtcacacatttggtaaaaaccaatgccaatttattatggacagactatacaactttagtaacactggtttacccgaccctactctcaacactacttatctccagacacttcgtgtacaatgtccccgtaatggtaaccagtccgtcttggtcgatttcgatctacgcacaccgacagtttttgacaacaaatactatgtgaatctgaaagagcacaagggacttatccagaccgatcaagagttgttctccagccctaatgccgctgatacaatccccttggtaagatcatatgctgatggcactcagaagttcttcaatgcttttatggaggccatgaacagaatgggaaacattacccctctcactggaactcaaggacagatcaggcaaaattgtagggtgatcaactccaactcgctgctccatgatattgttgaaatcgttgactttgtgagctctatgtaacaatagttgtctcaatatatgtggcaaccaaaattatatgttcttatgaaaataaaatgttctcgaaacattacttaag

SEQ ID NO: 7 - DNA, 1819 bp, Artificial Sequence
  HRP isoenzyme 22684
aaaatggggttttctccttcattttcttccagttctataggagtcctaatattgggttgccttctgcttcaagcttcaaactctaatgctaagttgaggcctgacttctacttaaagacatgtccatcagttttccaaatcattgggaatgtcatcgtcgatgaactgcagagtgatcctcgtattgcagctagtctccttcgccttcacttccatgactgttttgttcgtgtaaggacttaattactcaacttatctttttatccgaaaaagaaaaaaacatgacgtgttcaatggacaaaatgacttttcaatacggaagtagaggctaaaagcaatttttaattaataaatacaatcttcatgtattataatatggttttgtagggttgtgatgcatcggtcctgctcgacaattccacatcatttcagtccgagaaagatgctgctccaaacgcaaattcggctcgagggttcgacgtcgtagatagaatgaaagcagcccttgagaaagcttgtcctggaacagtgtcttgtgcagatgttcttgccatctccgctcaaatatcagtgcttttggtatgtacatatacctatatatgacttatatcgggtgaaactaatcaaaatatattatgaaattttgacgttatgttatatattatatagtcgggaggcccatggtggccggttttgttggggaggagagacggcgtagaagctttcttcgatttggctaatacagctcttcccaatccatttgcccctcttactgaacttaaagaaaaatttgctgacgttggcctaaagcgcgcctcagatctagttgctctttccggtaaaattttcatatttttcaatctttcttgttttggtcaaccatattgtttcaatctatataacctactctatgtattattgttttttttataatctaactagatataattcattttatctcgataactaggtagatatcgagttagtctggaattaacttagtaaagttatcttctaggtagatatcgagttagtctggaattaagatcaaatttgcaatgtcaacacaagaaaagtgacttgaaattaaagatgagttggtcaaacgacatgacatgagtcatctttaataaagttaaacatatatacaatgtatctaaactttacttttttttgtggggtcaaaatggaaggtgctcacacatttggaagagcacaatgtctacttgtgacacctcgtctctacaacttcagcggcaccaataaaccagacccaactctgaacccatcttacctcgtcgaactccgtcgattgtgccctcaaaacggaaacggcaccgttctgctcaacttcgatctcgtgactccaaatgctttcgatcgtcaatactacaccaatcttcgaaatgggaaaggtctgattcagagtgaccaagagctcttctcgactccaggagccgacacgatcccactagtaaacctatacagcaagaacacgttcgcgttcttcggtgcattcgttgacgcaataattaggatgggaaatattcaacctttgactggaactcaaggcgagataagacagaattgtagggttgtgaattcgcgaattaggggtatggagaacgatgatggagttgtgagttctatttgattatgttgggaatatggttatgtaacaaatcataaaatgtgtgggaacatgcatgtcgactaaataaaagctctcacgagttatgacttgtgagattacaactgaaaaaaccaaaggaaagaaaagatcagattttggatcaccagcccgggccgtcgaccacgc

SEQ ID NO: 8 - DNA, 1798 bp, Artificial Sequence
  HRP isoenzyme 01350
caactccaactccaagtctattcaaagtctttgtttaacctaaacatggcttcaaatcaacgtatttccattctagttctcgtagttacatttttagtgcaaggtaattacaataacgtcgttgaagcacaactgacgcccaatttctactcaacctcttgccctaacctcctctccaccgtccaatccgccgttaagtctgccgttaacagcgaggctcgaatgggtgcatctatcgtacgccttttcttccacgattgcttcgtcaacgttagtttttttttaacttttttttttgtttttagttttccttgcattccaagaaacttagcgtaatgttttttttttttaattttcttttctaatcatagtcataacttgcaaatatatataaaaatatagggatgcgatggttcgattttactagatgacacatcaagcttcacgggagaacaaaatgcgaacccaaaccgcaattccgctcgcgggtttaatgtgatcgacaacatcaaagcagcggtcgagaaagcatgtcccggggtcgtgtcttgtgctgatatcttagccatcgcagctagagactccgtcgtagtcgtaagctccctatgtcctcctctcttagtccggttttgcaaatttaaaagattaattaaacgggtctaaaaaatcgtctttgttctcgttgaaagcttggagggcctaactggactgtgaaagtaggaagaagagatgcgagaacggcgagtcaagcggcggcgaatagcaacattccggcgcccacttctagtctgagccaactcattagtagtttcagtgccgttggactctccaccagagatatggttgctctctccggtccgtttcatctctcttcttaactcaaatttttttttttatcatatatgcaaatatttgtttcatagattaaggctgattgcaattgttaacggttactaatatatacattactaaaattggtgacaataaatttcttttgttaatgtgtaattgctttcactaatttgatttacaaaaaagtaaaaaacaaaatcacaaaaataaatattcaaaggactaagagtcaacagaatcgacataaaaataaactaatatgattgtgtcgtttgatttttgtcgactctccccctttaagttcttgtttctttgtgattcttttttcaacacattttttgtaacgcaccaccgacttttctgtttcttaaccaaagttacgtaatcatatagattactgttacgcgacaccgacaaaacaattgactgattaatcaacaaataattaagtccactattaaactaattgctatatgttcatttttttcaggcgcgcacacgatcgggcaatcccgttgcacgagcttccgaacgagaatctataacgagacaaacatcaacgccgcattcgccacaacacgtcaacgaacttgccctagaacctccggctccggcgacgggaatttagctccacttgacgtcaccacggcggcttctttcgacaacaactatttcaagaatctcatgactcaaagaggtcttctccattccgaccaagagctcttcaacggcggctccactgactccatagtccgtggatacagcaacaatccgtcaagctttagctccgattttgcggcggcgatgattaaaatgggtgatattagccccttgaccggtagtagcggtgagatccggaaggtttgcgggaggaccaactgatatttctttttccctattggaatttgacttttgttagttgattcggtgagaataagatttgatc

SEQ ID NO: 9 - DNA, 1485 bp, Artificial Sequence
  HRP isoenzyme 02021
aagagtattgagaaacaagatcgaggaaactaatcaatgaggacgatgaagcgattgaacgtggcggtggcggttgcggttacagcgacggttcttatgggaatgttaggatcatcagaggctcagcttcaaatgaatttctacgcgaagagctgtccaaacgcagagaaaataatttcagatcatattcaaaagcatatccctagtggtccttctcttgcagctcctctcatcagaatgcacttccatgattgcttcgtcagggtatttaatctctaatctatctacatatatagttgcaagtgtttagatatattcgacttttatgtaacatatgtaggaaattagtattcacacatccagttcaataaatgatgggatagtccagaagatgtactaatgtatattttaaaaaaatggtaatttgatagtgaacatgaggtagatctagaatatttgatatttattgcattttttaaatattgacaatgtttttgaaaaaaaaaacataataatctagggatgtgatggatcggtgttgataaattcgacatcagggaacgcagagaaagattcagcaccaaatctaacacttagaggcttcggtttcgtagagaggattaagactcttcttgaagcagagtgtcctaagactgtttcttgcgccgacatcatcgcactgaccgctagagacgcagttgttgccaccgtaagtaaacaaattataacttcaagactcaaaacattatttaatctaattaatcgaaattataatctaattttttttaatagggaggtccttcatggaaagttccgacaggaagaagagacggtaggatctcaaatacgacggaggctttgaacaacattccaccgccgacgagtaatttcacgacgttacagcgacttttcgctaatcaaggccttaatctcaaagaccttgttctgctttccggtaagtttagtaaccggaaataaccagattgaatttaacaacctaacggtgtttaactttttgttgttgttgttgttgtttaggagctcacacgatcggtgtctcgcattgttcttccatgaatactcgtctctacaacttctcgacgacagtcaaacaagatccatctctggatagcgagtacgcagcaaatctaaaggctaacaaatgtaagagtcttaacgataacaccaccatcctcgagatggatcctggtagtagcaaaacctttgatctcagttattataggcttgtcttgaagaggagaggtttgtttcagtctgattctgccttaacgacaaactcagctacgttgaagatgatcaacgacttggtcaacggtcctgaaaagaagtttttaaaggctttcgctaagtcaatggagaagatggggagagttaaagtgaagacgggctcagccggtgtgattaggacacgttgttctgttgccggaagttagttagtttggtcggaaagtgatgttttctgtt

SEQ ID NO: 10 - DNA, 1085 bp, Artificial Sequence
  HRP isoenzyme 04791
tctcactttctctcttccgctacaacaatggcggaactcaaatctctctccctcatcctcctcttcacactcctcaccaccaccatcgaatctcgtttaaccacaaacttctactcaaaatcatgtccaagattcttcgacatagtcagagatacaatctcaaacaaacaaatcacaacaccaaccacggcagccgccacaatccgtctcttcttccacgactgtttccccaacggctgcgacgcctcaatcctaatctcctcaactgccttcaacaccgcagaacgtgactcatcaatcaatctctcacttcccggcgacggctttgacgtcatagtccgagctaaaaccgcaatcgaactcgcttgtcccaacactgtttcttgctccgatataatcaccgtcgctactcgtgaccttcttgtcaccgtcggtggtccttactacgacgtttacctcggccgtcgtgattcaagaatatctaaatcatctcttttaaccgatcttcttcctcttccttcatctccgatctcaaaaaccattcgtcagtttgaatctaaaggtttcactattcaagaaatggttgctcttagtggggcccactcaatcgggttttcacattgtaaagagtttgttaatcgggtcgccggtaataataccgggtataacccgagatttgctcaggcgttgaagcaagcttgttctaattacccgaaagatccgacgttatctgtgtttaatgatattatgactccgaataggtttgataatatgtattatcagaatattccaaagggtcttgggttacttgaatcggatcatgggttatattctgacccgagaacccgaccttttgttgatctttatgctagagatcaagatttgttctttaaagattttgctagagctatgcagaagttgagtctctttggtgttaagactggtcgacgaggagagatccgacgaaggtgcgatgcgattaactgagtttatgttttttcttttatgtatttattttttggattatatttggggaagaaatgtgagatttgatgaattgtgaatcttctgagtttttttttttt

SEQ ID NO: 11 - DNA, 1373 bp, Artificial Sequence
  HRP isoenzyme 06117
cttcttctttcttctggatctagtggaaactgacattatggcaagaattggaagctttctcgttgttatctctctcgcttgcgttcttactctctgcatctgcgacgacgagagtaattatggcggccaagggaaactcttcccaggtttctacagcagctcgtgccctaaagctgaggagatcgtgaggtctgttgtagccaaagctgttgcaagagagactcgtatggctgcttctctcatgaggctccattttcacgactgttttgttcaggtactcaagagctattttaagtaactttctcatcaagctaacgactaattttgagaattttaattaattatagctgtttgcataaaaatgtgtagggttgtgatggatcgttgcttctagacagcagtggaagtatagttactgagaagaactctaaccctaacagcagatcagctcgtgggtttgaagttgttgacgagatcaaagctgcattggagaatgaatgccctaacactgtttcttgcgctgacgccctaactctagccgctagagactcctctgttcttgtaagtcgccttattccactttctctctttaccgcttccttaagttctcaagagatctaattttgttcggtttgaatatagactggtggaccaagctggatggttcctttgggaagaagagattcgacaagtgcaagcttgagtggatcaaataacaacattcctgcaccaaacaacactttcaacacaattctctcgagatttaacagccaaggtctcgatctcaccaatgtcgttgctctctccggtaagcttacttaaaacacaaggaaaattttactttccttgctactcaagttaaagttaaatcctaaaaaggattttagtataacctgactcaattgtttctcagggagccacacaattggattctcaagatgtactagttttagacagagactttacaaccaatccggaaacggaagtcccgacacaaccttagagcaatcctacgctgctaacttgcgccatcggtgccctagatcaggtggggaccagaacctgtcggagcttgacatcaacagtgctggaaggtttgataacagctactttaagaatttgatcgagaacatgggactgttgaattccgaccaggtcttgttctctagcaacgacgaatcgagagagctagtgaagaagtatgcagaggatcaagaagagttcttcgagcagttcgcggaatcgatggtcaagatggggaatatctctcccttgactggttcaagtggtcaaatcaggaagaattgcaggaagattaactcttgatttcataatattgaattgggcgaaataaaaatgagagatgttttggg

SEQ ID NO: 12 - DNA, 1405 bp, Artificial Sequence
  HRP isoenzyme 17517
caacaacaacttttacaaagctcaaagagtttcttatttaccaaaacaaaaacaaaatgggtcgtggttataatttactattaattctagttacgtttttagtattggtcgcagctgtaaccgctcgaagaccacgagttgggttttatgggaatagatgccgaaaggtagagtctatcgtgagatcggtggttcgatctcatttccggtgtaatccggcaaatgcaccgggaattttgcgtatgtattttcacgattgttttgtcaatggctgcgatggctcgatcctcctcgctggtaacacttcggagagaactgcgggtcctaaccgttcattgagagggttcgaagctattgaagaagctaagactcggcttgagaatgcttgtcctaataccgtttcttgtgcggatatcctcacccttgctgctcgagacgccgtcgtttgggtaaaacatattgaaattagtttcagttttaagaaattttaattatatattgtgtgtttaatggtaatataattttgggtgatgcagaccggtggaaaaggttggtcggtgccattgggacgtcttgacggccgaagatcagaagcctcagatgtaaatttgcccggaccaagcgaccccgtcgctaagcagaagcaagactttgcagctaaaaatctcaacaccttggacctcgtaactcttgttggttcgttgtaattatatataatcaaacaatgtatattaatcaaatagttacatatttggtcggttgtattaaattttcgatacagttctgattacgtcggtatgtatgaggttatgaaatattttttttcttgatttaagtttagaaatttagtttcaatttgaaccgaattcggtttacatgttcaattttattagctaatattgcaaattgatattgatcaggtggacacacaattggaactgctggttgcggtttggtaagaggcagattctttaacttcaatggcacgggacaacctgacccatcaatcgacccgagtttcgttcctctagttcaggctcgttgccctcaaaacggtaacgcaacgacccgagtcgacttagacactggaagtgcaggtgatttcgatacatcgtacctaagtaacgtgaggtcaagccgcgtggttctccaatccgatctagtcctgtggaaggacaccgaaaccagagccatcatagaacgtttattaggcttacgccggcccgttttgaggttcggatcagaatttgggaagtcgatgaccaagatgagtctcatagaagttaaaactagactatcagatggggagattcgtagggtttgctctgcgatcaattaagtattaaaaacacacaaatgtttggtttgattttatcacttattttatggaataagcttgctatag

SEQ ID NO: 13 - DNA, 1258 bp, Artificial Sequence
  HRP isoenzyme 08562.1
gtaatggcaagactcactagcattctccttcttctttctcttttatgctttttccctctctgtctctgcgacaagagctatggaggcaaactcttccctggtttttacgcccactcatgcccacaagccggggaaatcgtgagatcagtcgtagctaaagctgttgctagagagacccgtatggctgcttctttgatgagacttcatttccacgactgtttcgttcaggtttggttaatttcttctacgcccactattctaaagatttttttattgagcaaggtaactgtgaaatgcagggttgtgatggctctttgcttctagacagcagtgggaaaatagtgagtgagaaaggctcaaaccctaacagcagatcagctcgtgggttcgacgtagttgaccaaatcaaagctgaattggagaaacaatgccctggaactgtttcttgcgctgatgctctaactctagccgctagagactcctctgttcttgtaagtcccctccatagtttccaaatcaaatttaaaacatcagctaactcggtgtggtttttgttttagaccggtggaccgagctgggtggtttcattaggaagaagagattcaagaagtgcaagcttgagtggttcgaacaacaatatccctgcaccaaacaacactttccagaccattctatcgaagtttaaccgtcaaggactcgatgtcaccgaccttgttgctctctccggtaagctttcttcacttgcacgcaacacagttaaaagaaaccccattgccttacttttttctcaacccaccacacttcttaactgtttctcagggagccacaccattggattctcgagatgcacgagtttcagacaaagattgtacaaccagtccggaaacggacgtccagacatgacattggaacaatccttcgctgctaacttgcgccaaaggtgtccgagatccggcggagaccagattctctcagtgttggacatcatcagcgccgcgaaattcgacaacagctacttcaagaacttgatagaaaacaagggtttgttgaactcggaccaggttttgttcagcagtaatgagaaatctagagagcttgtgaagaagtatgcagaggaccaaggagagttttttgagcagtttgcggaatcgatgatcaagatgggaaatatatctcccttgacgggttcgagtggcgaaatcagaaagaattgcaggaagataaactcttgaattcttgaaatgaggaaagtattggg

SEQ ID NO: 14 - DNA, 1258 bp, Artificial Sequence
  HRP isoenzyme 08562.4
gtaatggcaagactcactagcattctccttcttctttctcttctatgctttttccctctctgtctctgcgacaagagctatggaggcaaactcttccctggtttttacgcccactcatgcccacaagccggggaaatcgtgagatcagtcgtagctaaagctgttgctagagagacccgtatggctgcttctttgatgagacttcatttccacgactgtttcgttcaggtttggttaatttcttctacgcccactattctacagatttttttattgagcaaggtaactgtgaaatgcagggttgtgatggctctttgcttctagacagcagtgggagaatagtgagtgagaaaggctcaaaccctaacagcagatcagctcgtgggttcgacgtagttgaccaaatcaaagctgaattggagaaacaatgccctggaactgtttcttgcgctgatgctctaactctagccgctagagactcctctgttcttgtaagtcccctccatagtttccaaatcaaatttaaaacatcagctaactcggtgtggtttttgttttagaccggtggaccgagctgggtggtttcattaggaagaagagattcaagaagtgcaagcttgagtggttcgaacaacaatatccctgcaccaaacaacactttccagaccattctatcgaagtttaaccgtcaaggactcgatgtcaccgaccttgttgctctctccggtaagctttcttcacttgcacgcaacacagttaaaagaaaccccattgcttaacttctttctcaaaccgctacacttcttcactgtttcccagggagccacaccattggattctccagatgcacgagtttcagacaaaggttgtacaaccagtccggaaacggacgtccagacatgacactggaacaatccttcgctgctaacttgcgccaaaggtgtccgagatccggcggggaccagattctctcggtgctggacatcatcagcgccgcgaaattcgacaacagctacttcaagaacttgatagagaacaagggtttgttgaactcggaccaggttctgttcaacagtaacgagaaatctagagagcttgtgaagaagtatgcagaggaccaaggagagttttttgaacagtttgcggaatcaatgatcaagatgggaaatatatctcccttgacgggttcgagtggcgaaatcagaaagaattgcaggaagataaactcttgaattcttgaaatgaggaaagtattggg

SEQ ID NO: 15 - DNA, 1375 bp, Artificial Sequence
  HRP isoenzyme contig23190 length=1375 numreads=249
ccctatagtgagtcgtattacggccgggggaacaacaagaagcagagaagagagaggcttcgactgaaacaacaaaaaatggcaatgagttattcgatacgtgtcctgacgtttctgatgttgatctcgttaatggcagtgacactgaaccttctgtcaacggcggaagcaaagaagccgaggagagatgttcctatagtgaaaggtctctcttggaacttttaccagagagcatgtccgaaagtggaaaagattatcaaaaaagaactcaaaaaagtcttcaagagagatattggtttagccgcagccatccttcgtatacatttccatgactgcttcgttcaggggtgtgaagcatctgtgctgctagctggatcagcaagtggaccaggagaacaatcatcgatcccgaacctaacactccgtcaacaagcctttgttgtcatcaataacctgcgtgccctcgtccagaaacagtgtggtcaagtcgtctcttgctccgacatcctcgctctcgccgctcgcgattccatcgtcctttcaggagggccagactatgctgtgccacttggccgacgtgactcgctagcgtttgcgaccccggaaacgacgttagctaacttaccgccaccgtttgccaacgcaagccagctcatcagcgacttcaacgacagaaacctcaacatcaccgacttagtagcactttccggtggtcacaccatcggaattgcgcattgtccgtctttcacagaccggctctacccaaaccaagatccaaccatgaacaagtctttcgccaacagcctcaaacgcacctgtcccacggcgaactcgagcaacacgcaagtgaatgacataaggagtcctgacgtgtttgacaacaagtactatgttgatctcatgaaccgacaagggctgttcacttccgaccaggatctgttcgttgacaagaggacacgtggcatagtggaaagctttgcgatcgaccagaacttgttttttgatcatttcacggtggcaatgattaagatgggtcagatgagtgtcttgacggggacacaaggggagatccgttccaactgttcagccagaaacaccgcaagtttcatatccgttttggtagaaggcatagtcgaggaagctctttccatgatctaaaataaccataaatctcagacttttcttttctttaactttgtttttatttagttgtcgcacttgtggtttgtggaatgcctaagactatctcataaataagagcattgctttcatcttaatttcttcttcttttttttttctctagtttggtcaatgtctgggactataatgaataaagaatgttgctacatcttaaaaaaaaaaaaaaaaaaaaacg

SEQ ID NO: 16 - DNA, 1283 bp, Artificial Sequence
  HRP isoenzyme contig04663 length=1283 numreads=14
aactaattagattaaagtatcataagttctgaatcaaaatccgtcaaaggaaagattaatcaaagctctatcattatttgcaacaaactgattaatggctgcaacaagctcttctactacttgtgatggtctcttcatcattagccttcttgttatcgcttcttcattgtttgggacatcatctgcgcagttaaacgctacgttttactccgggacgtgccctaatgcctctgccatcgtacgcagcactatccagcaagctcttcaatccgacccgaggatcggagccagcctcatccgccttcattttcacgactgttttgttaatggctgcgacgggtcgctcttgcttgacgacactggaagtatccagagcgagaagaacgctcctgccaacgcaaactcagctagaggatttaatgttgtcgacgatatcaaaactgccctcgagaatgcttgtcccggcattgtctcttgctctgacattctagctcttgcctcagaggcttccgtgtctttggcaggaggtccttcatggactgtgttagtaggaagaagagatggtctcaccgcaaacctgtccggggccaattcgtcgcttccctctcccttcgaaggccttaacaacatcacatctaaatttttagctgtcgggctaaatacaaccgatgtagtagtcttgtctggagctcatacgtttgggcgtggccaatgtgtaaccttcaacaatagacttttcaacttcaacggaacaggaagtcccgacccgactctgaactcaacacttctcagcagtcttcaacagatatgtcctcaaaacggcagcggatcagcgatcaccaatctcgatctgactacacctgatgcatttgatagcaactactacacgaaccttcagagtaacaatgggcttcttcagtcagaccaagaactattctccaacaccggttcacccaccatcgcgattgtaatcctttgcaagtaaccaaaccctgttttttgaggcttttgctcagtctatgatcaagatgggtaacattagtcccctgactgggactagtggagagattagacaagattgtaaggcggttaatggacagtcatcagccactaaagcagaggacattcagatgcaatctgacggaccagtgagtttagcagatatgtgaacaataatgggatcagtttcaccgtttgtttatgatacatgaataattactcctaagctgaatcatttgtgtaaaagaataagtgttgttcgggaaaaaaaaaaaaaaaaaaaa

SEQ ID NO: 17 - DNA, 1180 bp, Artificial Sequence
  HRP isoenzyme contig06351 length=1180 numreads=22
aattaatctgattacaagatttaagatagaaaataataagatggttagggcaaatttagtgagcgtgattctgttaatgcatgttattgttgggtttccttttcatgcgaggggcttaagtatgacttattacatgatgagctgtcctatggctgaacaaattgtgaaaaacagtgttaacaatgctcttcaagccgatcccactttagccgcaggtcttatacgtatgttgttccacgactgtttcattgagggatgtgatgcgtcgattctgctagattcaacaaaagacaacactgcggaaaaggattctcctgcgaatctgagtctacgtggctacgagatcatagatgatgcaaaagagaaagttgagaatatgtgtccaggagttgtatcttgcgcagatattgttgccatggctgctagagatgctgtcttttgggctggtggtccatattatgacataccaaaaggaagatttgatggtaaaagatcgaagatagaagatacaagaaaccttccttcaccttttctcaatgcctctcaactcattcaaacctttggcaaccgtggcttctctccgcaagatgttgttgctctctctggagcacatacccttggagttgcacgatgctcctccttcaaggctagacttaccactccagattcttcactggactccacttttgcaaacactctcactagaacttgcaatgcgggggacaatgcagagcaaccctttgatgcgacccgcaacgatttcgacaatgcctacttcaatgcgcttcagaggaaatcaggagtcctcttttcagaccagaccttattcaacactccaaggaccaggaatcttgttaatggttatgcccttaatcaagctaagtttttctttgatttccaacaggccatgcgcaaaatgagcaatcttgatgttaaacttggctctcaaggtgaaatacgtcaaaattgccggactattaactaagcctaggccgatttttgtatttatgctcccacctttaattatacttacctactctgtcattaattcgagtcataatgtcctatgctaccatgtaaaattagtgtgcctaatgtgatatgcagctgtattgtacttattgtttgtgggtttcagatgtccatcatcaaaacgtaatatatatacttggtgatcttg

SEQ ID NO: 18 - DNA, 1212 bp, Artificial Sequence
  HRP isoenzyme contig06117 length=1212 numreads=26
cggccgggggaccatcacaaagtcacctcccttcttctttcttctggatctagtggaaactgacattatggcaagaattggaagctttctcgttgtcatctctctcgcttgcgttcttactctctgcatctgcgacgacgagagtaattatggcggccaagggaaactcttcccaggtttctacagcagctcgtgccctaaagctgaggagatcgtgaggtctgttgtagccaaagctgttgcaagagagactcgtatggctgcttctctcatgaggctccattttcacgactgttttgttcagggttgtgatggatcgttgcttctagacagcagtggaagtatagttactgagaagaactctaaccctaacagcagatcagctcgtgggtttgaagttgttgacgagatcaaagctgcattggagaatgaatgccctaacactgtttcttgcgctgacgccctaactctagccgctagagactcctctgttcttactggtggaccaagctggatggttcctttgggaagaagagattcgacaagtgcaagcttgagtggatcaaataacaacattcctgcaccaaacaacactttcaacacaattctctcgagatttaacagccaaggtctcgatctcaccaatgtcgttgctctctccgggagccacacaattggattctcaagatgtactagttttagacagagactttacaaccaatccggaaacggaagtcccgacacaaccttagagcaatcctacgctgctaacttgcgccatcggtgccctagatcaggtggggaccagaacctgtcggagcttgacatcaacagtgctggaaggtttgataacagctactttaagaatttgattgagaacatgggactgttgaattccgaccaggtcttgttctctagcaacgacgaatcgagagagctagtgaagaagtatgcagaggatcaagaagagttcttcgagcagttcgcggaatcgatggtcaagatggggaatatctctcccttgactggttcaagtggtcaaatcaggaagaattgcaggaagattaactcttgatttcataatattgaattgggcgaaataaaaatgagagatgttttgggaagaaatttatgtttgtgttctttgttttgtatgagtcatgagtgtatttccttgtgtcaaaaatttgaattttatgagttttttttcag

SEQ ID NO: 19 - DNA, 1066 bp, Artificial Sequence
  HRP isoenzyme contig03523 length=1066 numreads=16
gacccaacaaatctcactttctctcttccgctacaacaatggcggaactcaaatctctctccctcatcctcctcttcacactcctcaccaccaccatcgaatctcgtttaaccacaaacttctactcaaaatcatgtccaagattcttcgacatagtcagagatacaatctcaaacaaacaaatcacaacaccaaccacggcagccgccacaatccgtctcttcttccacgactgtttccccaacggctgcgacgcctcaatcctaatctcctcaactgccttcaacaccgcagaacgtgactcatcaatcaatctctcacttcccggcgacggctttgacgtcatagtccgagctaaaaccgcaatcgaactcgcttgtcccaacactgtttcttgctccgatataatcaccgtcgctactcgtgaccttcttgtcaccgtcggtggtccttactacgacgtttacctcggccgtcgtgattcaagaatatctaaatcatctcttttaaccgatcttcttcctcttccttcatctccgatctcaaaaaccattcgtcagtttgaatctaaaggtttcactattcaagaaatggttgctcttagtggggcccactcaatcgggttttcacattgtaaagagtttgttaatcgggtcgccggtaataataccgggtataacccgagatttgctcaggcgttgaagcaagcttgttctaattacccgaaagatccgacgttatctgtgtttaatgatattatgactccgaataggtttgataatatgtattatcagaatattccaaagggtcttgggttacttgaatcggatcatgggttatattctgacccgagaacccgaccttttgttgatctttatgctagagatcaagatttgttctttaaagattttgctagagctatgcagaagttgagtctctttggtgttaagactggtcgacgaggagagatccgacgaaggtgcgatgcgattaactgagtttatgttttttcttttatgtatttattttttggtattatattggggaagaaatgtgagatttgatg

SEQ ID NO: 20 - DNA, 1215 bp, Artificial Sequence
  HRP isoenzyme contig17517 length=1215 numreads=64
gagcgtattacggccggggacacaacaaacaacaacaacttttacaaagctcaaagagtttcttatttaccaaaacaaaaacaaaatgggtcgtggttataatttactattaattctagttacgtttttagtattggtcgcagctgtaaccgctcgaagaccacgagttgggttttatgggaatagatgccgaaaggtagagtctatcgtgagatcggtggttcgatctcatttccggtgtaatccggcaaatgcaccgggaattttgcgtatgcattttcacgattgttttgtcaatggctgcgatggctcgatcctcctcgctggtaacacttcggagagaactgcgggtcctaaccgttcattgagagggttcgaagctattgaagaagctaagactcggcttgagaatgcttgtcctaataccgtttcttgtgcggatatcctcacccttgctgctcgagacgccgtcgtttggaccggtggaaaaggttggtcggtgccattgggacgtcttgacggccgaagatcagaagcctcagatgtaaatttgcccggaccaagcgaccccgtcgctaagcagaagcaagactttgcagctaaaaatctcaacaccttggacctcgtaactcttgttggtggacacacaattggaactgctggttgcggtttggtaagaggcagattctttaacttcaatggcacgggacaacctgacccatcaatcgacccgagtttcgttcctctagttcaggctcgttgccctcaaaacggtaacgcaacgacccgagtcgacttagacactggaagtgcaggtgatttcgatacatcgtacctaagtaacgtgaggtcaagccgcgtggttctccaatccgatctagtcctgtggaaggacaccgaaaccagagccatcatagaacgtttattaggtttacgccggcccgttttgaggttcggatcagaatttgggaagtcgatgaccaagatgagtctcatagaagttaagactagactatcagatggggagattcgtagggtttgctctgcgatcaattaagtattaaaaacacacaaatgtttggtttgattttatcacttattttatggaataagcttgctatagcattttgtggtcgatggttacatgttgaatttcaagtgtaagattgtacttgtaacgaaaatgtatttataaaaatgtcgtttgtttcttta

SEQ ID NO: 21 - DNA, 1308 bp, Artificial Sequence
  HRP isoenzyme contig01350 length=1308 numreads=116
cggccgggggaagtacaataatcaactccaactccaagtctattcaaagtctttgtttaacctaaacatggcttcaaatcaacgtatttccattctagttctcgtagttacatttttagtgcaaggtaattacaataacgtcgttgaagcacaactgacgcccaatttctactcaacctcttgccctaacctcctctccaccgtccaatccgccgttaagtctgccgttaacagcgaggctcgaatgggtgcatctatcgtacgccttttcttccacgattgcttcgtcaacggatgcgatggttcgattttactagatgacacatcaagcttcacgggagaacaaaatgcgaacccaaaccgcaattccgctcgcgggtttaatgtgatcgacaacatcaaagcagcggtcgagaaagcatgtcccggggtcgtgtcttgtgctgatatcttagccatcgcagctagagactccgtcgtagtccttggagggcctaactggactgtgaaagtaggaagaagagatgcgagaacggcgagtcaagcggcggcgaatagcaacattccggcgcccacttctagtctgagccaactcattagtagtttcagtgccgttggactctccaccagagatatggttgctctctccggcgcgcacacgatcgggcaatcccgttgcacgagcttccgaacgagaatctataacgagacaaacatcaacgccgcattcgccacaacacgtcaacgaacttgccctagaacctccggctccggcgacgggaatttagctccacttgacgtcaccacggcggcttctttcgacaacaactatttcaagaatctcatgactcaaagaggtcttctccattccgaccaagagctcttcaacggcggctccactgactccatagtccgtggatacagcaacaatccgtcaagctttagctccgattttgcggcggcgatgattaaaatgggtgatattagccccttgaccggtagtagcggtgagatccggaaggtttgcgggaggaccaactgatatttctttttccctattggaatttgacttttgttagttgattcggtgagaataagatttgatcttcccccaagaaaaatcgaagaatgtttgttttccttgttttgtgttcaagtgtgtattttcgcattaaaatctaatgataattagctatgatttccctttacgaaattttttttattcgcatttttaattagtgattacgtgtctgaattccattggttatcataaataattaatcttttgaagcgaaaaaaaaaaaaaaa

SEQ ID NO: 22 - DNA, 1032 bp, Artificial Sequence
  HRP isoenzyme contig01350_ALTERNATIVE
aagtacaataatcaactccaactccaagtctattcaaagtctttgtttatcttaaacatggcttcaaatcaacgtatttccattctagttctcgtagttacacttttagtgcaaggtaattacaataacgtcgttgaagcacaactgacgcccaatttctactcaacctcttgccctaacctcctctccaccgtccaatccgccgttaagtctgccgttaacagcgaggctcgaatgggtgcatctatcgtacgccttttcttccacgattgcttcgtcaacggatgcgatggttcgattttactagatgacacatcaagcttcacgggagaacaaaacgcgaacccaaaccgcaattccgctcgcgggtttaatgtgatcgacaacatcaaagcagcggtcgacaaagcatgtcccggggtcgtgtcttgtgctgatatcttagccatcgcagctagagactccgtcgtagtccttggagggcctaactggactgtgaaagtaggaagaagagatgcgagaacggcgagtcaagcggcggcgaatagcaacattccggcgcccacttctagtctgagccaactcattagtagtttcagtgccgttggactctccaccagagatatggttgctctctccggcgcgcacacgatcgggcaatcccgttgcccgagcttccgaacgagaatctataacgagacaaacatcaacgccgcattcgccacaacacgtcaacgaacttgccctagaacctccggctccggcgacgggaatttagctccacttgacgtcaccacggcggcttctttcgacaacaactatttcaagaatctcatgactcaaagaggtcttctccattccgaccaagagctcttcaacggcggctccactgactccatagtccgtggatacagcaacaatccgtcaagctttagctccgattttgcggcggcgatgattaaaatgggtgatattagccccttgaccggtagtagcggtgagatccggaaggtttgcgggaggaccaactga

SEQ ID NO: 23 - DNA, 1051 bp, Artificial Sequence
  HRP isoenzyme contig05508 length=1051 numreads=21
tcactacaactaaaaacacacttgatcttctctaaaacatcgaaacataaatacaagatgggtttgattagatcattatgcgtattcataactttcctcagttgtatcatcagctcggcccatggccaagccatctcgatttctatcacaattaggatcgggttttacttgaccacgtgtcccacagctgaaatcattgttcgaaacgccgtgagagctggtttcaattctgacccgagaatcgcacccggaatattgagaatgcatttccacgactgcttcgttcaaggttgtgacggttcagtccttatatcaggaagtaacaccgagagaaccgccgttccaaacctcagcctccgtggatttgaagtcatagaaaacgccaaaacgcagctcgaagccgcgtgcccaggagttgtctcttgtgctgatattttagccttagctgctcgtgatactgtagtccttacgagagggataggctggcaagtaccaacgggacgtagagatggtcgagtttctgtggcctcgaacgctaataatcttccaggtccccgtgactccgtcgccgttcaacaacagaaattctccgctctcggactcaatacccgcgatctcgtcgtcctcgccggaggacacacgctcggaacagctggatgcggtgtattcagggacagactattcaataacacggatcctaacgtcgaccagccatttttgacgcagcttcaaacaaaatgtccccgaaacggagacggttcagtgcgcgtggatctcgataccggaagcggaaccacttttgataattcctacttcatcaacctaagtcgtggccgcggagtcctcgaatccgatcatgtactttggaccgatccagccactagacccatcgtgcaacagttgatgagttctagtggcaacttcaacgctgaatttgcgaggtcaatggtcaagatgagtaatatcggtgtggttacggggactaatggggaaattcgtaaggtttgctctgcgattaattaattaaccgattaaaatcagtggtgataaa

SEQ ID NO: 24 - DNA, 1130 bp, Artificial Sequence
  HRP isoenzyme contig08562 length=890 numreads=14
atcactcatcatcacaaagtcttcccatttcctcatcttcttattgaaagtaatggcaagactcactagcattctccttcttctttctcttctatgcttttttccctctctgtctctgcgacaagagctatggaggcaaactcttccctggtttttacgcccactcatgcccacaagccggggaaatcgtgagatcagtcgtagctaaagctgttgctagagagacccgtatggctgcttctttgatgagacttcatttccacgactgtttcgttcagggttgtgatggctctttgcttctagacagcagtgggaaaatagtgagtgagaaaggctcaaaccctaacagcagatcagctcgtgggttcgacgtagttgaccaaatcaaagctgaattggagaaacaatgccctggaactgtttcttgcgctgatgctctaactctagccgctagagactcctctgttcttaccggtggaccgagctgggtggtttcattaggaagaagagattcaagaagtgcaagcttgagtggttcgaacaacaatatccctgcaccaaacaacactttccagaccattctatcgaagtttaaccgtcaaggactcgatgtcaccgaccttgttgctctctccgggagccacaccattggattctccagatgcacgagtttcagacaaaggttgtacaaccagtccggaaacggacgtccagacatgacactggaacaatccttcgctgctaacttgcgccaaaggtgtccgagatccggcggggaccagattctctcggtgctggacatcatcagcgccgcgaaattcgacaacagctacttcaagaacttgatagagaacaagggtttgttgaactcggaccaggttctgttcaacagtaatgagaaatctagagagcttgtgaagaagtatgcagaggaccaaggagagttttttgagcagtttgcggaatcaatgatcaagatgggaaatatttctcccatgacgggttcgaatggcgaaatcaggaagaattgcaggaagataaactcttgaattcttgaaatgaggaaagtattggggcgaaaaaaaaatattttggagaagaaatgtgtgacatttgtttttactttggtt

SEQ ID NO: 25 - DNA, 1116 bp, Artificial Sequence
  HRP isoenzyme contig08562_ALTERNATIVE length=890 numreads=14
atcactcatcatcacaaagtcttcccatttcctcatcttcttattgaaagtaatggcaagactcactagcattctccttcttctttctcttctatgcttttttccctctctgtctctgcgacaagagctatggaggcaaactcttccctggtttttacgcccactcatgcccacaagccggggaaatcgtgagatcagtcgtagctaaagctgttgctagagagacccgtatggctgcttctttgatgagacttcatttccacgactgtttcgttcagggttgtgatggctctttgcttctagacagcagtgggagaatagtgagtgagaaaggctcaaaccctaacagcagatcagctcgtgggttcgacgtagttgaccaaatcaaagctgaattggagaaagaatgccctggaactgtttcttgcgctgatgttctaactctagctgctagagactcctctgttcttaccggtggaccaagctgggtcgttccattgggaagaagagattcgagaagtgcaagcttaagtggttctaacaacaacatccctgcaccaaataacactttccagaccattctatcgaagtttaatcgtcaaggactcgatgtcaccgaccttgttgctctctctgggagccacaccattggattctcgagatgcacgagtttcagacaaagattgtacaaccagtccggaaacggacgtccagacatgacattggaacaatccttcgctgctaacttgcgccaaaggtgtccgagatccggcggagaccagattctctcagtgttggacatcatcagcgccgcgaaattcgacaacagctacttcaagaacttgatagaaaacaagggtttgttgaactcggaccaggttctgttcagcagtaatgagaaatctagagagcttgtgaagaagtatgcagaggaccaaggagagttttttgagcagtttgcggaatcaatgatcaagatgggaaatatttctcccatgacgggttcgaatggcgaaatcaggaagaattgcaggaagataaactcttgaattcttgaaatgaggaaagtattggggcgaaaaaaaaatattttggagaagaaatgtgtgacatttg

SEQ ID NO: 26 - DNA, 1066 bp, Artificial Sequence
  HRP isoenzyme contig22489 length=1066 numreads=47
actacaactaaaaacacacttgatcttctctaaaacactaaaattatatatccaatatggagtttgttagatcattatgcgtattcataactttcctcggttgtctcatcagctcggcccatggccaagccgccgcaaggcgacctggtccgatttctggcacaaggatcgggttttacttgaccacgtgtcccaccgctgaaatcattgtccgaaacgccgtgagagctggtttcaattctgacccaagaatcgcacccggaatattgagaatgcatttccacgactgcttcgttctaggttgtgacggttcagtccttatatcaggaagtaacactgagagaaccgccgttccgaacctcaacctccgtggatttgaagtcatagacaacgccaaaacgcagctcgaagccacatgcccaggagttgtctcttgtgctgatattttagccttagctgctcgtgatactgtagtccttacgagagggttaggctggcaagtaccaacgggacgtagagatggtcgagtttctgtggcctcgaacgctaataatcttccaggtccccgtgactccgtcgccgttcaacaacagaaattctccgctgtcggactcaatacccgcgatcttgtcgtcctcgccggaggacacacgatcggaacagctggatgcggtgttttcagggacaggctattcaataacacggatcctaacgtcaaccagctatttttgacgcagcttcaaacacaatgtccccaaaacggagacggtgcagtgcgcgtggatctcgacaccggaagtggaaccacttttgacaattcctacttcatcaacctaagccgtggccgcggagtcctcgaatccgaccatgtactttggaccgatccagccactagaccgatcgtgcaacagttgatgagtcctagaggcaacttcaacgctgaatttgcgaggtcaatggtcaggatgagtaatatcggtgtggttacgggggctaatggggaaattcgtagggtttgctctgcggttaattaattaaccgattaaaatcagtggtgataaacaga

SEQ ID NO: 27 - DNA, 1111 bp, Artificial Sequence
  HRP isoenzyme contig02021 length=758 numreads=128
atcctgtctctcttcactgacacaaaaccaaagaagagtattgagaaacaagatcgaggaaactactcaatgaggacgatgaagcgattgaacgtggcggtggcggttgcggttacagcgacggttcttatgggaatgttaggatcatcagaggctcagcttcaaatgaatttctacgcgaagagctgtccaaacgcagagaaaataatttcagatcatattcaaaagcatatccctagtggtccttctcttgcagctcctctcatcagaatgcacttccatgattgcttcgtcaggggatgtgatggatcggtgttgataaattcgacatcagggaacgcagagaaagattcagcaccaaatctaacacttagaggcttcggtttcgtagagaggattaagactcttcttgaagcagagtgtcctaagactgtttcttgcgccgacatcatcgcactgaccgctagagacgcagttgttgccaccggaggtccttcatggaaagttccgacaggaagaagagacggtaggatctcaaatacgacggaggctttgaacaacattccaccgccgacgagtaatttcacgacgttacagcgacttttcgctaatcaaggccttaatctcaaagaccttgttctgctttccggagctcacacgatcggtgtctcgcattgttcttccatgaatactcgtctctacaacttctcgacgacagtcaaacaagatccatctctggatagcgagtacgcagcaaatctaaaggctaacaaatgtaagagtcttaacgataacaccaccatcctcgagatggatcctggtagtagcaaaacctttgatctcagttattataggcttgtcttgaagaggagaggtttgtttcagtctgattctgccttaacgacaaactcagctacgttgaagatgatcaacgacttggtcaacggtcctgaaaagaagtttttaaaggctttcgctaagtcaatggagaagatggggagagttaaagtgaagacgggctcagccggtgtgattaggacacgttgttctgttgccggaagttagttagtttggtcggaaagtgatgttttctgttcattgttgttctgtt

SEQ ID NO: 28 - DNA, 1399 bp, Artificial Sequence
  HRP isoenzyme contig15901 length=1294 numreads=33_C1B
attctcggttaaacacaattaattagttttctttttatctttaaaaatatgcattccccttcttctacttcgtttacttggatcttaatcacattgggatgtcttgcgttttatgcgtctttgtccgatgctcagcttacccctaccttctacgacacttcatgtcctaatgtctcaaacatcgtacgagacatcattattaatgagctacgatcggaccctcgtatcaccgcgagtatccttcgtcttcacttccacgactgctttgttaatggttgtgacgcatcgatattgttagacaacacaacatcatttctaacagagaaagatgcgcttggaaacgcaaactcggctagaggatttcctacagttgacagaatcaaggccgcggtggagagggcatgcccaagaacagtttcatgcgcagatgtgcttaccattgcagctcaacaatctgttaatttggcaggaggtccttcttggagggttcctttgggaagaagagacagcttacaagcatttttagatcttgctaatgcaaatcttccagctccattctttacacttccacaacttaaggatgcctttgcaaaagttggcctcgaccgtccttctgatctcgttgctctctccggtggtcacacatttggaaaaaatcagtgtagatttattatggacagattatacaacttcagcaacaccggactacccgaccctaccctcaacactacttaccttcaaactcttcgtcaacaatgtcccctaaatggaaaccaaagtgtattggtggatttcgatctgcgtacgccaacggttttcgataacaaatactatgtgaatcttaaagagcaaaaaggtctcattcagagtgaccaagagttgttctctagccccaatgccactgacacaatccccttggtgaggtcatttgctgatggcacacaaaaattcttcaatgcgtttgtggaggctatgaataggatgggaaatattacacctcttacaggaactcaaggagaaatcaggttgaattgtagggtggtgaactccaactctctactccatgatatagtggaggtcgttgactttgttagctctatgtgagaaaagttgagtcaatatctggctaccagagtacacgttaagataaataaagcgctctcaagatgttacttgagaaggagaagatatttattggtgtgtagagagtatctaagttgttctctgtttttatgtttgagttggcctttgaatgcgtttcgtgaatcggtctaaacttttatgggtttgtgtgtagagagtatctaagttgttctctgtttttatgtttgagttggcctttgaaatgcgtttcgtgaatcggtctaaacttttatgggtttgggacgttctatcctga

SEQ ID NO: 29 - DNA, 1301 bp, Artificial Sequence
  HRP isoenzyme contig25148 length=1241 numreads=25_C1C
atgcattccccttcttctacttcgtttacttgggcaaccttaatcacattgggatgtcttatgcttcatgcatctttttccaatgctcaacttacccctaccttctacgacaattcatgtcctaacgtctcaaacatagtacgggacatcattatcaatgagttacgatcggaccctcgtatcgccgcgagcatccttcgtcttcacttccacgactgctttgttaatggttgtgacgcatcgatcttgttagacaacacaacatcatttcgaacagagaaagatgcgtttggaaacgcaaactcggctcgaggatttcctgtggttgacagaatcaaggccgcggtggagagggcatgcccaagaactgtttcatgcgcagatgtgcttaccattgcagctcaacaatctgttaatttggcaggaggtccttcttggagggttcctttgggaagaagagacagccgacaagcatttttagatctcgctaatgcgaatcttccagctccatccttcacacttccagaacttaaggctgcttttgcaaatgttggcctcaaccgtccttctgatctcgttgctctctctggtggtcacacatttggtaaaaatcaatgtcgatttattatggacagattatacaacttcagcaacaccggactacccgaccctaccctcaacactacttaccttcaaactcttcgtcaacaatgtccccgtaatggtaaccaaagcgtcttggtggatttcgatctgcgtacgccaacagttttcgataacaaatactatgtgaatcttaaagagcaaaaaggtctcatccagagtgaccaagagttgttctctagccccaatgccactgacacaatccccttggtgagatcatatgctgatggcacacaaacattcttcaatgcctttgtggaggccatgaataggatgggaaacattacacctcttacaggaactcaaggagaaatcaggttgaactgtagggtggtgaactccaactctctactccatgatatagtggaggtcgttgactttgttagctctatgtgagaaaagttgactcaatatctggctaccagagtatacgttaagataaataaagcgctctcaagatgttacttgagaaggagaatatctttattggtgtgtagtgtgtagggtatctaagttgttctctgtttttatgtttgtgttggcctttgaatgcgtttcgtgaatcggtctaaacttgtatgggtttggacgttctatcctaataaagatgataaaataaaataaaaaaatgttactcg

SEQ ID NO: 30 - DNA, 1166 bp, Artificial Sequence
  HRP isoenzyme contig04627 length=1166 numreads=80_C2
cggccggggactcaacttcaaacctaaccaaagaattttatcttagagagcaaagaaaatgcattcctcttccagtttgataaaattgggatttcttcttcttcttcttaatgtatcattgtctcacgctcaactaagcccttcattttacgataaaacatgtccacaagtctttgacattgcaaccaataccattaaaactgcgctgagatcagaccctcgcatcgctgcgagcatccttcgtcttcatttccacgactgctttgttaatggatgtgatgcatcgatattgctagacaacactacatcatttaggactgagaaagatgcgtttgggaacgcaagatcagctcgaggcttcgacgtaatcgacacaatgaaggctgccgtggagaaagcatgtcctaaaaccgtttcatgtgctgatttgctcgccattgcagctcaaaaatctgtcgttttggcgggaggtccttcatggaaggttccaagtggaagaagagacagcttaagagggttcatggatctcgctaatgataaccttccaggtccatcctctacacttcaagtacttaaggacaaattcagaaatgtcggactcgaccgtccttctgatctcgttgctctttctggtggtcacacctttggcaaaaaccaatgtcagttcataatggatcggctttacaacttcagtaactccggtaaacccgacccaacccttgataaatcgtacctcagcacgctaagaaaacaatgcccacgtaatggaaacctgagtgtattggtagattttgatttacgtacaccgacaatctttgacaacaaatactatgtgaatctcaaagagaacaaaggtcttatccagagtgaccaagagttattctctagccctgatgcttctgacactatccctttggtccgagcatacgctgatggtcaaggaaagttttttgatgcattcgtggaggcaatgataaggatgggaaatctttcaccttcaactgggaaacaaggagaaattagattgaattgtagagtggtgaattctaaacctaaaatcatggatgtggttgatactaatgactttgccagctccatctgaagaaatgactttctcctaataataaatgatcaatgttatctttttctttcaaaaaaaaaaaaaa

SEQ ID NO: 31 - DNA, 1201 bp, Artificial Sequence
  HRP isoenzyme contig01805 length=1201 numreads=50
atcttctcaacttaaaccaataaaagataagtttcctcttaccaaaaatgcatttctctacttcttcttcttccttgtctacttggacaaccctaataacattggggtgtcttatgcttcattcatttaagtccagtgctcaactaacccctaccttttacgacagtacctgccccagcgtctttagcatcgtacgggacaccatcgtgaacgagctaagatcagatcctcgaattgctgcaagtatccttcgtcttcacttccacgactgcttcgttaatggctgtgatgcatcgattttgttagacaacacaacatcatttcgaacagagaaagatgcagctccaaacgcaaactcagctcgaggatttccagtgattgatacaatgaaagctgcagtggaaagagcatgtccaagaaccgtatcatgcgcagatttgcttaccatcgcagctcaacaatctgtgaatttggcaggaggtccttcttggagggttcctttggggagaagagacagcgtacaagcattttttgatcttgccaatacaaatcttcccgctccattcttcacgcttccacaacttaaggccagctttagtaatgttggacttgaccgtccagaagatctcgttgcactctctggtggtcacacatttggtaaaaaccaatgccaatttattatggacagactatacaactttagtaacactggtttacccgaccctactctcaacactacttatctccagacacttcgtgtacaatgtccccgtaatggtaaccagtccgtcttggtcgatttcgatctacgcacaccgacagtttttgacaacaaatactatgtgaatctgaaagagcacaagggacttatccagaccgatcaagagttgttctccagccctaatgccgctgatacaatccccttggtaagatcatatgctgatggcactcagaagttcttcaatgcttttatggaggccatgaacagaatgggaaacattacccctctcactggaactcaaggacagatcaggcaaaattgtagggtgatcaactccaactcgctgctccatgatattgttgaaatcgttgactttgtgagctctatgtaacaatagttgtctcaatatatgtggcaaccaaaattatatgttcttatgaaaataaaatgttctcgaaacattacttaagaatatttggt

SEQ ID NO: 32 - DNA, 1211 bp, Artificial Sequence
  HRP isoenzyme contig00938 length=1211 numreads=67_E5
aggccgggggaccatcagccacactcccaactcaaatcatagtctatcatcctcctaaaaattaaagagaaatggtggtttctcctttcttttcttgcagtgctatgggagccctaatattgggttgccttctgcttcaagcatctaatgctcagttgaggcctgacttctactctaggacttgcccatctgttttcaatattattaagaatgtcatcgtcgatgaactgcagactgatcctcgcattgccgctagtatccttcgccttcactttcatgactgctttgttcgtggttgtgatgcatcgatcctgctcgatacttccaaatcgttccgaaccgaaaaagatgctgctccaaacgtaaattcggctcgagggttcaatgtcatagatagaatgaaaacagcacttgagagagcttgtcctagaacagtgtcttgtgcagatattctcaccatcgcctctcaaatatcagtgcttttgtcgggaggtccatcttgggcagttccgttggggaggagagacagcgtagaagctttctttgacctagctaatacagctcttccctctccatttttcactcttgctcaacttaaaaaagcttttgctgacgttggtttaaaccgcccctcagatctagtcgctctttctggtggtcacacatttggaagagcacgatgcctatttgtgacagctcgtctctacaacttcaacggtacaaacagaccagacccaactctgaacccatcttacctcgccgacctccgtcgattgtgccctcgaaacggaaacggcaccgttctggtcaacttcgatgtcatgactccgaatactttcgataatcaattctacactaatcttagaaatgggaaaggtctgattcagagtgaccaagagctcttctcgactccaggagccgacacgatcccactagtaaacctatacagcagcaacacgttatcgttcttcggagcattcgctgatgcaatgattaggatgggaaatcttagacctttgactggaactcaaggcgagataagacagaattgtagggttgtgaattcgcgaattaggggtatggagaacgatgatggagttgtgagttctatgtgattatgttgggaatatatatcatatatggttatgtaacaaatcataaaatgtgtgggaacatgcatgtcgactaaataaaagttctaacgagttgtg

SEQ ID NO: 33 - DNA, 1173 bp, Artificial Sequence
  HRP isoenzymecontig22684 length=1173 numreads=70
aaaaaaaaaaaaaaacggaaccgcctccctataggtagtcgtattacggccggggaccatcagcaaaactcacgactcaaaccatagtctgtcttcctcttgaaaaaagaaaaatggggttttctccttcattttcttccagttctataggagtcctaatattgggttgccttctgcttcaagcttcaaactctaatgctaagttgaggcctgacttctacttaaagacatgtccatcagttttccaaatcattgggaatgtcatcgtcgatgaactgcagagtgatcctcgtattgcagctagtctccttcgccttcacttccatgactgttttgttcgtggttgtgatgcatcggtcctgctcgacaattccacatcatttcagtccgagaaagatgctgctccaaacgcaaattcggctcgagggttcgacgtcgtagatagaatgaaagcagcccttgagaaagcttgtcctggaacagtgtcttgtgcagatgttcttgccatctccgctcaaatatcagtgcttttgtcgggaggcccatggtggccggttttgttggggaggagagacggcgtagaagctttcttcgatttggctaatacagctcttcccaatccatttgcccctcttactgaacttaaagaaaaatttgctgacgttggcctaaagcgcgcctcagatctagttgctctttccggtgctcacacatttggaagagcacaatgtctacttgtgacacctcgtctctacaacttcagcggcaccaataaaccagacccaactctgaacccatcttacctcgtcgaactccgtcgattgtgccctcaaaacggaaacggcaccgttctgctcaacttcgatctcgtgactccaaatgctttcgatcgtcaatactacaccaatcttcgaaatgggaaaggtctgattcagagtgaccaagagctcttctcgactccaggagccgacacgatcccactagtaaacctatacagcaagaacacgttcgcgttcttcggtgcattcgttgacgcaataattaggatgggaaatattcaacctttgactggaactcaaggcgagataagacagaattgtagggttgtgaattcgcgaattaagggtatggagaacgacggtggagttgtgagttctatttgattatgttggg

SEQ ID NO: 34 - DNA, 477 bp, Artificial Sequence
  HRP isoenzyme contig01351
aaatacaataatcaactccaagtctattcaaagtctttgtttaacctaaccatgacttcaaatcaacttatttccattctagttctcgtagttacacttttagtgcaaggtaattacaataacatcgtcgaagcacaactcacggccaatttctactcaacctcttgccctaatctcctctctaccgtccaagccgccgttaagtctgccgttaacagcgaggctcgaatgggtgcatcgatcgtacgccttttcttccacgattgcttcgtcaacggctgcgacggttctattctactagatgacacatcaagcttcacgggagaacaaaacgcgaacccaaaccgcaattccgctcgcgggtttaatgtgatcgacaacatcaaatcagcggttgagaaagcatgtcccggggtcgtgtcgtgtgctgatatcttagccatcgcagctagagactccgtcgtactacttggaggg

SEQ ID NO: 35 - DNA, 1199 bp, Artificial Sequence
  HRP isoenzyme HRPC1AsynK232Q_K241N
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtaagaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaaccaatactatgtcaacttggaggaacagaacggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 36 - DNA, 1199 bp, Artificial Sequence
  HRP isoenzyme HRPC1AsynK232Q_K241F
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtaagaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaaccaatactatgtcaacttggaggaacagttcggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 37 - DNA, 1199 bp, Artificial Sequence
  HRP isoenzyme HRPC1AsynK232N
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtaagaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaacaactactatgtcaacttggaggaacagaagggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 38 - DNA, 1199 bp, Artificial Sequence
  HRP isoenzyme HRPC1AsynK232N_K241N
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtaagaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaacaactactatgtcaacttggaggaacagaacggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 39 - DNA, 1199 bp, Artificial Sequence
  HRP isoenzyme HRPC1AsynK232N_K241F
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtaagaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaacaactactatgtcaacttggaggaacagttcggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 40 - DNA, 1199 bp, Artificial Sequence
  HRP isoenzyme HRPC1AsynK174R_K241N
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtcgtaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaacaagtactatgtcaacttggaggaacagaacggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 41 - DNA, 1199 bp, Artificial Sequence
  HRP isoenzyme HRPC1AsynK174R_K241F
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtcgtaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaacaagtactatgtcaacttggaggaacagttcggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 42 - DNA, 1199 bp, Artificial Sequence
  HRP isoenzyme HRPC1AsynK174R_K232Q
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtcgtaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaaccaatactatgtcaacttggaggaacagaagggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 43 - DNA, 1199 bp, Artificial Sequence
  HRP isoenzyme HRPC1AsynK174R_K232N
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtcgtaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaacaattactatgtcaacttggaggaacagaagggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 44 - DNA, 1199 bp, Artificial Sequence
  HRP isoenzyme HRPC1AsynK174Q_K241N
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtcagaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaacaagtactatgtcaacttggaggaacagaacggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 45 - DNA, 1199 bp, Artificial Sequence
  HRP isoenzyme HRPC1AsynK174Q_K241F
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtcagaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaacaagtactatgtcaacttggaggaacagttcggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 46 - DNA, 1199 bp, Artificial Sequence
  HRP isoenzyme HRPC1AsynK174Q_K232Q
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtcagaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaaccagtactatgtcaacttggaggaacagaagggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 47 - DNA, 1199 bp, Artificial Sequence
  HRP isoenzyme HRPC1AsynK174Q_K232N
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtcagaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaacaattactatgtcaacttggaggaacagaagggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 48 - DNA, 1199 bp, Artificial Sequence
  HRP isoenzyme HRPC1Asyn_T110V
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttgtgttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtaagaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaacaagtactatgtcaacttggaggaacagaagggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 49 - DNA, 1199 bp, Artificial Sequence
  HRP isoenzyme HRPC1Asyn_K241F
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtaagaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaacaagtactatgtcaacttggaggaacagttcggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 50 - DNA, 1199 bp, Artificial Sequence
  HRP isoenzyme HRPC1Asyn
aacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttactccaaccttctacgataactcttgtcctaatgtgtccaacatcgttagagacaccattgtcaatgaattgagatcagatccacgtattgctgcatctatcttgagacttcactttcatgactgcttcgtcaacggttgtgatgcttccatcttgctggacaacactacctctttcagaactgagaaggacgctttcggtaatgccaactctgctagaggatttccagtcattgacagaatgaaggctgccgttgaatctgcatgtcctagaactgtgtcatgtgctgaccttctgactattgccgctcagcaatctgttaccttagctggtggaccatcctggagagttccattgggtcgtagagactcccttcaagcctttctggaccttgcaaatgctaacttgcctgctccattctttaccttacctcaattgaaagactctttcagaaacgttggtcttaacagatcatccgacttggttgccttatctggaggtcacacctttggtaagaaccaatgtagattcatcatggatcgtctgtacaacttctctaacaccggtttgccagatcctactctgaacaccacttacttgcaaaccttaagaggtttgtgcccacttaacggaaatctgtctgctctggttgacttcgatttgcgtactcctaccatcttcgacaacaagtactatgtcaacttggaggaacagaagggtcttatccaatctgaccaggagttgttctcctctcctaacgctactgataccattccattggtgagatccttcgcaaactccactcaaaccttctttaacgctttcgtcgaggcaatggacagaatgggtaacattactcctttgaccggtactcaaggacagattagattgaactgccgtgttgtcaactctaactcataat

SEQ ID NO: 51 - DNA, 2065 bp, Artificial Sequence
  HRP isoenzyme C1A
tcatcttctcagtaatatagttttcccctttaaaaatgcatttctcttcttcttctactttgttcacttgtataaccttaatcccattggtatgtcttattcttcatgcttctttgtctgatgctcaacttacccctaccttctacgacaattcatgtcctaatgtctctaacatcgtacgggatactattgtcaatgagctaagatcagaccctcgtattgccgcgagcatccttcgtcttcacttccacgactgctttgttaatgtaaattactacttttcatatttctatttcgttacgaattattatatgtttcatgtaactgatttttaaatgcttatctattcatactgtctaattacatttatcatttgaatttgattattaaatcagttttaatgtgattaatataaattttaaaaaaatgtgattaatatataaacattttgatgataaaatttttaatttgtttatttttctttttttttactgaataaaattttttttagtggatgacaaaaatgttacattgttgccgtggtaaagattcaactgtatatggatgtaatacattgaataataatttattaaatgatatattggatttttgaaagggttgtgacgcatcgatcttgttagacaacacaacatcatttcgaacagagaaagatgcgtttggaaacgcaaactcggcaagaggatttccagtgattgatagaatgaaagccgcggtggagagtgcatgcccaagaaccgtttcatgcgcagatttgctcaccattgcagctcaacaatctgtcactttggtatgctccattgatcccactaacttttattcattacaattattgcttttaattttaatatatcaaatagtcactttacatatcaacacggcaacctaagtttagaaaaacaaaaatcgggatatttttagctgtttgagaacgacatggaaattatttagttgtttcagaagggtcacaatcaatatatagtatcaactttccaatcatatgactagtattcaaaattaatcgggagatatttgaaacgcgtggtccccgtagagaaaacttgaaatgtttaatatcactgaaaaaaattaatctcattaacaagaatatacttacttggcaatactacgtttttagttaaccaaaaagaatggttttcatatttttcaagaaacagtggagacttaaaaacttcacttcaagcatatatatcatcaatgaatttaaattacacatatttttctcttaacacattgaaacttctaaatgaggaaaataataatcaaaacaaaatggttattattataggcgggaggtccttcttggagagttcctttgggcagaagagatagcttacaagcatttctggatcttgctaatgcaaatcttccagctccattcttcacacttccacaacttaaagacagctttagaaatgttggcctcaaccgttcttctgatctcgttgcactgtccggtaattaacaaaaatatattaaacacaccatttgatatagttgtatttagtgagtttattaaagatctctctttcttttgttagggggccacacatttggtaaaaatcagtgtcggtttattatggacagattatacaacttcagcaacaccggtttacccgatcctactctcaacactacttatctccaaactcttcgtggactatgtcccctcaatggtaatctaagcgctttggtggattttgatctacgtacgccaacgatttttgacaacaaatactatgtgaatctcgaagagcaaaaaggacttatccaaagcgaccaagagttgttctctagccccaatgccactgacacaatccctttggtgagatcatttgctaatagcacacaaacattcttcaatgcgtttgtggaggcgatggataggatgggaaacattacacctcttacaggaactcaaggacagatcaggttgaattgtagggtggtgaactccaactctctactccatgatatggtggaggtcgttgactttgttagctctatgtgagcatagtcgacgcca

SEQ ID NO: 52 - DNA, 1554 bp, Artificial Sequence
  HRP isoenzyme SynPDI_N314H_DNA
atgcaattcaactgggacatcaagacagttgcatccatcctttctgccttgactcttgcacaagcttctgaccaagaagctattgctccagaagactctcacgttgtgaagttgactgaggctactttcgagtccttcatcacctctaaccctcatgtattggcagagtttttcgcaccttggtgtggtcactgcaagaagttaggtcctgagttggtatctgctgctgagattttgaaggacaatgagcaagtcaagattgcccagattgactgcactgaggaaaaggagttgtgtcaaggttacgagattaagggttaccctaccttgaaggtgtttcacggtgaagttgaggttccatctgactaccaaggacaaagacagtcccaatccatcgtttcctacatgctgaagcaatctcttccacctgtatccgagatcaacgcaactaaggacttggatgacaccattgcagaggctaaagagccagttatcgtccaagtccttccagaagatgcatccaacttggagtccaatacgaccttttacggagttgcaggtacattgcgtgagaagttcacctttgtctctaccaagtctactgactacgctaagaagtacacctctgactctactccagcttacctgttggttagacctggagaagagccatccgtctactctggtgaggaattggatgagactcaccttgttcactggatagacatcgagtccaaacctctgtttggagacattgatggttccaccttcaagtcttatgctgaggctaacattcctcttgcctactatttctacgagaacgaagagcaaagagcagcagctgctgacatcattaagccatttgctaaggagcaaagaggcaagattaacttcgttggtttggatgcagtcaagttcggcaaacatgccaagaaccttcacatggatgaggagaagttgccactgttcgttatccatgacttggtttccaacaagaaattcggtgttcctcaagaccaagaactgaccaacaaggacgttacagagctgattgagaagttcattgcaggtgaagcagaacccattgtcaagtctgagcctattccagagatacaagaggagaaggtcttcaagttggtaggtaaagctcacgatgaagtcgttttcgacgaatccaaggacgttttggtcaagtactatgctccttggtgtggtcactgtaagagaatggcaccagcttacgaagagcttgctactttgtacgccaatgatgaggatgcatcttccaaggtggttattgccaagcttgatcacactcttaacgacgttgataacgtggacatacagggatacccaaccttgatcctttatcctgctggtgacaagtctaacccacagttatacgacggttccagagaccttgagtctttggctgagtttgtcaaggaaagaggtactcacaaggttgacgctttagctttgagacctgttgaagaggagaaagaagctgaggaagaagctgagtctgaagctgatgctcacgacgaattgtaa

SEQ ID NO: 53 - protein, 517 aa, Artificial Sequence
  HRP isoenzyme SynPDI_N314H
MQFNWDIKTVASILSALTLAQASDQEAIAPEDSHVVKLTEATFESFITSNPHVLAEFFAPWCGHCKKLGPELVSAAEILKDNEQVKIAQIDCTEEKELCQGYEIKGYPTLKVFHGEVEVPSDYQGQRQSQSIVSYMLKQSLPPVSEINATKDLDDTIAEAKEPVIVQVLPEDASNLESNTTFYGVAGTLREKFTFVSTKSTDYAKKYTSDSTPAYLLVRPGEEPSVYSGEELDETHLVHWIDIESKPLFGDIDGSTFKSYAEANIPLAYYFYENEEQRAAAADIIKPFAKEQRGKINFVGLDAVKFGKHAKNLHMDEEKLPLFVIHDLVSNKKFGVPQDQELTNKDVTELIEKFIAGEAEPIVKSEPIPEIQEEKVFKLVGKAHDEVVFDESKDVLVKYYAPWCGHCKRMAPAYEELATLYANDEDASSKVVIAKLDHTLNDVDNVDIQGYPTLILYPAGDKSNPQLYDGSRDLESLAEFVKERGTHKVDALALRPVEEEKEAEEEAESEADAHDEL

SEQ ID NO: 54 - DNA, 22 bp, Artificial Sequence
  Adaptor Primer1
gtaatacgactcactatagggc

SEQ ID NO: 55 - DNA, 19 bp, Artificial Sequence
  Adaptor Primer2
actatagggcacgcgtggt

SEQ ID NO: 56 - protein, 31 aa, Artificial Sequence
  N-terminal signal peptide of HRP isoenzyme 01805
MHFSTSSSSLSTWTTLITLGCLMLHSFKSSA

SEQ ID NO: 57 - protein, 29 aa, Artificial Sequence
  signal peptide of HRP isoenzyme 22684
MGFSPSFSSSSIGVLILGCLLLQASNSNA

SEQ ID NO: 58 - protein, 27 aa, Artificial Sequence
  signal peptide of HRP isoenzyme E5
MVVSPFFSCSAMGALILGCLLLQASNA

SEQ ID NO: 59 - protein, 15 aa, Artificial Sequence
  C-terminal peptide
LLHDMVEVVDFVSSM

SEQ ID NO: 60 - DNA, 1218 bp, Artificial Sequence
  HRP isoenzyme A2ACstrep
gaattcgaaacgatgagattcccatctattttcaccgctgtcttgttcgctgcctcctctgcattggctgcccctgttaacactaccactgaagacgagactgctcaaattccagctgaagcagttaggttactctgaccttgagggtgatttcgacgtcgctgttttgcctttctctaactccactaacaacggtttgttgttcattaacaccactatcgcttccattgctgctaaggaagagggtgtctcttgagaagagagaggccgaagctcaattgaatgcaactttctattccggaacctgcccaaacgcctctgcaatcgttagatccactattcaacaggccttccaatcagacactagaattggtgcatcttattagactgcactttcacgattgtttcgtgaacggttgtgacgcctctatcttgctggatgactcaggatctattcagtcagagaagaatgctggtccaaacgccaattccgctagaggtttcaatgtggttgataacatcaagaccgctttggaaaacacttgccctggtgtggtttctttccgacatcttggctttggcatctgaagcatccgtttctttgactggaggtccatcctggactgtcttattgggtagacgtgactctcttaccgctaacttagctggtgccaactcagctattcctcccatttgaaggtctttctaacattacctctaagttctccgctgtcggactgaacaccaacgaccttgttgcactgtctggtgcccatacctttggacgtgctagatgtggtgtcttcaacaataatgttcaacttttctggaactggtaacccagaccctaccttgaactccactttgctgtcttccttgcagcaactttgtccacagaacggttctgcttcaactatcactaacctggacttatctaccctgcgctttcgacaacaattactttgccaaccttcaatctaacaacggtttgttacaatccgatcaagagttgttctctaccactggttctgctactatcgcagttgtcacctctttcgcttctaacaaatttgttctttcaagccttcgctcaatcaatgatcaacatgggtaacatttctcctttgaccggttctaacggtgagattagacttgattgcaagaaagttaacggatcctctgcatggtccccccacgttcgagaagtaatgcggccgc

SEQ ID NO: 61 - DNA, 1658 bp, Artificial Sequence
  HRP isoenzyme 04663.2 >04663.1_gene_1077nt
atggctgcaacaagctcttcactacttgtgatggtctcttcatcattagccttcttgttatcgcttcttcattgtttgggacatcatctgcgcagttaaacgctacgttttactccgggacgtgccctaatgcctctgccatcgtacgcagcactatccagcaagctcttcaatccgacccgaggatcggagccagcctcatccgccttcattttcacgactgttttgttaatgtataagtcaataagaagctcagaagatctttaaataagcttcttggttttattttatcataaggccttttgatttctgttccactaattaaccgccctatttggcttctttacttttgacaagggctgcgacgggtcgctcttgcttgacgacactggaagtatccagagcgagaagaacgctcctgccaacgcaaactcagctagaggatttaatgttgtcgacgatatcaaaactgccctcgagaatgcttgtcccggcattgtctcttgctctgacattctagctcttgcctcagaggcttccgtgtctttggtaacaacactttcttcataaacatatcaaacaaaacagacacacatatatatttaaactcacacacatatatagggatattagtatggatcaaaagtccacggggttccaatcccggccaagaatcccaccggcttaattttaaaacaaatttatgttaaaacgacgtcattttggccagttaaaaatatgagaaaaaaaataaattacagttgactaaaaatattgcatgtgtttaatcgcactgatcctggccctgtgggccttcccacaggcttaatcgccaataatgagtttacataattacattcttatacacaaataaaaatatgcatgtttgatcatcttcaggcaggaggtccttcatggactgtgttagtaggaagaagagatggtctcaccgcaaacctgtccggggccaattcgtcgcttccctctcccttcgaaggccttaacaacatcacatctaaatttttagctgtcgggctaaatacaaccgatgtagtagtcttgtctggtaactcatcgacatatttaattacttgcggctcaattcaaacaaaaccttacctaatgacatatctcttgtgtatgttaaatgtgcataggagctcatacgtttgggcgtggccaatgtgtaaccttcaacaatagacttttcaacttcaacggaacaggaagtcccgacccgactctgaactcaacacttctcagcagtcttcaacagatatgtcctcaaaacggcagcggatcagcgatcaccaatctcgatctgactacacctgatgcatttgatagcaactactacacgaaccttcagagtaacaatgggcttcttcagtcagaccaagaactattctccaacaccggttcacccaccatcgcgattgttaattcctttgcaagtaaccaaaccctgttttttgaggcttttgctcagtctatgatcaagatgggtaacattagtcccctgactgggactagtggagagattagacaagattgtaaggcggttaatggacagtcatcagccactaaagcagaggacattcagatgcaatctgacggaccagtgagtttagcagatatgtgaacaataatgggatcag

SEQ ID NO: 62 - DNA, 1393 bp, Artificial Sequence
  HRP isoenzyme >05508.1
tacaagatgggtttgattagatcattatgcgtattcataactttcctcagttgtatcatcagctcggcccatggccaagccatctcgatttctatcacaattaggatcgggttttacttgaccacgtgtcccacagctgaaatcattgttcgaaacgccgtgagagctggtttcaattctgacccgagaatcgcacccggaatattgagaatgcatttccacgactgcttcgttcaaggttgtgacggttcagtccttatatcaggaagtaacaccgagagaaccgccgttccaaacctcagcctccgtggatttgaagtcatagaaaacgccaaaacgcagctcgaagccacgtgcccaggagttgtctcttgtgctgatattttagccttagctgctcgtgatactgtagtccttgtaagccctaatccataagcgcaattgcattaatactacttttcttgttatatatatatatatatatatatatatgtgagaaacgttttacgtgcgtggattgatttgcgtgcagacgagagggataggctggcaagtaccaacgggacgtagagatggtcgagtttctgtggcctcgaacgctaataatcttccaggtccccgtgactccgtcgccgttcaacaacagaaattctccgctctcggactcaatacccgcgatctcgtcgtcctcgccggtacgtagtagtttacactttacatacaatactatagattaccactaattcgaaaaattaacgttgagaaactcttcatgttaatttaaaaaaaaaaaaaaaaaaactcttcatgtgcatgttcgttaaacgattatttttttaaaaaatttaattttaactgatttcaccattttttttgtccgctttaaaacttctttgaaaatatagaaaactttgttaatattaggatgtgaataattacaacatatactgaattattcataaatttttaacaggaggacacacgctcggaacagctggatgcggtgtattcagggacagactattcaataacacggatcctaacgtcgaccagccatttttgacgcagcttcaaacaaaatgtccccgaaacggagacggttcagtgcgcgtggatctcgataccggaagcggaaccacttttgataattcctacttcatcaacctaagtcgtggccgcggagtcctcgaatccgatcatgtactttggaccgatccagccactagacccatcgtgcaacagttgatgagttctagtggcaacttcaacgctgaatttgcgaggtcaatggtcaagatgagtaatatcggtgtggttacggggactaatggggaaattcgtaaggtttgctctgcgattaattaattaaccgattaaaatcagtggtgaaact

SEQ ID NO: 63 - DNA, 1393 bp, Artificial Sequence
  HRP isoenzyme >05508.2
tacaagatgggtttgattagatcattatgcgtattcataactttcctcagttgtatcatcagctcggcccatggccaagccatctcgatttctatcacaattaggatcgggttttacttgaccacgtgtcccacagctgaaatcattgttcgaaacgccgtgagagctggtttcaattctgacccgagaatcgcacccggaatattgagaatgcatttccacgactgcttcgttcaaggttgtgacggttcagtccttatatcaggaagtaacaccgagagaaccgccgttccaaacctcagcctccgtggatttgaagtcatagaaaacgccaaaacgcagctcgaagccgcgtgcccaggagttgtctcttgtgctgatattttagccttagctgctcgtgatactgtagtccttgtaagccctaatccataagcgcaattgcattaatactacttttcttgttatatatatatatatatatatatatatgtgagaaacgttttacgtgcgtggattgatttgcgtgcagacgagagggataggctggcaagtaccaacgggacgtagagatggtcgagtttctgtggcctcgaacgctaataatcttccaggtccccgtgactccgtcgccgttcaacaacagaaattctccgctctcggactcaatacccgcgatctcgtcgtcctcgccggtacgtagtagtttacactttacatacaatactatagattaccactaattcgaaaaattaacgttgagaaactcttcatgttaatttaaaaaaaaaaaaaaaaaaactcttcatgtgcatgttcgttaaacgattatttttttaaaaaatttaattttaactgatttcaccattttttttgtccgctttaaaacttctttgaaaatatagaaaactttgttaatattaggatgtgaataattacaacatatactgaattattcataaatttttaacaggaggacacacgctcggaacagctggatgcggtgtattcagggacagactattcaataacacggatcctaacgtcgaccagccatttttgacgcagcttcaaacaaaatgtccccgaaacggagacggttcagtgcgcgtggatctcgataccggaagcggaaccacttttgataattcctacttcatcaacctaagtcgtggccgcggagtcctcgaatccgatcatgtactttggaccgatccagccactagacccatcgtgcaacagttgatgagttctagtggcaacttcaacgctgaatttgcgaggtcaatggtcaagatgagtaatatcggtgtggttacggggactaatggggaaattcgtaaggtttgctctgcgattaattaattaaccgattaaaatcagtggtgaaact

SEQ ID NO: 64 - DNA, 2258 bp, Artificial Sequence
  HRP isoenzyme >23190.2
atggcaatgagttattcgatacgtgtcctgacgtttctgatgttgatctcgttaatggcagtgacactgaaccttctgtcaacggcggaagcaaagaagccgaggagagatgttcctatagtgaaaggtctctcttggaacttttaccagagagcatgtccgaaagtggaaaagattatcaaaaaagaactcaaaaaagtcttcaagagagatattggtttagccgcagccatccttcgtatacatttccatgactgcttcgttcaggttctatctttttcgttccctaattttttgtattaaaacctaattaagaacatttaattttatgttctttaagcctactttaaacggttctacgtatgtgattaacataattttcactacttgaaaactccttttcttttagtattagaaaccatatccactcattagtcattactcatcatcatacccacacaaataaaataataggattcgcggtgtgtgtccggaatgcatgaaaatgaaattcgtaaaatcaataaacaagtttgacttttatattttaaaatccgaccaaaatagcctaaagaaaaaacacaactcgaaattctcacgaacaagaaattagagtattgtccgcttcttactttcacttcttttcaaacaaaagattttttagtgtaaacagtagttaagtgaccaagatattattgatcgaagagtattgtactattgtttaatactacagtagttgtacatgcgttttagaatagactcgaatataggacatgtctcattaaaactatttacccactttccgtggattagttgggcttcacttatggaacgtaaatatcttacataaatgaatatacatcgaaccaatggaagaaataataaatataaggatatgatagtatatacaggtttatatctagatattttttttaatcaggagatgatccatagtttaaaatccgaatcatatgataaatatatagcagtcgagtaacccattcaccagatatctatgtagttggataaaatatctatgtctatggtttttcgttccagattatatatacttataaaatcaacaatatagacttttctaactaaatacatataaaacaaaacaattacatactaatcagatatttatagtcttacttgcaagttgcaacaccttttactgaagtgtggcctattttgatcagataaatagtttcggagaactaaaagcttttattatgagagttcataaaataaataattcttttttattattttaaaaaaacaggggtgtgaagcatctgtgctgctagctggatcagcaagtggaccaggagaacaatcatcgatcccgaacctaacactccgtcaacaagcctttgttgtcatcaataacctgcgtgccctcgtccagaaacagtgtggtcaagtcgtctcttgctccgacatcctcgctctcgccgctcgcgattccatcgtccttgtaaccaactatctctcgtctataaatcaatcacacaataatgggtttaaatatggattataacacggcagtgacactaatatgaaccattagcttatacaacatcggtttgtgtcgatatatgcctttttctaattaataaaaaaatgtgtcaatgtagtcaggagggccagactatgctgtgccacttggccgacgtgactcgctagcgtttgcgaccccggaaacgacgttagctaacttaccgccaccgtttgccaacgcaagccagctcatcagcgacttcaacgacagaaacctcaacatcaccgacttagtagcactttccggtggtcacaccatcggaattgcgcattgtccgtctttcacagaccggctctacccaaaccaagatccaaccatgaacaagtctttcgccaacagcctcaaacgcacctgtcccacggcgaactcgagcaacacgcaagtgaatgacataaggagtcctgacgtgtttgacaacaagtactatgttgatctcatgaaccgacaagggctgttcacttccgaccaggatctgttcgttgacaagaggacacgtggcatagtggaaagctttgcgatcgaccagaacttgttttttgatcatttcacggtggcaatgattaagatgggtcagatgagtgtcttgacggggacacaaggggagatccgttccaactgttcagccagaaacaccgcaagtttcatatccgttttggtagaaggcatagtcgaggaagctctttccatgatctaa

SEQ ID NO: 65 - DNA, 2258 bp, Artificial Sequence
  HRP isoenzyme >23190.1
atggcaatgagttattcgatacgtgtcctgacgtttctgatgttgatctcgttaatggcagtgacactgaaccttctgtcaacggcggaagcaaagaagccgaggagagatgttcctatagtgaaaggtctctcttggaacttttaccagagagcatgtccgaaagtggaaaagattatcaaaaaagaactcaaaaaagtcttcaagagagatattggtttagccgcagccatccttcgtatacatttccatgactgcttcgttcaggttctatctttttcgttccctaattttttgtattaaaacctaattaagaacatttaattttatgttctttaagcctactttaaacggttctacgtatgtgattaacataattttcactacttgaaaactccttttcttttagtattagaaaccatatccactcattagtcattactcatcatcatacccacacaaataaaataataggattcgcggtgtgtgtccggaatgcatgaaaatgaaattcgtaaaatcaataaacaagtttgacttttatattttaaaatccgaccaaaatagcctaaagaaaaaacacaactcgaaattctcacgaacaagaaattagagtattgtccgcttcttactttcacttcttttcaaacaaaagattttttagtgtaaacagtagttaagtgaccaagatattattgatcgaagagtattgtactattgtttaatactacagtagttgtacatgcgttttagaatagactcgaatataggacatgtctcattaaaactatttacccactttccgtggattagttgggcttcacttatggaacgtaaatatcttacataaatgaatatacatcgaaccaatggaagaaataataaatataaggatatgatagtatatacaggtttatatctagatattttttttaatcaggagatgatccatagtttaaaatccgaatcatatgataaatatatagcagtcgagtaacccattcaccagatatctatgtagttggataaaatatctatgtctatggtttttcgttccagattatatatacttataaaatcaacaatatagacttttctaactaaatacatataaaacaaaacaattacatactaatcagatatttatagtcttacttgcaagttgcaacaccttttactgaagtgtggcctattttgatcagataaatagtttcggagaactaaaagcttttattatgagagttcataaaataaataattcttttttattattttaaaaaaacaggggtgtgaagcatctgtgctgctagctggatcagcaagtggaccaggagaacaatcatctatcccgaacctaacactccgtcaacaagcctttgttgtcatcaataacctgcgtgccctcgtccagaaacagtgtggccaagtcgtctcttgctccgacatcctcgctctcgccgctcgcgattccatcgtccttgtaaccaactatctctcgtctataaatcaatcacacaataatgggtttaaatatggattataacacggcagtgacactaatatgaaccattagcttatacaacatcggtttgtgtcgatatatgcctttttctaattaataaaaaaatgtgtcaatgtagtcaggagggccagactatgctgtgccacttggccgacgtgactcgctagcgtttgcgaccccggaaacgacgttagctaacttaccgccaccgtttgccaacgcaagccagctcatcagcgacttcaacgacagaaacctcaacatcaccgacttagtagcactttccggtggtcacaccatcggaattgcgcattgtccgcctttcaccgaccggctctacccaaaccaagatccaaccatgaacaagtctttcgccaacagcctcaaacgcacctgtcccacggcgaactcgagcaacacgcaagtgaatgacataaggagtcctgacgtgtttgacaacaagtactatgttgatctcatgaaccgacaagggctgttcacttccgaccaggatctgttcgttgacaagaggacacgtggcatagtggaaagctttgcgatcgaccagaacttgttttttgatcatttcacggtggcaatgattaagatgggtcagatgagtgtcttgacggggacacaaggggagatccgttccaactgttcagccagaaacaccgcaagtttcatatccgttttggaagaaggcatagtcgaggaagctctttccatgatctaa

SEQ ID NO: 66 - DNA, 1887 bp, Artificial Sequence
  HRP isoenzyme >06351
atggttagggcaaatttagtgagcgtgattctgttaatgcatgttattgttgggtttccttttcatgcgaggggcttaagtatgacttattacatgatgagctgtcctatggctgaacaaattgtgaaaaacagtgttaacaatgctcttcaagccgatcccactttagccgcaggtcttatacgtatgttgttccacgactgtttcattgaggtatagtaatgtttttttttcttttcttaattagtttccaatattctaattgtgtcgtttacgtagagaaccccttaaattagttatttttggtcttattaagggatgtgatgcgtcgattctgctagattcaacaaaagacaacactgcggaaaaggattctcctgcgaatctgagtctacgtggctacgagatcatagatgatgcaaaagagaaagttgagaatatgtgtccaggagttgtatcttgcgcagatattgttgccatggctgctagagatgctgtcttttgggtaattatataagctgatcaaaattgacattgtgattacattaagataatctttttaattacttaatataattactagtgattggtttgttgattaggctggtggtccatattatgacataccaaaaggaagatttgatggtaaaagatcgaagatagaagatacaagaaaccttccttcaccttttctcaatgcctctcaactcattcaaacctttggcaaccgtggcttctctccgcaagatgttgttgctctctctggtgagttctttactacatacgtacttggattccgtatatgtcgatatttttgtatatctactaggtcttatagctcaatgaagaaaaatattggaacattaacaagaagaaaattaatttattatgaatacgtttaaaattaaatttctaaattttagaacttaattatatgttttgcatgaccatcagatgtttttaaaaaaaaattgtatatatacaaataacgtaccacaaattaattttagtagcaccatttttaaaaaattttccaaaataccacaaatgttaggctttttcgtaattttaaaattgtggtgtttttggaattctagaattttggtggtaacctagaaaataaaacatcaaatatgtatattttcgtagtttcaaaattgatatattgttttaacttttattaattcaaaatttgttgattaattttttttctatataattatttcactggccaaattaaaaagtagttttggagaacaaaatttgtttatgtttctcatttgtaccttctttttagtaatataagatcttattaatttctataatcatataaaattattattattattctatatgacatttttcacatcacttgggatgaagtccatataaaatgtttagcatgacagtgaatgccccacaagatgttaattttggttgtaacttaacttgtggaatatattataggagcacatacccttggagttgcacgatgctcctccttcaaggctagacttaccactccagattcttcactggactccacttttgcaaacactctcactagaacttgcaatgcgggggacaatgcagagcaaccctttgatgcgacccgcaacgatttcgacaatgcctacttcaatgcgcttcagaggaaatcaggagtcctcttttcagaccagaccttattcaacactccaaggaccaggaatcttgttaatggttatgcccttaatcaagctaagtttttctttgatttccaacaggccatgcgcaaaatgagcaatcttgatgttaaacttggctctcaaggtgaaatacgtcaaaattgccggactattaactaagcctaggccgatttttgtatttatgctcccacctttaatt

SEQ ID NO: 67 - DNA, 1239 bp, Artificial Sequence
  HRP isoenzyme >C1A_optPpDNA2.0 = C1A_new_optPP
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaattgacacctactttctatgataactcttgtccaaatgtttccaacattgttagagatacaatagtgaacgagttgagatccgatccacgtattgctgcctcaatcctcagactgcatttccacgactgttttgttaatggttgtgatgcttccatcttacttgataacacgacatctttcagaacagagaaggacgcatttggtaatgctaattcggcaagaggatttcctgtaatagaccgtatgaaagcagcagtggaatctgcttgtcctagaactgtttcgtgtgctgatttgttaactatcgcagctcaacaatcagtaactcttgccggaggaccctcttggagagttccactaggacgtcgtgactctttacaggcttttctggatctggcaaatgctaacctgccagcaccctttttcactttgccccaacttaaggatagttttagaaatgttggccttaatcgttcaagcgatttggtcgctctatccggtggtcacactttcggcaaaaaccaatgcagattcattatggacagactatacaacttttcgaatactggacttcctgatccaactttgaataccacttacctgcaaacactcaggggtttgtgccctcttaatggtaatctgagtgccttggtggacttcgatttgagaacccctacaatctttgacaacaaatactatgttaacttagaagagcaaaagggtctgattcagagcgatcaggagttgttctcctccccaaatgctacagacactattccattggtaaggagtttcgcaaactcaacgcaaacctttttcaatgccttcgtggaagctatggataggatgggtaacatcaccccgttaaccggtacccaaggacagattagactgaattgtcgtgtcgtcaactcaaattccttgttgcatgacatggtggaagttgtggactttgttagcagcatgtga

SEQ ID NO: 68 - DNA, 1239 bp, Artificial Sequence
  HRP isoenzyme >C1B_optPpDNA2.0 = C1B_15901_ optPp
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaactgacacccacattctacgacactagctgtcctaatgtgagtaacatcgtgagagacatcatcatcaacgaattgcgttccgaccccagaattactgcttcaatcttaagactgcactttcatgattgtttcgttaatggttgcgatgcatccatacttttggataacacaacatcttttctgactgagaaagatgcattgggaaatgctaactcagccaggggttttccaactgttgatagaatcaaagctgctgttgaaagggcatgtcctagaacagtttcatgcgcagacgtactcacaattgcagctcaacagagcgtgaacttggctggaggaccatcctggagagtccctctgggtcgtagagactccctacaggccttccttgacttggctaatgctaatctgccagcaccctttttcacgttgccacagttgaaagatgctttcgcaaaagtaggtttggatcgtccttcagaccttgtagcattgtctggtggtcacactttcggcaaaaaccaatgtagattcatcatggatcgtttgtataactttagtaataccggattgccagatcctacgctcaatacaacatatctacaaactttgcgtcaacagtgtccattgaatggaaatcaaagtgtattagtcgatttcgaccttagaaccccgactgttttcgacaataagtactacgtgaacttaaaggaacagaagggtctaatacagagcgaccaagagctgtttagctcgcctaatgcaacggatacgattccattggtgaggtcctttgctgatggtactcaaaagtttttcaacgcttttgttgaagccatgaacagaatgggtaacatcaccccattaaccggcactcaaggtgagattagattgaattgcagagttgtcaattctaattcccttctgcatgacattgtcgaagttgttgacttcgtttcgtctatgtga

SEQ ID NO: 69 - DNA, 1239 bp, Artificial Sequence
  HRP isoenzyme >C1C_optPpDNA2.0 = C1C_25148_ optPp
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcagttgactcctaccttctacgacaacagttgtcctaatgtttcaaacattgttcgtgacattatcatcaacgagttgcgttccgatccgagaatcgcagcatcgattttgagattgcattttcatgactgtttcgttaatggatgtgatgcttcaatacttttagacaatacaactagctttcgtaccgaaaaggatgcctttggtaacgccaattctgctagaggttttccagttgtggatagaatcaaagccgcagtggaaagggcttgtcccagaacagttagctgcgctgacgtcttgactattgcagcacaacaatccgtgaatttggctggcggtccaagctggagagtgccactgggtagaagagattcacgtcaggcattcctggacctagccaatgctaaccttcctgcaccatctttcacactgccagagttgaaagctgcttttgctaatgttggtttgaatagaccgtccgacctcgtggcattgtcaggtggacacacatttggcaaaaaccaatgtagattcatcatggatagactctacaatttctctaatactggattacccgaccctactctgaacactacctacttacagacactgagacaacaatgtcctagaaatggtaatcaatctgtgctagttgatttcgatttgagaactccaactgtatttgacaataagtactatgtcaacttaaaggaacaaaagggtcttatccaatccgaccaggagttgttcagttccccaaatgccacagacacgattcctcttgtaagatcctatgctgacggtacacagacctttttcaacgcatttgttgaggctatgaacaggatgggaaacattactcccctaacgggtactcaaggagaaattcgtcttaactgcagggtagttaactccaatagtctgctgcacgatatagtcgaggtcgtcgatttcgtatcttcgatgtga

SEQ ID NO: 70 - DNA, 1239 bp, Artificial Sequence
  HRP isoenzyme >C1D_optPpDNA2.0 = C1CvarD_optPp
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcagttgactcctaccttctacgacaacagttgtcctaatgtttcaaacattgttcgtgacattatcatcaacgagttgcgttccgatccgagaatcgcagcatcgattttgagattgcattttcatgactgtttcgttaatggatgtgatgcttcaatacttttagacaatacaactagctttcgtaccgaaaaggatgcctttggtaacgccaattctgctagaggttttccagttgtggatagaatcaaagccgcagtggaaagggcttgtcccagaacagttagctgcgctgacgtcttgactattgcagcacaacaatccgtgaatttggctggcggtccaagctggagagtgccactgggtagaagagattcacgtcaggcattcctggacctagccaatacaaaccttcctgcaccatctttcacactgccacaattgaaagctgcttttgctaatgttggtttgaatagaccgtccgacctcgtggcattgtcaggtggacacacatttggcaaaaaccaatgtagattcatcatggatagactctacaatttctctaatactggattacccgaccctactctgaacactacctacttacagacactgagacaacaatgtcctagaaatggtaatcaatctgtgctagttgatttcgatttgagaactccaactgtatttgacaataagtactatgtcaacttaaaggaacaaaagggtcttatccaatccgaccaggagttgttcagttccccaaatgccacagacacgattcctcttgtaagatcctatgctgacggtacacagacctttttcaacgcatttgttgaggctatgaacaggatgggaaacattactcccctaacgggtactcaaggagaaattcgtcttaactgcagggtagttaactccaatagtctgctgcacgatatagtcgaggtcgtcgatttcgtatcttcgatgtga

SEQ ID NO: 71 - DNA, 1239 bp, Artificial Sequence
  HRP isoenzyme >C2_optPpDNA2.0 = C2_04627_optPp
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcagttgagtccatcattctacgataagacctgcccccaagtctttgacatcgcaaccaataccatcaaaaccgccttgcgttcagatccgagaatcgctgcttcaatcttgcgtctacatttccacgattgtttcgtgaatggatgtgacgcttcaatcttacttgacaatactacgtcttttagaactgagaaagacgcttttggtaacgcacgtagtgctagaggattcgatgttatcgacacgatgaaagcagccgtcgagaaggcttgtccaaaaacagtttcctgcgccgatttgttggcaattgctgcacaaaagtcagtggtcttggcaggtggtcctagctggaaggttccatctggtagaagagactcattacgtggtttcatggacctggccaatgacaacctacccggtccatcaagcaccttgcaagtgctgaaggacaagtttcgtaacgtaggtttagatagaccttcggacctggtcgctttgtcaggtggtcacacttttggcaaaaaccaatgccagtttatcatggatagactttacaacttttcaaattcaggaaagccagatcctaccttggacaagtcatacctctccactttgaggaagcagtgtccccgtaacggtaacttgtcagtgctggtggatttcgatctgagaacacccactatcttcgacaacaagtactatgttaatctgaaggagaacaaaggactaatccaatctgatcaggaactgttcagtagcccggatgcttcggacacgatccctcttgtaagagcttacgcagatggtcaaggcaagtttttcgacgccttcgttgaggccatgatcagaatgggtaacctttcaccatcaacaggaaaacaaggtgagattcgtttgaattgtagagttgttaactccaaacctaagataatggacgtggttgacacgaatgacttcgcttccagtatctga

SEQ ID NO: 72 - DNA, 1230 bp, Artificial Sequence
  HRP isoenzyme >C3_optPpDNA2.0 = C3_P17180_optPp
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcagttgaggcctgatttctatttccgtacatgtccgtccgtattcaatatcatcggtgacattatcgttgacgagttgagaactgacccacgtatagcagcatccctgttgagattgcacttccatgattgctttgtcagaggttgtgacgcctccatcttgctggacaactctacgagctttagaactgagaaggatgcagctccaaatgcaaattcggctcgtggcttcggagtcattgatagaatgaaaacttcattagaaagggcttgtcctagaacagttagctgtgctgacgttctaaccattgcttctcaaatctccgtcttgctatctggaggtccttggtggccagttcctctgggacgtagggatagtgtggaagcctttttcgacttggctaacactgccttgcctagcccctttttcactttagcacaattgaaaaaggccttcgctgatgttggacttaatcgtccatcagacttagtcgctctatcaggtggtcatacctttggtagggctcaatgtcagttcgttacaccccgtttgtataactttaacggtacaaataggccagatcctacgttggacccaacttacctggttcaattgagagcactttgcccacagaacggaaatggaaccgttctcgtaaactttgacgtcgtaacgccgaatactttcgatagacaatactataccaatctgagaaatggtaagggtcttattcaatctgatcaggaattgttttccactcccggtgcagatacaatcccattagtgaatctgtactcttcgaacacatttgcctttttcggcgctttcgtagatgctatgattagaatgggtaatcttagacctctgactggtactcaaggtgagataagacaaaactgcagagttgtgaacagtcgtattcgtggtatggagaatgacgatggagtggtgtcctcaatctga

SEQ ID NO: 73 - DNA, 1185 bp, Artificial Sequence
  HRP isoenzyme >A2A_optPpDNA2.0 = A2_ manualassembly_optPP
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaattgaatgctacgttctactctggtacttgtccaaatgcttctgcaattgtccgttccacgattcaacaagcatttcagtccgatacccgtattggagcttccctgattagattacattttcatgattgttttgtgaatggttgtgacgcttcaatacttctagacgattcaggttcaatccagtcagagaaaaacgctggacccaatgctaatagcgccagaggtttcaacgtggtagacaacattaagactgctttagagaacacttgtccaggtgttgtttcctgttctgacattctggccttggcatctgaagcatccgtcagtttgactggaggaccctcgtggacagtgctgttgggtaggagagattcactgacagcaaacttggcaggtgcaaatagcgcaatcccatcccctttcgaaggtttatccaatatcacaagcaaattctccgcagttggcctgaatacaaatgacttggtagctctttctggagcacacaccttcggtagagctaggtgcggcgttttcaacaatcgtcttttcaacttctcaggtacaggaaaccctgatcctactttgaattctaccttgttgagctccttacaacagttgtgtccacaaaacggctcggccagtacaataactaacctcgatctgtctaccccagatgcctttgataacaattactttgccaatcttcaaagcaataacggtttgctccaatcagaccaagagttgttttcgacaactggttccgctacaatcgctgttgttacttcctttgctagtaatcagactttgtttttccaggcttttgctcaatcgatgatcaatatgggtaacattagtccgctgactggtagtaacggtgaaatcagactagactgcaaaaaggtcaatggatcttga

SEQ ID NO: 74 - DNA, 1185 bp, Artificial Sequence
  HRP isoenzyme >A2B_optPpDNA2.0 = A2var_ manualassembly_optPp
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaattgaatgctacgttctactctggtacttgtccaaatgcttctgcaattgtccgttccacgattcaacaagcatttcagtccgatacccgtattggagcttccctgattagattacattttcatgattgttttgtgaatggttgtgacgcttcaatacttctagacgattcaggttcaatccagtcagagaaaaacgctggacccaatgctaatagcgccagaggtttcaacgtggtagacaacattaagactgctttagagaacacttgtccaggtgttgtttcctgttctgacattctggccttggcatctgaagcatccgtcagtttgactggaggaccctcgtggacagtgctgttgggtaggagagattcactgacagcaaacttggcaggtgcaaatagcgcaatcccatcccctttcgaaggtttatccaatatcacaagcaaattctccgcagttggcctgaatacaaatgacttggtagctctttctggagcacacaccttcggtagagctaggtgcggcgttttcaacaatcgtcttttcaacttctcaggtacaggaaaccctgatcctactttgaattctaccttgttgagctccttacaacagttgtgtccacaaaacggctcggccagtacaataactaacctcgatctgtctaccccagatgcctttgataacaattactttgccaatcttcaaagcaataacggtttgctccaatcagaccaagagttgttttcgacaactggttccgctacaatcaccgttgttacttcctttgctagtaatcagactttgtttttccaggcttttgctcaatcgatgatcaatatgggtaacattagtccgctgactggtagtaacggtgaaatcagactagactgcaaaaaggtcaatggatcttga

SEQ ID NO: 75 - DNA, 1188 bp, Artificial Sequence
  HRP isoenzyme >E5_optPpDNA2.0 = E5_ manualassembly_optPp
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaattgcgtcccgacttttactccaggacgtgtccgtcagttttcaacattatcaaaaacgtcatcgtcgatgaactgcagactgatcctagaatagctgcctccatcttgagactgcattttcacgactgtttcgttagaggttgcgatgcttcaattctactcgatacaagcaagagtttcagaactgagaaggacgcagctcctaatgttaactctgctaggggcttcaacgtgattgacaggatgaaaactgcccttgaaagagcatgtccacgtactgtctcttgtgcagatatactcaccatcgcatcacaaatctccgttttactgtccggtggtccatcttgggctgttccattaggtcgtagagattcggttgaggcctttttcgacttggctaatacagctctacctagtccctttttcacattagcacaattgaaaaaggcattcgctgatgtgggattgaataggccatctgacctagtcgcattgtctggaggtcatactttcggtagagctagatgtttgttcgtaacagcaagattgtataacttcaatggtacaaacagaccggatccaaccctgaatccttcctacctggctgaccttcgtagattatgccctcgtaatggtaatggtacggtgttggtgaactttgatgtaatgacgccaaatacttttgacaaccagttctacaccaatcttagaaatggaaaaggcttgattcagtcagatcaagagttgttttcgactcctggtgccgacacaattccacttgtgaatctgtatagtagcaacactctttcctttttcggagcttttgcagacgctatgattagaatgggaaatcttagacctttgacaggaacacagggtgaaatccgtcaaaactgccgtgttgtcaatagcaggtga

SEQ ID NO: 76 - DNA, 1239 bp, Artificial Sequence
  HRP isoenzyme >01805_optPpDNA2.0 = 01805B1_ optPp
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaattgacacctacgttctacgactccacctgtccaagcgttttctctatcgtgagagacacgatcgtaaacgagttacgttcggacccaaggatagctgcctccattttgagattgcatttccatgactgctttgtcaacggttgtgatgcctcaatcctattggataacacaacttccttcagaactgaaaaagatgcagctccaaatgctaatagtgctagaggtttcccagttattgatacaatgaaagcagcagttgaaagagcttgtccacgtacagtgagctgtgcagatttgttgaccatcgctgcccaacaatcggtgaacttggctggaggaccttcctggcgtgttcctttaggtagaagagacagtgtacaagcctttttcgatctggctaatacaaatctaccagctccctttttcactttacctcagttgaaggccagcttttcaaatgttggattagataggccagaggatcttgtcgctttatcaggaggtcacacattcggcaaaaaccaatgtcagtttatcatggacaggctatacaatttctctaatacaggtctacctgaccccactctcaacactacttacctgcaaacattgagagtacaatgccctcgtaatggtaaccaatcagttctcgtggattttgacttgcgtactccgactgtgtttgataacaagtattacgtcaatcttaaggagcacaaaggtctgattcaaaccgaccaagagttgttttctagtcccaatgcagctgatactattccacttgtcagatcttatgcagatggtacacagaagtttttcaatgcattcatggaagctatgaatagaatgggcaacatcactccactgacaggtacccagggacaaattcgtcagaactgtagagttatcaattctaattccctgcttcatgacattgtagaaatagttgattttgtctctagcatgtga

SEQ ID NO: 77 - DNA, 1230 bp, Artificial Sequence
  HRP isoenzyme >22684.1_optPpDNA2.0 = 22684B2_optPp
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctaaacttagaccagacttttacttaaagacatgcccatctgtgtttcaaatcattggtaatgtcattgttgatgaactgcagtccgacccacgtatagcagcttcgctcttaagattgcatttccacgattgtttcgtgcgtggatgcgatgcaagtgtcctattggacaatagcactagttttcaatcagaaaaagatgctgctcctaacgctaactccgcaagaggtttcgacgtggtggatagaatgaaagctgcactagaaaaggcttgtccaggtacagtctcctgtgcagatgtcttagctatatcggcacaaatctcagttctactgtctggtggtccttggtggcccgtattgctgggcaggagggatggagttgaggcctttttcgacttagcaaatactgctttgcctaatcctttcgcaccattgaccgaactgaaggagaagtttgccgacgttggtcttaagagggcctctgatttggttgctttgtcaggagctcatacattcggtagagcccaatgtctgttggtcacacctagattgtacaacttttccggtaccaacaaaccggatcccaccctcaatccaagctacttagtagagctgcgtagattgtgccctcaaaatggaaatggtactgtacttctgaacttcgatcttgtgacgcctaatgcattcgaccgtcagtactatactaatcttcgtaatggaaaaggacttattcaatccgatcaagagttgttttctacacccggtgctgacactatccccttggttaatctgtatagcaaaaacacgttcgcctttttcggcgcttttgttgacgccattatcagaatgggtaacattcaaccattaactggaacacagggtgaaatcagacagaattgtcgtgtggttaactccagaatcaaaggtatggagaacgacggaggtgttgtatcatccatttga

SEQ ID NO: 78 - DNA, 1230 bp, Artificial Sequence
  HRP isoenzyme >22684.2_optPpDNA2.0 = 22684B2gvar_optPp
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctaaacttagaccagacttttacttaaagacatgcccatctgtgtttcaaatcattggtaatgtcattgttgatgaactgcagtccgacccacgtatagcagcttcgctcttaagattgcatttccacgattgtttcgtgcgtggatgcgatgcaagtgtcctattggacaatagcactagttttcaatcagaaaaagatgctgctcctaacgctaactccgcaagaggtttcgacgtggtggatagaatgaaagctgcactagaaaaggcttgtccaggtacagtctcctgtgcagatgtcttagctatatcggcacaaatctcagttctactgtctggtggtccttggtggcccgtattgctgggcaggagggatggagttgaggcctttttcgacttagcaaatactgctttgcctaatcctttcgcaccattgaccgaactgaaggagaagtttgccgacgttggtcttaagagggcctctgatttggttgctttgtcaggagctcatacattcggtagagcccaatgtctgttggtcacacctagattgtacaacttttccggtaccaacaaaccggatcccaccctcaatccaagctacttagtagagctgcgtagattgtgccctcaaaatggaaatggtactgtacttctgaacttcgatcttgtgacgcctaatgcattcgaccgtcagtactatactaatcttcgtaatggaaaaggacttattcaatccgatcaagagttgttttctacacccggtgctgacactatccccttggttaatctgtatagcaaaaacacgttcgcctttttcggcgcttttgttgacgccattatcagaatgggtaacattcaaccattaactggaacacagggtgaaatcagacagaattgtcgtgtggttaactccagaatcagaggtatggagaacgacgacggagttgtatcatccatttga

SEQ ID NO: 79 - DNA, 1158 bp, Artificial Sequence
  HRP isoenzyme >01350_optPp DNA2.0 = my01350_ optPp
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaattgacacccaatttctatagtaccagttgtccgaatcttctgtcaacagtccaatccgctgtgaaaagtgccgttaactcagaagcaaggatgggtgcttccatcgtcagattgtttttccacgattgtttcgtcaacggttgcgatggtagcatcctgttggatgatacttcatcctttactggtgagcaaaatgccaatccaaatagaaactctgctagaggattcaatgttattgacaatatcaaggcagctgtcgaaaaggcttgtcctggagtggtttcctgcgctgatattctcgcaatcgcagctcgtgactcagttgttgtgctgggaggacccaattggacggtaaaggtgggtagaagggacgccagaacagcttcgcaagctgctgccaacagcaacattcctgccccaaccagctcattgtcccagttgatatcttcattttctgcagttggcttatcaactagagatatggttgctctatccggtgctcatacaattggccagtcgagatgcacttcctttagaacacgtatctacaatgagactaacataaacgcagctttcgctactaccaggcaacgtacttgtcctagaactagcggttcgggtgatggcaatctggcacctttggacgtcaccactgctgcatcttttgacaataactatttcaaaaacctaatgacgcagcgtggtttgcttcactctgaccaggaattgttcaatggtggttctacggattctatcgtaagaggttacagtaacaatccatcctcctttagttccgattttgctgctgcaatgattaagatgggagacatctccccattaacaggatcaagcggagagattagaaaagtttgtggtcgtacaaactga

SEQ ID NO: 80 - DNA, 1176 bp, Artificial Sequence
  HRP isoenzyme >02021_optPpDNA2.0 = my02021_ optPp
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctcaacttcagatgaatttctacgctaagtcctgtcctaatgccgagaaaatcatctccgaccacatccaaaaacacatcccttccggtccatcactagcagctccactgattagaatgcattttcatgattgtttcgtccgtggttgtgatggttccgttttgatcaacagcacctcaggaaatgccgaaaaagactctgcaccaaatcttactctcagaggtttcggcttcgttgaacgtatcaaaactttgttagaagcagaatgtccaaagactgtcagttgcgcagacattatcgcattaactgctagagatgctgtcgtggctacaggaggtccaagttggaaagttcccactggtagacgtgatggtaggatttccaacacaacggaggccttgaataacattcctccgcctacctctaactttactactttacagaggctgttcgcaaaccagggtttgaatctgaaggaccttgtattgctgtccggtgcccacacaataggtgtgtctcattgttcatccatgaataccaggctctataacttctcaactaccgtgaaacaagatcctagtttggatagcgagtatgcagctaacttgaaggctaacaaatgcaaatcattgaatgacaataccacaatcctggaaatggacccaggaagctctaaaacatttgatctgtcctactacagattggtactaaagagaagaggattgtttcaatcagattcggcattaacaactaacagcgctacattgaagatgatcaatgaccttgttaatggacctgagaaaaagtttttgaaggcttttgccaagtccatggagaaaatgggaagagttaaggttaagacgggcagtgctggagtgatacgtacaagatgttctgttgctggttcgtga

SEQ ID NO: 81 - DNA, 990 bp, Artificial Sequence
  HRP isoenzyme >03523_optPpManual = 03523_optPp
ctcgagaagagagaggccgaagctgctgaactgaagtccttgtccttgattctgcttttcactttgctgactactaccattgagtccagactgactaccaacttctactctaagtcctgtcctcgtttctttgacattgttagagacactatctccaacaaacagattaccactcctaccaccgctgctgcaactattcgtctgttcttccacgattgttttcctaatggttgcgacgcatccatcttgatttcttctactgccttcaatactgctgaaagagactcctctatcaacttgtctcttcctggagacggattcgacgttatcgtcagagctaagactgccattgagcttgcatgtcctaacactgtgtcttgttccgacatcattaccgttgcaaccagagacttgttagttaccgtcggtggtccatactacgacgtgtatcttggtcgtagagattccagaatctctaagtcttctttgcttaccgacttgttaccattgccatcatctcctatctccaagaccattagacagtttgaatctaagggtttcaccattcaagagatggtggctttgtctggtgcccactctattggtttctcccattgtaaagagtttgtcaacagagttgctggtaacaatactggatacaacccaagattcgctcaagccttgaagcaggcttgttctaactacccaaaagacccaaccttgtctgttttcaacgacatcatgactccaaacagattcgacaacatgtactatcaaaacattcctaagggtttgggtttgttagagtcagatcacggactttactctgatccaagaaccagaccatttgttgacttgtacgctagagatcaagaccttttcttcaaggactttgccagagctatgcagaagttatctttgtttggtgtcaaaaccggtagacgtggagagatcagacgtagatgcgatgcaatcaactaatgcggccgc

SEQ ID NO: 82 - DNA, 921 bp, Artificial Sequence
  HRP isoenzyme >04663_optPpManual = syn04663_ optPp
ctcgagaagagagaggccgaagctgctgctacctcatcatctactacttgtgacggattgttcatcatttctctgcttgtcattgcttcctctttgtttggtacttcttctgctcagttgaacgcaaccttctactccggtacttgccctaacgcctctgctatcgttagatccaccattcaacaagcacttcagtctgaccctagaattggtgcttccttgatccgtttgcacttccatgactgcttcgtcaatggatgcgacggttctttgcttctggacgatactggatctattcaatccgagaagaacgcaccagctaacgctaactcagctagaggtttcaatgtcgttgacgacatcaagaccgctcttgagaacgcttgtccaggtattgtttcttgctcagacatcttagctcttgcttctgaggcttccgtttcacttgctggaggtccatcttggactgtcttggttggaagaagagatggtcttactgccaacttgtccggtgccaactcttctttgccatcacctttcgagggattgaacaacattacctctaagttccttgccgttggattgaacactactgacgttgtggtcttgtctggtgcccacacctttggtagaggtcagtgtgttactttcaacaaccgtttgttcaacttcaatggaaccggatctcctgaccctaccttgaactctactcttttgtcatccttgcagcaaatctgtccacagaacggttctggttctgccattaccaacttggacctgactactcctgacgcattcgattccaactactatactaacctgcaatccaacaacggtttgcttcaatctgaccaagagttgttctcaaacaccggttctccaaccattgctatcgtgatcttgtgtaagtaatgcggccgc

SEQ ID NO: 83 - DNA, 996 bp, Artificial Sequence
  HRP isoenzyme >05508_optPpManual = syn05508_ optPp
ctcgagaagagagaggccgaagctggattgatccgttctctgtgtgtgttcattaccttcttgtcttgtatcatttcttcagcccacggtcaagctatctccatttccattaccatcagaattggtttctaccttactacctgtcctactgccgagatcattgttagaaatgcagttagagctggtttcaactctgacccaagaattgcaccaggtatccttagaatgcactttcacgactgttttgttcagggttgtgacggttctgtcctgatttcaggatctaacaccgagagaaccgcagttccaaacttgtctcttagaggatttgaagttatcgaaaacgctaagacccaattggaagcagcttgcccaggagttgtttcctgtgctgacatcttggccttagctgctagagatactgttgttctgacccgtggtattggttggcaggtcccaaccggaagaagagacggtcgtgtctccgtggcctccaatgcaaacaacttgcctggtcctagagactccgttgctgttcaacagcaaaagttttctgcccttggattgaataccagagacttggtcgttctggctggtggtcatactttgggtactgctggttgcggtgtcttcagagatagactgttcaacaacactgacccaaacgtggaccaacctttcttaacccagttgcaaactaagtgcccacgtaatggtgatggttccgttagagtggacttagatactggttctggtactaccttcgataactcttacttcatcaacttgtccagaggtagaggtgttttggaatctgaccacgttctttggactgaccctgccaccagacctattgtccaacaacttatgtcttcctctggtaacttcaacgcagagtttgctagatcaatggtgaagatgtctaacatcggagttgtcactggaactaacggtgagattagaaaagtctgttcagctatcaactaatgcggccgc

SEQ ID NO: 84 - DNA, 975 bp, Artificial Sequence
  HRP isoenzyme >06351_optPpManual = 06351_optPp
ctcgagaagagagaggccgaagctgttagagcaaaccttgtctctgtcatcttactgatgcacgtcattgtcggtttcccattccacgccagaggtttgtccatgacttactacatgatgtcctgccctatggcagaacagatcgtcaagaactctgtcaacaatgccttgcaagctgatcctaccttggctgctggattgattcgtatgcttttccacgattgcttcatcgagggttgcgacgcctctattctgttggattctaccaaagacaataccgctgagaaagactctccagccaacctgtccctgagaggatatgagatcattgacgatgctaaggaaaaggttgagaacatgtgtccaggagttgtgtcttgtgcagacattgtggctatggctgccagagacgctgttttctgggctggtggaccttactatgacattccaaagggtagattcgacggtaagcgttcaaagatcgaagacactagaaacttgccttctccattcctgaatgcctctcaactgattcaaacctttggaaaccgtggtttctctccacaagatgttgtcgctttgtctggtgctcatactttgggtgttgctagatgttcttcattcaaagctcgtctgaccactcctgactcttccttggactctacttttgccaacacccttaccagaacctgtaacgctggtgataacgctgagcagccttttgatgcaactagaaacgatttcgacaacgcctacttcaacgcacttcaaagaaagtctggtgtcttgttttccgaccaaactttgttcaacacccctagaactcgtaacttagtcaatggttacgcattgaaccaggcaaagttcttctttgactttcagcaagctatgagaaagatgtcaaaccttgacgttaagttgggttctcaaggtgagatcagacagaattgtagaaccatcaactaatgcggccgc

SEQ ID NO: 85 - DNA, 1110 bp, Artificial Sequence
  HRP isoenzyme >23190_optPpManual = syn23190_ optPp
ctcgagaagagagaggccgaagctgccatgtcttactctatcagagtcttgactttccttatgctgatttcattgatggcagtcactttgaacttgctttctactgctgaagccaagaagccacgtagagacgttccaattgtgaagggtttgtcatggaacttctaccaaagagcttgtccaaaggttgagaagatcatcaagaaagagctgaagaaggttttcaaacgtgacattggtttggctgctgccatccttcgtattcactttcacgattgctttgttcaaggttgtgaggcatccgtcttgcttgctggttctgcttccggacctggtgaacagtcttctattccaaacttgaccctgagacaacaggcattcgttgtcatcaacaatctgagagccttggttcagaagcaatgtggtcaggttgtttcttgttccgacatcttagcacttgctgctagagactccattgttctgtctggtggtcctgactacgctgttcctttgggtagaagagactctcttgccttcgcaactccagagactactttggctaaccttccacctcctttcgctaacgcttctcaactgatttccgacttcaacgaccgtaacttgaacatcaccgatcttgttgctctgtccggaggacacactatcggaattgcccactgcccttctttcactgacagactgtacccaaaccaggacccaactatgaacaagtctttcgctaactccttgaagagaacttgtccaaccgcaaactcctctaacacccaagtgaatgacatcagatcccctgacgtgtttgacaacaagtactatgtggacttgatgaacagacaaggtttgttcacctctgaccaggacttgttcgttgacaagagaactcgtggtattgtcgaatctttcgccattgatcagaacttgttcttcgaccacttcaccgtcgcaatgatcaagatgggtcagatgtccgtcttgactggaactcaaggtgagattcgttctaactgctccgctagaaacactgcatctttcatctccgttcttgttgaaggaatcgtcgaggaagcactttctatgatctaatgcggccgc

SEQ ID NO: 86 - DNA, 1008 bp, Artificial Sequence
  HRP isoenzyme >22489.1_optPpManual = 22489_1_ optPp
ctcgagaagagagaggccgaagctgagtttgttagatccttatgcgtcttcattacctttctgggttgcttgatttcctctgctcatggacaggctgccgctagaagaccaggtccaatctctggtactagaattggtttctacttaaccacttgccctactgccgagatcattgttagaaatgccgttcgtgctggattcaactctgacccaagaattgctcctggaatcctgagaatgcactttcacgactgtttcgtgttgggttgtgacggttctgtcctgatttccggttctaacactgagagaactgctgttcctaacttgaaccttcgtggattcgaagtgatcgacaatgccaagacccaattggaagccacttgtccaggtgttgtgtcctgtgcagacattcttgccttagccgctagagacaccgttgtcttgactagaggacttggttggcaagttcctaccggtcgtagagatggtagagtttctgtcgcatccaatgcaaacaacttaccaggtccacgtgactctgttgctgttcagcaacaaaagttctccgctgttggtttgaacaccagagaccttgtggttttggcaggtggtcacactatcggtactgccggttgtggtgtgtttagagatcgtctgttcaacaatactgaccctaacgttaaccaacttttcttgacccaattgcaaacccaatgccctcagaacggtgacggagctgtgagagtcgatcttgacactggttctggtactaccttcgataactcctacttcatcaacctgtctcgtggtagaggagttttggagtctgaccacgtcttgtggactgatccagctaccagaccaattgtccaacagttgatgtcaccaagaggtaacttcaacgccgagtttgctcgttctatggtccgtatgtccaacatcggagttgtcaccggagctaacggtgagattagaagagtgtgttcagcagtcaactaatgcggccgc

SEQ ID NO: 87 - DNA, 1008 bp, Artificial Sequence
  HRP isoenzyme >22489.2_optPpManual = 22489_2_ optPp
ctcgagaagagagaggccgaagctgagtttgttagatccttatgcgtcttcattacctttctgggttgcttgatttcctctgctcatggacaggctgccgctagaagaccaggtccaatctctggtactagaattggtttctacttaaccacttgccctactgccgagatcattgttagaaatgccgttcgtgctggattcaactctgacccaagaattgctcctggaatcctgagaatgcactttcacgactgtttcgtgttgggttgtgacggttctgtcctgatttccggttctaacactgagagaactgctgttcctaacttgaaccttcgtggattcgaagtgatcgacaatgccaagacccaattggaagccacttgtccaggtgttgtgtcctgtgcagacattcttgccttagccgctagagacaccgttgtcttgactagaggacttggttggcaagttcctaccggtcgtagagatggtagagtttctgtcgcatccaatgcaaacaacttaccaggtccacgtgactctgttgctgttcagcaacaaaagttctccgctgttggtttgaacaccagagaccttgtggttttggcaggtggtcacactatcggtactgccggttgtggtgtgtttagagatcgtctgttcaacaatactgaccctaacgttaaccaacttttcttgacccaattgcaaacccaatgccctcagaacggtgacggatctgtgagagtcgatcttgacactggttctggtactaccttcgataactcctacttcatcaacctgtctcgtggtagaggagttttggagtctgaccacgtcttgtggactgatccagctaccagaccaattgtccaacagttgatgtcaccaagaggtaacttcaacgccgagtttgctcgttctatggtccgtatgtccaacatcggagttgtcaccggagctaacggtgagattagaagagtgtgttcagcagtcaactaatgcggccgc

SEQ ID NO: 88 - DNA, 990 bp, Artificial Sequence
  HRP isoenzyme >04791(03523)_optPpManual = 04791_optPp
ctcgagaagagagaggccgaagctgctgagttgaagtctctttccttgatcttactgtttacccttcttactaccactattgagtcaagattgactaccaacttctactctaagtcttgtcctagattctttgacattgtcagagataccatctccaacaagcagattaccactcctactaccgctgccgcaactatcagactgttctttcacgattgtttcccaaacggttgtgacgcctcaattctgatctcttccactgctttcaacaccgcagaaagagattcatccatcaacttgtctttgccaggtgatggtttcgacgtcattgttagagctaagaccgctattgagttagcctgtcctaacactgtctcttgttccgacatcatcaccgttgcaactcgtgacttgcttgttaccgtgggtggaccatactacgacgtttaccttggtagaagagattcaagaatctctaagtcctctttgctgactgacttacttcctctgccatcttctccaatctccaagactattcgtcaattcgagtctaagggtttcactattcaggaaatggtggctctttctggtgctcactctattggattctcacactgtaaggagttcgttaacagagttgctggtaacaacactggttacaacccacgttttgctcaagctctgaaacaagcctgttccaactaccctaaggacccaactttgtccgttttcaacgacatcatgacccctaacagattcgataacatgtactaccagaacattccaaagggtcttggtttgttggagtctgaccacggactttactctgatccacgtactagaccttttgtggacttgtacgctcgtgaccaagacttgttcttcaaggacttcgccagagccatgcaaaagttgtccttgttcggtgtcaagactggaagaagaggtgaaatcagacgtagatgtgacgctatcaattaatgcggccgc

SEQ ID NO: 89 - DNA, 1038 bp, Artificial Sequence
  HRP isoenzyme >06117_optPpManual = 06117_optPp
ctcgagaagagagaggccgaagctgccagaattggttccttcttggttgtcatttcacttgcttgcgtgcttaccttgtgcatttgtgacgacgagtccaactacggtggtcagggtaagttgttccctggattctactcttcatcctgtccaaaggctgaggagattgttagatccgttgtggccaaagctgtcgctagagagactagaatggccgcttctcttatgagattgcactttcacgactgtttcgttcaaggttgtgatggttctctgttgcttgactcttctggttccatcgttactgagaagaactccaatcctaactctcgttctgctagaggtttcgaagtcgttgacgagatcaaggctgctttagagaacgaatgtccaaacactgtgtcatgtgctgacgcactgactcttgctgccagagactcctctgtcttgactggaggaccatcttggatggttccattaggacgtagagactcaacttctgcatccttgtctggttccaacaacaacattccagctccaaacaataccttcaacaccattctgtccagattcaactctcagggtttagaccttactaacgttgttgcattgtccggttctcacaccattggtttctctcgttgtacctcattcagacaaagactgtacaaccaatctggtaacggttctccagacactaccttagaacagtcttacgcagctaatctgcgtcacagatgtcctagatccggaggagatcagaacttgtctgagttggacatcaactctgctggacgttttgacaactcctacttcaagaacttgatcgagaacatgggattgcttaactccgatcaggtcttgttctcttccaatgacgagtcccgtgaattggtcaagaagtacgctgaggatcaggaagagttcttcgaacaattcgctgagtctatggtgaagatgggtaacatttctcctttgactggttcttccggacaaatcagaaagaactgccgtaagatcaactcttaatgcggccgc

SEQ ID NO: 90 - DNA, 1002 bp, Artificial Sequence
  HRP isoenzyme >17517.1_optPpManual = 17517_ 1_optPp
ctcgagaagagagaggccgaagctggtcgtggatacaatctgttgctgatcttggtgaccttcttggtcttggttgctgctgttactgccagaagacctagagttggtttctacggtaacagatgcagaaaggtcgagtcaattgtcagatccgttgtgagatcccacttcagatgcaaccctgcaaatgctccaggtattctgagaatgtactttcacgactgtttcgtgaacggttgtgatggttcaatcttacttgctggaaacacttctgagagaaccgctggtcctaacagatccttgagaggattcgaagctattgaggaagcaaagactcgtttagagaacgcttgtcctaacaccgtctcttgcgctgacatcttgaccttggctgccagagacgcagtggtttggactggtggtaaaggttggtctgtccctttaggtagattggacggaagacgttctgaagcctcagatgttaacttgccaggtccttctgacccagttgcaaaacagaagcaagacttcgctgccaagaacctgaacactttagacttggttaccctggttggaggtcataccattggtactgccggttgtggtttagtcagaggtcgtttcttcaacttcaacggtactggtcaaccagacccatctattgacccatcttttgttcctttggtgcaggctcgttgcccacaaaacggtaacgctactactagagtcgatttggatactggatctgctggtgacttcgatacctcttacctttccaacgtcagatcctctcgtgttgtcttgcagtctgatttggttctttggaaggacaccgaaactagagccatcattgagagattgttgggtcttagacgtccagtgttaagatttggatctgagttcggtaagtctatgaccaagatgtcccttatcgaagttaagaccagattgtctgacggtgagattcgtagagtttgttccgccatcaactaatgcggccgc

SEQ ID NO: 91 - DNA, 1002 bp, Artificial Sequence
  HRP isoenzyme >17517.2_optPpManual = 17517_ 2_optPp
ctcgagaagagagaggccgaagctggtcgtggatacaatctgttgctgatcttggtgaccttcttggtcttggttgctgctgttactgccagaagacctagagttggtttctacggtaacagatgcagaaaggtcgagtcaattgtcagatccgttgtgagatcccacttcagatgcaaccctgcaaatgctccaggtattctgagaatgcactttcacgactgtttcgtgaacggttgtgatggttcaatcttacttgctggaaacacttctgagagaaccgctggtcctaacagatccttgagaggattcgaagctattgaggaagcaaagactcgtttagagaacgcttgtcctaacaccgtctcttgcgctgacatcttgaccttggctgccagagacgcagtggtttggactggtggtaaaggttggtctgtccctttaggtagattggacggaagacgttctgaagcctcagatgttaacttgccaggtccttctgacccagttgcaaaacagaagcaagacttcgctgccaagaacctgaacactttagacttggttaccctggttggaggtcataccattggtactgccggttgtggtttagtcagaggtcgtttcttcaacttcaacggtactggtcaaccagacccatctattgacccatcttttgttcctttggtgcaggctcgttgcccacaaaacggtaacgctactactagagtcgatttggatactggatctgctggtgacttcgatacctcttacctttccaacgtcagatcctctcgtgttgtcttgcagtctgatttggttctttggaaggacaccgaaactagagccatcattgagagattgttgggtcttagacgtccagtgttaagatttggatctgagttcggtaagtctatgaccaagatgtcccttatcgaagttaagaccagattgtctgacggtgagattcgtagagtttgttccgccatcaactaatgcggccgc

SEQ ID NO: 92 - DNA, 1026 bp, Artificial Sequence
  HRP isoenzyme >08562.4_optPpManual = 08562_ 4_optPp
ctcgagaagagagaggccgaagctgccagattgacctctatcttacttttgctgtctctgttgtgcttctttccattgtgcctttgtgacaaatcctacggtggaaagttgttcccaggtttctacgctcactcttgtcctcaagccggagagattgtcagatcagttgtcgccaaggcagttgctagagaaactcgtatggctgcatctttgatgagacttcactttcacgactgttttgtccaaggttgcgatggttccttgcttctggattcctctggtcgtattgtctctgagaagggttctaacccaaactcccgttccgcacgtggtttcgatgtggttgaccagatcaaggctgaacttgagaagcaatgtcctggtactgtttcttgtgctgatgccttgacccttgcagctagagactcatctgttctgactggaggtccttcttgggtcgtttctttgggaagacgtgactcaagatccgcatccttatctggttctaacaacaacattcctgctccaaacaacaccttccagactatcttgtctaagttcaatagacaaggacttgacgttaccgatctggttgctttgtcaggttctcacactattggtttctctcgttgtacctcctttagacagagattgtacaaccaatcaggtaatggaagaccagacatgaccttagaacagtctttcgctgctaacctgagacaaagatgccctcgttcaggtggtgaccagattctgtctgtgttggacatcatttctgctgctaagttcgacaactcctacttcaagaacttgattgagaacaaaggattgctgaactcagatcaagtcttgttcaactctaacgagaagtccagagaacttgtcaagaagtacgctgaagaccaaggagagttctttgagcaattcgctgagtccatgatcaagatgggtaacatctctccattgactggttcctctggagaaatcagaaagaactgtcgtaagatcaactcttaatgcggccgc

SEQ ID NO: 93 - DNA, 1026 bp, Artificial Sequence
  HRP isoenzyme >08562.1_optPpManual = 08562_ 1_optPp
ctcgagaagagagaggccgaagctgccagattgacctctatcttacttttgctgtctctgttgtgcttctttccattgtgcctttgtgacaaatcctacggtggaaagttgttcccaggtttctacgctcactcttgtcctcaagccggagagattgtcagatcagttgtcgccaaggcagttgctagagaaactcgtatggctgcatctttgatgagacttcactttcacgactgttttgtccaaggttgcgatggttccttgcttctggattcctctggtaagattgtctctgagaagggttctaacccaaactcccgttccgcacgtggtttcgatgtggttgaccagatcaaggctgaacttgagaagcaatgtcctggtactgtttcttgtgctgatgccttgacccttgcagctagagactcatctgttctgactggaggtccttcttgggtcgtttctttgggaagacgtgactcaagatccgcatccttatctggttctaacaacaacattcctgctccaaacaacaccttccagactatcttgtctaagttcaatagacaaggacttgacgttaccgatctggttgctttgtcaggttctcacactattggtttctctcgttgtacctcctttagacagagattgtacaaccaatcaggtaatggaagaccagacatgaccttagaacagtctttcgctgctaacctgagacaaagatgccctcgttcaggtggtgaccagattctgtctgtgttggacatcatttctgctgctaagttcgacaactcctacttcaagaacttgattgagaacaaaggattgctgaactcagatcaagtcttgttctcttctaacgagaagtccagagaacttgtcaagaagtacgctgaagaccaaggagagttctttgagcaattcgctgagtccatgatcaagatgggtaacatctctccattgactggttcctctggagaaatcagaaagaactgtcgtaagatcaactcttaatgcggccgc

SEQ ID NO: 94 - DNA, 2005 bp, Artificial Sequence
  HRP isoenzyme >C3
cagccacactctcaactgatcaaatcatagtttgtcttcttcctaaaaaagaaaaagaaaaatggggttttctcctctcatttcctgcagtgctatgggagccctaatattgagttgccttctgcttcaagcttcaaactctaatgctcagttgaggcctgacttctactttaggacttgcccatctgttttcaatattattggggatatcattgtcgatgaactgaggactgatcctcgtattgccgctagccttcttcgccttcactttcatgactgctttgttcgtgtaagtgtaaggacttaactttttttttttaaactatgacgtgttcattggacgtaactacttttcaccatttaattcacatatagaatagaggccaaaaggaatattcgaatcaataaatacaagcgtcatataatgtcatatatatatatatataattttgtagggttgtgatgcatcgatcctgcttgacaattccacgtcgttccgaaccgaaaaagatgctgctccaaacgcaaattcagctagaggatttggtgtcatagatagaatgaaaacatcccttgagagagcttgcccaagaacagtgtcttgtgcagatgttctcaccatcgcctctcaaatatcagtgcttttggtatgtacatgatttataacggatgatattaatccaatatgttatggatttgacgtcaatgctttataagttatgaaatttgattcaaaatgttatgaatttgatgtcaattctttatatatatgttatagtcgggaggtccatggtggccggttccgttggggaggagagacagcgtcgaagctttcttcgatttggctaatacagctcttccctctccatttttcactcttgctcaacttaaaaaagctttcgctgacgttggcctaaaccgcccctcagatctagtcgctctttctggtaaaatattcatgatgtttctaatataagtgtttttgatctagctagatctatgcaattcattttatataatgatagctaaatggatgcactcctccactaagtctggaatttacatattaatttataagttatagaattacaaatttataattcaaattttattattttgaattctattaatatgatctactattataattcatatttattttgtaatttcttaggaatgatttgcttttggctttgaaatgcatgaccaagtataaaataaattaaaaaaggataatataattaaataataaataataccatctcaaacaaaatgtcttcaagcacgtgattttgaattaaaacatgaattagtaaaacgaaaaaatcatgacttttttgttgtcaaactttttgttttttttttttaattttccaactgacaaaccataaaaacttaaacatgcaatgtatctaaaccttagttattgtcaaaatggtaggtggtcacacatttggaagagcacaatgccaatttgtgacacctcgtctctacaacttcaacggtacaaacagaccagacccaactctggacccaacttaccttgtccaactccgtgcattgtgccctcaaaacggaaacggcaccgttctggtcaacttcgatgtcgtgactccgaatacttttgatcgtcaatactacaccaatcttcgtaatgggaaaggtctgattcagagtgaccaagagctcttctcgactccaggagccgacacgatcccactagtaaacctatacagcagcaacacgttcgcgttcttcggagcattcgttgatgcaatgattaggatgggaaatcttagacctttgactggaactcaaggcgagataagacagaattgtagggttgtgaattcgcgaattaggggtatggagaacgatgatggagttgtgagttctatttgattatgttgggaatatggttatgtaacaaatcataaaatgtgtgggaacatgcatgtcgactaaataaaagctctcacgagttatgacttgtgagattacaact

(19) United States
(12) Patent Application Publication
(10) Pub. No.: US 2014/0363837 A1
(43) Pub. Date: Dec. 11, 2014

(54) Horseradish Peroxidase Isoenzymes

(71) Applicants: Technische Universitaet Graz (Graz University of Technology), Graz (AT); Acib GmbH, Graz (AT); Karl-Franzens-Universitaat Graz, Graz (AT)

(72) Inventors: Florian Krainer, Graz (AT); Laura Naeaetsaari, Graz (AT); Anton Glieder, Gleisdorf (AT); Martin Kulterer, Graz (AT); Victoria Reichel, Graz (AT)

(73) Assignee: Technische Universitaet Graz (Geaz University of Technology, Graz (AT)

(21) Appl. No.: 14/352,278

(22) PCT Filed: Oct. 19, 2012
(86) PCT No.: PCT/EP2012/070751

§ 371 Date: Apr. 16, 2014

(30) Foreign Application Priority Data
Oct. 19, 2011 (EP) 11185833.8
Jun. 28, 2012 (EP) 12174133.4

(51) Int. Cl. C12N 9/08; C12Q 1/28

(52) U.S. Cl. CPC C12N 9/0065; C12Q 1/28; C12Y 111/01; C12Y 111/01007
USPC 435/28; 435/192; 435/320.1; 435/252.33; 435/252.31; 435/254.23

(57) ABSTRACT

The present invention relates to recombinant heme-containing horseradish peroxidase isoenzymes with improved properties. In particular, the present invention relates to a plant enzyme kit comprising recombinant peroxidase isoenzymes, preferably horseradish peroxidase isoenzymes.

The present invention relates to recombinant heme-containing horseradish peroxidase isoenzymes with improved properties. In particular, the present invention relates to a plant enzyme kit comprising recombinant peroxidase isoenzymes, preferably horseradish peroxidase isoenzymes.

### BACKGROUND

Horseradish (Armoracia rusticana) is a herb cultivated worldwide in temperate regions, mainly due to the culinary use of its roots. However, the roots of A. rusticana are also used for the acquisition of highly versatile enzymes, the horseradish peroxidases (HRP) (Veitch, N.C. (2004) Phytochemistry 65, 249-259).

Peroxidases use hydrogen peroxide or various organic hydroperoxides as electron acceptors and are subsequently able to catalyze several oxidative reactions. They are encoded by various multigenic families, which are classified not only according to specific catalytic characteristics, but also according to structural properties and sequence information. A major mean for peroxidase taxonomy is the discrimination between heme-containing peroxidases and non-heme peroxidases. The latter contain five superfamilies, whereas the heme-containing peroxidases contain six superfamilies (Passardi, F., et al., (2007), Phytochemistry 68, 1605-11; Hofrichter, M., et al., (2010), Applied microbiology and biotechnology 87, 871-97).

All heme-containing peroxidases share some common features:
A ferriprotoporphyrin IX prosthetic group at the active site with the key catalytic residues,
Essential structural elements, such as two buried calcium-binding sites, four cysteine bridges and a buried salt-bridge,
Hydrogen bonds and structural water molecules from the heme pocket to the distal calcium-binding site.

The non-animal heme peroxidases (synonymously referred to as plant peroxidases) comprise the majority of the so far identified heme peroxidase sequences. Several HRP isoenzymes have been described in literature. The horseradish peroxidase isoenzymes are referred to codes depending on their calculated isoelectric point (e.g. HRP A-isoenzymes have acidic isoelectric points, B- and C-isoenzymes are neutral or neutral-basic, D- and E-isoenzymes are basic).

The isoenzyme HRP C as mentioned in literature corresponds to the Peroxidase C1A chain which is part of the peptide encoded by the gene prxC1a (Gen Bank: M37156.1; (Fujiyama, K., et al., (1988) European journal of biochemistry 173, 681-687)). The corresponding C1A UniProt entry annotates a N-terminal hydrophobic leader peptide (positions 1-30), the Peroxidase C1A chain (31-338) and a C-terminal propeptide part (339-353) that is believed to convey vacuolar targeting (UniProtKB: P00433) (Veitch, N. et al., (2004) Phytochemistry 65, 249-259). Further HRP nucleotide sequences published in the GenBank database encoded the following known HRP isoenzymes: C1C (M60729.1), C1B (M37157.1), C3 (D90116.1), C2 (D90115.1) and N (X57564.1) (Fujiyama, K., et al., (1988) European journal of biochemistry 173, 681-687; Fujiyama, K., et al. (1990) Gene 89, 163-169; Bartonek-Roxa, E., et al. (1991) Biochimica et biophysica acta 1988, 245-250). These isoenzymes plus HRP A2 and E5 were also published at UniProt. The genes of nowadays published genomic DNA sequences encoding HRP isoenzymes are structured into four exons and three introns with identical splice site positions (Veitch, N. et al., (2004) Phytochemistry 65, 249-259). Table 1 summarizes all isoenzymes published at UniProt or GenBank (effective March 2011):

TABLE 1

| isoenzyme | GenBank | UniProt | calculated IEP | calculated MW kDa |
|---|---|---|---|---|
| C1A | M37156.1 | P00433 | 5.67 | 38.82509 |
| C1B | M37157.1 | P15232 | 5.74 | 38.64586 |
| C1C | M60729.1 | P15233 | 6.21 | 36.54824 |
| C2 | D90115.1 | P17179 | 8.70 | 38.03538 |
| C3 | D90116.1 | P17180 | 7.50 | 38.17950 |
| A2 | — | P80679 | 4.72 | 31.89938 |
| E5 | — | P59121 | 9.13 | 33.72242 |
| N | X57564.1 | Q42517 | 7.48 | 35.12629 |

Applications of horseradish peroxidases are numerous and diverse. HRP is used in bioscience and biotechnology as well as in studies for medical applications. Thus, there is a need for HRP with improved properties.

### SUMMARY OF THE INVENTION

The present invention relates to recombinant heme-containing horseradish peroxidase isoenzyme with improved technological properties such as altered glycosylation, improved catalytic properties or improved stability and different ranges of pH optima and improved surface interactions. In addition, the gene sequences of these new isoenzymes allow unlimited availability of pure isoenzymes by recombinant production, in contrast to environmentally dependent isoenzyme mixtures from plants or pure isoenzymes from expensive chromatographic purification. In particular, the present invention relates to a plant enzyme kit comprising recombinant peroxidase isoenzymes, preferably horseradish peroxidase isoenzymes. Recombinant HRP isoenzymes show different biochemical properties, such as for example different catalytic activities towards four different substrates. A kit of HRP isoenzymes allows the identification of an HRP isoenzyme that suits the needs of any application best, in a way which is both convenient and efficient.

The expression of recombinant plant peroxidases has been a matter of investigation since the early 1990s with the main focus lying on E. coli as expression host. However, the extremely low yields and formation of inclusion bodies turned out to be a major issue in the expression of eukaryotic proteins in E. coli and a lot of effort has been put into the dealing with this issue.

Alternatively to E. coli, various eukaryotic organisms have been used as expression hosts for plant peroxidases. Recombinant HRP has been expressed in an insect tissue culture via a baculovirus transfer vector as well as in Saccharomyces cerevisiae. In 2000, Morawski et al. (Protein engineering 13, 377-84) published the expression of HRP variants in S. cerevisiae and Pichia pastoris with significantly increased HRP activity in the culture supernatant. However the yields were still in the range of a few mg/L yeast culture. Further hosts for HRP expression are Nicotiana tabacum, Spodoperta frugiperda and Beta vulgaris.

Recombinantly expressed HRP has been subject to directed evolution in order to improve its expression and enzymatic properties such as specific activity or thermal stability. Morawski et al. (2000, 2001) described a 40-fold increase in specific activity after three rounds of random point mutagenesis and screening in a S. cerevisiae culture supernatant. The mutations were mainly located in either loop or surface regions. Even though the specific activity could be improved, the thermal stability was decreased. However, an additional single site mutation (N175S) led to a significant improvement in thermal stability.

Expression systems for expression of exogenous foreign genes in eukaryotic and prokaryotic cells are basic components of recombinant technology. Despite the abundance of expression systems and their wide-spread use, they all have characteristic disadvantages. For example, while expression in E. coli is probably the most popular as it is easy to grow and is well understood, eukaryotic proteins expressed therein are not properly modified. Moreover, those proteins tend to precipitate into insoluble aggregates and are difficult to obtain in large amounts.

The present invention encompasses the expression of the HRP isoenzymes (or functional derivatives thereof) in either prokaryotic or eukaryotic cells.

Any cultivated prokaryotic or eukaryotic host cell, e.g. bacterial, fungal, plant, human and animal host cells, may be used as a host cell.

Preferred prokaryotic host cells may be e.g. derived from bacteria, such as Escherichia coli, B. subtilis, Salmonella, Pneumococcus, etc. Alternatively, eukaryotic host cells, e.g. from algae, fungi, etc., cells from multi-cellular organisms, may be selected. Compared to prokaryotic expression systems, the advantages of eukaryotic systems are adequate folding including correct disulphide bridge formation, processing and the ability to perform posttranslational modifications of heterologously expressed eukaryotic proteins. Cells from eukaryotic organisms are particularly preferred, if posttranslational modifications, e.g. glycosylation of the encoded proteins, are required (N and/or O linked). Typical examples of suitable eukaryotic host cells include yeasts such as Saccharomyces cerevisiae, Schizosaccharomyces pombe, Klyveromycis lactis, Yarrowia lipolytica, Arxula adenivorans, Pichia angusta and Pichia pastoris. Yeast host cell systems offer an attractive alternative to prokaryotic host cells, because they combine many advantages of other eukaryotic host cells, like folding, processing and posttranslational modification of heterologous proteins. Compared to higher eukaryotes, protein yields obtained in Pichia pastoris expression cultures are relatively high, ranging up to grams per liter culture volume. In Pichia pastoris, heterologously expressed proteins can be secreted into the culture medium by fusion to the yeast mating type α-factor signal peptide. Other examples of suitable eukaryotic cells include mammalian host cells and further host cells, in particular host cells established for laboratory use, such as HEK293-, Sf9-, CHO(Chinese hamster ovary)- or COS-cells.

A number of plant expression systems exist as well. One advantage of plants or algae in an expression system is that they can be used to produce pharmacologically important proteins and enzymes on a large scale and in relatively pure form. In addition, micro-algae have several unique characteristics that make them ideal organisms for the production of proteins on a large scale.

Due to following advantages P. pastoris is the most preferred host for recombinant protein expression
A broad knowledge base of this expression system due to a decision of Phillips Petroleum (continued by RCT) to release the P. pastoris system to academic research laboratories (Gregg, J. M., et al., (2000) Molecular Biotechnology 16, 23-52).
A high similarity of the methods for molecular genetic manipulation of P. pastoris to those applied to Saccharamoyces cerevisiae which is one of the best-characterized systems in this field of science.
Known and publicly available high quality annotated genome sequences of several most frequently used strains.
A strong preference for respiratory growth facilitates the ability to culture P. pastoris to high cell density, which further enables the expression of foreign proteins for basic laboratory research, as well as for industrial manufacture up to concentrations of several grams per liter.
An eukaryotic protein synthesis pathway and the ability to perform higher eukaryotic protein posttranslational modifications (correct polypeptide folding, O- and N-linked glycosylations, acylation, methylation, disulfide bond formation, proteolytic processing, targeting to subcellular compartments).
The AOX1 promoter of the gene encoding alcohol oxidase 1 has properties highly desirable for the controlled high-level expression of foreign protein: Strong repression on carbon sources like glucose or glycerol, but over 1000-fold induction when confronted with methanol as a sole carbon source.
AOX1 promoter variants which allow high levelprotein expression without the use of methanol.
The possibility to produce both, secreted and intracellular recombinant protein. Since P. pastoris only secretes very low levels of endogenous proteins, secreted recombinant protein comprises the vast majority of the total protein in the medium. Thus, the secretion of the heterologous protein serves as valuable first step in purification.
The absence of endotoxins, oncogenic and viral DNA in P. pastoris products.
The GRAS status of Pichia pastoris.

The ability to process signal sequences plays a major role when a foreign gene is fused to the S. cerevisiae α-factor prepro signal sequence which facilitates the secretion of the foreign protein.

Another important aspect of posttranslational modifications by P. pastoris is glycosylation. The glycosylation patterns of mammalian cells and P. pastoris differ greatly, which might have an impact on the activity of the recombinant protein and constitute a significant problem in the use of recombinant proteins by the pharmaceutical industry since they might be highly antigenic or affected in their biological activity. Efforts are made to genetically engineer P. pastoris to humanize its glycosylation behavior (Cregg et al. (2000)).

P. pastoris adds O-oligosaccharides to the hydroxyl groups of serine and threonine residues of secreted proteins. Unlike mammals, P. pastoris solely adds Man residues. O-glycosylation starts in the endoplasmatic reticulum (ER) where one Man residue is transferred from Man-P-dolichol to a Ser/Thr residue. The Ser/Thr residues used for O-glycosylation in P. pastoris are not necessarily the same residues as in the native host. In the Golgi apparatus, sugar transferases add further α1,2-linked Man residues. The extent of O-glycosylation by P. pastoris differs among the produced recombinant proteins.

Asparagine residues found in the conserved amino acid pattern Asn-X-Ser/Thr are subject to possible N-glycosylation. The initial steps of N-linked glycosylation are the same in yeast and most higher eukaryotes: N-acetylglucosamine is transferred from uridine diphosphate-GlcNAc onto dolichol phosphate on the cytoplasmic face of the ER membrane. Further addition of GlcNAc and Man leads to the structure Man₅GlcNAc₂-P-dolichol. A flipase facilitates the translocation to the luminal face of the ER membrane where the Man5GlcNAc2-P-dolichol is further extended to Glc₃Man₉GlcNAc₂-P-dolichol. The sugars from this structure are transferred cotranslationally to the Asn residue at the Asn-X-Ser/Thr motive by an oligosaccharyl-transferase complex. After removal of three Glc residues and one Man residue to Man₈GlcNAc₂, the glycopeptide is transported to the Golgi apparatus, which is where the glycosylation pathways of yeasts and mammals diverge. Contrary to mammals, P. pastoris does not reduce the Man₈GlcNAc₂ structure, but rather further extends it with additional Man residues catalyzed by Golgi mannosyltransferases. Also mannose phosphate diesters have been found in P. pastoris N-glycans.

An approach to increase the yield of functionally expressed recombinant protein in P. pastoris is the coexpression of proteins which either lead to an improvement in the host cell's metabolism or whose activity supports the functional expression of the actual target protein.

For example, Schroer et al. (2010, Metabolic engineering 12, 8-17) showed a significantly improved whole-cell biotransformation reaction (using the NADH-dependent butanediol dehydrogenase) by overexpressing the MUT pathway enzyme FLD, which facilitates NADH regeneration.

The tremendously high induction of AOX1 promoter-driven heterologous genes can lead to extremely high amounts of protein. These tend to activate the cell's unfolded protein response, indicating that correct folding of the peptides and disulfide bridge formation are limiting factors during high-level expression. In order to approach this bottleneck, protein disulfide isomerase (PDI) and other helper proteins can be coexpressed. PDI is described as an ER-residing protein of the thioredoxin superfamily with chaperone- and peptide binding functions. PDI overexpression has been shown to further improve the expression of secretory recombinant proteins by extending the secretory capacities of cells that already reached their limit in the production of functional recombinant protein without coexpressed PDI.

Horseradish peroxidase isoenzymes form a large group of extremely versatile enzymes with numerous applications these days and many more potential applications in future. Nonetheless, only a small number of isoenzymes has been characterized, published or even identified on the level of either amino acid sequence or nucleotide sequence.

In an aspect of this invention, novel recombinant HRP isoenzyme sequences on genome DNA level are provided.

In an aspect of this invention a kit comprising at least five recombinant peroxidase isoenzymes is provided.

Depending on the intended use, the kit preferably comprises at least 8, 10, 15, 20, or 25 recombinant peroxidase isoenzymes.

In a further aspect of this invention a kit as described above is provided, wherein said isoenzymes are horseradish peroxidase isoenzymes.

In a further aspect of this invention a kit as described above is provided, wherein said isoenzymes are encoded by a nucleic acid comprising a sequence selected from the group consisting of SEQ ID NO:1 to SEQ ID NO:51 and SEQ ID NO:60 to SEQ ID NO:94 and functionally active variants thereof.

In a further aspect of this invention a kit as described above is provided, wherein said isoenzymes are selected from isoenzymes which are encoded by a nucleic acid comprising a sequence selected from the group consisting of SEQ ID NO:67 to SEQ ID NO:93.

In a further aspect of this invention a kit as described above is provided, wherein said isoenzymes are selected from isoenzymes which are encoded by a nucleic acid comprising a sequence selected from the group consisting of SEQ ID NO:67 to SEQ ID NO: 93, preferably at least five isoenzymes.

In a further aspect of this invention a kit as described above is provided, wherein said isoenzymes are selected from isoenzymes which are encoded by a nucleic acid comprising a sequence selected from the group consisting of SEQ ID NO:67 to SEQ ID NO:93 and at least one isoenzyme is selected from isoenzymes which are encoded by a nucleid acid comprising a sequence selected from the group consisting of SEQ ID NO:1 to SEQ ID NO:51 and SEQ ID NO:60 to SEQ ID NO:66 and SEQ ID NO:94.

Depending on the intended use, the kit preferably comprises at least 4, 5, 7, 9, 11, 13, 15, 17, 19 or 21 isoenzymes which are selected from isoenzymes which are encoded by a nucleic acid comprising a sequence selected from the group consisting of SEQ ID NO:67 to SEQ ID NO: 93, and at least 1, 2, 3, 4, or 5 isoenzymes which are encoded by a nucleid acid comprising a sequence selected from the group consisting of SEQ ID NO:1 to SEQ ID NO:51 and SEQ ID NO:60 to SEQ ID NO:66 and SEQ ID NO:94.

In a further aspect of this invention a kit as described above is provided, wherein said isoenzymes have a thermal stability of at least 2 weeks at 20-25° C. and at least several months at 4° C.

In a further aspect of this invention a kit as described above is provided, wherein said isoenzymes have a biological activity of at least 500 U/mg, preferred of at least 750 U/mg, more preferred of at least 1.000 U/mg and most preferred of at least 1.200 U/mg in the ABTS assay.

In a further aspect of this invention a kit as described above is provided, wherein said isoenzymes have a calculated isoelectric point of less than 5 or greater than 6.

In a further aspect of this invention a kit as described above is provided, wherein said isoenzyme has a lower Km when compared to the isoenzyme C1A.

In a further aspect of this invention an isoenzyme encoded by a nucleic acid comprising a sequence selected from the group consisting of SEQ ID NO:1 to SEQ ID NO:51 and SEQ ID NO:60 to SEQ ID NO:94 and functionally active variants thereof, for use in a kit as described above is provided.

In a further aspect of this invention use of a kit for identifying a horseradish isoenzyme as a reagent for organic synthesis and biotransformation, preferably in coupled enzyme assays, chemiluminescent assays, and immunoassays is provided.

In a further aspect of this invention use of a kit for identifying a horseradish isoenzyme which is used in a biosensor, in diagnostics, in bioremediation or in chemical synthesis is provided.

In a further aspect of this invention, novel recombinant HRP isoenzymes are heterologously expressed.

A further aspect of this invention is a recombinant heme-containing horseradish peroxidase isoenzyme encoded by a nucleic acid comprising a sequence selected from the group consisting of SEQ ID NO:1 to SEQ ID NO:51 and SEQ ID NO:60 to SEQ ID NO:94 and functionally active variants thereof.

A further aspect of the invention is an isoenzyme, wherein said isoenzyme is less glycosylated as compared to the isoenzyme C1A.

A further aspect of the invention is an isoenzyme, wherein said isoenzyme has a thermal stability of at least 2 weeks at 20-25° C. and at least several months at 4° C.

The term several months as used herein refers to a period of 1 to 24 months, preferred to a period of 3 to 18 months and most preferred to a period of 6 to 12 months.

A further aspect of the invention is an isoenzyme, wherein said isoenzyme has a biological activity of at least 500 U/mg, preferred of at least 750 U/mg, more preferred of at least 1.000 U/mg and most preferred of at least 1.200 U/mg in the ABTS assay. 1 Unit is defined as the amount of enzyme that converts 1 μmol ABTS per minute with K_{M}=0.44 mM ABTS.

A further aspect of the invention is an isoenzyme, wherein said isoenzyme has a calculated isoelectric point of less than 5 or greater than 6.

A further aspect of the invention is an isoenzyme, wherein said isoenzyme has a lower Kₘ when compared to the isoenzyme C1A.

A further aspect of the invention is a vector comprising a polynucleotide encoding an isoenzyme.

A further aspect of the invention is a host cell comprising a vector encoding a mutant of an isoenzyme displaying a reduced number of lysine residues.

A further aspect of the invention is a host cell comprising a vector encoding an isoenzyme.

A further aspect of the invention is a host cell, wherein said host cell is a prokaryotic cell, preferably E. coli or B. subtilis or eukaryotic cell, preferably a yeast cell.

A further aspect of the invention is a host cell, wherein said host cell is a Pichia cell, preferably a P. pastoris or P. angusta cell.

A further aspect of the invention is a method for producing a recombinant heme-containing horseradish peroxidase isoenzyme which comprises

a. providing a recombinant host cell engineered to express a polynucleotide,

b. culturing the host cell under conditions suitable for obtaining the isoenzyme, wherein the expression product of the host cell is reacting with heme; and

c. recovering the isoenzyme from the culture.

A further aspect of the invention is the use of a horseradish peroxidase isoenzyme as a reagent for organic synthesis and biotransformation, preferably in polymerizations, coupled enzyme assays, chemiluminescent assays, and immunoassays.

A further aspect of the invention is the use of a horseradish peroxidase isoenzyme in a biosensor, in diagnostics, in bioremediation or in chemical synthesis.

A further aspect of the invention is a horseradish peroxidase isoenzyme for use in targeted cancer therapy.

A further aspect of the invention is a recombinant synPDI enzyme encoded by a nucleic acid comprising a sequence of SEQ ID NO:52 and functionally active variants thereof.

The HRP isoenzymes of the A2 group have the so far most acidic isoelectric point. A low isoelectric point is thought to indicate increased enzymatic activity and elevated stability at lower pH, which renders HRP A2 isoenzymes highly interesting isoenzymes, since (bio-) catalysis at low pH is of considerable industrial interest.

Thus, in one aspect of the invention the recombinant HRP isoenzyme is HRP A2A.

In a further aspect of this invention a protocol for the purification of the expressed HRP isoenzyme was established that allows enzyme purities comparable to or higher than commercially available HRP preparations.

In a further aspect of this invention the purified isoenzyme was characterized in terms of posttranslational modifications, its enzymatic activity towards the two standard substrates 2,2′-azino-bis(3-ethylbenzthiazoline-6-sulphonic acid) (ABTS) and guaiacol, and its stability in different buffers, at different temperatures and at different protein concentrations.

### DESCRIPTION OF THE DRAWINGS

FIG. 1: Aligned Sanger sequencing reads of C1C: The double peak signals A/G1828 and C/G1861 can be seen in three out of seven reads.

FIG. 2: Phylogenetic tree of aligned HRP isoenzymes. The published HRP isoenzymes are closer related to each other than to most of the newly discovered HRPs.

FIG. 3: Sequence logos of signal peptide cleavage sites predicted by SignalP 3.0.

a) Sequence logo of all predicted signal peptide cleavage sites.

b) Sequence logo of the predicted signal peptide cleavage sites of the isoenzymes C1A, C1B, C1C, C2, A2A, E5, 01805, 22684 and 02021.

FIG. 4: RNA secondary structure of the synthetic HRP A2A gene. The predicted RNA secondary structure with a total free energy of −167 kcal/mol.

FIG. 5: GC content of the synthetic HRP A2A gene. The GC content over the full sequence; calculated with an averaging window of 30 bp, sliding over the sequence with a stepping value of 1.

FIG. 6: HRP activities from the first screen of A2A and coexpressed PDI genes. G4, B5, D6, A9 were done as duplicates. A2AMutSF5 shows the starting strain's activity. +empty plasmid shows the influence of the cotransformed empty plasmid. +PDI704, +synPDI, +synPDI N314H show the average of the measured HRP activity under PDI coexpression. These results have been obtained from one 96-DWP cultivation per PDI.

FIG. 7: HRP activities from the rescreen of A2A+synPDI N314H coexpression. The activity control strains G4, B5, D6, A9 were done as duplicates. A2AMutSF5 and +empty plasmid represent the starting strain activity and its activity plus “coexpressed” empty plasmid, respectively. +synPDI N314H shows the measured activities of clones coexpressing A2A+synPDI N314H (average of quadruplicates).

FIG. 8: Total HRP activities of the cultivated strains. The MutS and MutS synPDI N314H strains were measured as negative controls for HRP activity. The A2AMutSF5 and A2AMutSF5 synPDI N314H strains showed increasing activity over time. The measured activity from A2AMutSF5 synPDI N314H was slightly higher than the ones from the A2AMutSF5 strains.

FIG. 9: Total HRP activities of the fractions from StrepTactin affinity chromatography. HRP A2ANstrep did not bind to the StrepTactin column and eluted in the first fractions. Already in the flowthrough fraction, collected during the loading of the sample, HRP activity was measured. In the subsequent two washing fractions the remaining HRP activity was detected.

FIG. 10: HIC chromatogram and measured HRP activities. Functional HRP eluted from ˜200 mL-475 mL. Proteins without HRP activity eluted from ˜560-600 mL.

FIG. 11: The 10 different pHs (9.5-5.0) are indicated above the columns (2-11). In the rows A and B, HRP A2A was changed to the respective buffers (without QFF), then the standard ABTS assay was performed. In the rows C and D, 15 μL of the test tube supernatant were used for the ABTS assay: Only at pH 9.5, no HRP activity could be detected. In the rows E and F, 15 μL of the supernatant of the buffers at pH 9.5, 9.0 and 8.5 were used for the ABTS assay after eluting HRP A2A from the QFF material.

FIG. 12: Anion exchange chromatogram and measured HRP activities. The fractions exposing HRP activity are enlarged. HRP A2A eluted from ˜70-115 mL.

FIG. 13: HRP activities and Rz values of the fractions from ammonium phosphate precipitation. (a) shows the HRP activities measured from the pellets of the various precipitation steps redissolved in buffer QFF-A and the supernatant of the last precipitation step. (b) shows the Rz values of the fractions.

FIG. 14: Size exclusion chromatogram and measured HRP activities. The fractions exposing HRP activity are enlarged below. HRP A2A eluted from ˜80-90 mL.

FIG. 15: SDS-PAGE gel picture of the purified HRP A2A. The PageRuler prestained Protein Ladder was used as protein standard and was run until the ˜25 kDa band reached the lower end of the gel. The band at ˜35 kDa in the second gel lane was the size exclusion chromatography fraction that contained the purified HRP A2A.

FIG. 16: IEF gel picture of commercially available HRP preparations and the purified HRP A2. The IEF Marker 3-10, Liquid Mix from SERVA Electrophoresis GmbH was used as standard and was run in the flanking lanes. HRP A2A showed an IEP of pH 3.5-4.2. The Sigma preparations II, VI, VI-A and XII seemed to contain a similar HRP isoenzyme, among others. Type I contained predominantly isoenzymes with an IEP at ˜pH 5. The Toyobo HRP seemed to contain an isoenzyme slightly more basic than A2A (MS-MS analysis of the Toyobo HRP revealed the sequence of isoenzyme C1a as the major component of this commercial preparation from the plant. This isoenzyme has a calculated isoelectric point of 5.67).

FIG. 17: pH profile of HRP A2A with ABTS. pH 4.5 was identified as optimum for the catalysis of ABTS by HRP A2A. By the photometric assay no activity was measured lower than pH 3.5 or higher than pH 9.0. All points were measured as triplicates.

FIG. 18: Standard curves from BSA and HRP VI-A dilutions. BSA and HRP VI-A gave comparable results in the performed BCA assay for protein quantitation and were equally suitable for determining the concentration of HRP A2A. Each point was measured as triplicate.

FIG. 19: Kinetics of HRP A2A with ABTS as saturation curve and as Lineweaver-Burk plot. (a) The calculated ABTS units/mg were plotted against the respective ABTS concentration. (b) The reciprocal values of the calculated reaction rates were plotted against the reciprocal respective ABTS concentration. The trendline crosses the x-axis at the reciprocal negative value of K_{M} and the y-axis at the reciprocal value of Vₘₐₓ. All points were measured as triplicates.

FIG. 20: pH profile of HRP A2A with guaiacol. The optimal pH for the conversion of guaiacol by HRP A2A was found to be at pH 5.0. The activity stayed high until pH 7.0. No activity was measured lower than pH 3.5 or higher than pH 9.0. All points were measured as triplicates.

FIG. 21: Kinetics of HRP A2A with guaiacol as saturation curve and as Lineweaver-Burk plot. (a) The calculated guaiacol units/mg were plotted against the respective guaiacol concentrations. (b) The reciprocal values of the calculated reaction rates were plotted against the reciprocal respective guaiacol concentrations. The trendline crosses the x-axis at the reciprocal negative value of K_{M} and the y-axis at the reciprocal value of Vₘₐₓ. All points were measured as triplicates.

FIG. 22: Influence of pH on the stability of HRP A2A. The optimal pH range for storing HRP A2A was pH 7.0-10.0. At lower pH the activity was significantly decreased over time. All points were measured as triplicates.

FIG. 23: Influence of temperature on the stability of HRP A2A. At 50° C. and 65° C. the activity was completely abrogated within 10 min and 3 min, respectively. At 37° C., HRP activity could be detected for 30 min. At 20° C., the activity decreased to 27% of the initial activity in 60 min. At 4° C., 75% of the starting activity could still be measured after 60 min. All measuring points were done as triplicates.

FIG. 24: Influence of protein concentration on the stability of HRP A2A. HRP A2A stayed stable at concentrations ≧0.6 ng/μL. At lower concentrations, the HRP activity decreased within one day. All points were measured as triplicates.

FIG. 25: HRP sequences from next-generation sequencing. The HRP sequences found in the transcriptome and their corresponding published sequences are shown. Differences between the contig-derived translated transcriptome sequences and the published sequences are marked in grey.

FIG. 26: Annealed adaptor for genome walking: Adaptor strand 1 was annealed either to adaptor strand 2.a, 2.b or 2.c, depending on the desired 5′-overhang (red). The last nucleotide at the 3′-end of the adaptor strand 2 did not match to adaptor strand 1, in order to prevent elongation from that 3′-end on. Marked in yellow+green is the binding site of the Adaptor Primed, marked in green+blue is the binding site of the Adaptor Primer2.

FIG. 27: The two Sanger-verified sequences 08562.1 and 08562.4 are shown in an alignment with the corresponding transcriptome contigs.

FIG. 28: Sequence alignment of 5′-UTR (marked in grey) of the Sanger verified C1C plus its N-terminus to the protein sequence of the N-terminus of C1B.

FIG. 29: Sequence alignment of synPDI, synPDI N314H and PDI704.

FIGS. 30 to 123: Sequences of HRP isoenzymes.

FIG. 124a-d: HRP isoenzymes showing peroxidase activity in different assays.

The term “variant” or “functionally active variant” of an enzyme as used according to the invention herein means a sequence resulting from modification of the parent sequence by insertion, deletion or substitution of one or more amino acids or nucleotides within the sequence or at either or both of the distal ends of the sequence, and which modification does not affect (in particular impair) the activity of this sequence. In a preferred embodiment the functionally active variant

a) is a biologically active fragment of the amino acid or the nucleotide sequence, the functionally active fragment comprising at least 50% of the sequence of the amino acid or the nucleotide sequence, preferably at least 60%, preferably at least 70%, more preferably at least 80%, still more preferably at least 90%, even more preferably at least 95% and most preferably at least 97%, 98% or 99%;
b) is derived from the amino acid or the nucleotide sequence by at least one amino acid substitution, addition and/or deletion, wherein the functionally active variant has a sequence identity to the amino acid or the nucleotide sequence or to the functionally active fragment as defined in a) of at least 50%, preferably at least 60%, preferably at least 70%, preferably at least 80%, still more preferably at least 90%, even more preferably at least 95% and most preferably at least 97%, 98% or 99%; and/or
c) consists of the amino acid or the nucleotide sequence and additionally at least one amino acid or nucleotide heterologous to the amino acid or the nucleotide sequence, preferably wherein the functionally active variant is derived from or identical to any of the variants of any of the sequences of SEQ ID NO: 1-49, ID NO: 61-66 or ID NO: 94.

“Percent (%) nucleotide sequence identity” with respect to the polynucleotide sequences identified herein is defined as the percentage of nucleotide residues in a candidate sequence that are identical with the nucleotide residues in the specific polynucleotide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

The functionally active variant may be obtained by sequence alterations in the amino acid or the nucleotide sequence, wherein the sequence alterations retains a function of the unaltered amino acid or the nucleotide sequence, when used in combination of the invention. Such sequence alterations can include, but are not limited to (conservative) substitutions, additions, deletions, mutations and insertions.

In a specific embodiment of the invention the polypeptide or the nucleotide sequence as defined above may be modified by a variety of chemical techniques to produce derivatives having essentially the same activity (as defined above for fragments and variants) as the modified polypeptide or the nucleotide sequence, and optionally having other desirable properties. Other desirable properties are, for example, the increase in stability as measured by the pH stability and/or temperature stability of the enzyme, the increase in activity as measured by the ABTS assay or increased stability at elevated hydrogen peroxide concentrations.

The variant of the polypeptide or the nucleotide sequence is functionally active in the context of the present invention, if the activity of the composition of the invention including the variant (but not the original) amounts to at least 50%, preferably at least 60%, more preferred at least 70%, still more preferably at least 80%, especially at least 90%, particularly at least 95%, most preferably at least 99% of the activity of the enzyme as used according to the invention including the amino acid or the nucleotide sequence without sequence alteration (i.e. the original polypeptide or the nucleotide sequence).

Functionally active variants may be obtained by changing the sequence as defined above and are characterized by having a biological activity similar to that displayed by the respective sequence of SEQ ID NO: 1-51 and SEQ ID NO: 60-94 from which the variant is derived, including the ability of HPR isoenzymes to show higher stability in technical applications, the ability to react with substrates at low concentrations, to show improved interaction with other proteins and surfaces, higher catalytic efficiency, advantages for commercial manufacturing and improved properties for in vivo applications.

Applications of horseradish peroxidases are numerous and diverse. HRP is used in bioscience and biotechnology as well as in studies for medical applications.

A very common application of HRP is nowadays in biosensors, predominantly for the real-time quantification of H₂O₂ but also for the in vivo detection of glucose, ethanol or tumor markers via co-immobilization with a H₂O₂-producing enzyme.

HRP is further used in numerous diagnostic applications. Generally, a chromogen is oxidized by HRP with H₂O₂, which is generated by a substrate-specific oxidase (e.g. glucose oxidase in colorimetric diagnostic kits for the determination of glucose in blood serum or plasma). Alternatively HRP is conjugated to specific antibodies to be used in diagnostic applications, generating a dye or fluorescence signal in case the antibodies bind to a target.

There is also a trend for miniaturization in diagnostics, for example, a portable sequential cross-flow immunoanalytical device using HRP for signal generation and hereby simplified the complex traditional ELISA procedure.

Another application for horseradish peroxidases is in bioremediation, such as the detoxification of phenolic wastewater. Efforts are made to immobilize HRP in order to facilitate reusability and increase enzyme stability: HRP was used for the removal of natural and synthetic estrogens from wastewater.

Chemical synthesis is another field in which HRP-catalyzed reactions can be applied. Oxidative reactions, such as oxidative dehydrogenation and polymerization of aromatic compounds, heteroatom oxidation and epoxidation constitute interesting enzymatic abilities for potential applications. Also N- and O-dealkylation or selective hydroxylation are nowadays used for small-scale organic synthesis.

A highly interesting reaction of HRP is the formation of cytotoxic radical species from the non-toxic plant hormone indole-3-acetic acid (IAA), also known as auxin, via a one-electron oxidation step without additional H₂O₂. An application of this reaction lies in targeted cancer therapy (e.g. antibody-, gene- or polymer-directed enzyme-prodrug therapies), where the glycosylation of HRP is an important factor regarding the clearance of the antibody from the cells.

### EXAMPLES

### 1. Kits, Protocols and Methods

1.1 Agarose Gel Electrophoresis

1% agarose gels were made with 250 mL 1×TAE buffer and approximately 2 μL EtBr (≧98% ethidium bromide). DNA samples were mixed with 6× or 2× Orange Loading Dye and pipetted to the wells of the gel, next to 5 μL or 10 μL of 0.1 μg/μL GeneRuler™ 1 kb DNA Ladder. Electrophoresis was run for approximately 50 min at a voltage of 120 V. After the run, the samples were analyzed with the GelDoc-It™ Imaging System. Preparative agarose gels were run for approximately 2 h at a voltage of 90 V. The samples were cut out off the gel at the Chroma 43.

1.2 SDS-PAGE

Protein samples were prepared as recommended by Invitrogen™ life technologies and pipetted to the wells, next to 5 μL PageRuler™ Prestained Protein Ladder. Electrophoresis was run for approximately 1 h at a voltage of 200 V. Staining of the gels was done with approximately 100 mL SDS gel staining solution for 20 min after heating the staining solution plus the gel close to the boiling point in the microwave. Destaining was done three times with 100-200 mL SDS gel destaining solution for 20 min each after heating the destaining solution plus the gel close to the boiling point.

1.3 Isoelectric Focusing Gel Electrophoresis

For isoelectric focusing, Novex IEF gels were used. Protein samples were handled in accordance to the recommendations from Invitrogen™ life technologies. As a reference, 5 μL of the IEF Marker 3-10, Liquid Mix from SERVA Electrophoresis GmbH were used. Buffers and run conditions were used as recommended. After the run, the gel was fixed for 15 min in 12% TCA (50.0 g≧99.5% trichloroacetic acid dissolved in 500 mL dH₂O). For staining, the SimplyBlue SafeStain solution from Invitrogen life technologies was used as recommended.

1.4 Plasmid Minilysate Preparation

The GeneJET Plasmid Miniprep Kit (Fermentas GmbH, St. Leon-Rot, Germany) was used for plasmid isolation according to the manufacturer's recommendations with the following deviations: Cells were abraded from half an agar plate with a toothpick, all incubation times were doubled, plasmid elution was done with 40 μL dH₂O (heated to 65° C.).

1.5 DNA Purification

DNA purification was done after PCR, preparative agarose gel electrophoresis and DNA restriction with the Wizard SV Gel and PCR Clean-Up System (Promega GmbH, Mannheim, Germany). Applied deviations from the manufacturer's recommendations were doubled incubation times and elution of DNA with 40 μL dH₂O (heated to 65° C.).

1.6 Molecular Cloning

Standard Ligations

Ligation of an insert to a vector was done overnight at 16° C. with T4 DNA Ligase (400 u/μL, 10× T4 DNA Ligase Reaction Buffer/New England BioLabs® Inc., Ipswich, Mass., USA) according to the manufacturer's recommendations.

Cloning to the pJET1.2Blunt Vector

The CloneJET™ PCR cloning Kit (Fermentas GmbH, St. Leon-Rot, Germany) was used for cloning PCR products to the pJET1.2blunt vector (see 3.4.2 Plasmid constructs) according to the manufacturer's recommendations. The incubation time for ligation was 30 minutes at room temperature with a molar vector:insert ratio of 1:3. Desalting was done for 20 min against dH₂O at room temperature prior to electroporation (see below).

Applied PCR Techniques

Standard PCR

Standard PCRs were performed with Phusion® High-Fidelity DNA-Polymerase, and genomic DNA from A. rusticana was used as template DNA. The annealing temperature was by default chosen to be 2° C. below the primer DNA melting temperature, calculated with EditSeq (Davis, Botstein, Roth melting temperature). Primers were used in a final concentration of 400 nM, dNTPs in a final concentration of 200 μM. The elongation time was calculated in dependence of the expected PCR product length, considering a processivity of 1 kb per 10-30 s of the Phusion® High-Fidelity DNA-Polymerase.

In case the PCR did not yield any product under standard conditions (either buffer HF or buffer GC), the reaction conditions were changed: The reaction was repeated with addition of 100% DMSO and 50 mM MgCl₂ (final concentration of 2% DMSO and 2 mM MgCl₂). Further, the annealing temperature was varied +/−5° C. of the primer DNA melting temperature calculated with EditSeq (Davis, Botstein, Roth melting temperature).

The PCR products were cut from a preparative agarose gel, purified and sent to LGC Genomics GmbH, Berlin, Germany for Sanger sequencing.

Genome Walking

Approximately 2 μg genomic DNA from A. rusticana were digested over night with Bsp143I, BsaWI, PsuI, XhoII, HindIII with the corresponding buffer in order to get fragments of 1-5 kb size. The digestion was stopped by heat inactivation of the enzymes, the fragmented DNA was precipitated with EtOH and the pellet was dissolved in 30 μL dH₂O.

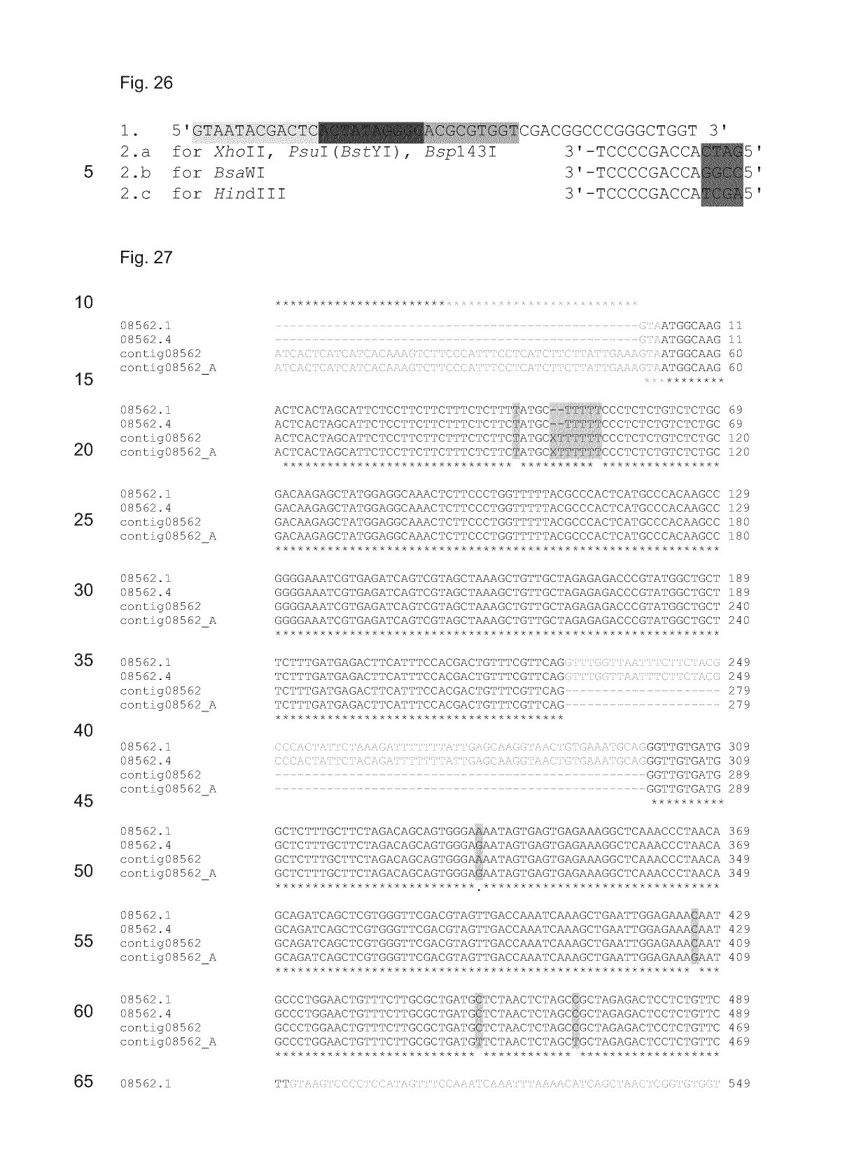

An adaptor was created by annealing adaptor strand 1 (primer gWalkingAdaptor Strand1) either to adaptor strand 2.a (primer gWalkingAdaptor Strand2a), 2.b (primer gWalkingAdaptor Strand2b) or 2.c (primer gWalkingAdaptor Strand2c), depending on the 5′-overhang created by the respective restriction enzyme (FIG. 26).

Adaptor strand 1 was annealed either to adaptor strand 2.a, 2.b or 2.c, depending on the desired 5′-overhang (red). The last nucleotide at the 3′-end of the adaptor strand 2 did not match to adaptor strand 1, in order to prevent elongation from that 3′-end on. Marked in yellow+green is the binding site of the Adaptor Primer1, marked in green+blue is the binding site of the Adaptor Primer2.

In the annealing reaction, adaptor strand 1 was mixed in 1:1 molar ratio with adaptor strand 2.a/2.b/2.c (i.e. 13.7 μL 100 μM adaptor strand 1+4.0 μL 100 μM adaptor strand 2) and heated to 95° C. for 5 min. Afterwards, the thermomixer was turned off and the mixtures were left to cool to room temperature overnight. The three differently annealed adaptors (1+2a, 1+2b, 1+2c) were ligated for 3 h at room temperature with T4 DNA Ligase to the digested gDNA fragments, considering the specific 5′ overhangs that have been created by the used restriction enzymes (i.e. adaptor 1+2a was ligated to gDNA digested with either XhoII, PsuI or Bsp143I, adaptor 1+2b was ligated to gDNA digested with BsaWI, adaptor 1+2c was ligated to gDNA digested with Hind III). The ligation reaction was stopped by incubation for 5 min at 70° C. and 70 μL TE buffer were added.

Two gene-specific primers and two adaptor primers were designed. The gene-specific primers were designed to bind approximately 100 bp away from the end of the known sequence, considering that no restriction site of the restriction enzymes used for gDNA digestion lied between the primer-binding site and the end of the known sequence. Adaptor Primer1 (5′GTAATACGACTCACTATAGGGC3′) (SEQ ID NO:54) and GeneSpecificPrimer1 were used as a primer pair for a first PCR with 1 μL of the gDNA+adaptor ligation product as template DNA. 1 μL of the first PCR mix was used as template for a second PCR with Adaptor Primer2 (5′ACTATAGGGCACGCGTGGT3′) (SEQ ID NO:55) and GeneSpecificPrimer2 as primer pair. This second primer pair was designed to bind within the first PCR product. Both PCR steps were performed with an elongation time of 50 s.

A gene-specific DNA fragment as product from the second PCR was isolated from a preparative agarose gel, purified and sent to Sanger sequencing (LGC Genomics GmbH, Berlin, Germany), using Adaptor Primer2 and the corresponding GeneSpecificPrimer2.

Colony PCR

Template DNA was isolated from single P. pastoris colonies using the Bust n′Grab protocol ((2004) BMC biotechnology 4, 8-13). GoTaq® polymerase was used in accordance to the manufacturer's recommendations with addition of MgCl₂ to a final concentration of 2 mM MgCl₂ in the PCR mix. The primers were used at a final concentration of 100 nM each. The PCR product was analyzed via agarose gel electrophoresis.

qPCR

Quantitative PCR was performed similarly to the protocol described by Abad et al. ((2010) Biotechnology journal 5, 413-20):

Genomic DNA isolated from P. pastoris as described below was used as template DNA. The 2× Power SYBR Green Master Mix (Applied Biosystems, Foster City, Calif., USA) was used for the PCR reactions. The ARG4 gene was used as an internal reference, the KanMX6 gene and the synPDI gene were used in parallel as target genes for determination of the PDI copy number, the Zeocin syn gene was used as an indirect target gene for the determination of the HRP copy number (all vectors used for HRP transformation also carried the Zeocin syn gene). Strains with verified single copy integration of the target gene were used as reference strains. The used primers and their concentrations are depicted in Table 2. All reactions were done as duplicates.

TABLE 2

| gene | primer number | final concentration mM |
|---|---|---|
| ARG4 | P09-478 | 200 |
| | P09-479 | 250 |
| Zeocin syn | P09-338 | 250 |
| | P09-337 | 250 |
| synPDI | P07-515 | 200 |
| | P07-514 | 250 |
| KanMX6 | P10-828 | 250 |
| | P10-829 | 250 |

Primers used for qPCR with ARG4 as reference gene and Zeocin syn/synPDI/KanMX6 as target genes and final concentrations of the various primers in the PCR reaction mix.

1.7 Transformation to Escherichia coli Top 10F′

DNA (approximately 100 ng, maximum volume of 20 μL) and competent cells (80 μL E. coli Top 10F′) were mixed and incubated for 5 min on ice in electroporation cuvettes. The cells were pulsed with of 2.5 kV, 200 Ω, 25 μF. 500 μL SOC medium were added immediately. The cell suspension was incubated for 1 h, 37° C., 600 rpm. 10 μL, 100 μL and the rest of the cell suspension were plated on LB-agar plates containing the respective antibiotic. Incubation of the plates was done at 37° C. overnight.

1.8 Transformation to Pichia pastoris

Transformation of P. pastoris was done as described by Lin-Cereghino et al. ((2005) Bio Techniques 38, 44-48):

Competent cells were prepared by inoculating a single colony of the respective strain to 50 mL YPD medium and grown over night at 28° C., 110 rpm. From this preculture a main-culture was grown from OD₆₀₀=0.2 to a final OD₆₀₀ of 0.8-1.0 (again in YPD medium). The cells were harvested and 80 μL of them were mixed with 3-4 μg DNA (maximum volume of 25 μL) and incubated for 5 min in electroporation cuvettes on ice. After pulsing the cells with 2.0 kV, 200 Ω, 25 μF, 500 μL 1 M sorbitol were added immediately, followed by 500 μL YPD medium. The cells were incubated for 2 h, 28° C., 110 rpm for regeneration, then plated on YPD-agar plates (10 μL, 100 μL, rest) containing the respective antibiotic(s) and incubated at 28° C. for 2-3 days.

1.9 Micro-Scale Cultivation of Pichia pastoris in 96-Deep Well Plates

In order to screen for P. pastoris clones expressing active HRP, a micro-scale cultivation in 96-deep well plates (DWP) was done similar to the protocol described by Weis et al. ((2004) FEMS yeast research 5, 179-89):

Cells from a single colony were transferred to 250 μL BMD1% per well. Two wells per plate were left empty as negative controls for cell growth, two wells were inoculated with P. pastoris MutS as negative controls for the recombinant enzyme's activity and two wells per strain were inoculated with P. pastoris strains already successfully expressing the inquired recombinant enzyme as positive controls for its activity. They were incubated at 28° C., 320 rpm, 80% humidity. After approximately 60 h, 250 μL BMM2 were added per well for the induction of recombinant protein expression. 12 h, 24 h and 36 h after the start of induction, 50 μL BMM10 were added per well. The cultivation was stopped 48 h after the start of induction, the cells were centrifuged at 3.000×g for 10 min and the supernatant was checked for HRP activity with the ABTS assay.

1.10 Small Scale Cultivation of Pichia pastoris in 2 L-Shake Flasks

A single colony was inoculated to 200 mL BMD1% and incubated for 60 h at 28° C., 110 rpm, 80% humidity. Induction of protein expression was done by addition of 20 mL BMM10 and further additions of 2 mL MeOH 12 h, 24 h and 36 h after the start of induction. 48 h after induction start, the cells were pelleted by centrifugation at 3.000×g for 10 min. The supernatant was checked for HRP activity by applying the ABTS assay.

1.11 Cultivation of Pichia pastoris in 1.5-L Bioreactors Using the DASGIP System

One single colony per strain of interest was inoculated to 50 mL BMGY as a pre-pre-culture and incubated for 24 h at 28° C., 110 rpm, 80% humidity. The pre-precultures were used to inoculate pre-cultures with a starting OD₆₀₀=0.2 in 60 mL BMGY. The pre-cultures were incubated over night at 28° C., 110 rpm, 80% humidity to reach OD₆₀₀=10-20. Corresponding volumes of the pre-cultures were added to 450 mL of sterile BSM to a final OD₆₀₀=1.0 in the DASGIP reactors.

Using these culture conditions the Batch phase was started with the following settings: Minimum stirring of 500 rpm and minimum of 30% dissolved oxygen (DO) in the fermentation medium. The stirring was coupled to the oxygen level and set to increase, in case the DO went below 30%. The Batch phase was run until all nutrients in the starting medium were used by the cells, indicated by the “starvation peak” in the oxygen levels.

During the FedBatch phase, the cultures were controlledly grown to high cell densities by setting a glycerol feed medium (˜65% glycerol) flow of 5 mL/h per reactor.

Approximately 6 h after the start of the FedBatch phase, the Induction phase was started. A methanol feed medium flow of 3 mL/h was set and the Induction phase was run for 90 h.

1.12 Genomic DNA Isolation and Determination of gDNA Quality and Quantity

Armoracia rusticana

Genomic DNA was isolated from leaves of A. rusticana with the Nexttec™ Genomic DNA Isolation Kit for Plants maxi (nexttec Biotechnologie GmbH, Leverkusen, Germany) according to the manufacturer's recommendations.

The quantity of the isolated gDNA was assessed by estimation from an agarose gel. The quality was assessed by verification of the absorption ratios A260:A280>1.8, A260:A230=2.1 and A260:A270=1.2, measured on the NanoDrop 2000c Spectrophotometer.

Pichia pastoris

In order to isolate gDNA from P. pastoris for qPCR the following protocol was applied (Hoffman et al. ((1987) Gene 57, 267-272)):

A single colony of the desired strain was inoculated to 25 mL sterile YPD and incubated over night at 28° C., 110 rpm. The grown culture (OD₆₀₀<10) was centrifuged for 5 min at 500×g, the pellet was resuspended in 500 μL sterile dH₂O and transferred to a sterile micro-centrifuge tube. The cells were spinned down and the pellet was resuspended in 200 μL yeast lysis buffer. 200 μL phenol:chloroform:isoamyl alcohol (25:24:1) plus approximately 300 mg acid-washed glass beads were added and the suspension was vortexed for 5 min. After addition of 200 μL TE buffer, the suspension was centrifuged for 5 min, 16.100×g and the aqueous phase was transferred to a new microcentrifuge tube. The DNA was precipitated by addition of 1 mL ice cold absolute EtOH and incubation for 10 min at −20° C., spinned down and air dried. The DNA pellet was resuspended in 400 μL TE plus 5 μL 2 mg/mL RNaseA and incubated for >4 h at 37° C. 10 μL 4 M ammonium acetate and 1 mL absolute EtOH were added and the DNA was precipitated as described above. The DNA pellet was washed with 70% EtOH, spinned down and dissolved in a final volume of 50 μL TE buffer.

In order to isolate gDNA from P. pastoris for colony PCR, the Bust n′ Grab protocol described by Harju et al. ((2004) BMC biotechnology 4:8) was applied with the following modifications:

Instead of pelleted cells of an overnight culture, a P. pastoris colony was dissolved in 200 μL lysis buffer. In order to lyse the cells, liquid nitrogen was used instead of dry ice-ethanol. After the isolation steps, RNase treatment was done with 0.125 μL 2 μg/μL RNaseA for 1 h at 37° C.

The assessment of quality and the quantity of the isolated gDNA from P. pastoris was done applying the same criteria as for the isolated gDNA from A. rusticana.

1.13 HRP Activity Assay and Evaluation of HRP Purity ABTS Assay

This standard assay for analyzing the enzymatic activity of HRP was done similar to the ABTS assay described by Morawski et al. (2000):

15 μL of supernatant from either micro-scale cultivation, fermenter culture or purified enzyme were mixed with 140 μL ABTS assay solution (1 mL 20×ABTS stock, 19 mL 50 mM NaOAc, pH 4.5, 1.75 μL 30% (v/v) H₂O₂) and the increase in absorption at 405 nm (ε of oxidized ABTS is 34,700 M⁻¹ cm⁻¹) was followed with the Spectramax Plus 384. The assay conditions were varied in order to characterize A2A. These reactions were performed as triplicates. In case of obvious outliers, one out of three measured points was discarded.

Guaiacol Assay

The guaiacol assay was done similar to the guaiacol assay described by Morawski et al. (2000):

15 μL of HRP solution were mixed with 140 μL guaiacol assay solution (11.1 μL guaiacol, 20 mL 20 mM phosphate buffer, pH 7.0, 0.605 μL 30% (v/v) H₂O₂) and the increase in absorption at 470 nm (ε of oxidized guaiacol is 26,000 M⁻¹ cm⁻¹) was followed with the Spectramax Plus 384. The characterization of the enzymatic performance of HRP A2A with guaiacol as substrate was done with various deviations from the assay conditions.

Assessment of HRP Purity by Verifying the Rz Value

As a criterium of purity the Rz value was measured as described by Morawski et al. (2000): The absorption at 404 nm and at 280 nm was measured on the NanoDrop 2000c Spectrophotometer in order to calculate the Rz value, being the ratio A404:A280.

1.14 Protein Quantitation

In order to quantify dissolved protein, the microplate procedure protocol of the Pierce® BCA Protein Assay Kit (Thermo Scientific Fisher, Waltham, Mass., USA) was applied. 250.0, 125.0, 50.0, 25.0, 5.0, 2.5 and 1.0 ng/μL BSA dilutions and 250.0, 120.0, 62.5, 31.25, 15.625 and 7.8125 ng/μL HRP VI-A dilutions for the generation of standard curves were used. The suggested incubation time was prolonged to 1 h at 37° C. prior to measure the absorption at 562 nm.

1.15 Protein Purification

Buffer Change and Concentration of Protein Solution

The Sartorius Vivaflow 50 system (30,000 MWCO cut-off) was used for sample concentration and buffer change. In order to concentrate samples of volumes smaller than 50 mL, the Sartorius Vivaspin 20 system (3,000 MWCO cut-off) was used.

Hydrophobic Interaction Chromatography

For hydrophobic interaction chromatography, the buffer HIC-A (20 mM Tris-HCl, pH 7.0, 1 M (NH₄)₂SO₄) was used as starting buffer. Buffer HIC-B (20 mM Tris-HCl, pH 7.0) was used for elution.

Cleaning and equilibration of Phenyl Sepharose® 6 Fast Flow (packed in a XK26/20 column) was done as recommended by the manufacturer. Elution was done by replacing buffer HIC-A with buffer HIC-B in a linear gradient from 0% HIC-B to 100% HIC-B over 23× column volumes (CV) with a flowrate of 15 mL/min (˜169.5 cm/h). Changes in absorption at 280 nm and 404 nm, conductivity and the concentration of buffer HIC-B on the column were followed with the UNICORN™ software. The collected fractions were checked for HRP activity with the ABTS assay.

Anion Exchange Chromatography

For determination of the pH of the QFF-buffer A at which HRP A2A binds completely to the Q Sepharose® Fast Flow material, a test tube experiment was performed similarly to the protocol described by GE Healthcare (http://www.gelife-sciences.com/aptrix/upp00919.nsf/Content/TT %3ATest+tube+metho %28150431776-C520%29?OpenDocument&hometitle=tech_support_service (24.Feb.2011)):

About 200 μL Q Sepharose® Fast Flow (QFF) material were transferred to 10 microcentrifuge tubes and spinned down. The supernatant was discarded and the sedimented beads were washed ten times for equilibration. The following 10 different buffers were used in the 10 tubes: Tris-HCl, pH 9.5, Tris-HCl, pH 9.0, Tris-HCl, pH 8.5, Tris-HCl, pH 8.0, Tris-HCl, pH 7.5, citrate-phosphate, pH 7.6, citrate-phosphate, pH 7.0, citrate-phosphate, pH 6.4, citrate-phosphate, pH 5.8, citrate-phosphate, pH 5.0. After equilibration of the beads with 285 μL of the corresponding buffers, 15 μL of HRP solution were added to each microcentrifuge tube. This mixture was vortexed and incubated for 1 min at 4° C. The beads were spinned down and 15 μL of the supernatant were checked for HRP activity by applying the standard ABTS assay at pH 4.5.

In case no HRP activity could be detected in the supernatant, a 1 M NaCl solution of the respective buffer was added and mixed with the HRP-binding beads. After another 1 min incubation at 4° C., the supernatant was checked again for restored HRP activity due to HRP elution from the beads.

For anion exchange chromatography, the buffer QFF-A (50 mM Tris-HCl, pH 9.5) was used as starting buffer and buffer QFF-B (50 mM Tris-HCl, pH 9.5, 1 M NaCl) was used for elution.

20 mL Q Sepharose® Fast Flow material were packed to a XK26/20 column, washed and equilibrated with buffer QFF-A in accordance with the manufacturer's recommendations. Elution was done by changing the ratio of QFF-A:QFF-B in a step-gradient.

Changes in absorption at 280 nm and 404 nm, conductivity and the concentration of buffer QFF-B on the column were followed with the UNICORN™ software. Collected fractions were checked for HRP activity via the ABTS assay and the fractions with highest HRP activity were checked for their Rz values.

Size Exclusion Chromatography

For size exclusion chromatography, the Superdex buffer (3.5 mM citrate, 32.9 mM Na₂HPO₄, pH 7.0) was used.

The HiLoad™ 16/60 Superdex™ 200 prep grade column, prepacked with 120 mL matrix material, was washed and equilibrated as recommended by the manufacturer. The sample was concentrated using the Vivaspin 20 system and loaded onto the column with a flowrate of 0.3 mL/min (˜9.0 cm/h). The same flowrate was applied for the size exclusion run over 2×CV. Changes in absorption at 280 nm and 404 nm and conductivity were followed with the UNICORN™ software. The fractions were checked for HRP activity with the ABTS assay and the fractions with highest HRP activity were assessed for their Rz values.

Fractional Precipitation with Ammonium Sulfate

100 μL samples were incubated in (NH₄)₂SO₄ solutions of stepwise increasing molarity at 4° C. for 45 min per step. After incubation, the samples were spinned down at 16,100×g for 15 min. The supernatant was used for the next precipitation step, the pellet was resuspended in buffer QFF-A. The buffer conditions of the various steps are shown in Table 3:

TABLE 3

| molarity mol/L | saturation at 4° C. % |
|---|---|
| 0.00 | 0 |
| 0.39 | 10 |
| 0.79 | 20 |
| 1.18 | 30 |
| 1.57 | 40 |
| 1.96 | 50 |
| 2.36 | 60 |
| 2.75 | 70 |
| 3.14 | 80 |
| 3.54 | 90 |

The fractions, being the resuspended pellets and the supernatant of the last precipitation step, were checked for HRP activity by applying the ABTS assay and their Rz values were determined.

Affinity Chromatography

For affinity chromatographic purification via a fused StrepTagII, the Strep-tag® purification protocol by IBA GmbH was applied according to the manufacturer's recommendations.

### Example 1

### HRP Sequences from Next-Generation Sequencing 454 Sequencing of the Armoracia rusticana Transcriptome

In order to identify and verify nucleotide sequences of horseradish peroxidase isoenzymes, a search for GenBank- or UniProt-published HRP sequences in the transcriptome was conducted.

High quality total RNA has been isolated from horseradish, normalized in terms of RNA abundance and length, and sequenced by LGC Genomics GmbH (Berlin, Germany) by using the Roche Applied Science GenomeSequencer FLX Titanium technology. A de novo assembly using the Newbler Assembler 2.01 was done by LGC Genomics that provided an alignment of approximately 590,000 reads to a total of ˜27,000 contigs with ˜13,000 being longer than 500 bp.

The BLAST-NCBI (default settings) was used implemented in the ClusterControl system at the Institute of Genomics and Bioinformatics at the Graz University of Technology to check the transcriptome contigs for HRP sequences published either at GenBank or UniProt. Furthermore, also the published HRP sequences were BLASTed against the transcriptome raw read sequences; the hits isolated and manually assembled using ClustalW2.

In order to identify new, so far unknown HRP isoenzyme sequences, the total contig number of approximately 27,000 contigs was minimized by performing a tBLASTn search with the known HRP protein sequences as well as 90% identity-clustered Arabidopsis thaliana peroxidase sequences against all contigs. An e-value of 10⁻⁵ and the BLOSUM62 matrix were chosen in order to include many false positives. The longest open reading frame in each of the found contigs was translated and the encoded protein sequence was classified using the NCBI Conserved Domains Database (CDD). The contigs identified as secretory peroxidases were manually analyzed with the DNAstar/SeqMan software, presumedly misassembled reads were split to separate contigs, trimmed reads were extended if possible.

454 Sequencing of the Armoracia rusticana Genome

In addition to next-generation sequencing of the horseradish transcriptome the 454 sequencing of a shot-gun FLX Titanium library as well as a 3 kb paired-end library of the horseradish genome at the ZMF Molecular Biology Core Facility (Center for Medical Research, Graz, Austria) were ordered. Assuming the horseradish genome to be of similar size as the Arabidopsis thaliana genome (157 Mbp), which most probably underestimates the true genome size; a putative double coverage in average was expected.

The genomic DNA (gDNA) was isolated from the leaves of horseradish and sequenced by 454 sequencing.

The sequencing provided a total of approximately 286 Mb (linker/primer sequences excluded) in ˜676,000 shotgun reads and ˜464,000 paired-end reads. Assembling of the reads was approached with the Newbler assembler (standard settings, primer/linker trimming), as well as with MIRA (after Newbler extraction and trimming of the reads). Since both approaches featured just a small part of the total assumed genome size, the previously identified transcriptome contigs were sought directly in the genome sequencing reads via tBLASTn (e-value 10-5, matrix PAM30) of the protein sequences derived from the transcriptome. By displaying the matches of all nearly identical high-scoring segment pairs (>10 residues) and by using the GeneWise tool (http://www.ebi.ac.uk/Tools/Wise2/index.html, 01.Mar.2011) for intron finding and the Needleman-Wunsch algorithm (http://www.ebi.ac.uk/Tools/emboss/align/index.html, 01.Mar.2011) for the alignments (gap open penalty 100.0, gap extension penalty 0.0005), exons with adjacent intron sequences were supposed to be found.

Verification of HRP Sequences with Sanger Sequencing

In addition to the identification of published and new HRP isoenzymes from next-generation sequencing approaches, those sequences have been verified on genomic DNA level via Sanger sequencing, which allows the determination of full gene sequences (i.e. exonic and intronic sequences) at highly reliable quality.

Primer pairs that bind in the 3′- and 5′-untranslated regions (UTR) of those isoenzymes published at GenBank (i.e. C1A, C1B, C1C, C2, C3, N) were designed. In order to design primers for isoenyzmes published at UniProt, those sequences that were successfully deduced from the transcriptome (i.e. E5, A2) could be used. Primers for two transcriptome contigs encoding new HRPs (i.e. 22684, 01805) that were found in the BLAST search with the published HRP sequences were designed.

For some isoenzymes, there was too little distinctive sequence information available in the UTRs to design specific primers. A Genome Walking approach in order to verify the C-terminal encoding regions of E5 and contig 22684 as well as the N-terminal encoding regions of C1C and N was chosen. The respective gene-specific primers were: B2CtermSpecific1a and B2CtermSpecific2, ESCtermSpecific1 and ESCtermSpecific2, newC1CNtermSpecific1a and newC1 CNtermSpecific2, newNNtermSpecific1a and newNNtermSpecific2. The primers labelled “B2” correspond to 22684.

Further primers were designed to bind within the HRP genes, in order to ensure an overall sequence coverage of at least two times. These primers were used in PCRs and/or for Sanger sequencing. In case a certain region of an isoenzyme could not be amplified unambiguously or did not yield explicit sequencing data due to unspecific primer bindings, the PCR product was ligated to the pJET1.2blunt vector, transformed to Escherichia coli and the clonally amplified plasmids from approximately eight different clones were isolated and sent to Sanger sequencing separately, in order to guarantee distinct sequence information of the inquired isoenzyme. Otherwise, the PCR product was sent directly to Sanger sequencing. In order to verify the full gene sequences of the new additional isoenzymes that were found in the CDD-associated approach (i.e. contigs 23190 (SEQ ID NO:15), 04663 (SEQ ID NO:16), 06351 (SEQ ID NO:17), 06117 (SEQ ID NO:18), 04791, (SEQ ID NO:10), 03523 (SEQ ID NO:19), 17517 (SEQ ID NO:20), 01350 (SEQ ID NO:21), 02021 (SEQ ID NO:27), 05508 (SEQ ID NO:23), 08562 (SEQ ID NO:24), 22489 (SEQ ID NO:26)), gene-flanking primers were used for PCR amplification. In case the primer pairs yielded distinctive PCR products, they were ligated to pJET1.2blunt vector, amplified in E. coli and eight plasmid isolations per isoenzyme were sent to Sanger sequencing.

The sequence reads were aligned to a reference sequence if available (i.e. published sequences and transcriptome sequences) or assembled de novo using SeqMan. Intron finding was achieved by applying the PlantGDB GeneSeger web tool.

Analysis of Verified HRP Sequences

The whole encoded protein sequences of the Sanger-verified isoenzymes were aligned to each other using ClustalW2 in order to get a picture of how closely the isoenzymes are related to each other. Moreover, SignalP 3.0 was used for the prediction of signal peptide cleavage sites and WebLogo 3 for a visual representation of the identified cleavage sites. Furthermore, a theoretical isoelectric point (IEP) was calculated with the Expasy Compute pl/Mw tool of all verified HRP isoenzymes using the whole protein sequences, in order to allow an assignment of them to an isoenzyme group.

454 Sequencing of the Armoracia rusticana Transcriptome

By BLASTing the published HRP sequences separately against the transcriptome contigs, the contigs 15901 (SEQ ID NO:28), 25148 (SEQ ID NO:29), 04627 (SEQ ID NO:30) and 00938 (SEQ ID NO:32) were successfully identified to which the HRP isoenzymes C1B, C1C, C2 and E5 were assigned, respectively. Further, two contigs, 01805 (SEQ ID NO:31) and 22684 (SEQ ID NO:33), encoding yet unpublished HRP isoenzymes were found. Contig 01805 showed 84% identity to the published coding sequence of C1A, contig 22684 was 90% identical with the published coding sequence of HRP C3. The identity percentage of the published coding sequences of C1A and C1B, C1A and C1C, C1B and C1C was 89%, 90%, 94%, respectively.

BLASTing published HRP sequences against all transcriptome sequencing raw reads allowed the identification of reads that could be manually assembled to match the published HRP isoenzyme A2.

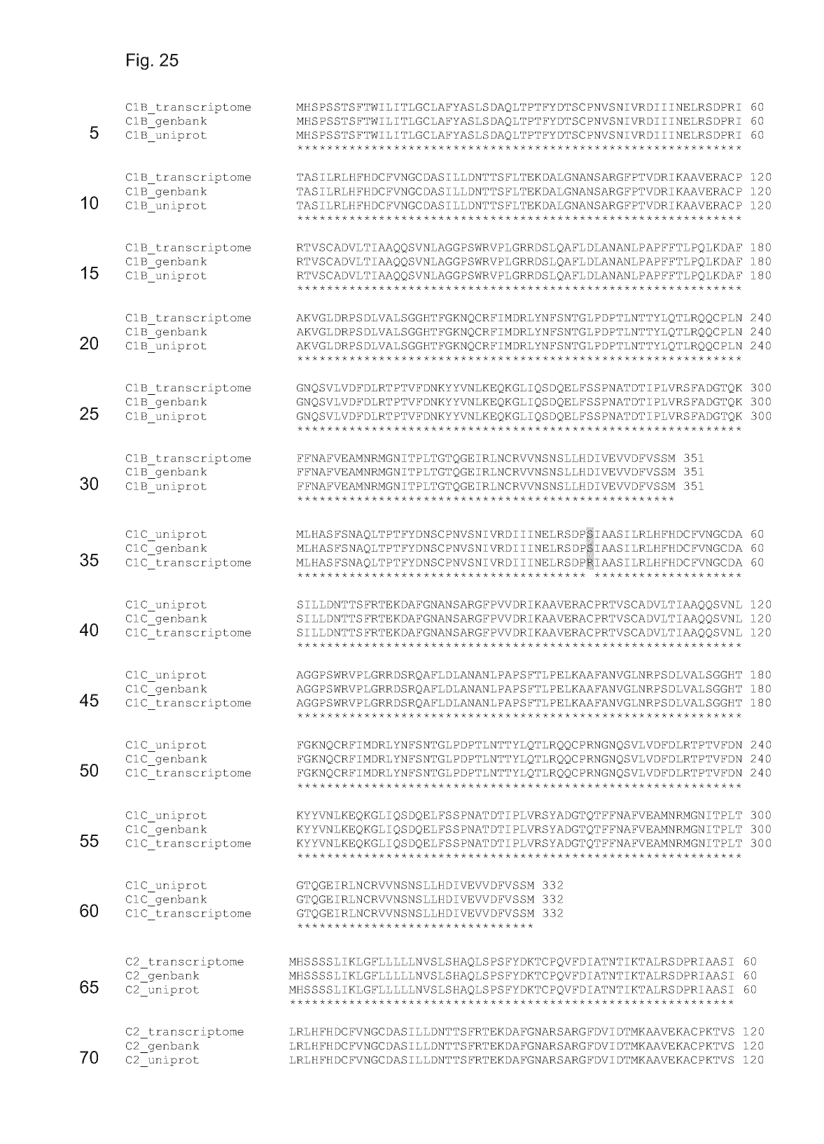

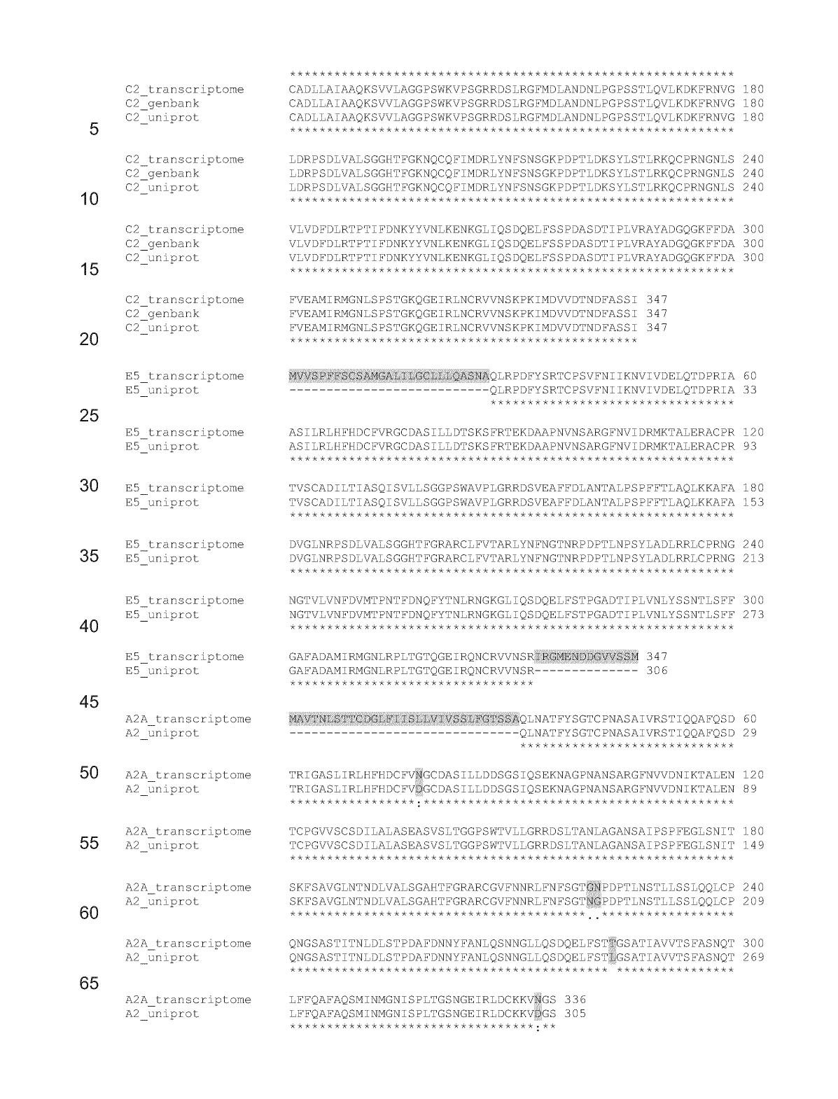

The alignments of the identified translated isoenzyme sequences against the amino acid sequences from UniProt and—if available—the translated sequences from Gen Bank are shown in FIG. 25. The published and transcriptome-derived coding sequences of C1B and C2 were identical. Since the A2 sequence at UniProt lacks the 31 as N-terminal signal peptide sequence, the positions of the affected amino acids in the table differ by the number 31. All differences are described in Table 4.

TABLE 4

| | translated transcriptome | | UniProt/translated | |
|---|---|---|---|---|
| HRP | sequence | | GenBank sequence | |
| C1B | = | | = | |
| C1C | Arg40 | | Ser40 | |
| C2 | = | | = | |
| E5 | extra N-terminal signal peptide and | | only main chain | |
| | C-terminal propeptide sequence | | | |
| A2 | extra N-terminal signal peptide | | only main chain | |
| | Asn78 | Thr284 | Asp47 | Leu253 |
| | Gly221 | Asn334 | Asn190 | Asp303 |
| | Asn222 | | Gly191 | |

In the search for new HRP sequences, 48 contigs could be identified after the first BLAST search step. Checking for a CDD classification as secretory peroxidase of the protein sequences encoded in these 48 contigs further reduced the contig number to 16. Manual reviewing of these contigs facilitated the identification of a total number of 19 contigs that featured a full secretory peroxidase domain (i.e. contig23190 (SEQ ID NO:15), contig04663 (SEQ ID NO:16), contig06351 (SEQ ID NO:17), contig06117 (SEQ ID NO:18), contig03523 (SEQ ID NO:19), contig17517 (SEQ ID NO:20), contig00938 (SEQ ID NO:32), contig01805 (SEQ ID NO:31), contig04627 (SEQ ID NO:30), contig15901 (SEQ ID NO:28), contig22684 (SEQ ID NO:33), contig01350 (SEQ ID NO:21), contig01350_ALTERNATIVE (SEQ ID NO:22), contig02021 (SEQ ID NO:27), contig25148 (SEQ ID NO:29), contig05508 (SEQ ID NO:23), contig08562 (SEQ ID NO:24), contig08562_ALTERNATIVE (SEQ ID NO:25), contig22489 (SEQ ID NO:26)). Among these were again the contigs encoding the isoenzymes E5, C2, C1B and C1C and the two contigs 01805 and 22684 which have already been found before. Inconsistencies of assembled reads in one contig were mainly considered to be due to allelic variations.

454 Sequencing of the Armoracia rusticana Genome

The assemblies of the genome sequencing reads featured ˜64,000 contigs with 23 Mb from the Newbler approach, and ˜23,000 contigs with 12 Mb from the MIRA approach. The following results were achieved by checking the genome sequencing reads directly for HRP isoenzymes: One full exonic sequence and two partial intronic sequences corresponding to the C1B-encoding transcriptome contig 15901 were found in 12 reads of the genome Newbler contig 38504. One full exon and 2 partial intronic sequences belonging to the transcriptome contig 08562 could be identified on a single genome sequencing read. Three genome reads represented the sequences of two full exons, one full and one partial intron associated to the contig 08562_ALTERNATIVE (SEQ ID NO:25). One genome sequencing read could be identified to feature a partial exonic and a partial intronic sequence belonging to the transcriptome contig 01351 (SEQ ID NO:34) which has been characterized by CDD criteria to contain a C-terminally truncated part of a conserved domain of the plant peroxidase-like superfamily.

Verification of HRP Sequences with Sanger Sequencing

A total of 20 HRP gene sequences on genomic DNA level via Sanger sequencing with double coverage at the minimum were successfully verified. The verified isoenzymes and the deviations of the Sanger sequences from the transcriptome sequences, as well as from GenBank and UniProt sequences, and the influence of these deviations on the amino acid sequence are shown in Table 5:

TABLE 5

| | Sanger sequence | | transcriptome | | GenBank sequence | | UniProt |
|---|---|---|---|---|---|---|---|
| HRP | | aa (exon)/ | sequence | | | aa (exon)/ | sequence |
| gene | nt | intron | nt | aa | nt | intron | aa |
| C1A | TA | Y37 | — | — | AT | I37 | Y37 |
| (SEQ ID | 109-110 | | | | 109-110 | | |
| NO: 51) | C1159 | intron | — | — | G1159 | intron | — |
| C1B | T/C253 | intron | — | — | T253 | intron | — |
| (SEQ ID | T/C859 | intron | — | — | C859 | intron | — |
| NO: 1) | | | | | | | |
| C1C | ss | ss | ss | ss | * | * | * |
| (SEQ ID | nt1-60 | aa1-20 | nt1-60 | aa1-20 | | | |
| NO: 2) | C178 | R60 | C178 | R60 | A118 | S40 | S40 |
| | A/T1335 | intron | — | — | — | — | — |
| | A/G1888 | T/A165 | G493 | A165 | G433 | A145 | A145 |
| | C/G1921 | Q/E176 | G526 | E176 | G466 | E156 | E156 |
| C2 | CT | intron | — | — | * | intron | — |
| (SEQ ID | 1250-1251 | | | | | | |
| NO: 3) | A1334 | intron | — | — | * | intron | — |
| C3 | G/T1294 | intron | — | intron | G1294 | intron | — |
| (SEQ ID | A/T1323 | intron | — | intron | A1323 | intron | — |
| NO: 94) | T/C1484 | L231 | — | — | T1484 | L231 | L231 |
| | C/T1541 | F250 | — | — | C1541 | F250 | F250 |
| A2A | ss | ss | ss | ss | — | — | * |
| (SEQ ID | nt1-93 | aa1-31 | 1-93 | aa1-31 | | | |
| NO: 4) | AAT | N78 | AAT | N47 | — | — | D47 |
| | 231-234 | | 231-234 | | | | |
| | GGA | G220 | GGA | G220 | — | — | N189 |
| | 996-998 | | 661-663 | | | | |
| | AAT | N221 | AAT | N221 | — | — | G190 |
| | 999-1001 | | 664-666 | | | | |
| | ACG | T284 | ACG | T284 | — | — | L253 |
| | 1185-1187 | | 850-852 | | | | |
| | G/A1203 | A/T290 | A868 | A290 | — | — | A259 |
| | AAT | N334 | AAT | N334 | — | — | D303 |
| | 1335-1337 | | 999-1002 | | | | |
| E5 | ss | ss | ss | ss | — | — | * |
| (SEQ ID | nt1-81 | aa1-27 | nt1-81 | aa1-27 | | | |
| NO:)5 | T419 | L82 | C246 | L82 | — | — | L55 |
| | C422 | D83 | T249 | D83 | — | — | D56 |
| | C545 | C124 | T372 | C124 | — | — | C97 |
| 01805 | none | none | none | none | — | — | — |
| (SEQ ID | | | | | | | |
| NO: 6) | | | | | | | |
| 22684 | G1611 | R337 | A1010 | K337 | — | — | — |
| (SEQ ID | TGA | D343 | CGG | G343 | — | — | — |
| NO: 7) | 1627-1629 | | 1026-1028 | | | | |
| 01350 | none | none | none | none | — | — | — |
| (SEQ ID | | | | | | | |
| NO: 8) | | | | | | | |
| 02021 | none | none | none | none | — | — | — |
| (SEQ ID | | | | | | | |
| NO: 9) | | | | | | | |
| 03523 | none | none | none | none | — | — | — |
| (SEQ ID | | | | | | | |
| NO: 19) | | | | | | | |
| 06117 | T30 | V10 | C30 | V10 | — | — | — |
| (SEQ ID | C1088 | I269 | T807 | I269 | — | — | — |
| NO: 11) | | | | | | | |
| 17517 | T190 | Y64 | C190 | H64 | — | — | — |
| (SEQ ID | C1157 | G282 | T846 | G282 | — | — | — |
| NO: 12) | A1232 | K307 | G921 | K307 | — | — | — |
| 08562.1 | none | none | none | none | — | — | — |
| (SEQ ID | | | | | | | |
| NO: 13) | | | | | | | |
| 08562.4 | none | none | none | none | — | — | — |
| (SEQ ID | | | | | | | |
| NO: 14) | | | | | | | |
| 23190 | T327 | S109 | G327 | S109 | — | — | — |
| (SEQ ID | C405 | G135 | T405 | G135 | — | — | — |
| NO: 15) | C672 | T224 | A672 | T224 | — | — | — |
| | A1043 | E348 | T1043 | V348 | — | — | — |
| 04663 | none | none | none | none | — | — | — |
| (SEQ ID | | | | | | | |
| NO: 16) | | | | | | | |
| 06351 | none | none | none | none | — | — | — |
| (SEQ ID | | | | | | | |
| NO: 17) | | | | | | | |
| 05508 | G/A346 | A/T116 | G346 | A116 | — | — | — |
| (SEQ ID | | | | | | | |
| NO: 23) | | | | | | | |
| 22489 | — | — | G/A597 | T199 | | | |
| (SEQ ID | . | . | G/T715 | A/S239 | | | |
| NO: 26) | | | | | | | |

This direct comparison of the data from Sanger sequencing using a gDNA template and 454 sequencing from a transcriptome cDNA library reveals that only 3 raw reads representing the HRP isoenzyme C1A could be found in the transcriptome reads. Also only a few reads representing a minor part of isoenzyme C3 could be found. This finding was highly unexpected, since C1A has been described in literature as the most abundant isoenzyme in horseradish. It might be possible that the presumedly highly abundant C1A transcript was almost completely degraded during the cDNA normalization via kamchatka crab duplex-specific nuclease (Zulidov et al., (2004) Nucleic acid research 32, e37). In order to prove this hypothesis, primers specific for C1A could be used for a PCR using the cDNA library as template for the verification of the presence or absence of C1A transcripts. Alternatively, the used horseradish variety might show different expression of isoenzymes than the plant material studied so far or the expression of C1A is dependent on the season or specific environmental conditions such as wounding, parasites, temperature or day length in the natural environment.

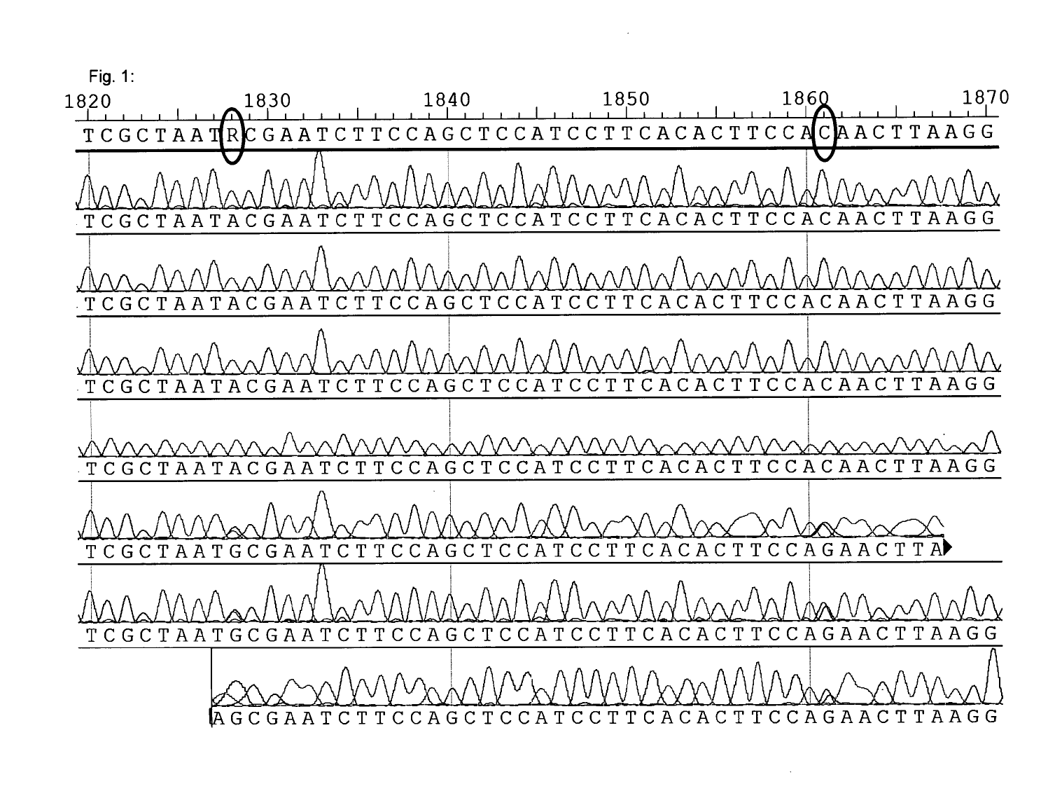

The double peak signals A/G1828 and C/G1861 (see Table 5) in the alignment of the Sanger sequencing reads matched to isoenzyme C1C are shown in FIG. 1. All allelic sequences seem to have been assembled as a mixture in the transcriptome contig identified as C1C. Underlining this assumption, both putatively allelic sequences could be found in the transcriptome raw reads as separate reads.

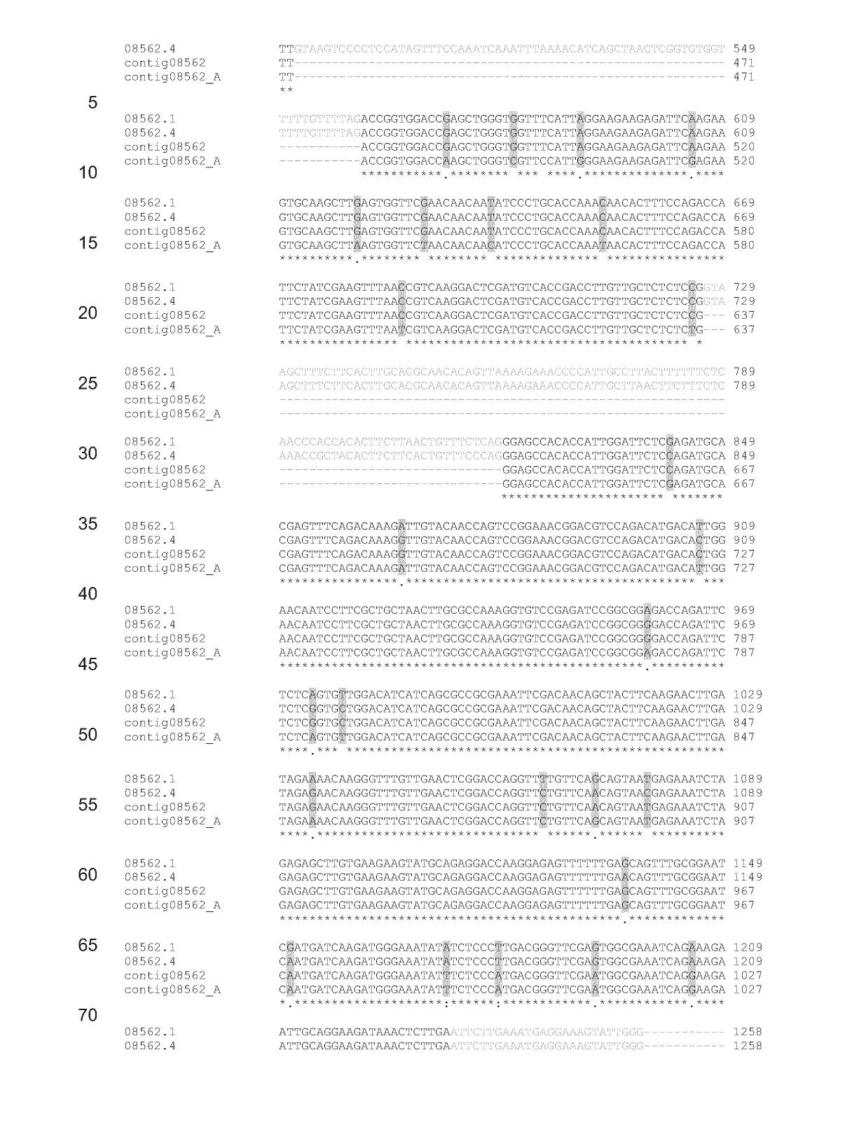

The two Sanger-verified sequences 08562.1 and 08562.4 are shown in an alignment with the corresponding transcriptome contigs. All observed differences and the homopolymeric region from position 98-104 of contig 08562 and contig 08562_ALTERNATIVE are marked in grey. Non-coding sequence is written in grey letters (FIG. 27).

Identified differences between 08562.1 and 08562.4 on amino acid level were Lys88 versus Arg88 and Ser279 versus Asn279 in 08562.1 versus 08562.4, respectively.

The alignment of the presumably allelic sequences of 08562.1 and 08562.4 from Sanger sequencing with the transcriptome contigs 08562 and 08562_ALTERNATIVE illustrates important issues in the assembly of next-generation sequencing reads of isoenzyme sequences. Moreover, it emphasizes the clear need for manual sequence verification in order to clarify assembly mistakes and homopolymeric regions, which constitute a known problem that is inherent to the 454 sequencing method.

The transcriptome reads from the two verified isoenzymes 08562.1 and 08562.4 have been found to be mixed and misassembled. Hence, more restrictive assembly settings might be helpful in order to distinguish between these two isoenzymes and assemble them to the two correct contigs. However, by applying more restrictive settings, the tolerance for sequencing mistakes decreases in an inversely proportional way which might lead to the identification of further “isoenzymes” that actually originate from errors in the sequencing procedure. Separate PCR amplifications of 08562.1 and 08562.4 from the cDNA library and subsequent Sanger sequencing could be done to prove the assumed misassembly.

Furthermore, the T-oligomeric region at position 98-104 of the two transcriptome contigs, which at first has been manually “corrected” with an insertion X in order to account for a frameshift mutation, has been shown to actually consist of a pentamer instead of the assumed heptamer.

The existence of allels might be an explanation for the observed variations in the sequences of (putatively) one isoenzyme gene. Even though there is no information available on the actual degree of ploidy of A. rusticana, this is a very likely possibility, especially taking the obtained sequencing data of C1C or 08562 into consideration. Bearing this in mind, the high number of different isoenzymes observed via isoelectric focusing could be explained by the detection of two (or more) expressed allels of “one” isoenzyme gene. This illustrates an issue in the nomenclature and classification of isoenzymes: In general, all HRP molecules present in an extract from horseradish have been separated by isoelectric focusing and counted as individual isoenzymes. Hence, the verified sequence of A2A for example, might be both, an allel of the published A2 gene, but just as well one of the also observed isoenzymes A1 or A3. And again, A1 and A2 might be allels of the same gene or actually two separate genes on one chromosome. This question remains unanswered due to the low quality of the obtained genome sequence.

Since gene duplication is a proposed mechanism for the evolution of HRP isoenzymes, highly similar HRP gene sequences can also be explained hereby and do not necessarily have to be allels.

Further explanations for the observed differences in all verified sequences might be a variance in the HRP genes among locally separated A. rusticana populations or mistakes produced by the reverse transcription of mRNA in the library generation.

The combination of transcriptome sequencing and Sanger sequencing of amplified genomic DNA revealed 38 variable positions in the coding sequences of 11 genes. For 9 positions thereof (in 4 transcripts), the corresponding nucleotide could not be found in the genomic DNA.

In summary, 28 HRP isoenzymes including putative allelic variants were successfully identified, using the data from the 454 transcriptome sequencing, Sanger sequencing of amplified genomic DNA, Genbank and UniProt. Moreover, at least 15 of the 28 HRPs are new isoenzymes that have not been published yet.

Analysis of Verified HRP Sequences

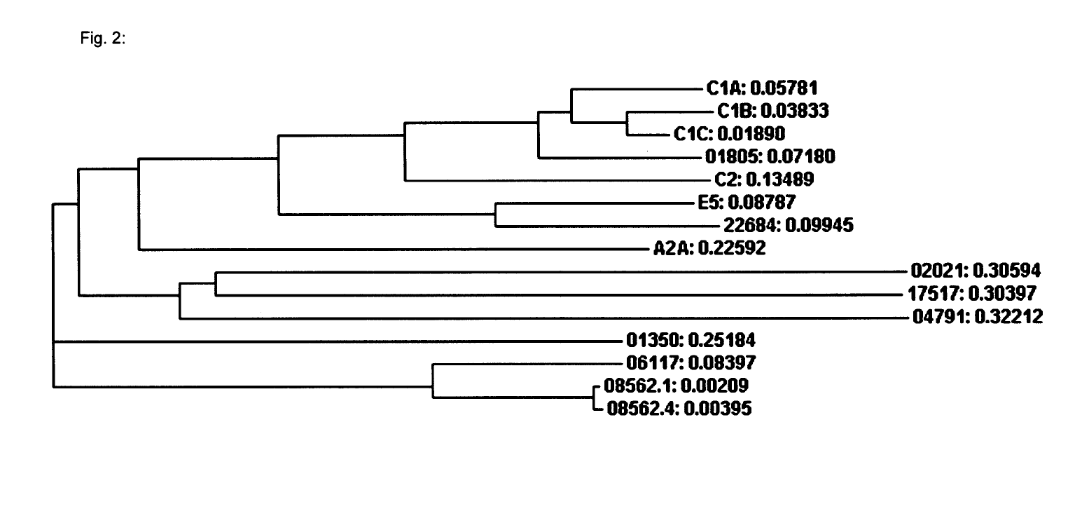

The data provided from the multiple sequence alignment of the verified HRP amino acid sequences could be visualized in a phylogenetic tree (FIG. 2).

The published HRP isoenyzmes are closer related to each other than to the new isoenzymes. A2A seems to be the most distant isoenzyme of the published ones. Just 01805 and 22684 of the new isoenzymes are closely related to the published HRPs. The other new isoenzymes cluster to two groups: 02021, 17517 and 04791 form one group, 01350, 06117, 08562.1 and 08562.4 form the second group.

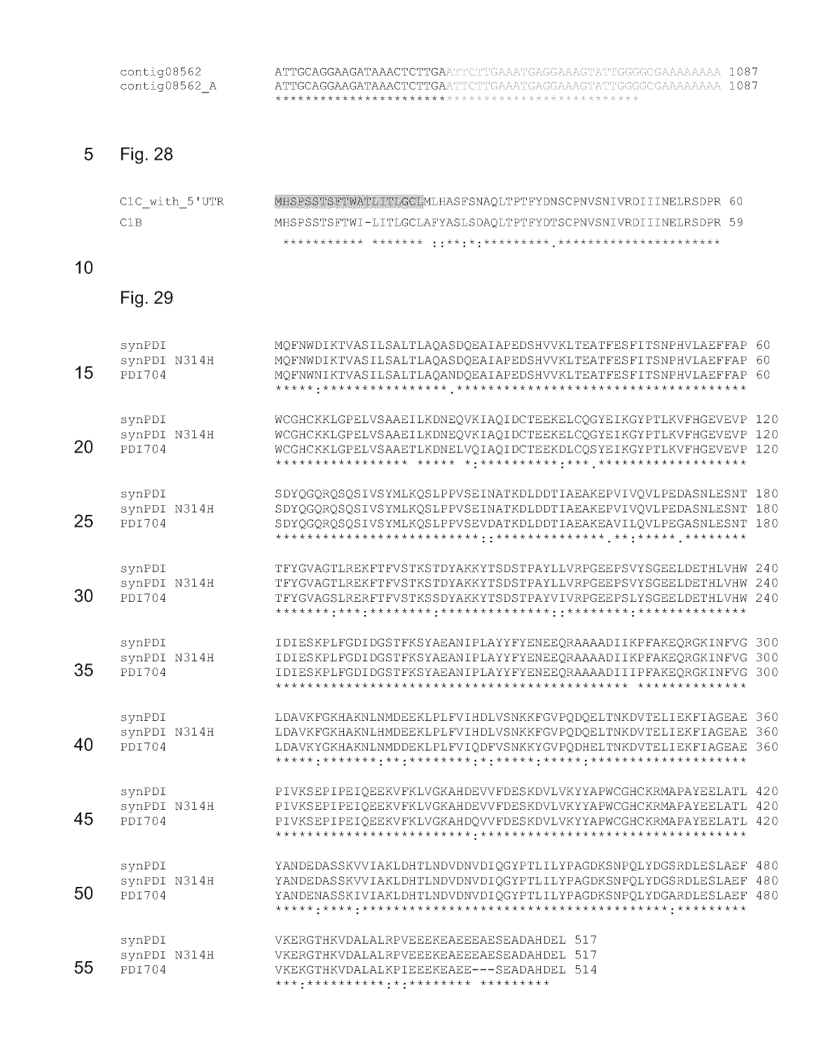

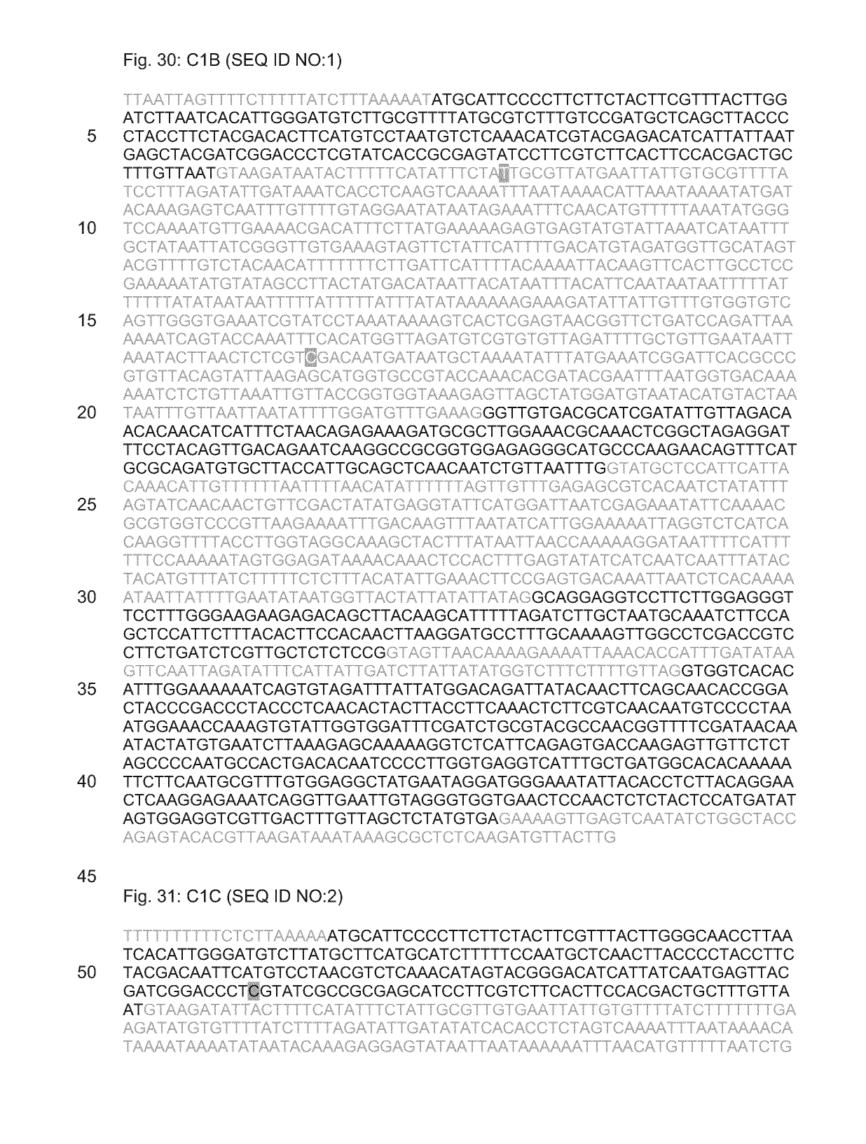

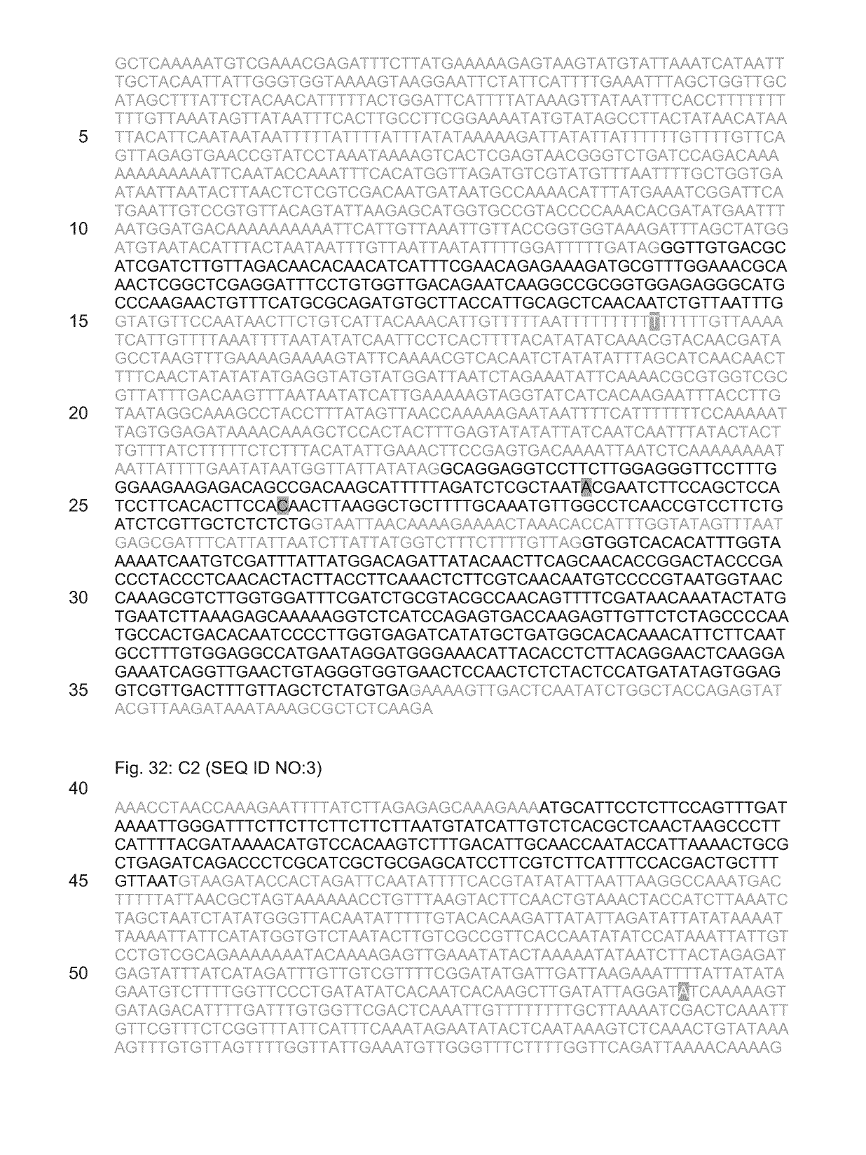

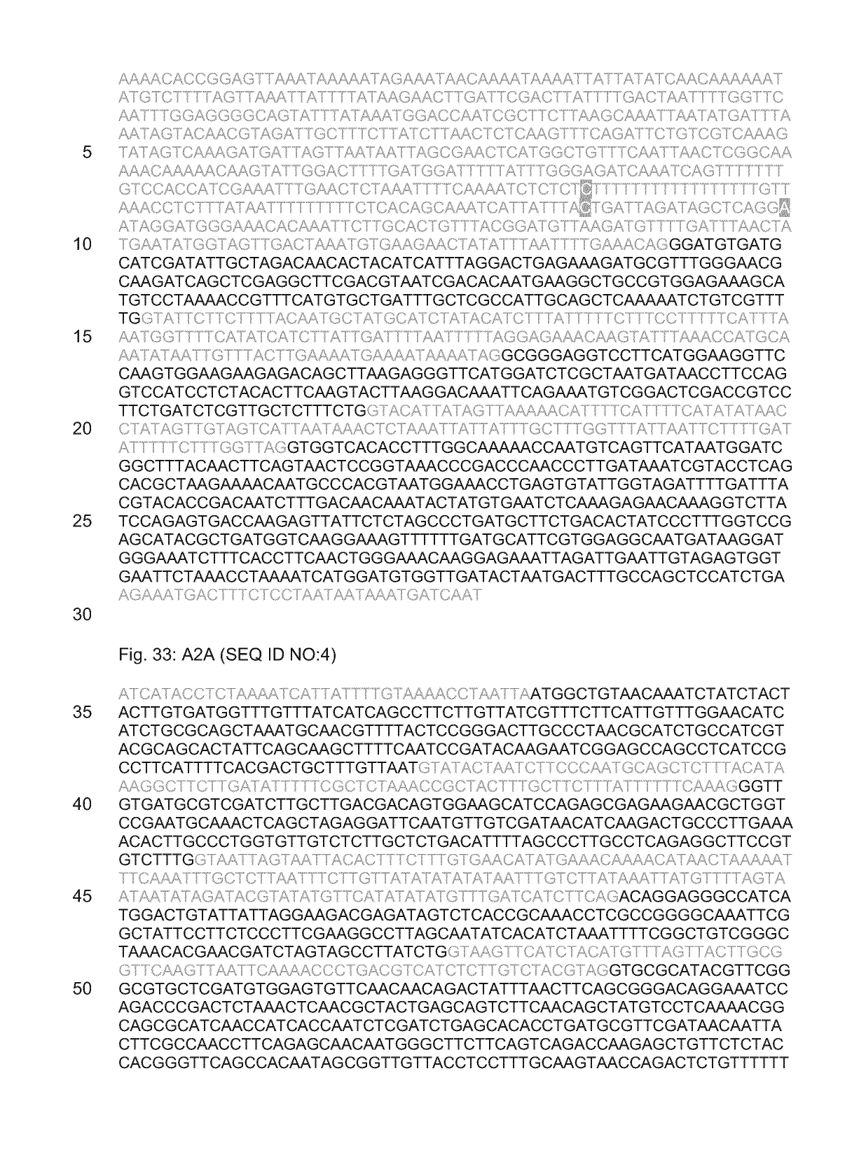

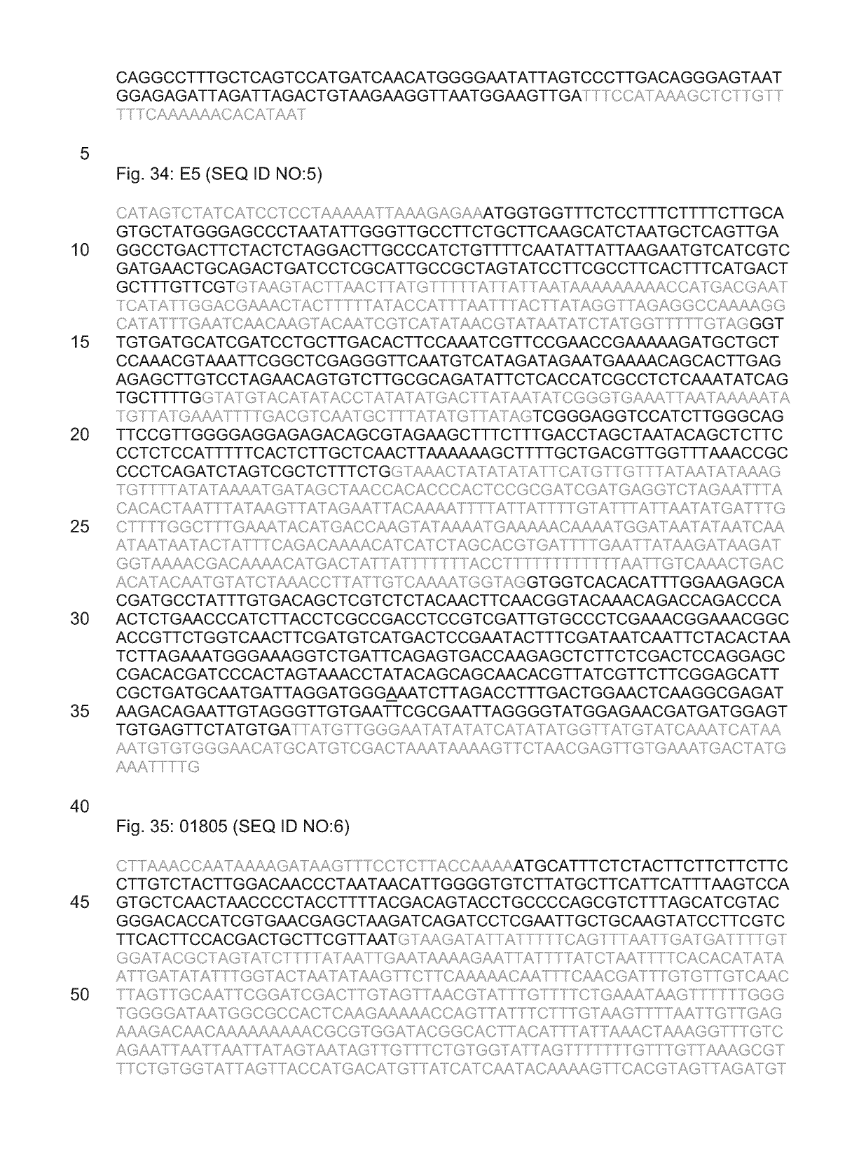

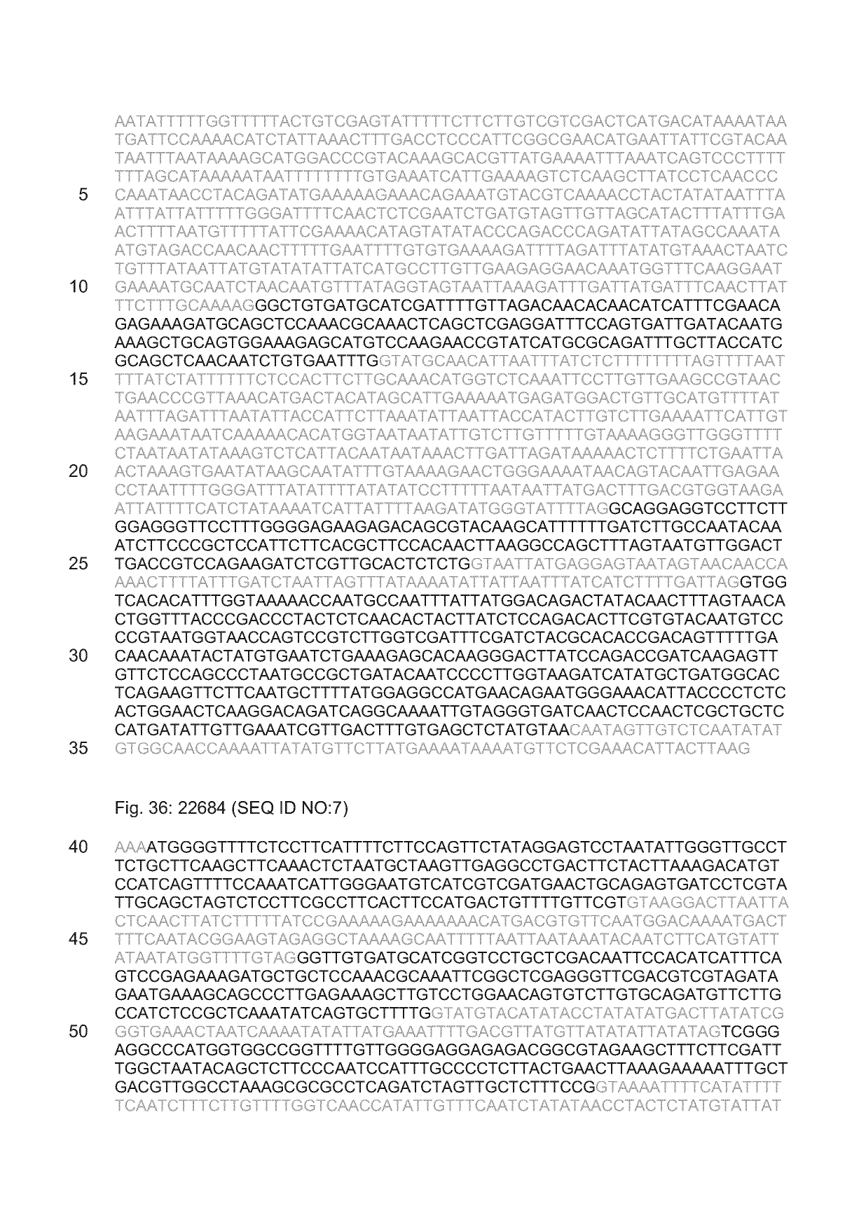

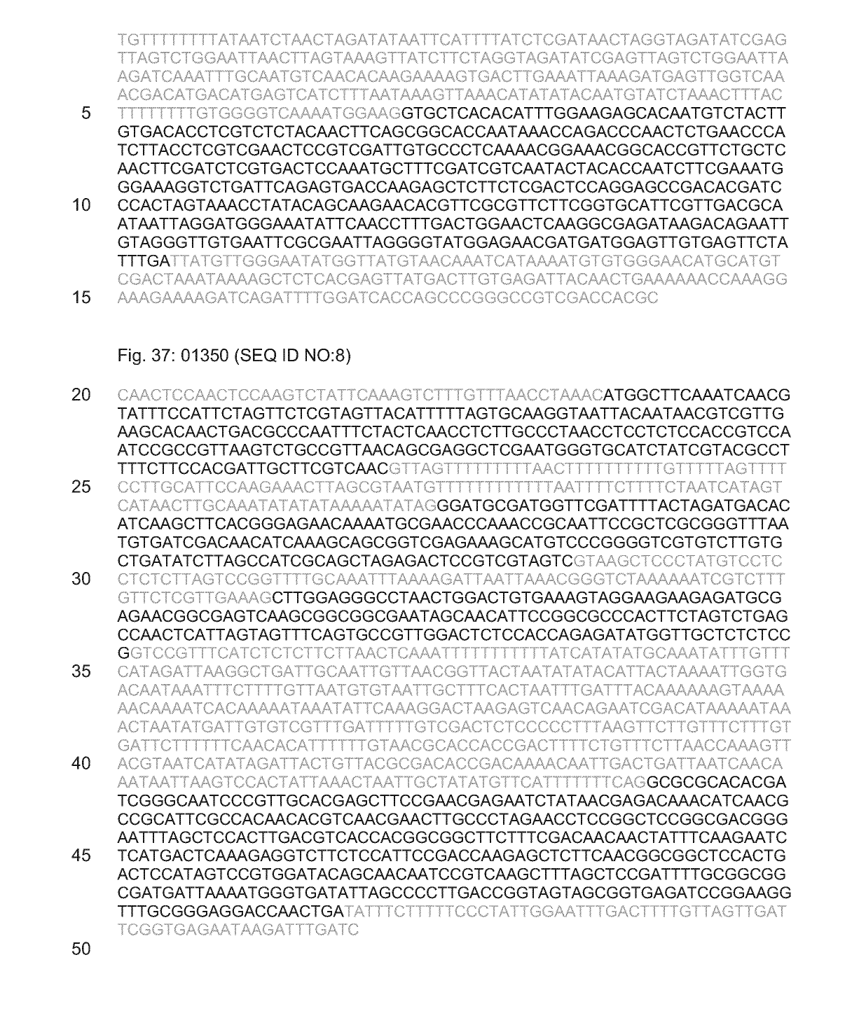

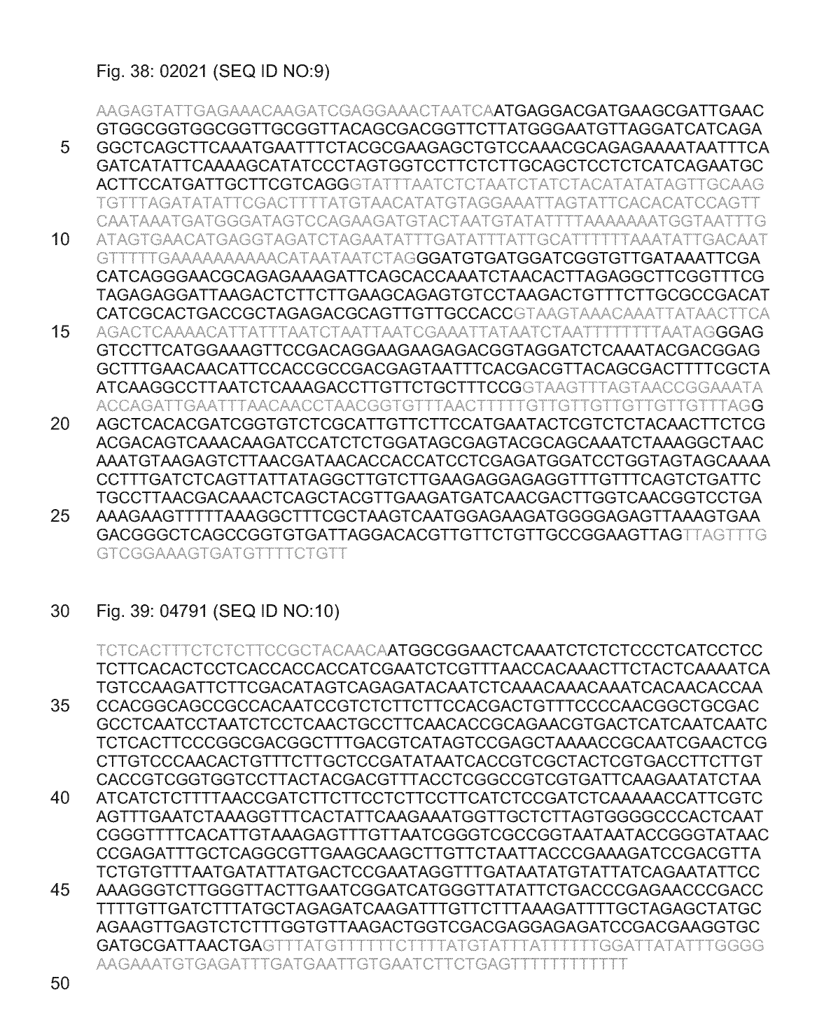

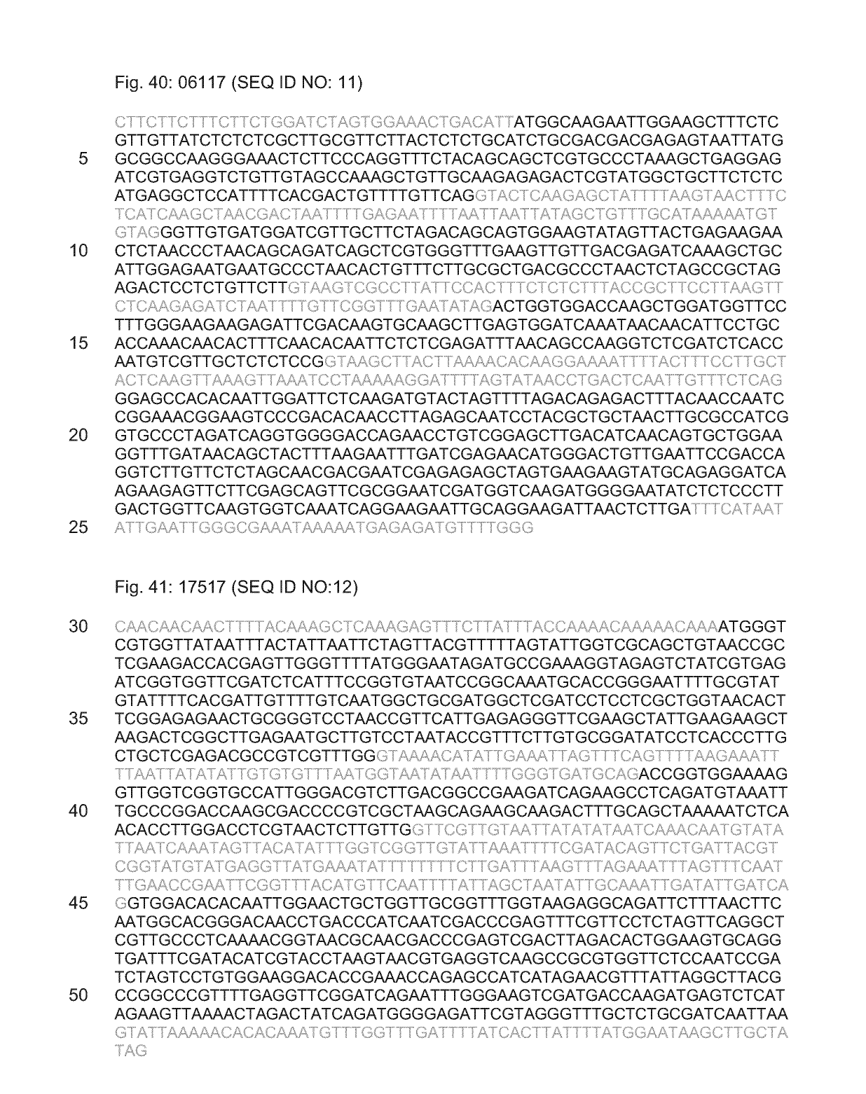

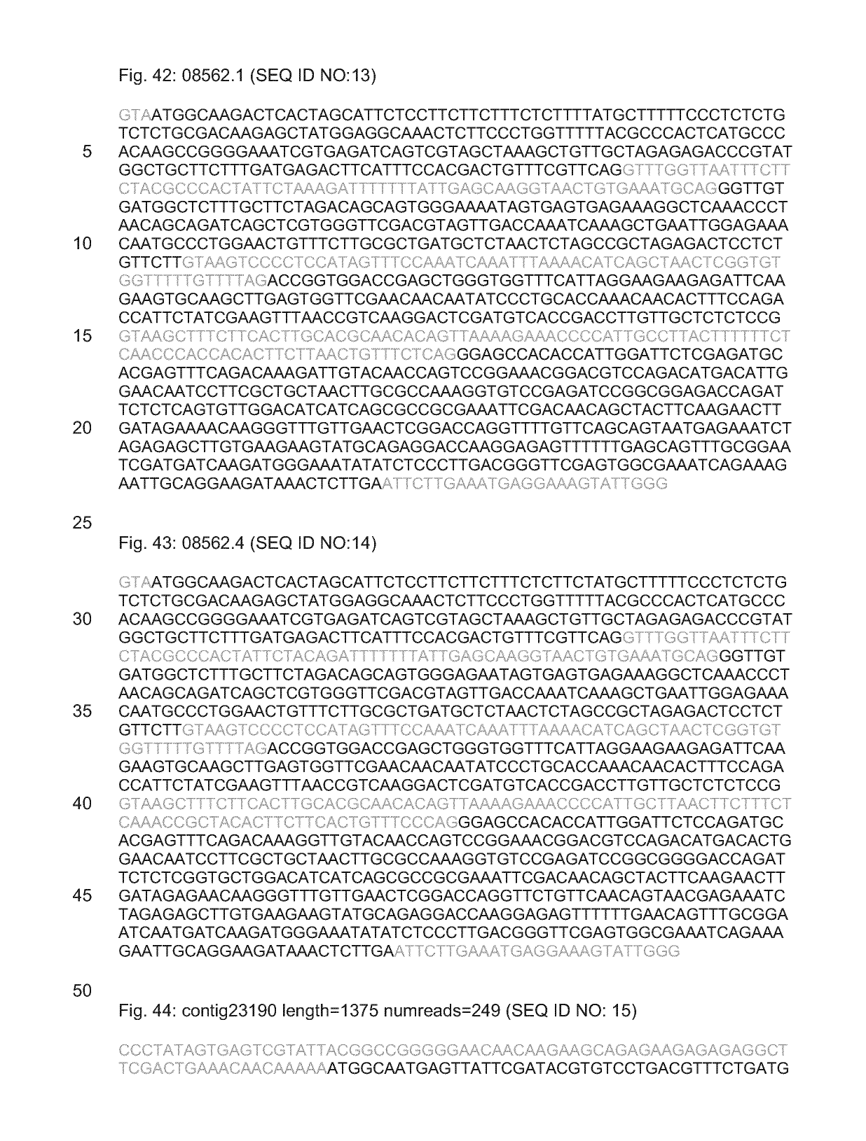

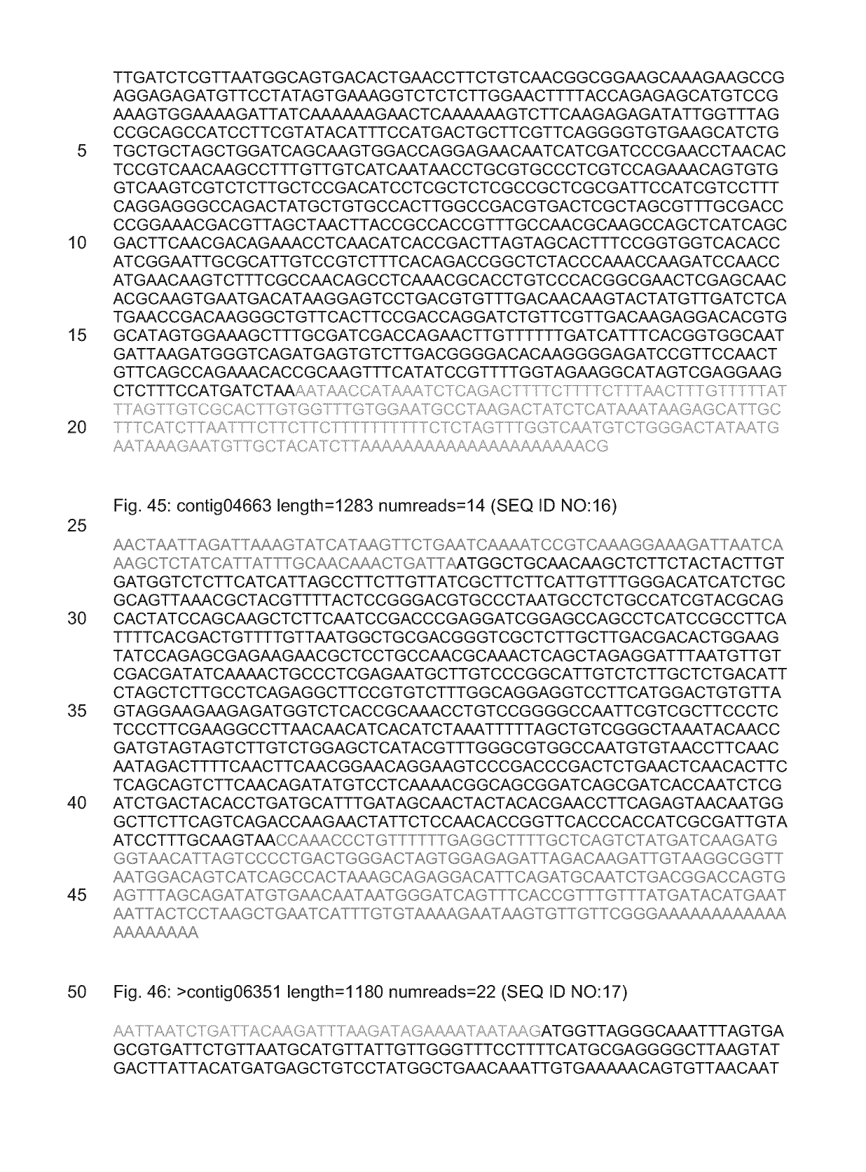

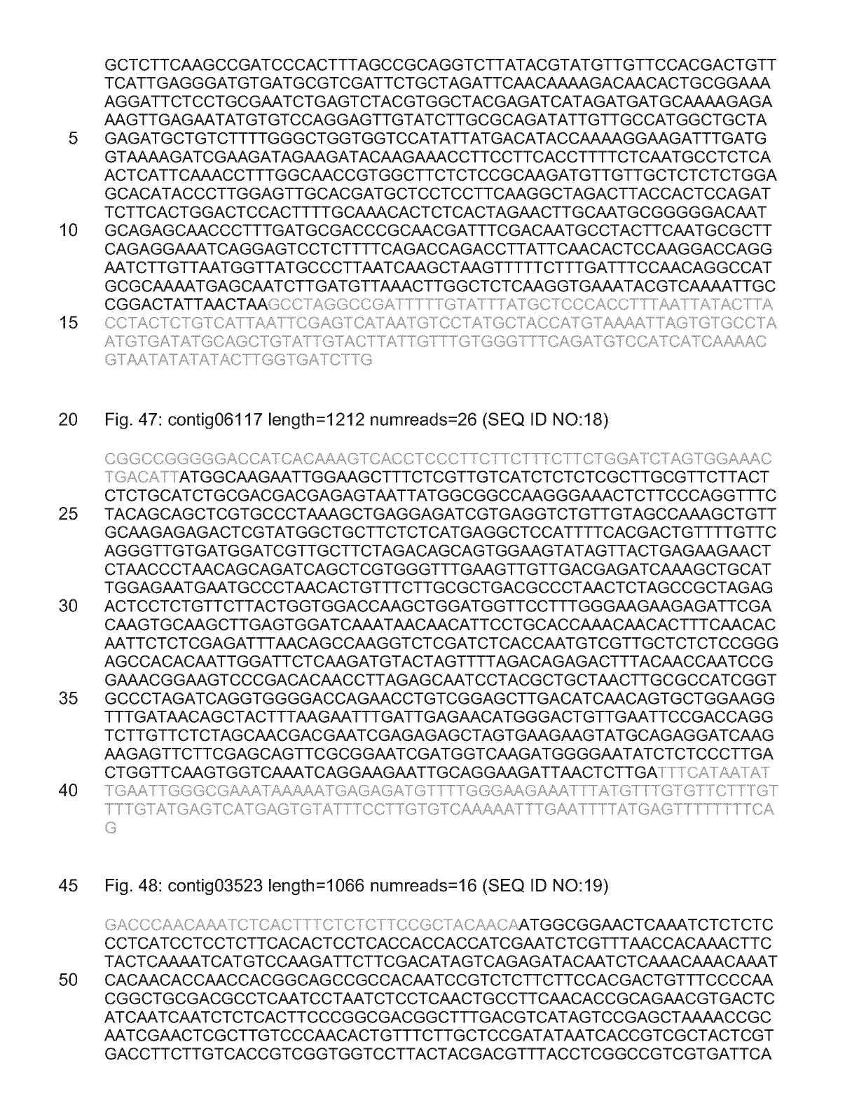

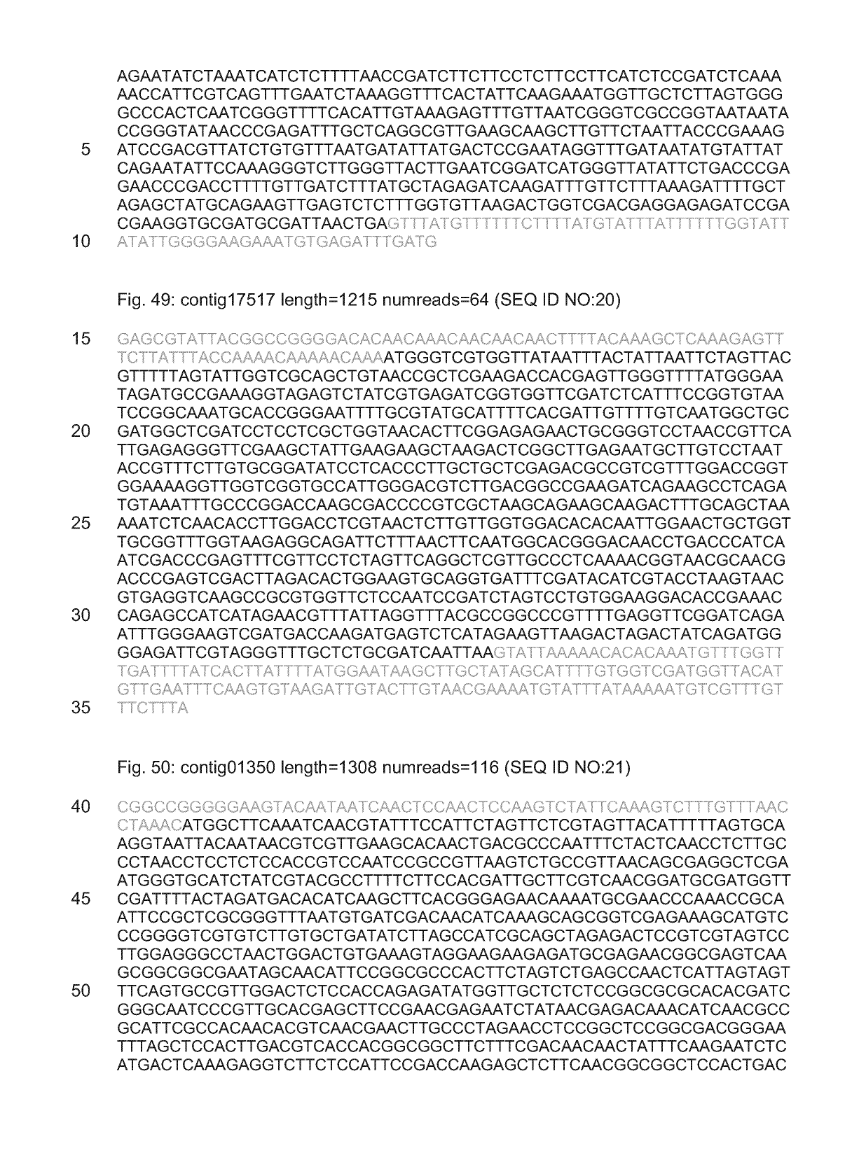

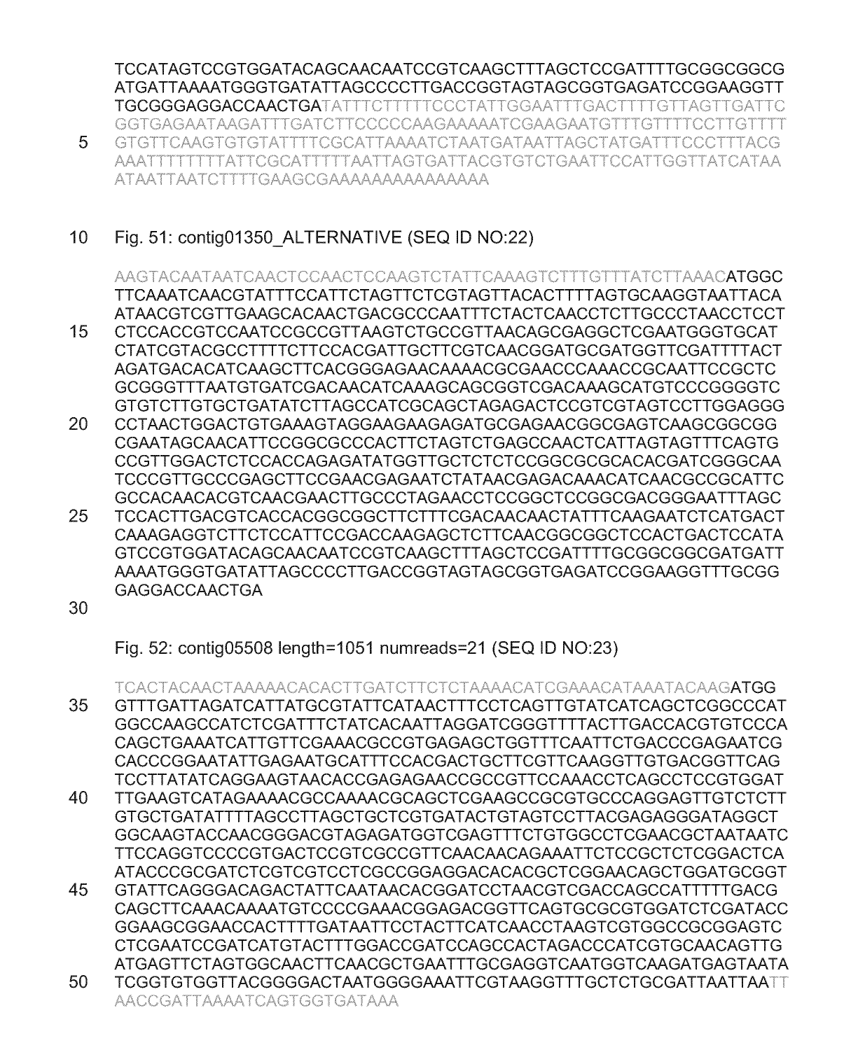

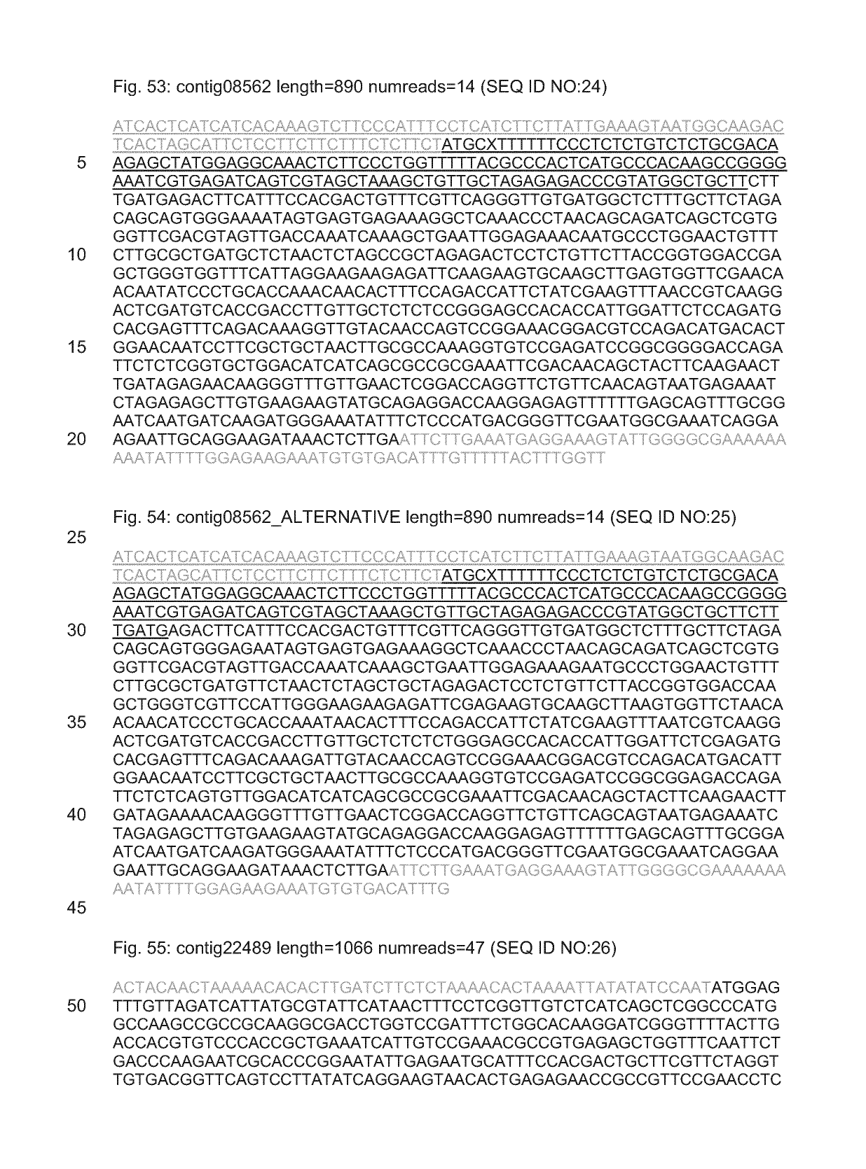

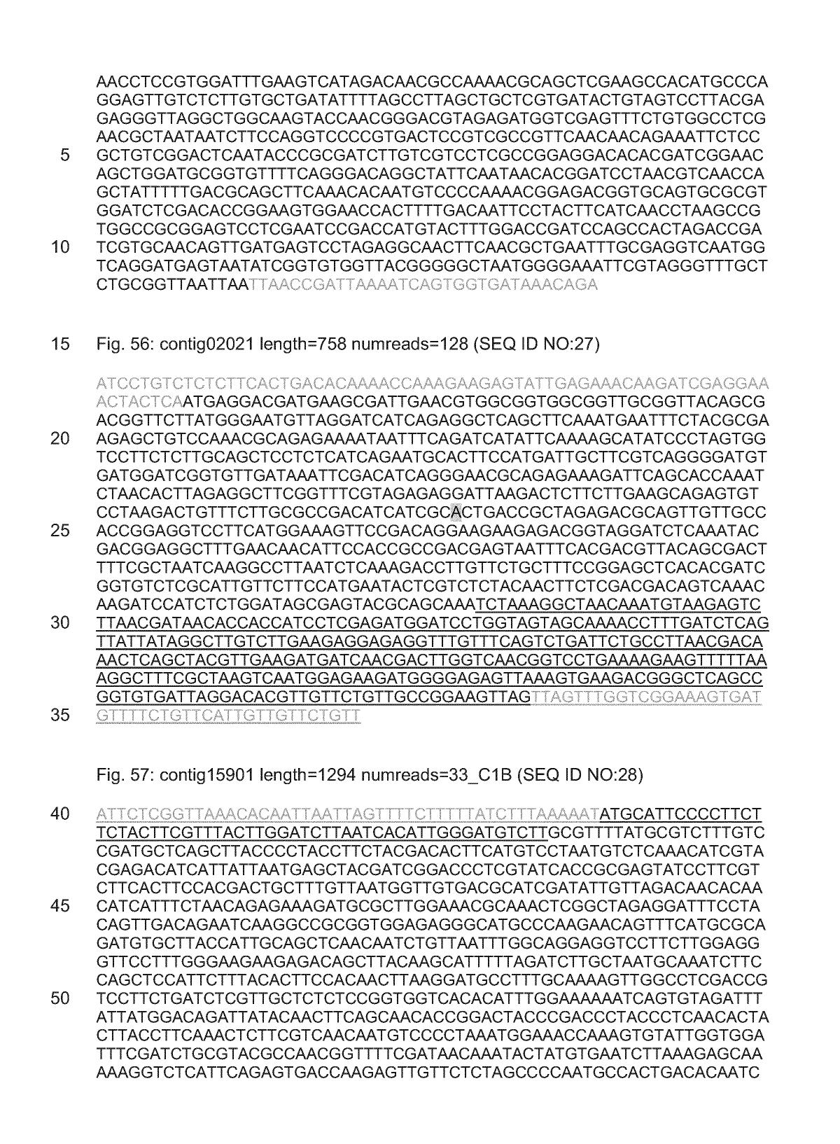

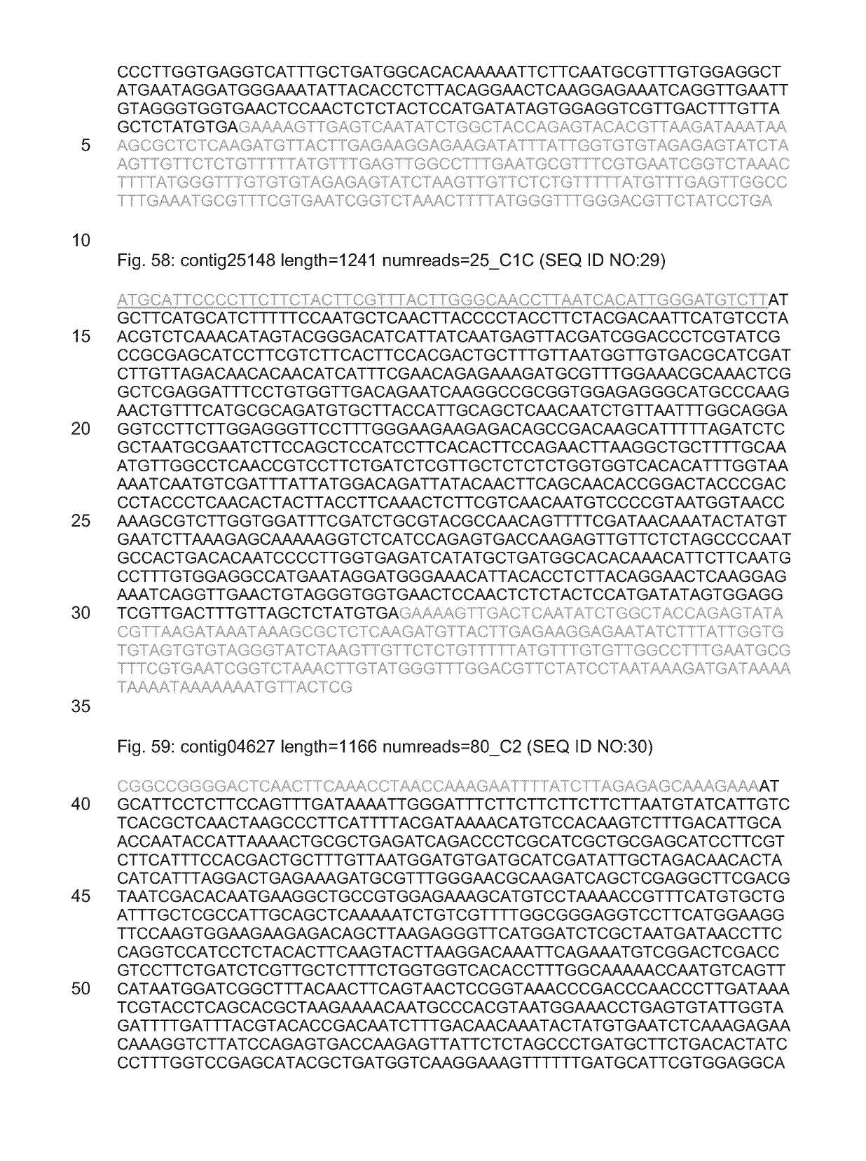

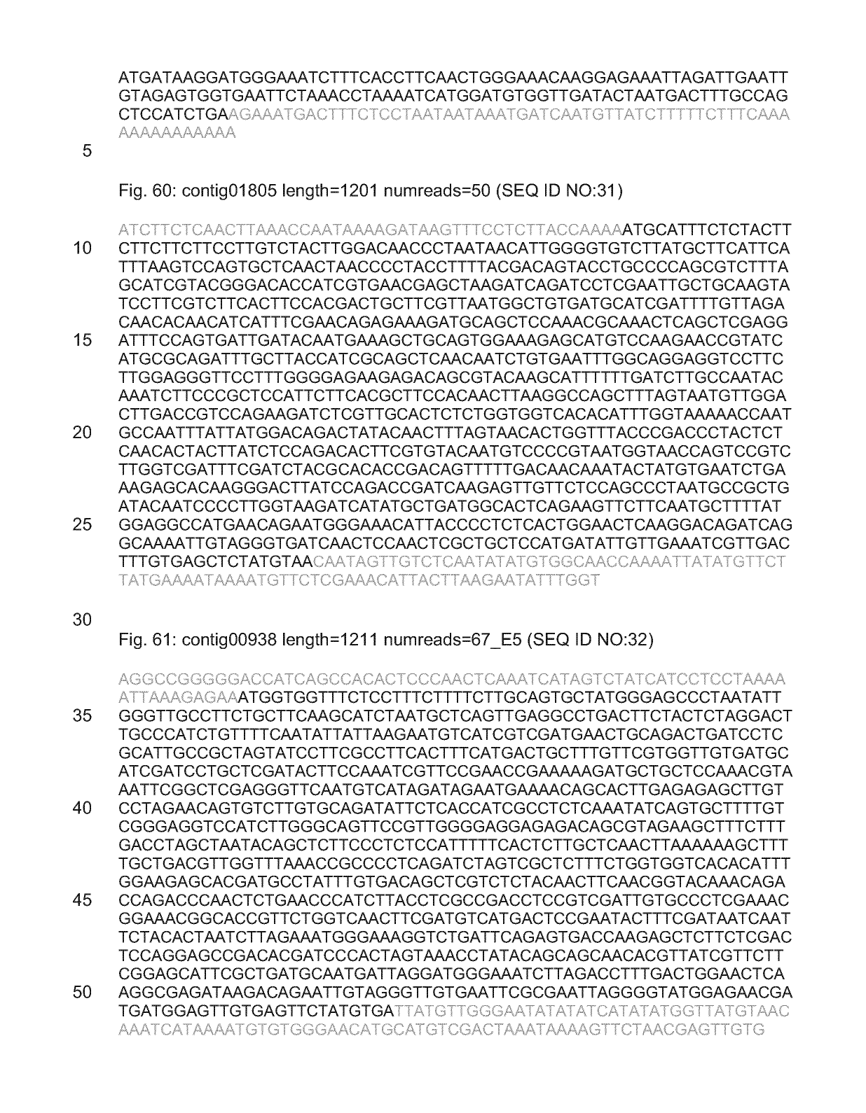

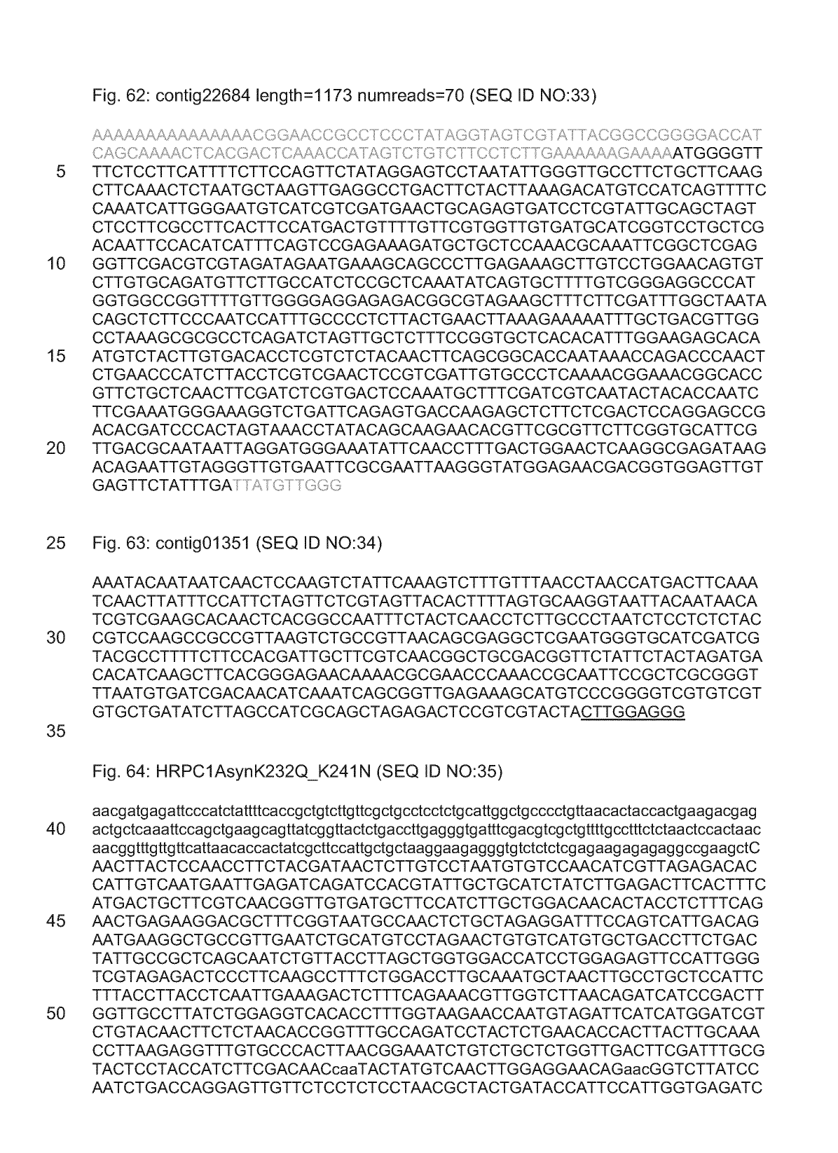

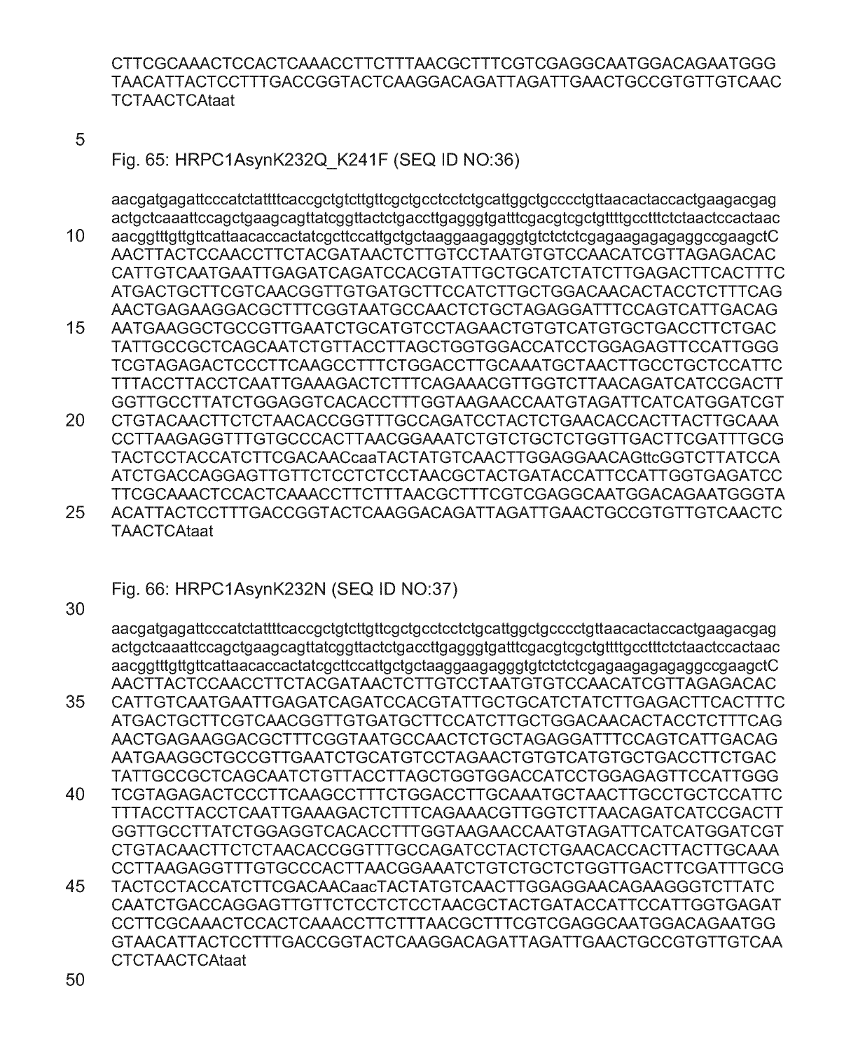

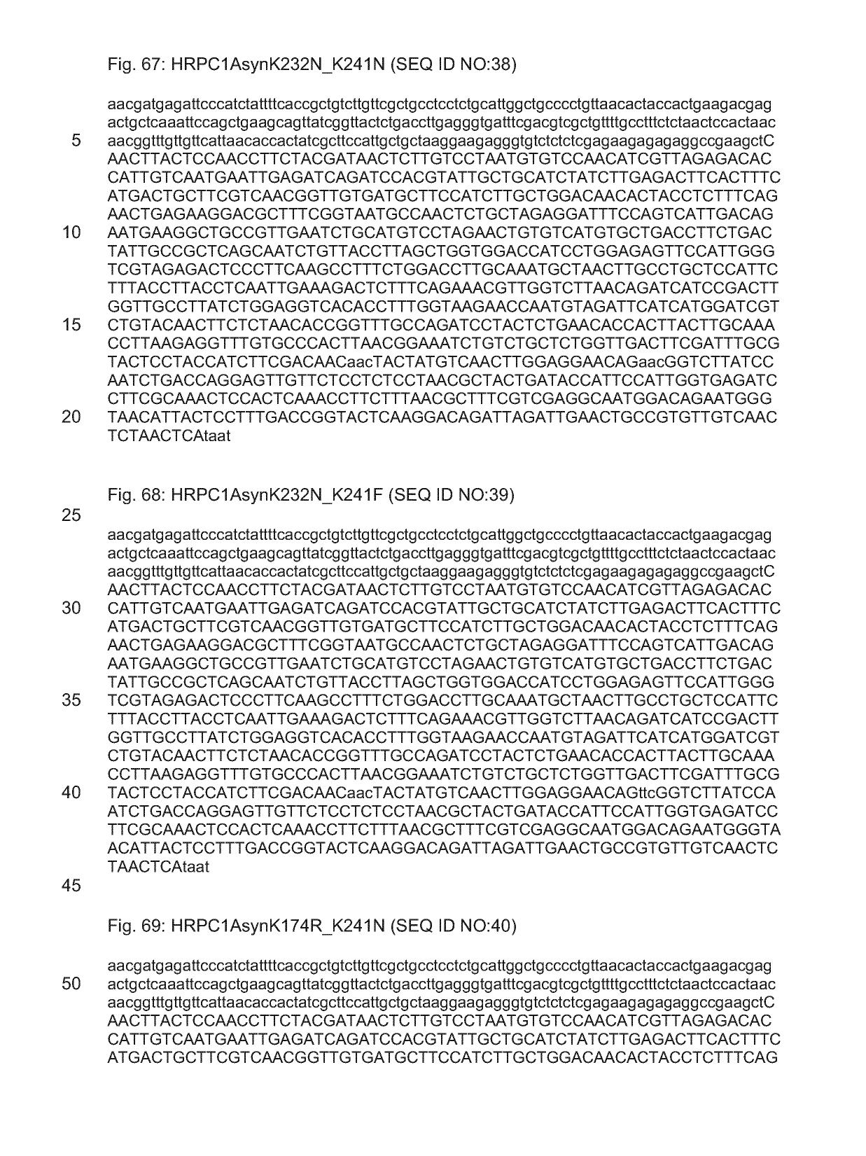

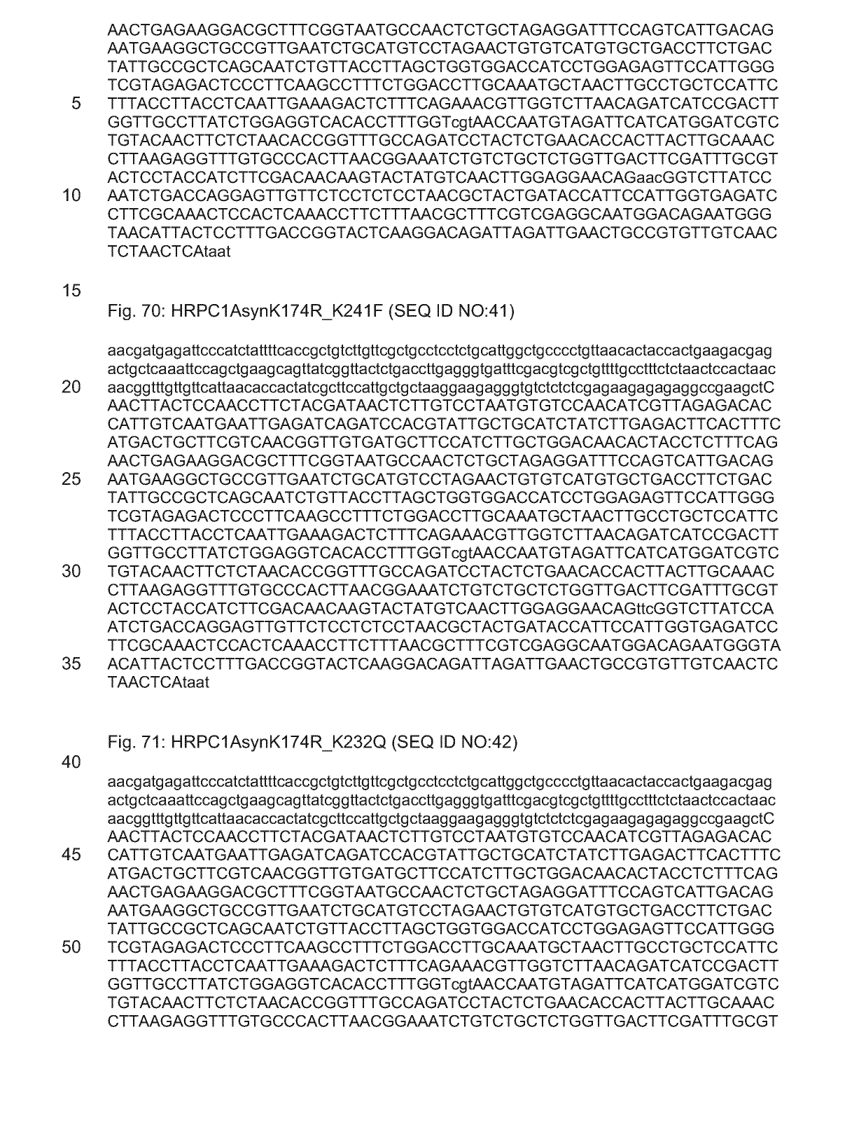

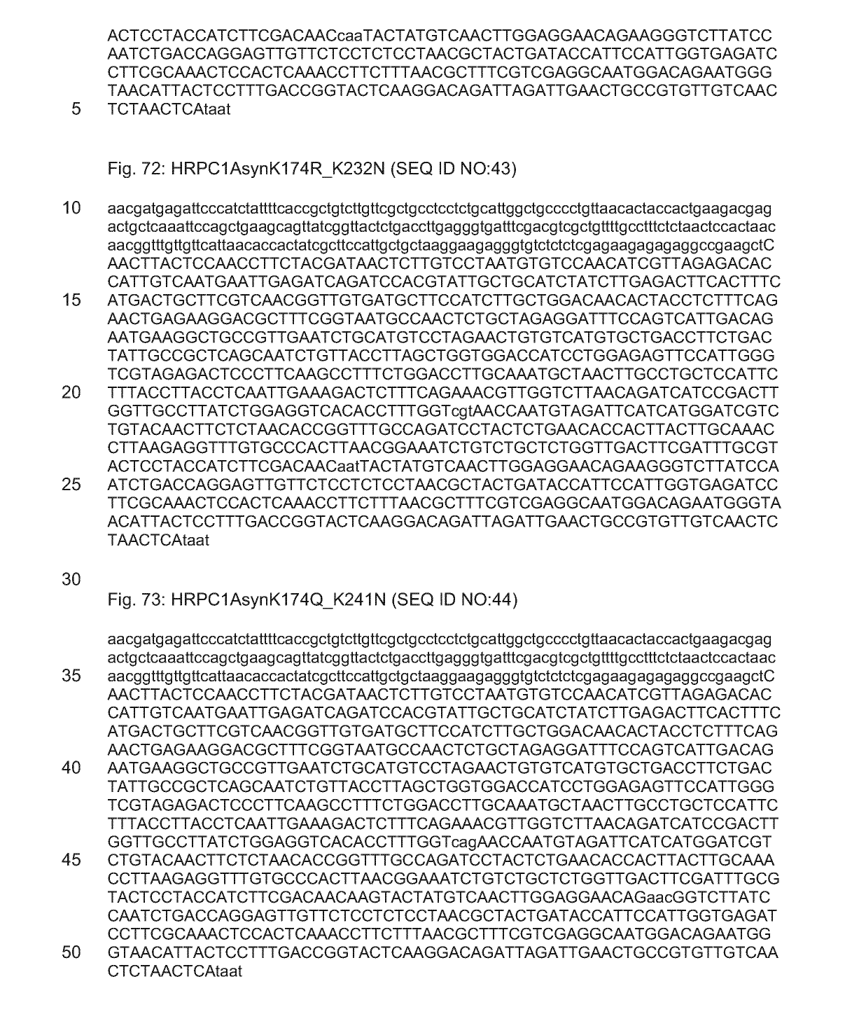

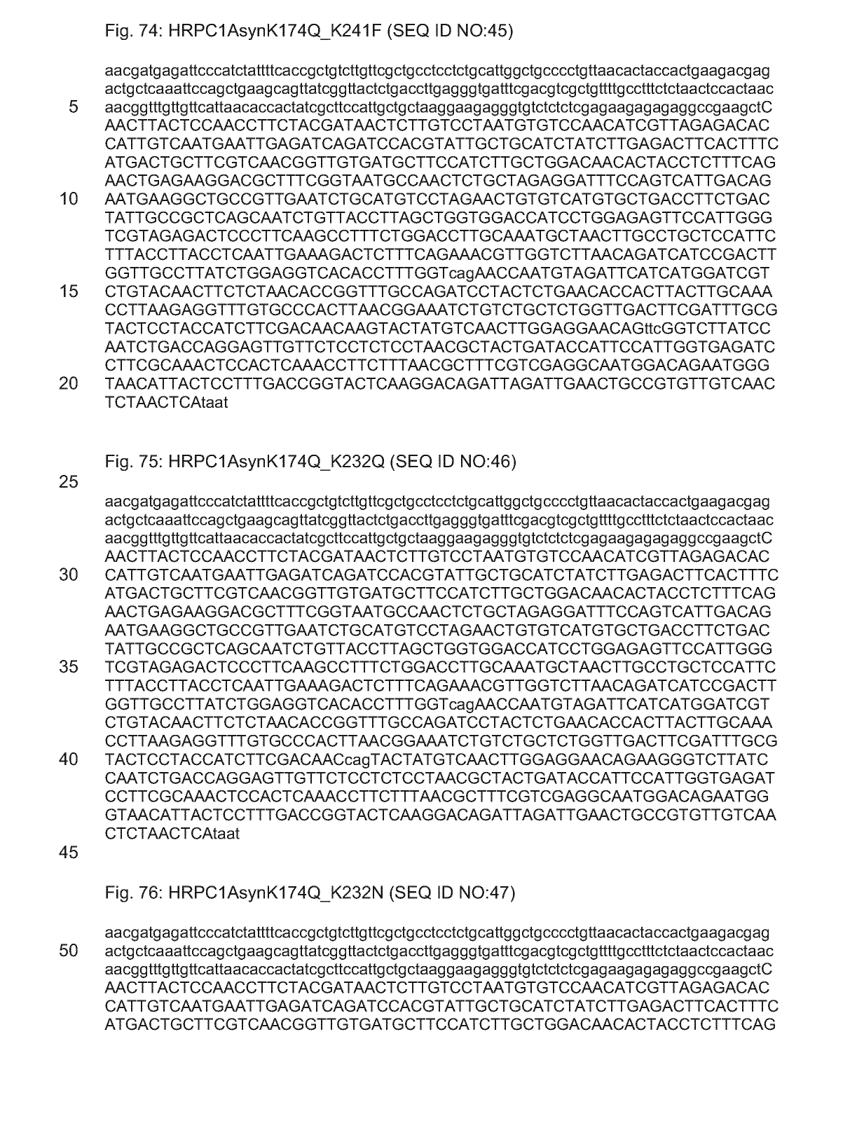

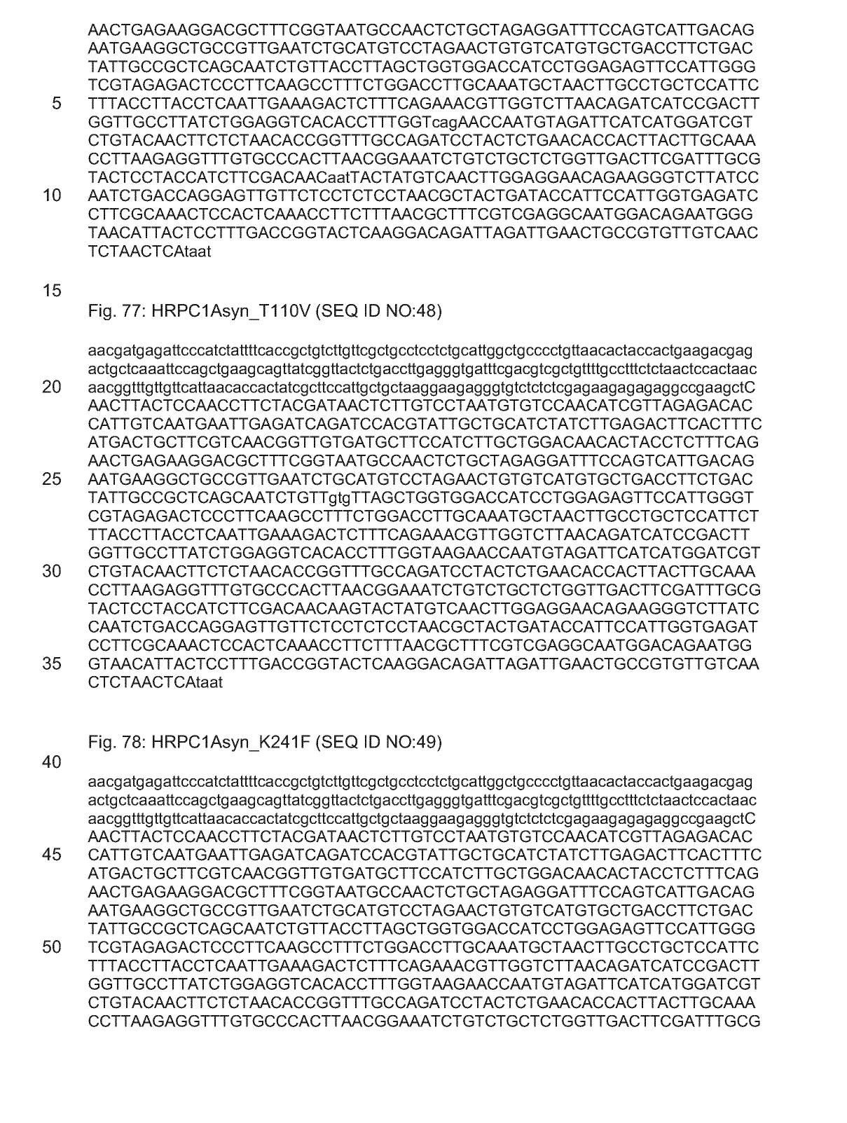

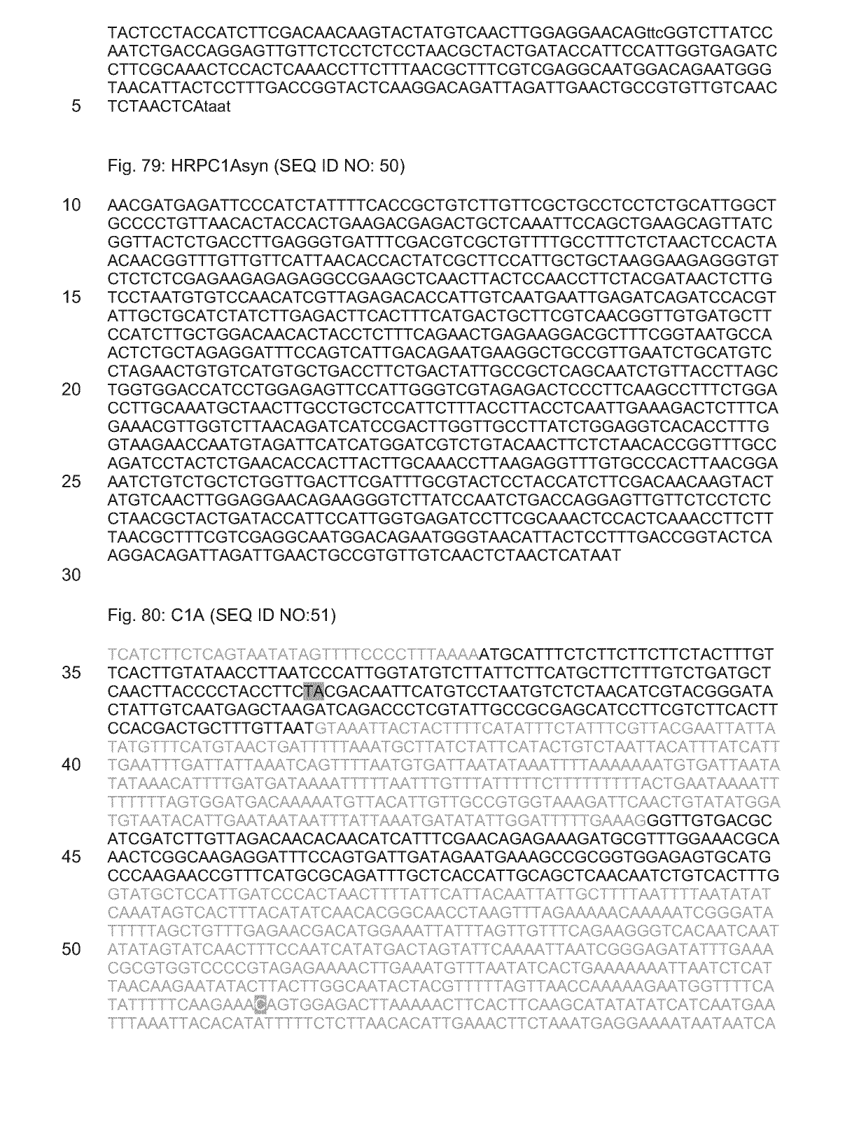

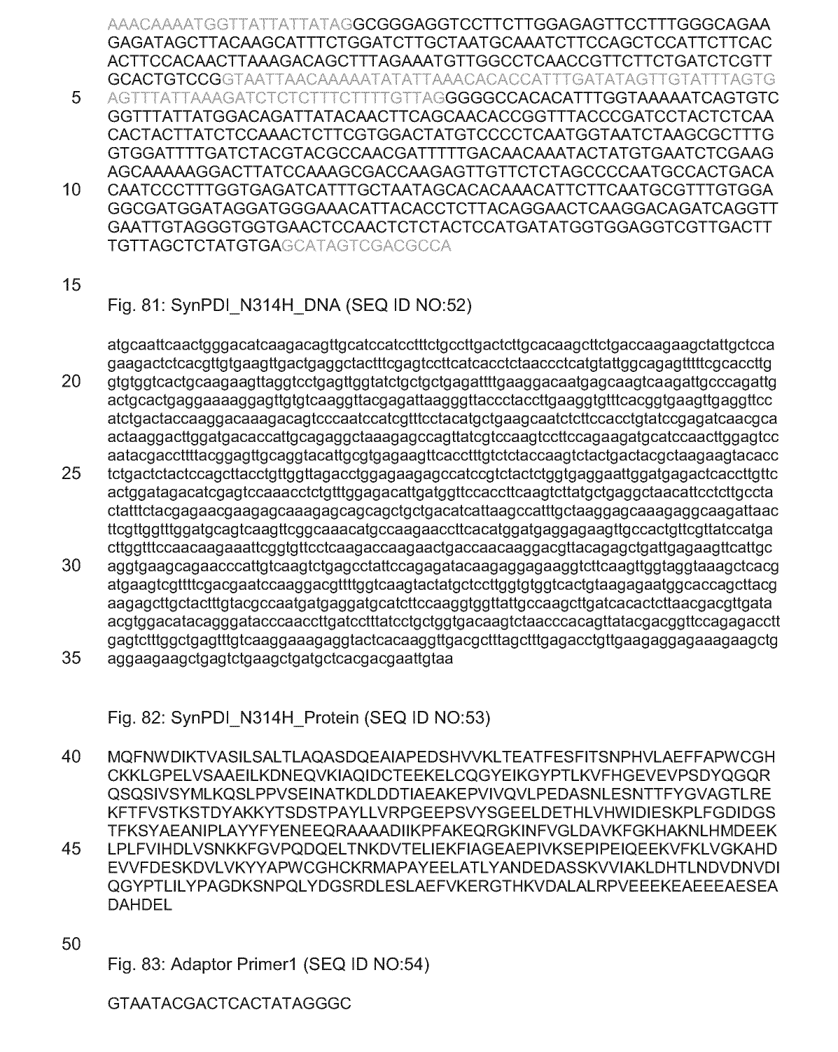

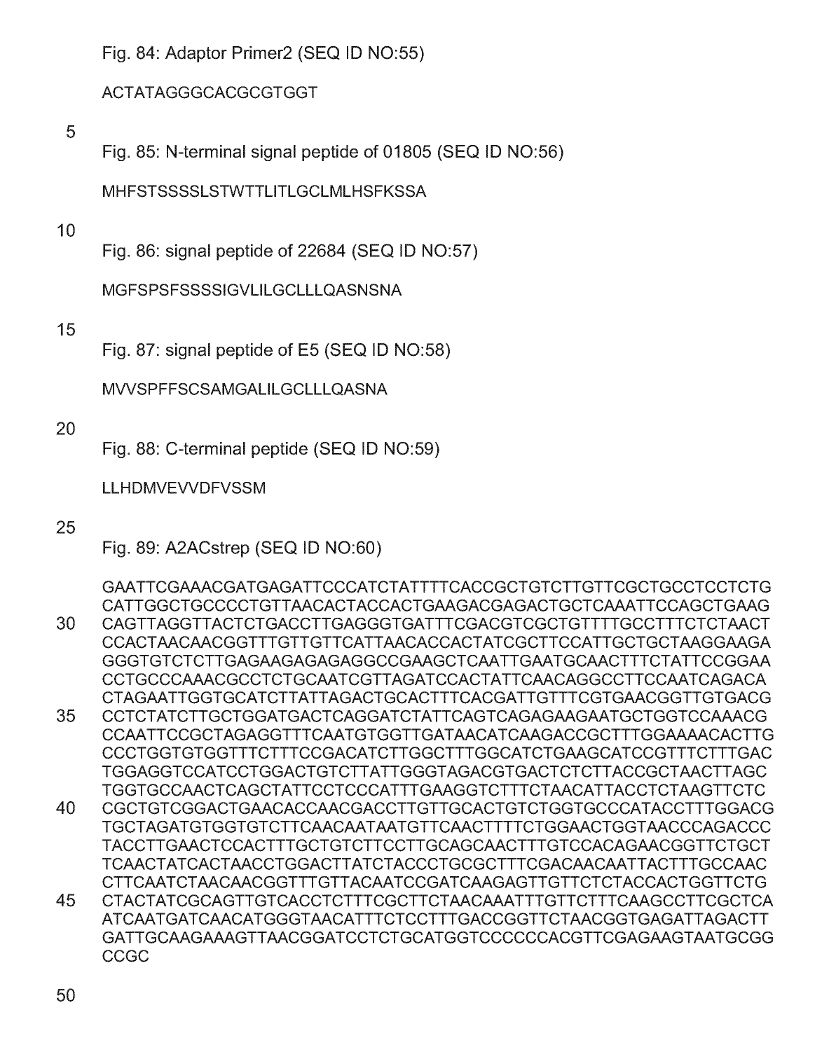

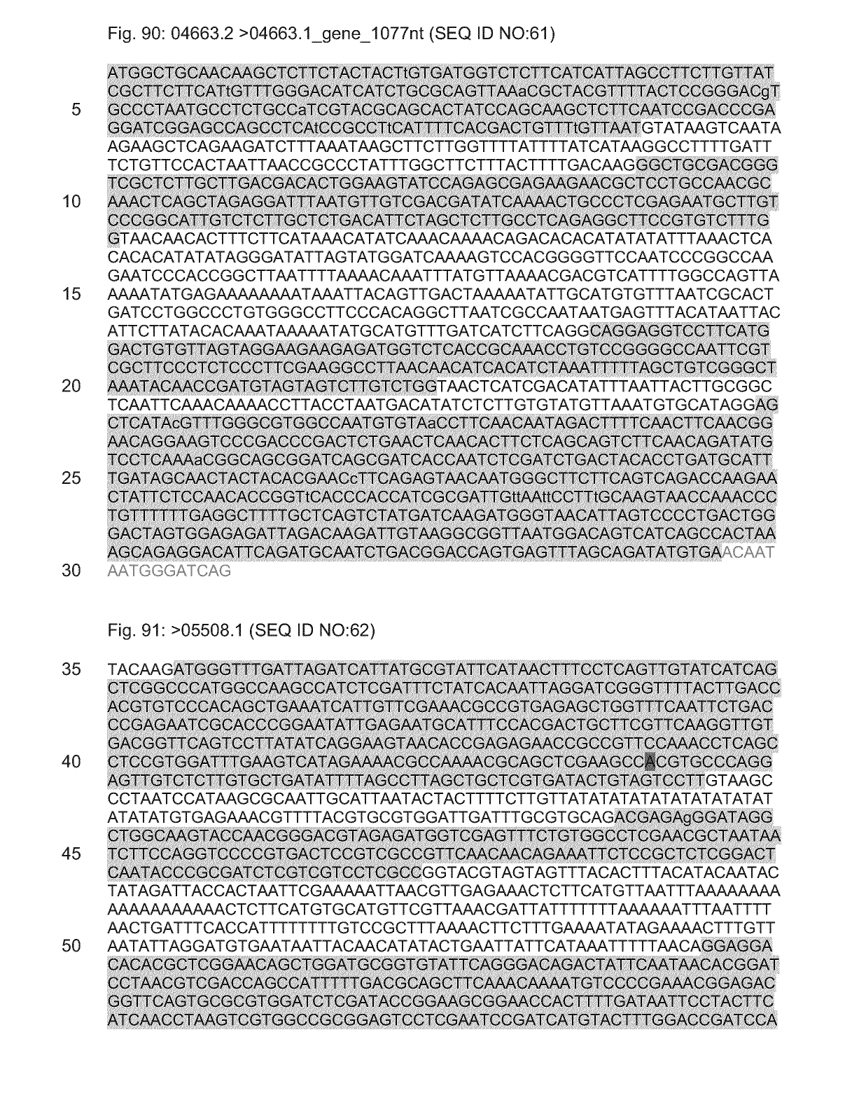

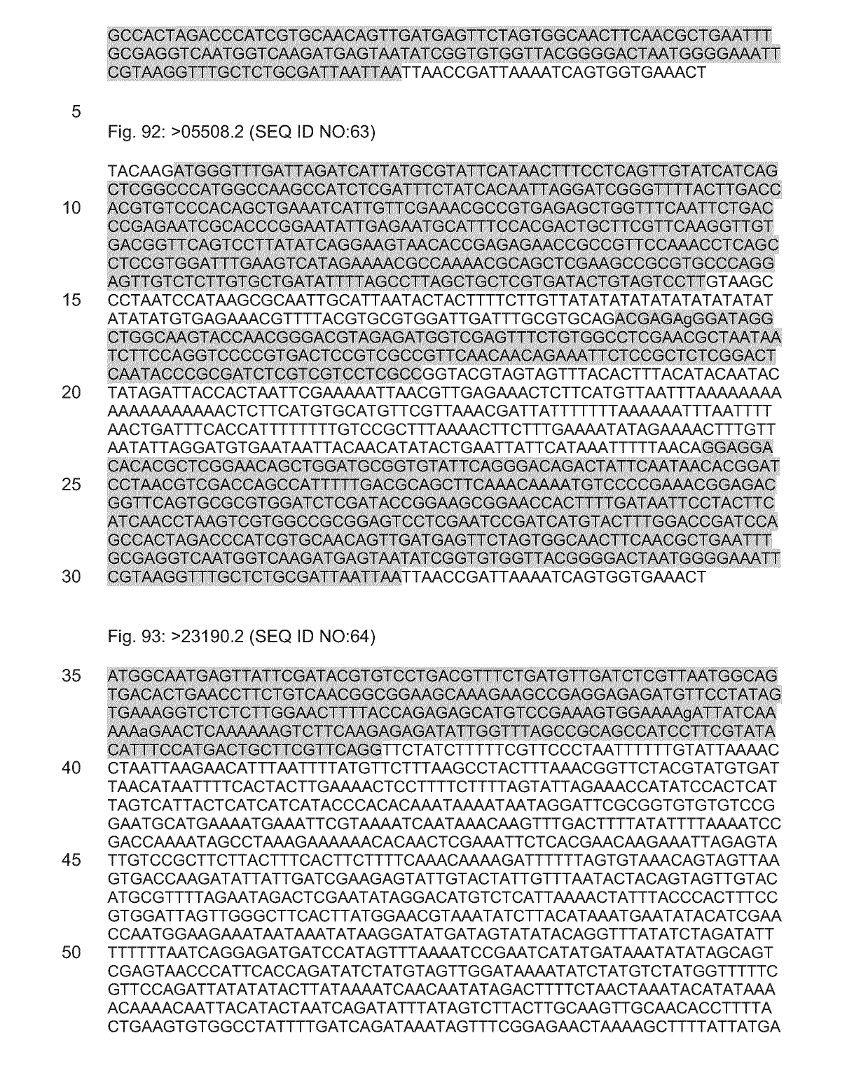

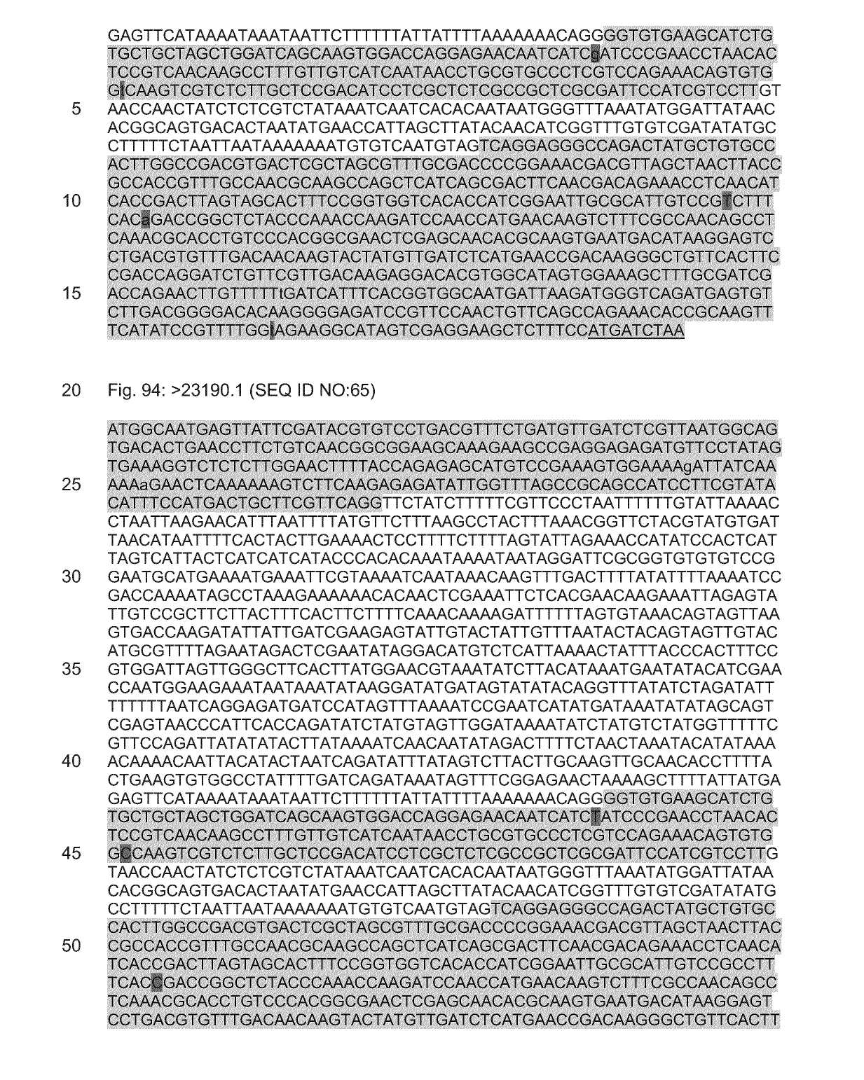

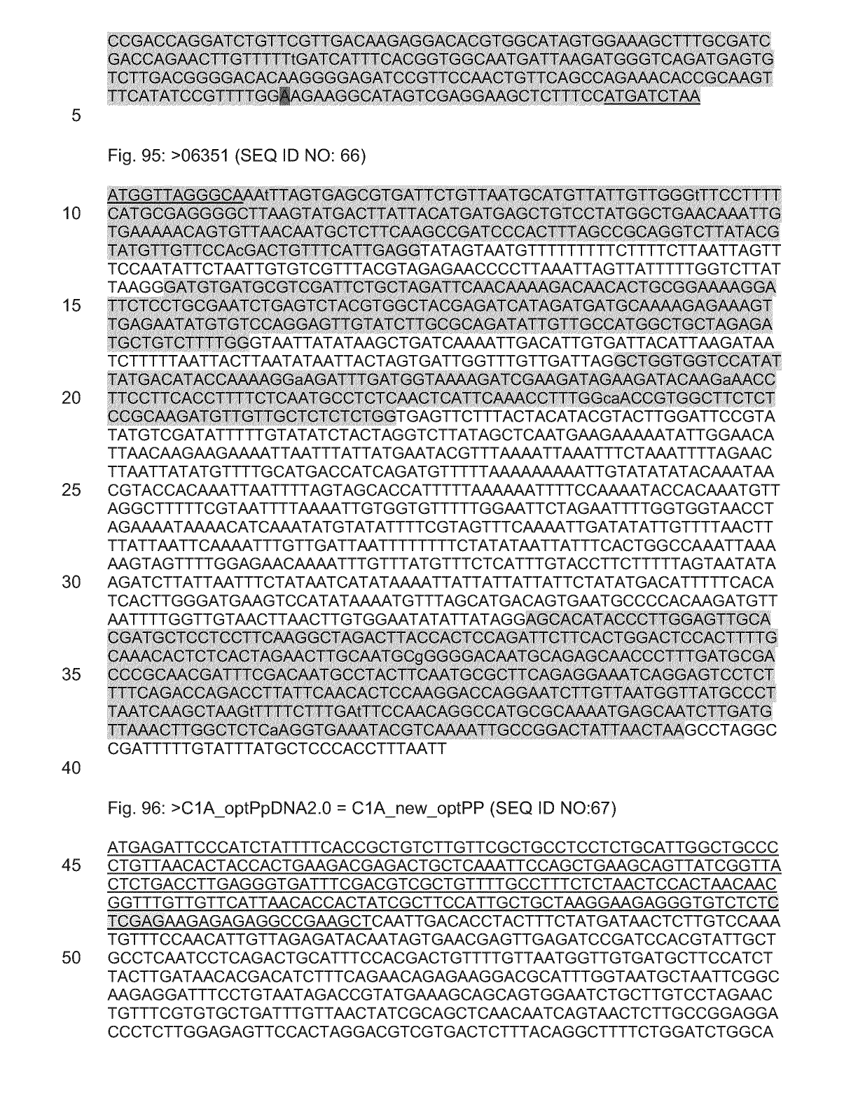

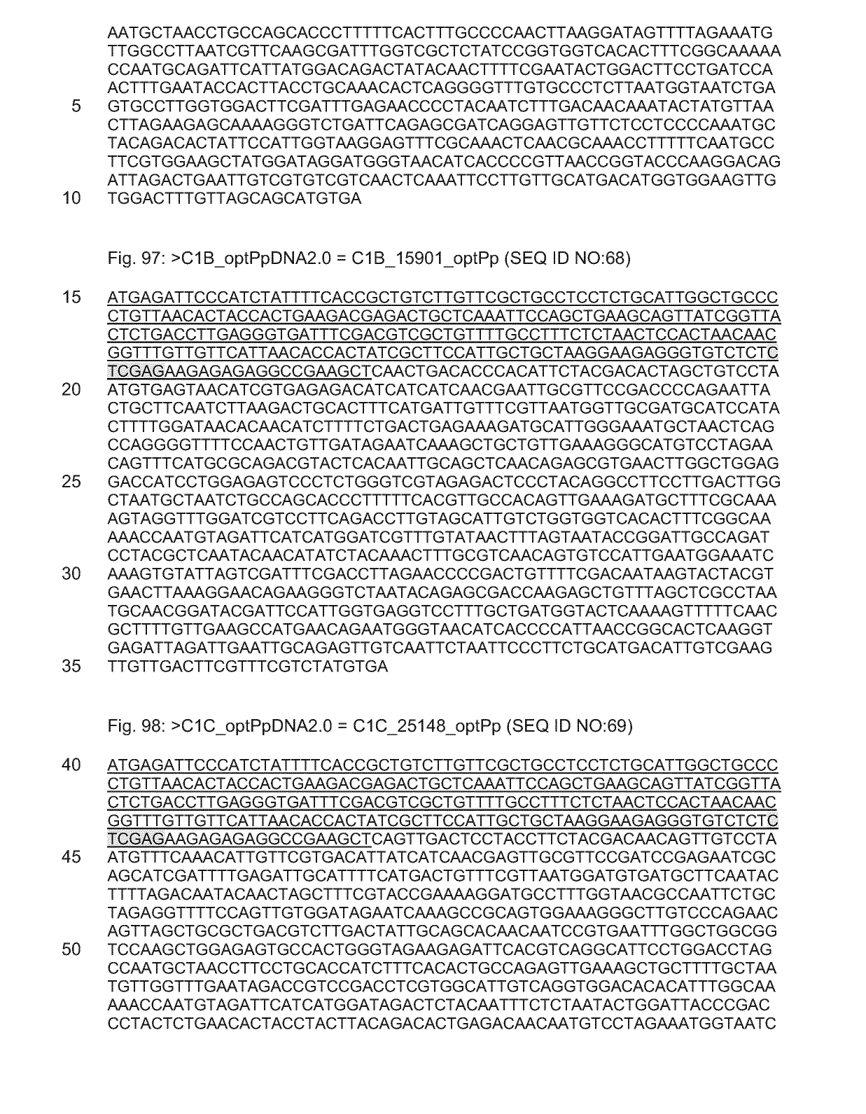

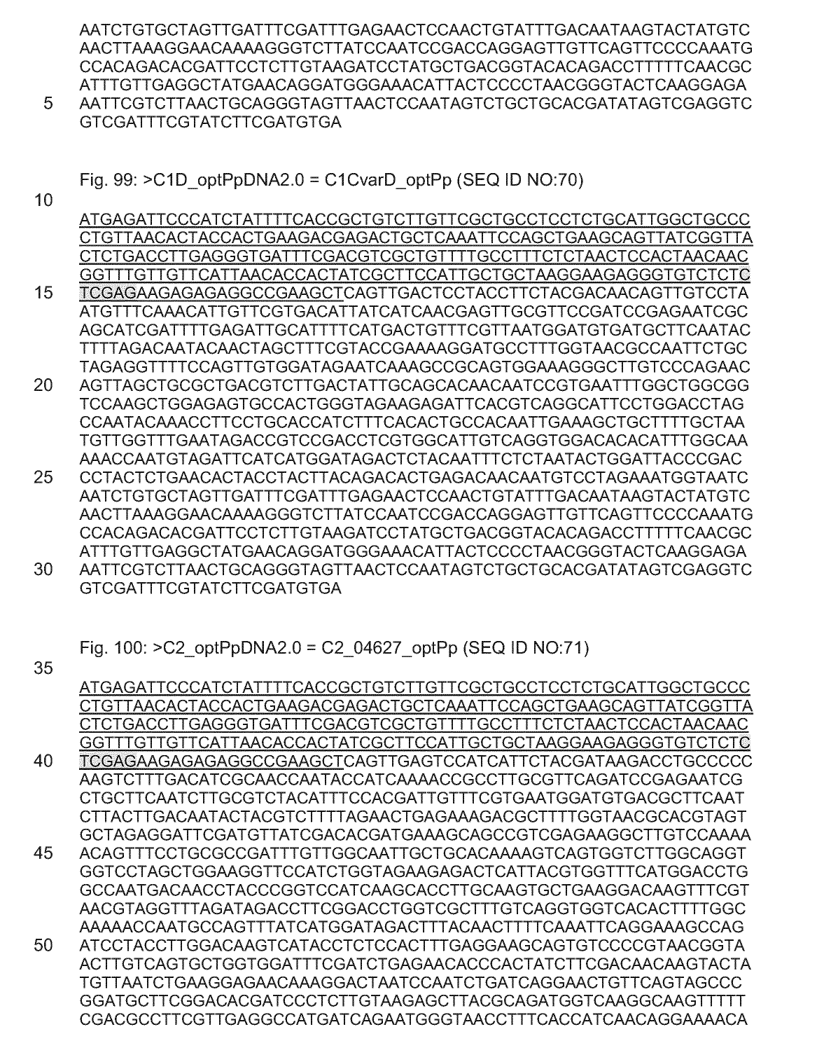

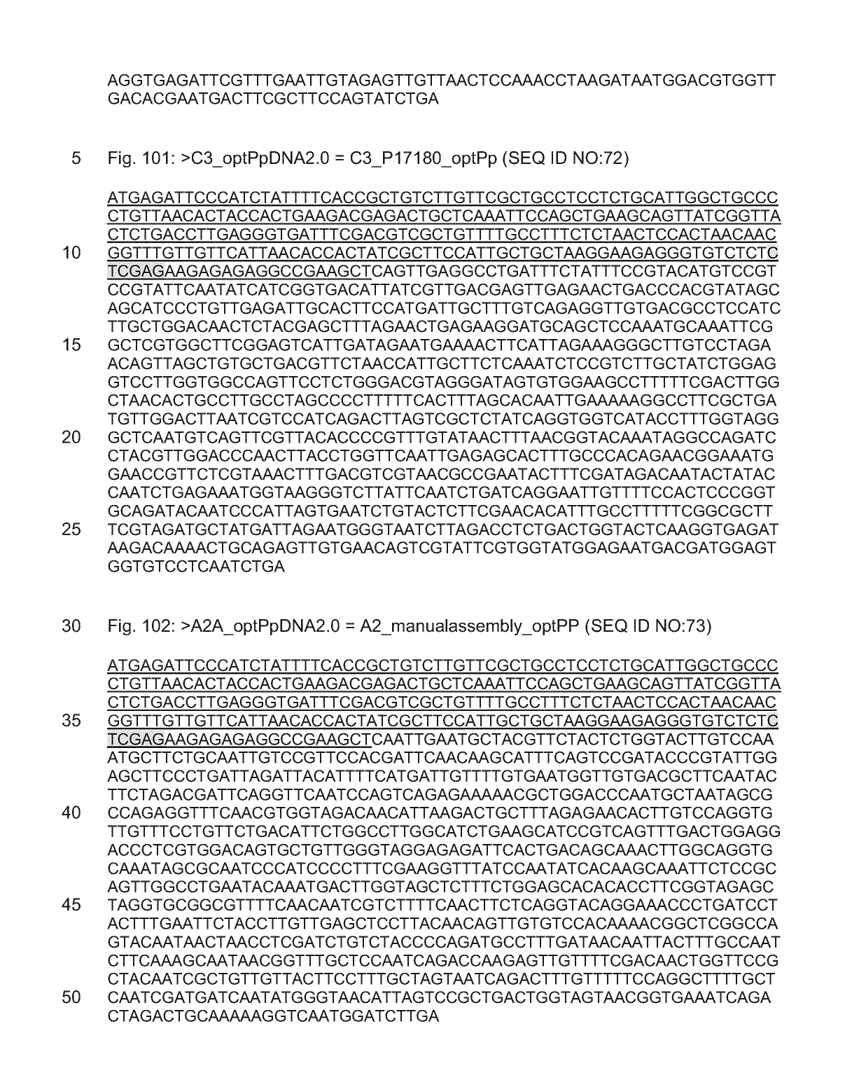

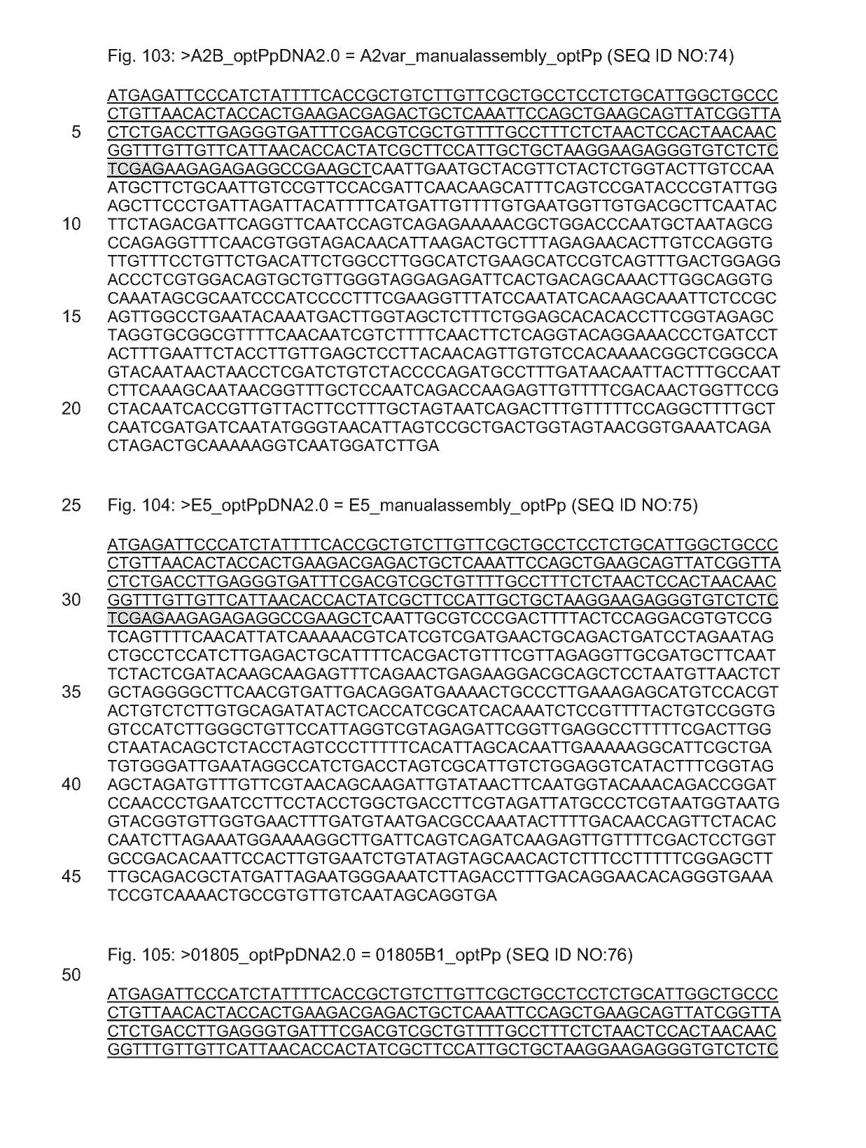

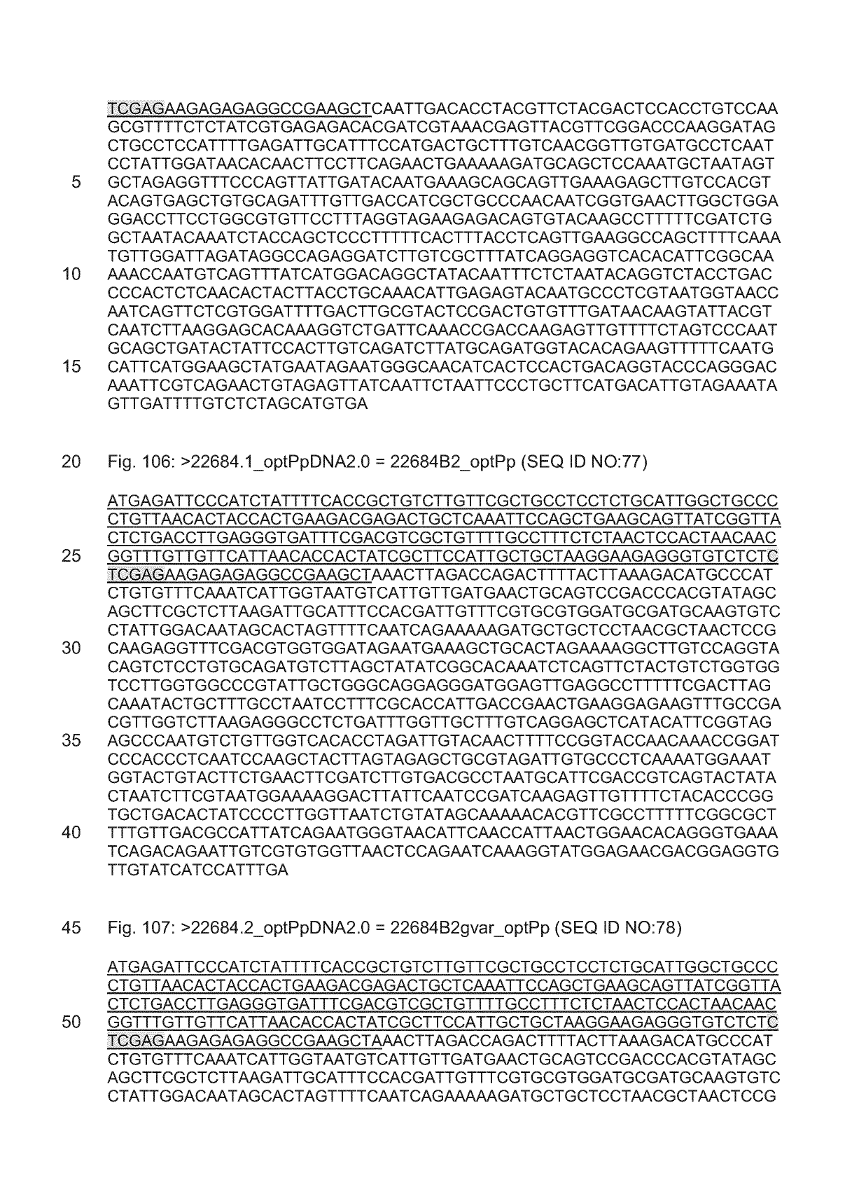

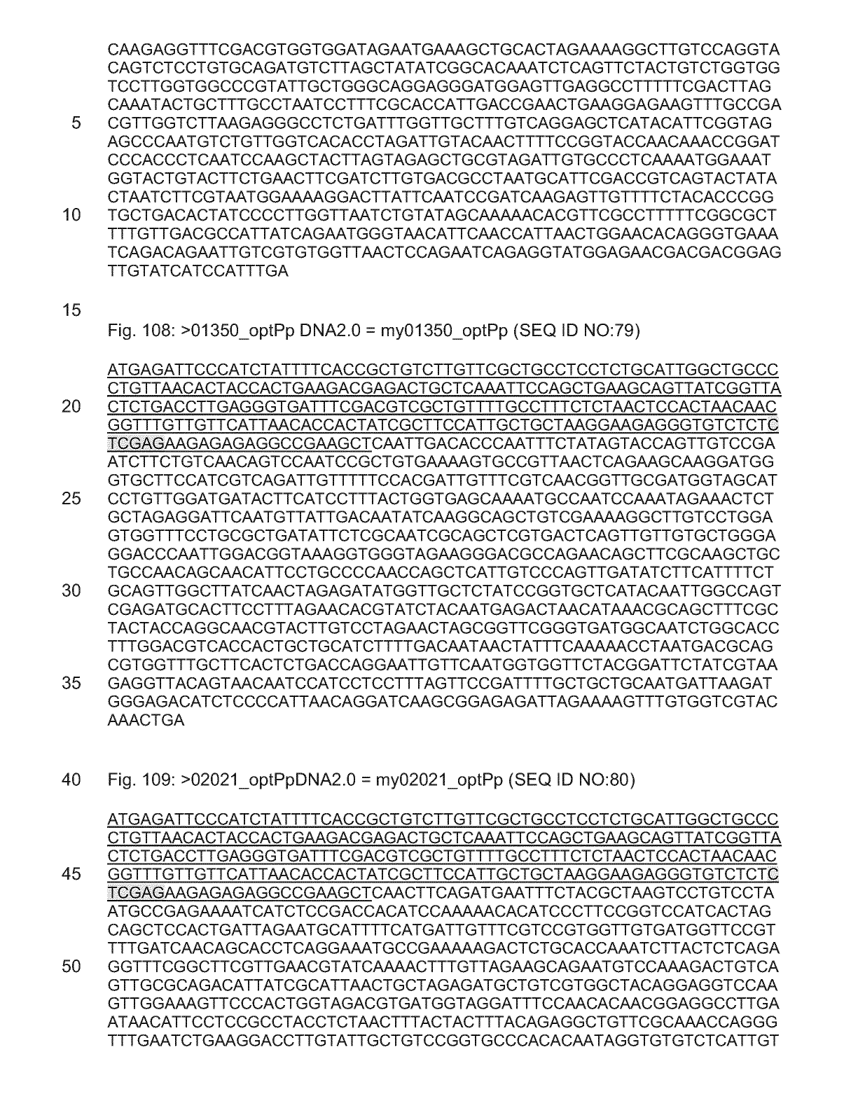

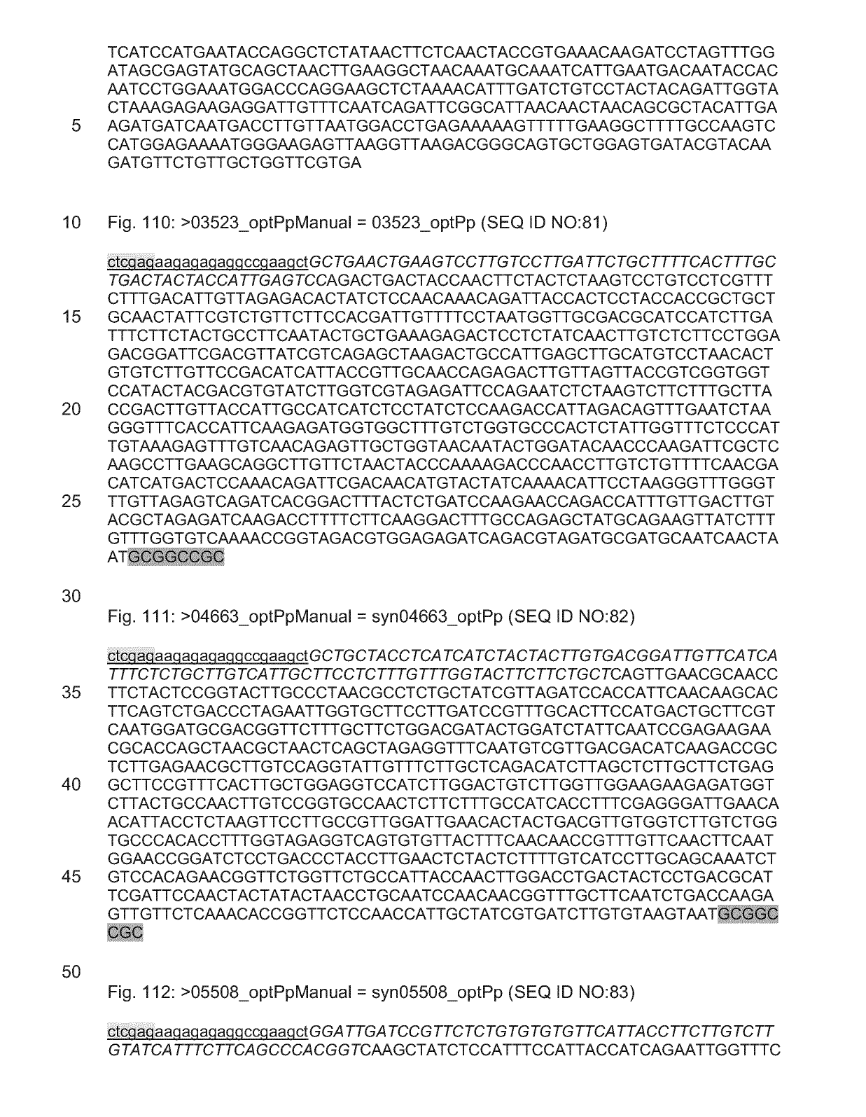

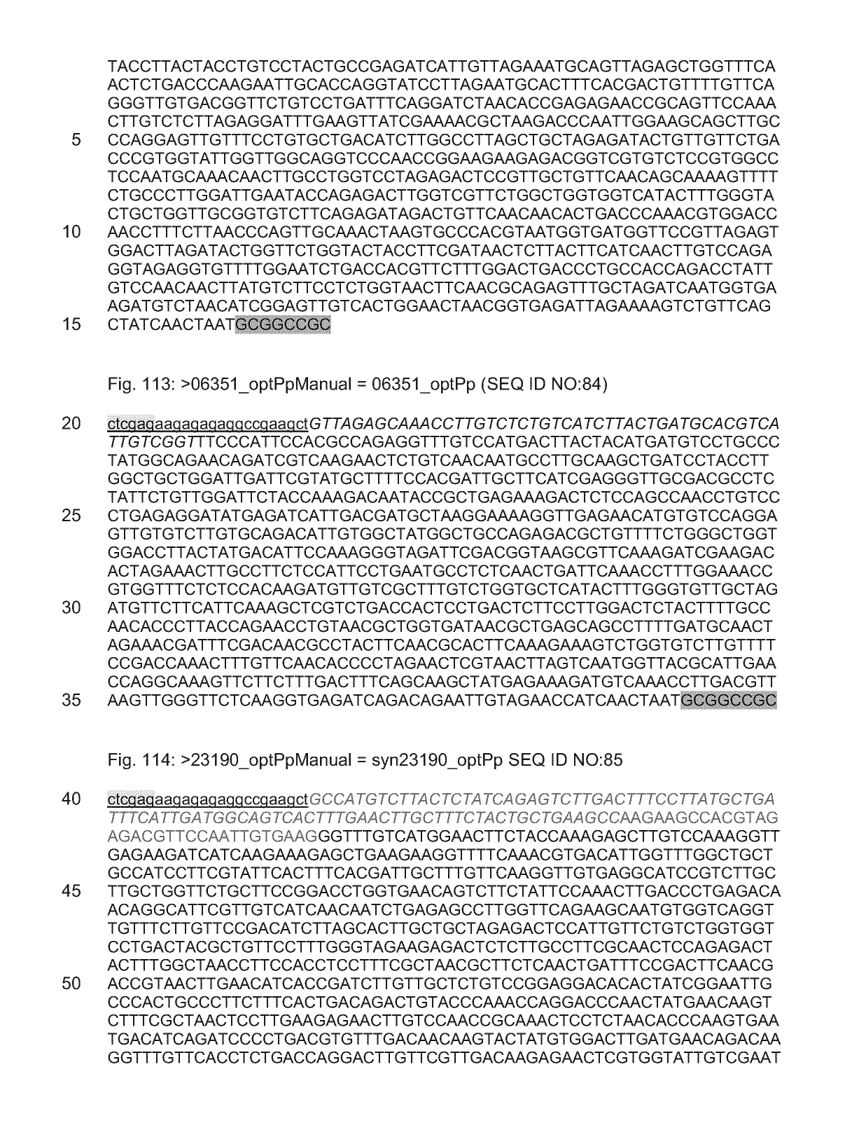

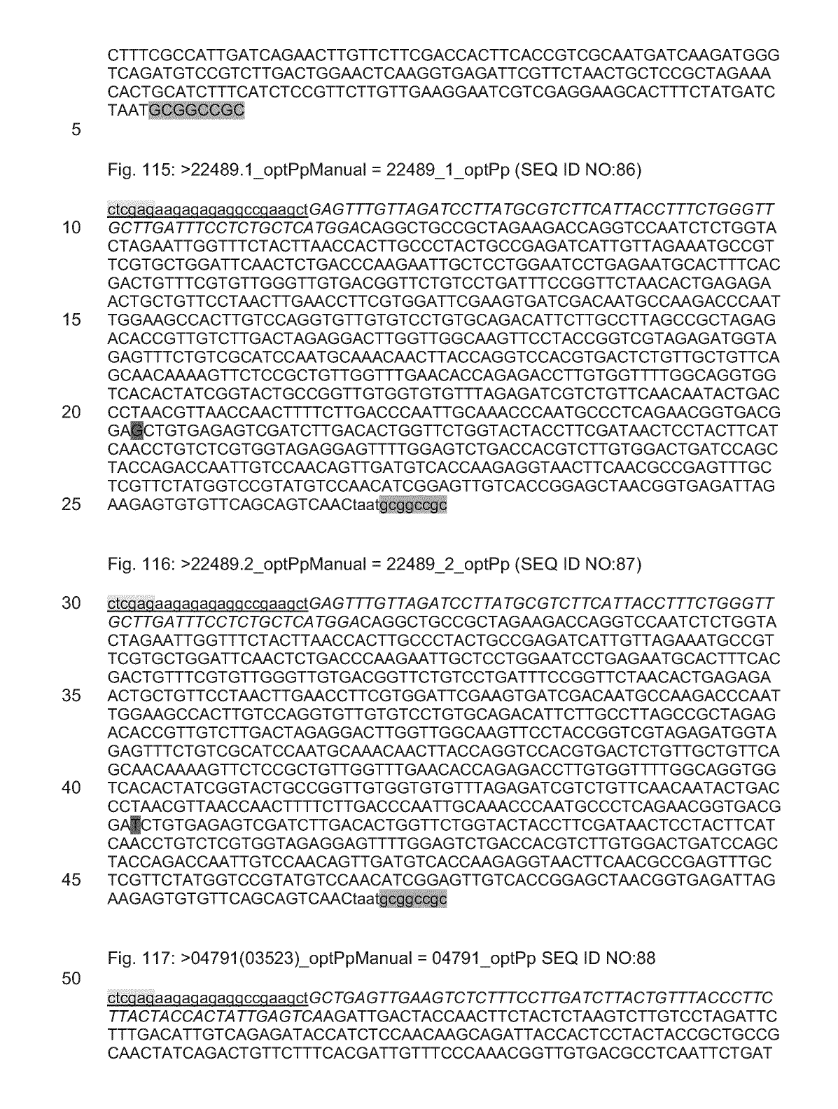

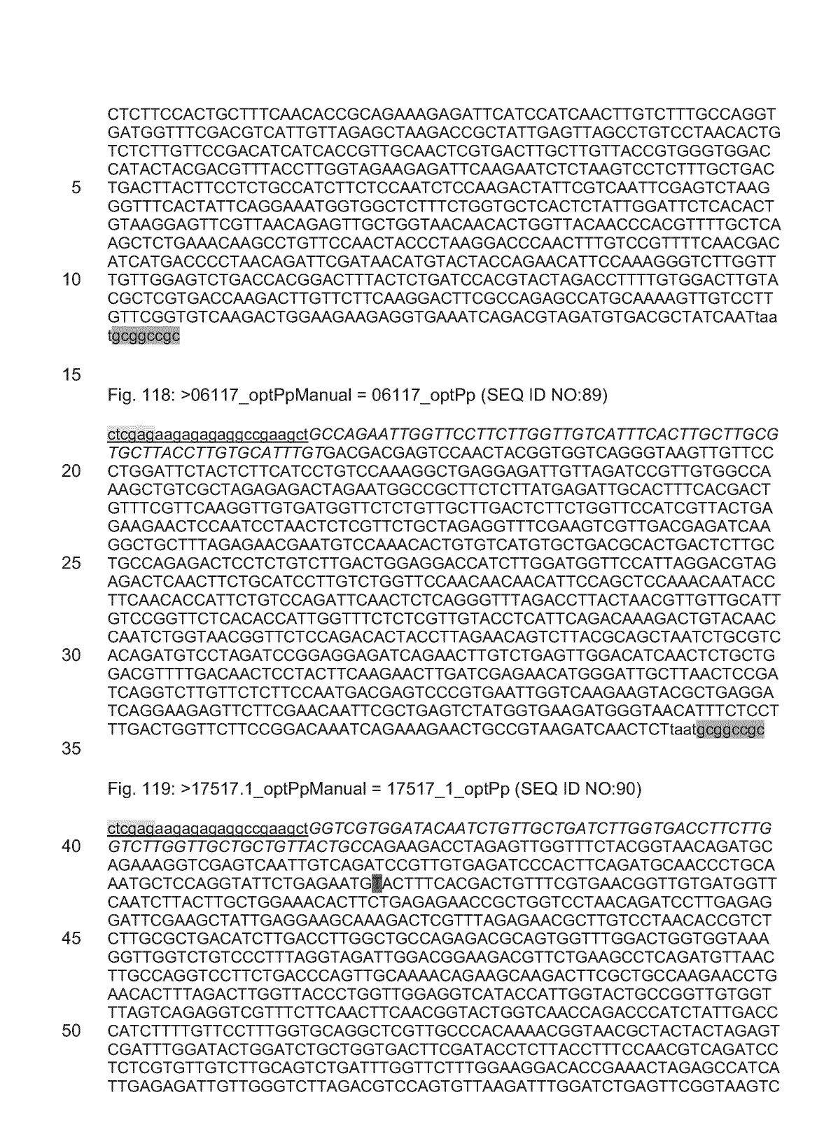

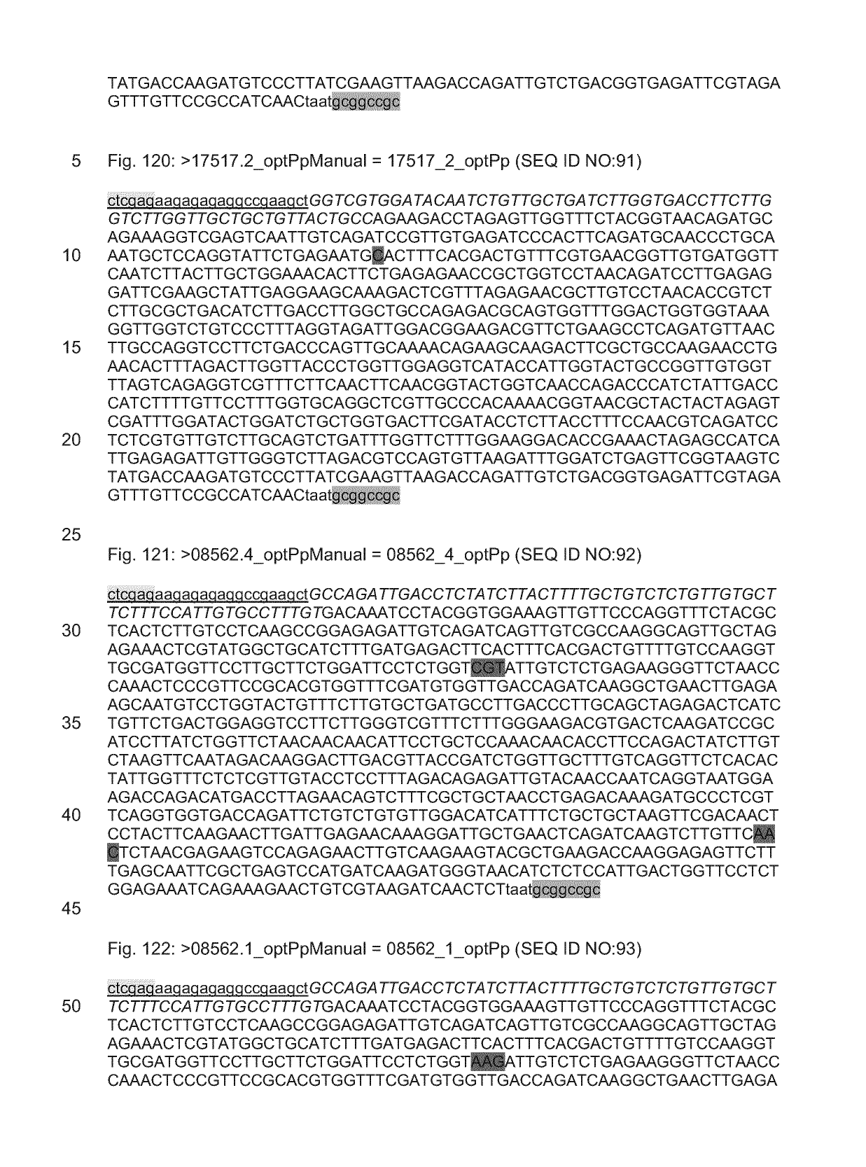

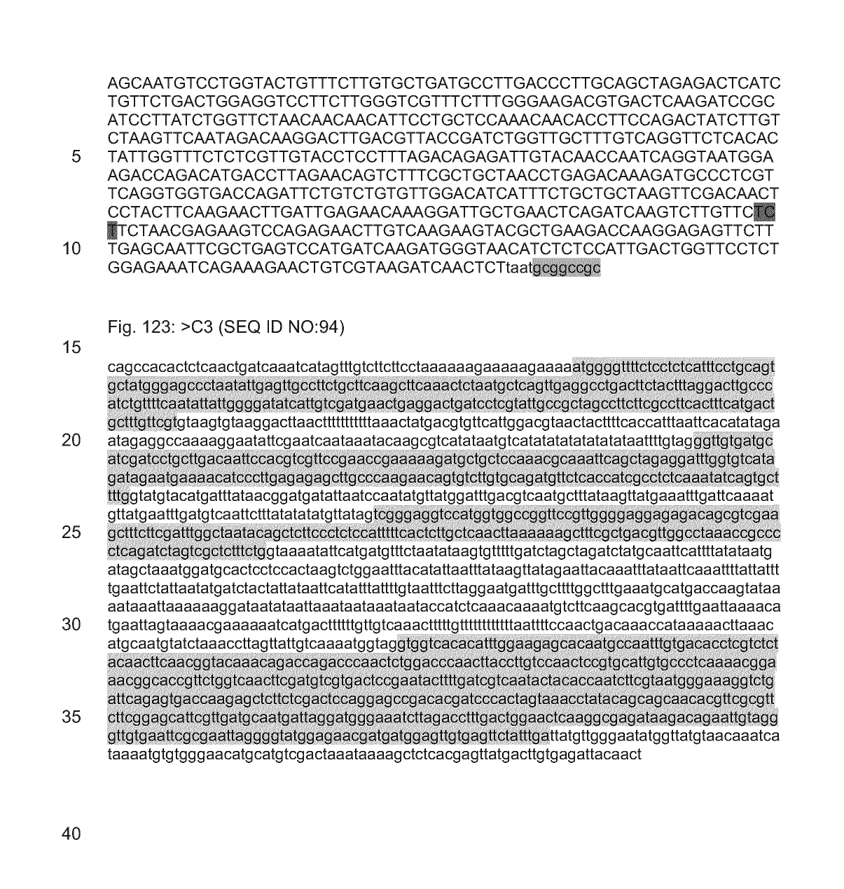

By aligning a translation of the (presumed) 5′-UTR (marked in grey) of the Sanger-verified C1C plus its N-terminus to the protein sequence of the N-terminus of C1B, a strikingly high similarity could be observed (FIG. 28).

This is most probably not part of the 5′-UTR of C1C, but rather an N-terminal signal peptide, whose full amino acid sequence has not been published at UniProt. Considering the high similarity to the signal peptide of C1B, it can be assumed that C1C and C1B target to the same destination.

Additionally, MHFSTSSSSLSTWTTLITLGCLMLHSFKSSA (SEQ ID NO:56) was identified as N-terminal signal peptide of 01805 with 51% similarity to the newly found C1C signal peptide, MGFSPSFSSSSIGVLILGCLLLQASNSNA (SEQ ID NO:57) as signal peptide of 22684 with 75% similarity to the annotated signal peptide of the C3 isoenzyme at UniProt, and MVVSPFFSCSAMGALILGCLLLQASNA (SEQ ID NO:58) as signal peptide of E5 with 77% similarity to the C3 signal peptide.

Moreover, the C-terminal peptide LLHDMVEVVDFVSSM (SEQ ID NO:59) of C1A was found, which is annotated as propeptide at UniProt, not only present in C1A, but also in C1B and C1C with 93% similarity and in 01805 with 86% similarity. It is thus very likely, that these isoenzymes are processed to mature enzymes in a similar way as C1A.

In addition to these similarity-based findings of signal peptides, the predicted signal peptides and the corresponding cleavage sites from the SignalP 3.0 server are shown in Table 6 (the predicted cleavage sites of the verified isoenzymes. 1: Most likely cleavage site position. 2: Cleavage site sequence from neural networks prediction. 3: Cleavage site probability from hidden Markov models prediction):

TABLE 6

| | cleavage site | | |
|---|---|---|---|
| isoenzyme | pos¹ | seq² | prob³ |
| C1A | 30/31 | SDA/QL | 0.849 |
| C1B | 28/29 | SDA/QL | 0.897 |
| C1C | 29/30 | SNA/QL | 0.903 |
| C2 | 24/25 | SHA/QL | 0.891 |
| A2A | 31/32 | SSA/QL | 0.738 |
| E5 | 27/28 | SNA/QL | 0.920 |
| 01805 | 31/32 | SSA/QL | 0.647 |
| 22684 | 29/30 | SNA/KL | 0.613 |
| 01350 | 20/21 | VQG/NY | 0.550 |
| 02021 | 29/30 | SEA/QL | 0.810 |
| 04791 | 22/23 | IES-RL | 0.877 |
| 06117 | 22/23 | CIC/DD | 0.805 |
| 17517 | 23/24 | VTA/RR | 0.919 |
| 08562.1 | 22/23 | CLC/DK | 0.989 |
| 08562.2 | 22/23 | CLC/DK | 0.989 |

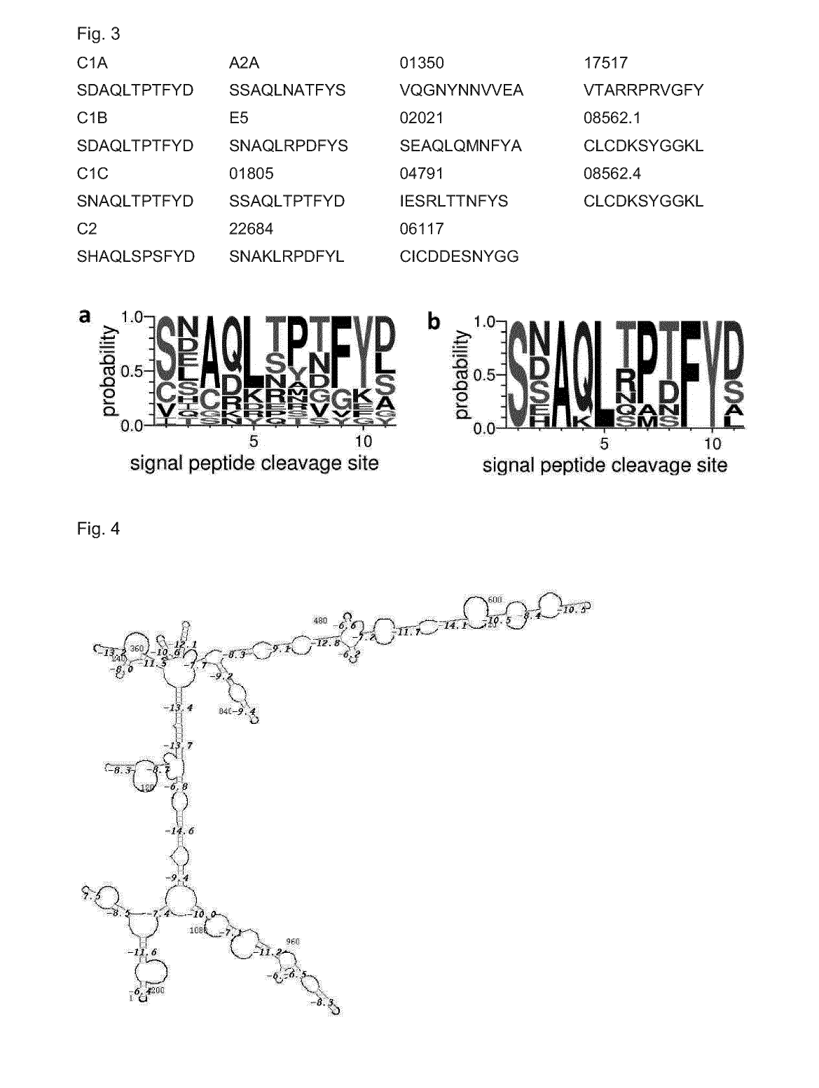

The following eleven amino acids surrounding the predicted signal peptide cleavage sites were used to create a sequence logo (FIG. 3).

The cleavage sites of C1A, C1B, C1C, C2, A2A, E5, 01805, 22684 and 02021 were found to be highly alike and were thus used for the creation of another sequence logo (FIG. 3b) which allows the fast identification of highly conserved amino acids in this signal peptide cleavage site, e.g. the alanine (position 4 in FIG. 3b) before the glutamine after which the signal peptide is cleaved.

By examining the gene structure it was observed that all published HRP isoenzyme sequences showed a structure of four exons and three introns. Most of the sequences presented here match this structure, however isoenzyme 04791 does not have any introns and 17517 has only two introns. Both genes are within a cluster of three closely related HRP genes (FIG. 2).

The calculation of the theoretical IEP showed that most of the newly identified isoenzymes have a basic theoretical IEP. 01350, 04791, 08562.1 and 08562.4 may be assigned to the basic HRP isoenzyme groups D and E. 06117, 22684 and 01805 might be further members of the isoenzyme group C, or belong to group B (i.e. neutral, neutral-basic). HRP A2A showed the lowest IEP so far with 4.82, whereas the highest IEP was found for HRP 17517 with 9.57.

The calculated IEPs of the verified HRP isoenzymes are shown in Table 7:

TABLE 7

| isoenzyme | IEP |
|---|---|
| A2A | 4.82 |
| C1A | 5.67 |
| 06117 | 5.69 |
| C1B | 5.74 |
| 22684 | 6.32 |
| 01805 | 6.37 |
| C1C | 6.94 |
| 01350 | 8.66 |
| C2 | 8.70 |
| E5 | 8.72 |
| 04791 | 8.86 |
| 08562.1 | 8.95 |
| 08562.4 | 8.96 |
| 02021 | 9.57 |
| 17517 | 9.57 |

### Example 2

### Expression of HRP A2A in Pichia pastoris

2.1 Experimental

2.1.1 Optimizing the HRP A2A Gene for Heterologous Expression

HRP isoenzyme A2A was expressed in Pichia pastoris due to its acidic isoelectric point.

In order to maximize the yield of expressed HRP the A2A gene was optimized, using its protein sequence derived form the Sanger-verified nucleotide sequence. Upstream the mature A2A sequence, an EcoRI restriction site was added, the P. pastoris Kozak sequence and the α-factor signal sequence to facilitate secretion. For later purification, the StrepTagII sequence was fused via a Ser-Ala linker to the C-terminus of the mature A2A, followed by a Stop codon and a NotI restriction site.

The Gene Designer software from DNA2.0 Inc., Menlo Park, Calif., USA was used. The codon usage table designed for high level expression during methanol induction in Pichia pastoris published by Abad et al. ((2010) Microbial cell factories 9, 24) was applied. Further, common restriction sites were excluded and pentameric or higher NT combinations in order to allow unhindered cloning and to avoid destabilizing repeats, respectively. Further it was assured a balanced GC content (i.e. min. 30%, max. 70%) over the whole sequence, as well as a free energy higher than −15 kcal/mol in RNA secondary structure predictions for the avoidance of any impairment with the ribosomes' processivity. The GC content was determined with the online EMBOSS 6.3.1: freak tool (averaging window: 30 bp, stepping value: 1), RNA secondary structure prediction was done with the GeneBee web tool.

The gene was synthesized and cloned into a pUC vector.

2.1.2 Cloning of the HRP A2A Gene

Primers (A2StrepRemovalfw, A2StrepRemovalrv) were designed for the removal of the StrepTagII sequence from A2Cstrep via PCR and to end up with an untagged A2A sequence. Furthermore, primers were designed to fuse the StrepTagII sequence via a Ser-Ala linker in between the α-factor signal sequence and the N-terminus of the mature HRP A2A protein sequence and thus create A2ANstrep in an overlap PCR: The first PCR was done with the primers A2_Nterm_Strepfw1 plus A2StrepRemovalrv. The PCR product was cut from a preparative agarose gel, purified and used as template in a second PCR with the primers A2_Nterm_Strepfw2 plus A2StrepRemovalrv.

A2ACstrep and A2A were cloned to pPpT4_Smil, A2ANstrep was cloned to pPpT4Alpha_Smil via their EcoRI and NotI restriction sites. The three plasmids were transformed to E. coli Top 10F′ for plasmid amplification and subsequently sent to Sanger sequencing for sequence verification, prior to their transformation to P. pastoris MutS for recombinant protein expression.

2.1.3 Expression of HRP A2A in P. pastoris and Coexpression of PDI

The P. pastoris strains expressing A2ACstrep and A2ANstrep were screened for HRP activity via the ABTS assay subsequent to micro-scale cultivation in 96-DWP. The two best expressing A2ANstrep clones were picked for small scale cultivation in 2 L-shake flasks.

In order to screen for P. pastoris A2AMutS clones expressing sufficiently high levels of functional HRP, micro-scale cultivations in 96-DWPs was done and the HRP activity of approximately 200 clones evaluated via the ABTS assay. Moreover, the PDI genes synPDI, synPDI N314H and PDI704 were transformed in the pPpKan_Smil plasmid to the A2AMutS clone that yielded the highest HRP activity and also studied the influence of the coexpressed genes on HRP activity in 96-DWPs. The PDI704 gene was isolated from gDNA of P. pastoris CBS704 at the Research Centre Applied Biocatalysis, Graz, Austria. synPDI represents the gene for a synthetic PDI gene based on the sequence of a P. pastoris CBS7435 PDI. synPDI N314H is a mutant of the synPDI gene with a N314H spontaneous mutation and was identified at the Institute of Molecular Biotechnology, Graz, Austria. The aligned protein sequences of the mentioned PDIs are shown in FIG. 29.

In order to evaluate the mere influence of a second transformation event itself (e.g. molecular interactions between the first and the second plasmid or alterations in the host genome by the integration of the second plasmid), the empty pPpKan_Smil plasmid was transformed. Approximately 200 clones were screened and a selection of active clones was picked for rescreening. Hereto, these candidate clones were streaked to single colonies and four clones per candidate were picked for a second micro-scale cultivation and subsequent ABTS assay.

Selection of transformants was performed by streaking the cells onto YPD agar plates containing either Zeocin™ (from pPpT4_Smil or pPpT4Alpha_Smil), kanamycin (from pPpKan_Smil) or both. The HRP C1A expressing P. pastoris strains 110G4, 110B5, 107D6 and 107A9 were co-cultivated as positive controls for HRP activity. Two wells per 96-DWP were not inoculated with any strain as sterile controls. A duplicate of the non-transformed starting strain P. pastoris MutS was also co-cultivated as negative control for HRP activity. In the rescreen of the coexpressing strains, the starting A2AMutS strain was co-evaluated in quadruplicate in order to be able to assess an increase in the HRP activity of the coexpression strains.

2.1.4 Cultivation of P. pastoris Expressing HRP A2A in DASGIP Bioreactors

In order to enable future transcriptome analyses of HRP expressing strains, four strains were chosen for cultivation in DASGIP bioreactors. A2AMutSF5 and A2AMutSF5 synPDI N314H were used as HRP expressing strains. MutS ZeoR and MutS synPDI N314H were used as reference strains. The latter was generated by transforming pPpKan_Smil_synPDI N314H to P. pastoris MutS. The hereby resulting clones were verified for containing the synPDI N314H gene via colony PCR with the primers RT-synPDI-fw and AOXseq_rv.

Prior to the actual cultivation process, the copy numbers of A2A and synPDI N314H were determined in the respective strains via qPCR (see 1.7), in order to ensure comparable genetic conditions and thus comparability of the obtained HRP yields.

The cultivation procedure was performed as described in 1.12. Samples for the assessment of HRP activity and future RNA isolation were drawn at the end of the FedBatch phase (0 h) and four times during the Induction phase (4 h, 24 h, 70 h, 90 h). At every time point, approximately 1.4 mL of the culture were drawn from each reactor. The OD₆₀₀ was measured and aliquots corresponding to a number of 3×10⁸ cells (under the assumption that 3×10⁸ cells correlate with 15 mL culture of OD₆₀₀=1) were transferred to three microcentrifuge tubes per reactor and time point. The samples were spinned down for 2 min, 12,000×g, 4° C., the supernatant was discarded and the pellet was flash frozen in liquid nitrogen and stored at −80° C. for eventual total RNA isolation. The remaining sample culture was spinned down for 5 min, 3,000×g, 4° C. and the supernatant was frozen at −20° C. for later assessment of HRP activity via the ABTS assay. All handling was done on ice.

After the full cultivation procedure the cultures expressing HRP were transferred to 500 mL PPCO centrifuge bottles and spinned down for 15 min at 3,000×g, 4° C. in the Beckman Coulter Avanti centrifuge J-20 XP with the Beckman Coulter JA-10 Rotor.

The supernatant was stored at −20° C. for upcoming analyses.

2.2 Results and Discussion

2.2.1. Expression of the Optimized HRP A2A Gene

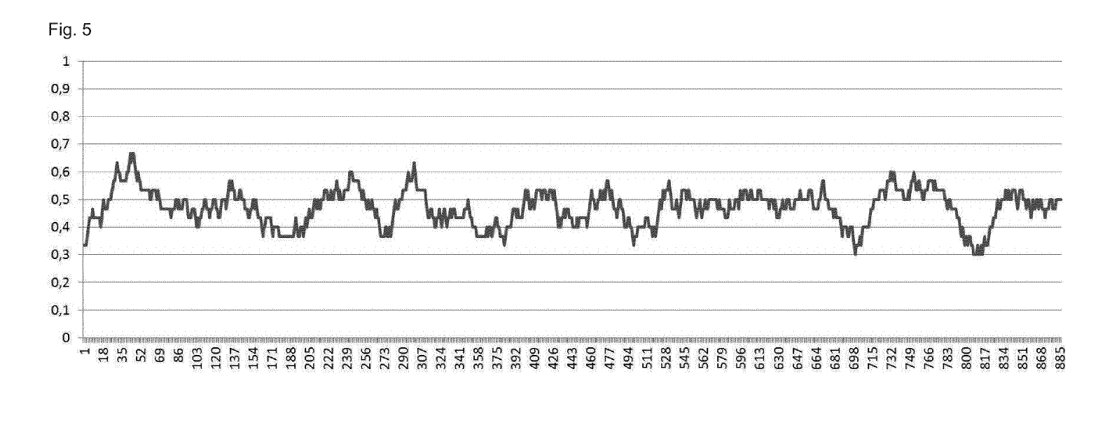

The sequence of A2ACstrep was successfully optimized for expression in Pichia pastoris during methanol induction. The predicted RNA secondary structure and the GC distribution over the optimized sequence are shown in FIG. 4 and FIG. 5, respectively. The optimized sequence of A2ACstrep and the applied codons are depicted in SEQ ID NO:60.

The synthetic A2ANstrep was successfully modified to the untagged A2A and the C-terminally tagged A2ACstrep and these three genes were successfully transformed to P. pastoris.

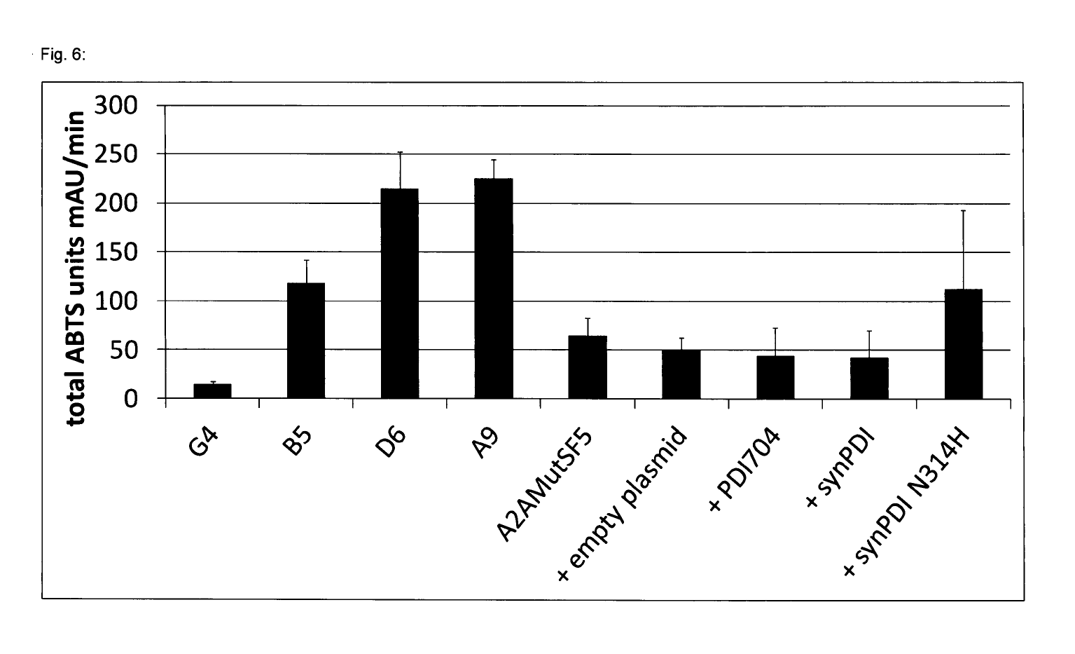

Coexpression of the PDI genes PDI704, synPDI and synPDI N314H yielded interesting results in the screening in 96-DWPs. Coexpressed PDI704 and synPDI did not seem to affect the HRP activity significantly. However, coexpressed synPDI N314H increased the measured activity up to four fold, compared to the activity of the starting strain A2AMutSF5 (FIG. 6).

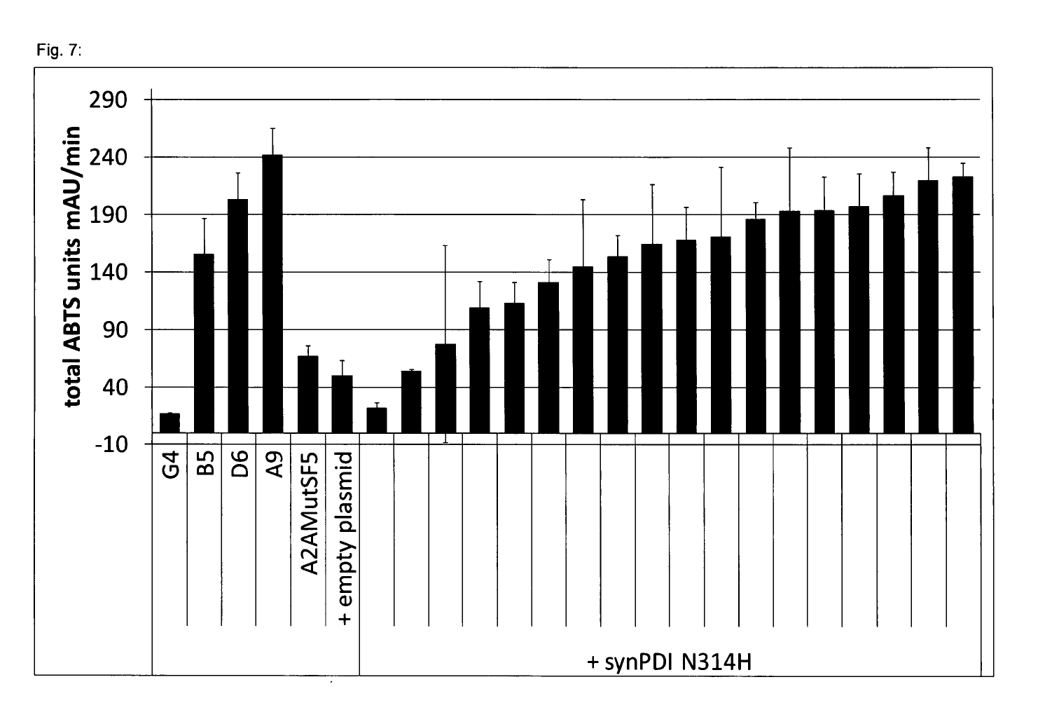

SynPDI N314H, being the most promising candidate for coexpression, was chosen for rescreening, the results are depicted in FIG. 7. The obtained HRP activities of A2AMutSF5 strains coexpressing synPDI N314H were significantly increased from the starting strain and are comparable to the activities of the optimized HRP C1A expressing control strains.

The mechanism underlying the specific increase in HRP activity of the N314H mutant of synPDI is not yet understood and remains to be elucidated. Classification of the PDI amino acid sequence applying the NCBI Conserved Domain Search web tool, showed position 314 to lie in a domain classified to the PDI b′ family (redox inactive TRX-like domain b′) which is a member of the thioredoxin-like superfamily. The b′ domain is described as the primary substrate binding site and to be implicated in chaperone activity. Probably, the N314H mutation in this domain increased the recognition of HRP as a substrate and promoted its correct folding, resulting in elevated yields of functional HRP A2A.

By screening P. pastoris clones expressing A2ACstrep for HRP activity, it was noticed that the HRP activity was abolished by the C-terminally fused StrepTagII, which probably interfered with the enzyme's folding to its native conformation. Thus it was focused on the expression of A2ANstrep, which showed HRP activity comparable to the untagged A2A. The two clones with the highest activity were picked and A2ANstrep was successfully expressed in small scale cultivation. The hereby obtained A2ANstrep-containing supernatant could be used for an affinity purification approach.

2.2.2 Cultivation of P. pastoris Expressing HRP A2A in DASGIP Bioreactors

The generation of the strain MutS synPDI N314H was successfully verified via agarose gel electrophoresis of the colony PCR product. By applying qPCR the gene copy numbers of HRP A2A was determined as well as of PDI in the corresponding strains. The qPCR results are shown in Table 8. The Arg/Zeo data represents the HRP copy number, Arg/Pdi and Arg/Kan both represent the PDI copy number and are equally suitable for this purpose. The average values were calculated from the two values obtained from absolute and relative copy number quantification. Normalized data accounts for fluctuations in the method and normalizes the raw data to the reference's value.

TABLE 8

| | | | | |
|---|---|---|---|---|
| sample | absolute | relative | average | stdv |

Arg/Zeo

| | | | | |
|---|---|---|---|---|
| raw data | | | | |
| A2AMutSF5 | 0.85 | 0.89 | 0.87 | 0.03 |
| A2AMutSF5 synPDI N314H | 0.89 | 0.91 | 0.90 | 0.02 |
| reference | 0.84 | 0.85 | 0.85 | 0.01 |
| normalized data | | | | |
| A2AMutSF5 | 1.01 | 1.04 | 1.02 | 0.02 |
| A2AMutSF5 synPDI N314H | 1.06 | 1.07 | 1.06 | 0.01 |
| reference | 1.00 | 1.00 | 1.00 | 0.00 |

Arg/Pdi

| | | | | |
|---|---|---|---|---|
| raw data | | | | |
| A2AMutSF5 synPDI N314H | 0.59 | 0.59 | 0.59 | 0.00 |
| MutS synPDI N314H | 1.20 | 1.20 | 1.20 | 0.00 |
| reference | 0.96 | 0.96 | 0.96 | 0.00 |
| normalized data | | | | |
| A2AMutSF5 synPDI N314H | 0.62 | 0.61 | 0.61 | 0.00 |
| MutS synPDI N314H | 1.24 | 1.24 | 1.24 | 0.00 |
| reference | 1.00 | 1.00 | 1.00 | 0.00 |

Arg/Kan

| | | | | |
|---|---|---|---|---|
| raw data | | | | |
| A2AMutSF5 synPDI N314H | 0.62 | 0.65 | 0.64 | 0.02 |
| MutS synPDI N314H | 0.99 | 1.00 | 1.00 | 0.01 |
| reference | 1.02 | 1.04 | 1.03 | 0.01 |
| normalized data | | | | |
| A2AMutSF5 synPDI N314H | 0.61 | 0.63 | 0.62 | 0.01 |
| MutS synPDI N314H | 0.97 | 0.97 | 0.97 | 0.00 |
| reference | 1.00 | 1.00 | 1.00 | 0.00 |

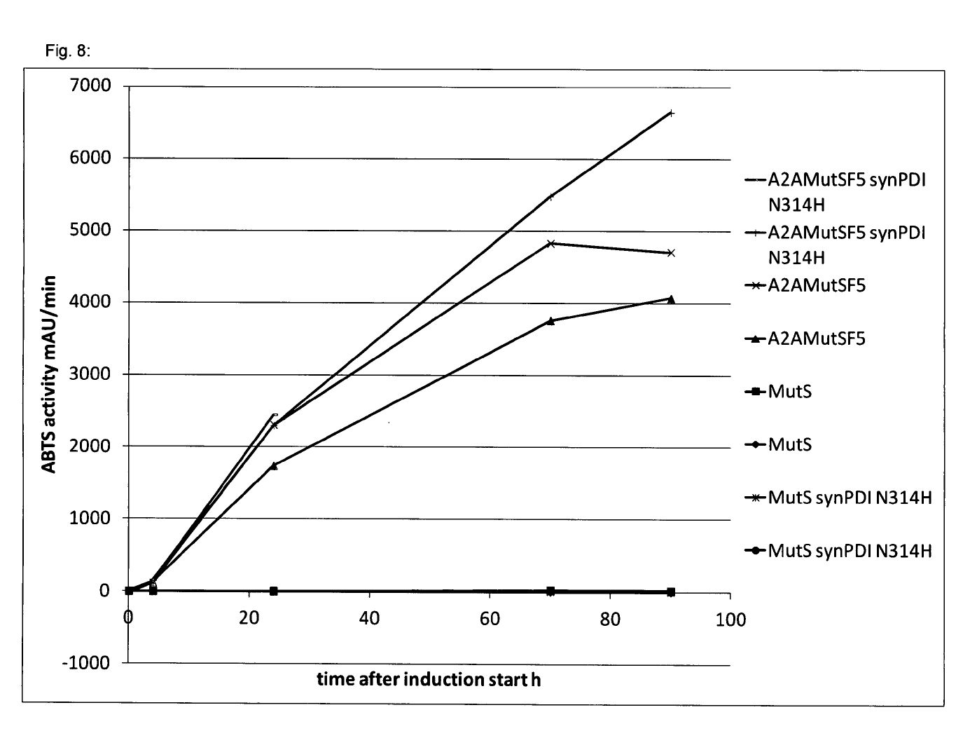

The analyzed strains were successfully used in the DASGIP cultivation, the measured HRP activities are shown in FIG. 8.

The coexpression of A2A plus synPDI N314H yielded the highest activities, yet not as strikingly higher than sole A2A, as in micro-scale cultivations.

### Example 3

### Purification of HRP A2A

### 3.1 Experimental

3.1.1 Affinity Chromatography

Using the Vivaspin 20 system, the Strep-tagged HRP A2A sample from small scale cultivation supernatant was concentrated to ˜1000 μL and mixed with 14 U avidin in order to bind interfering biotin from the cultivation medium. The protein solution was dialyzed over night at 4° C. against 1 L of the IGA GmbH buffer W (100 mM Tris-HCl, pH 8.0, 150 mM NaCl, no EDTA), using a dialysis tube with 8,000-10,000 MWCO cut-off. The same preparation was performed without the preceding avidin treatment in order to exclude the possibility of column blockage by the used avidin.

The dialyzed enzyme solution was concentrated with the Vivaspin 20 system to 500 μL-1000 μL and loaded onto the Gravity flow Strep-Tactin® MacroPrep® column.

The collected fractions of the run were analyzed for HRP activity by applying the ABTS assay.

3.1.2 Hydrophobic Interaction Chromatography

The supernatant of A2AMutSF5 was concentrated from the cultivation in the 5 L-BIOSTAT bioreactor to a volume of 50 mL and changed the buffer to HIC-A, subsequent to tangential flow filtration using the Centramate 500 S system with a corresponding filtration membrane cassette with a 30 kDa cut-off.

These 50 mL of HRP A2A in buffer HIC-A were loaded onto the HIC column with a flowrate of 5 mL/min (˜56.5 cm/h) and the purification run was performed as described in 1.16. During the run, fractions of 5.0 mL to 15 mL PP-tubes were collected.

3.1.3 Anion Exchange Chromatography

Prior to the actual anion exchange chromatography run, the test tube experiment was performed as described in 1.16, in order to identify the pH at which HRP A2A binds to the QFF material.

For the anion exchange chromatography, all HIC fractions were pooled that showed HRP activity, changed the buffer to QFF-A and concentrated the enzyme solution to approximately 50 mL.

This volume was loaded onto the anion exchange column with a flowrate of 5 mL/min (˜56.5 cm/h). Throughout the run, fractions of 1.2 mL were collected to 96-DWPs.

3.1.4 Fractional Precipitation with Ammonium Sulfate

The QFF fraction with the highest Rz value was picked for fractional precipitation with ammonium sulfate as described in 1.16.

3.1.5 Size Exclusion Chromatography

All QFF-fractions were pooled that showed HRP activity, changed to Superdex buffer and concentrated the sample to a volume of approximately 800 μL. This volume was loaded onto the column with a flow of 0.3 mL/min (˜9.0 cm/h). Fractions of 800 μL were collected to 96-DWPs throughout the whole run.

The fraction giving the highest Rz value was stored at 4° C. and evaluated its purity via SDS-PAGE.

### 3.2 Results and Discussion

3.2.1 Affinity Chromatography

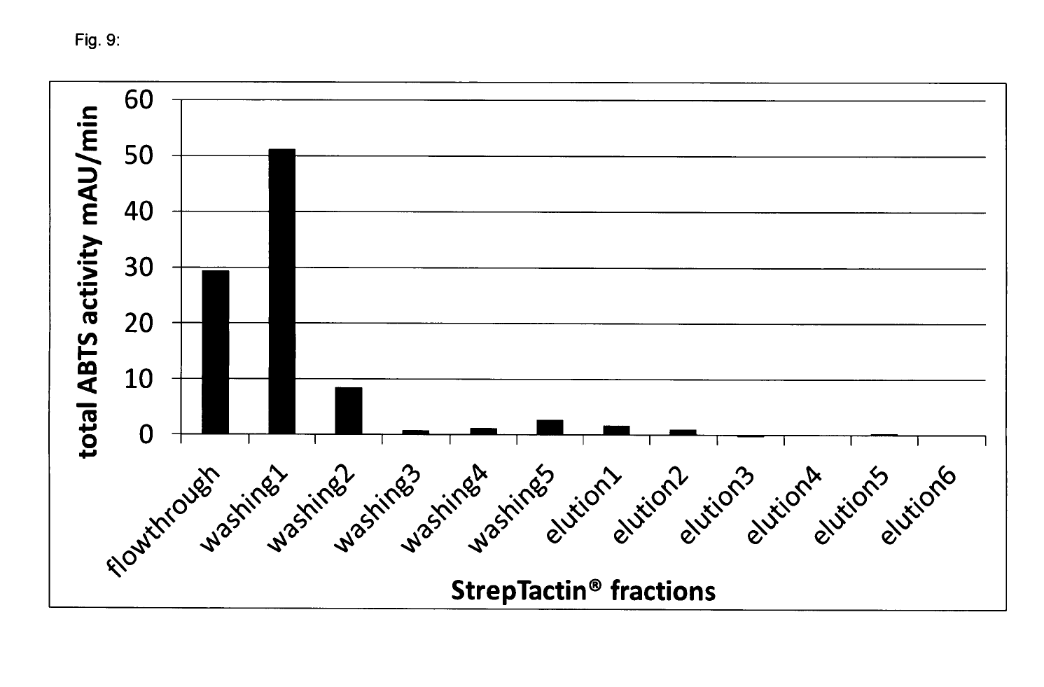

By applying the StrepTactin protocol to HRP A2ANstrep, no purification could be achieved. HRP activity was predominantly measured in the flowthrough fraction collected during sample loading and in the first washing fractions. The actual elution fractions did not exhibit any significant HRP activity any more (FIG. 9). HRP A2ANstrep did not bind to the StrepTactin column and eluted in the first fractions. Already in the flowthrough fraction, collected during the loading of the sample, HRP activity was measured. In the subsequent two washing fractions the remaining HRP activity was detected. The sample without avidin treatment provided the same results.

3.2.2 Hydrophobic Interaction Chromatography

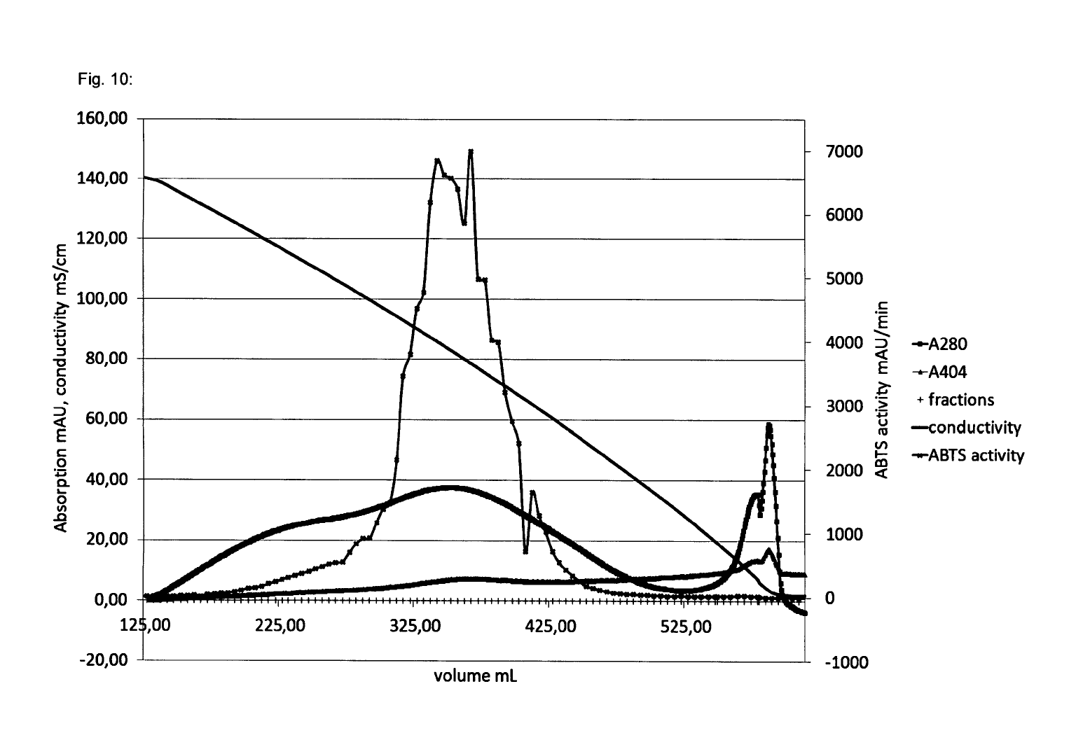

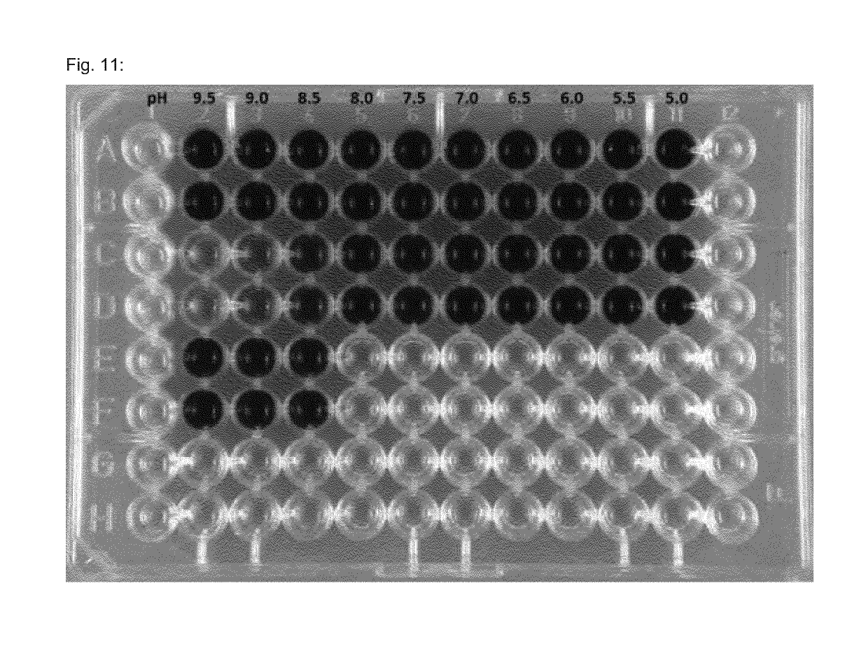

By using the HIC approach, it was possible to separate a lot of undesirable proteins from HRP in the cultivation supernatant. The chromatogram and the measured HRP activities are depicted in FIG. 10.

The measured HRP activity exposed a double peak, which might represent monomeric and aggregated oligomeric HRP A2A species that featured slightly different binding behaviors.

No significant amounts of HRP were lost in this purification step. The obtained Rz value after HIC purification was <0.2. The fractions from 300 mL-425 mL were used for further purification. Hereto, they had to be pooled, concentrated and changed to the buffer of the following purification step.

3.2.3 Anion Exchange Chromatography

The test tube experiment provided the following data: By incubating A2A at different pH with the QFF material, full HRP activity was verified in the supernatants at pH 5.0-8.5. However, decreased HRP activity in the supernatant at pH 9.0, and no activity in the supernatant at pH 9.5 could be measured, indicating binding of A2A to QFF. By washing the QFF material and the bound HRP A2A with the same buffer and additional 1 M NaCl, HRP was successfully eluted from the QFF material and thus the HRP activity in the supernatant restored. Incubation of A2A in all 10 buffers without QFF material and subsequent activity verification via ABTS assay was successful in all buffers and thus loss of HRP activity at a certain pH could be excluded (FIG. 11).

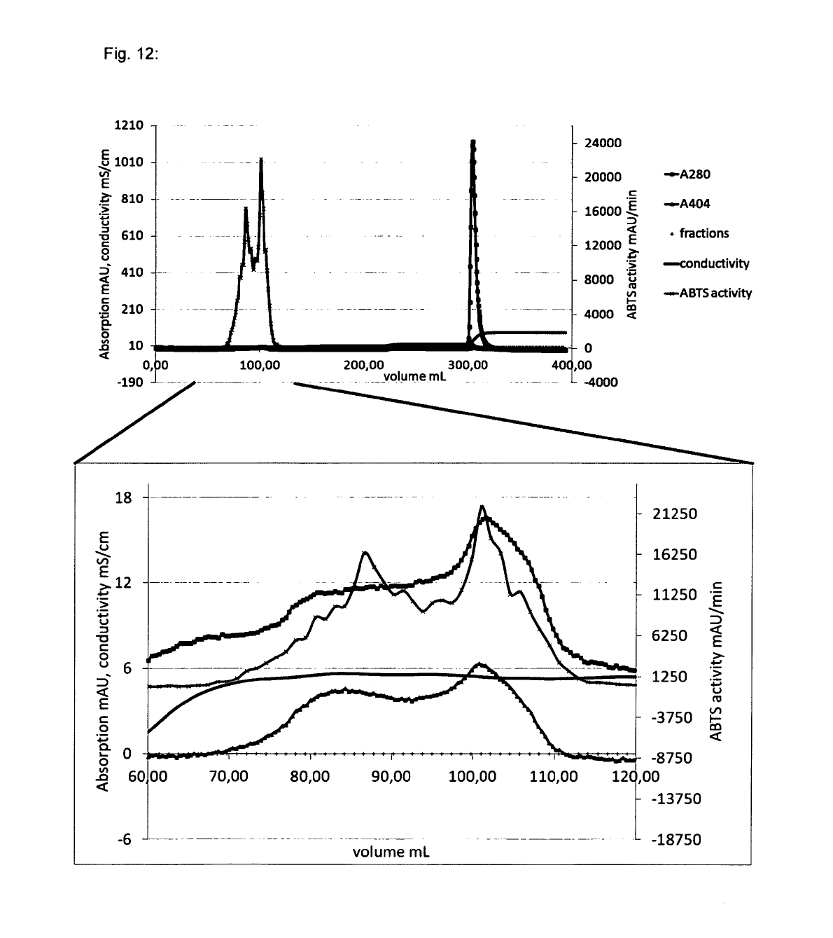

By running the anion exchange chromatography at pH 9.5, HRP A2A was further purified and a max. Rz value of approximately 0.5 achieved (FIG. 12).

Again, HRP A2A eluted in two peaks, pointing to two conformational species. For maximum recovery of HRP A2A, all fractions exposing HRP activity (˜70-115 mL) were used for final purification via size exclusion chromatography. Moreover, the fraction at 100 mL run volume was partially used for fractional precipitation with ammonium sulfate.

3.2.4 Fractional Precipitation with Ammonium Sulfate

By performing fractional precipitation with ammonium sulfate of the purest anion exchange chromatography fraction HRP A2A was further purified to a Rz value of ˜1.8, which is already comparable to a commercially available HRP preparation (Peroxidase from horseradish Type II/Sigma-Aldrich Handels GmbH, Vienna, Austria; denoted Rz≧1.8).

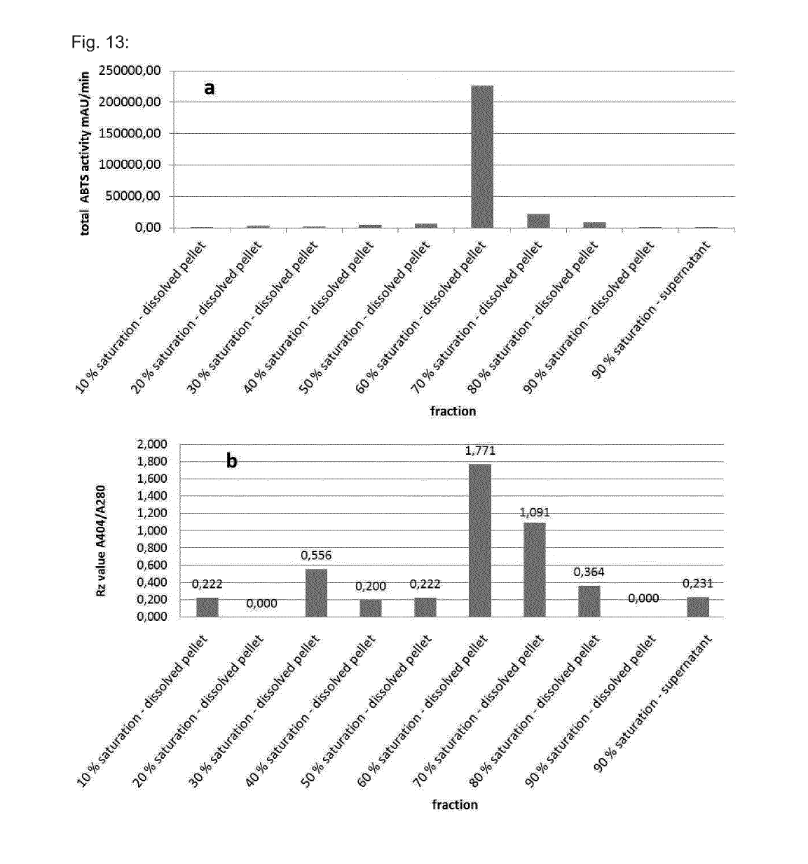

The ABTS activities of the obtained fractions and the corresponding Rz values are depicted in FIG. 13.

The fractional precipitation with ammonium sulfate constitutes a fast approach for the purification of HRP A2A to a medium level of purity.

3.2.5 Size Exclusion Chromatography

Alternatively to the “quick-and-dirty” precipitation approach, size exclusion chromatography was performed that yielded a high level of purity of HRP A2A with Rz value ≧3.5, which is even higher than the denoted Rz ˜3.0 of the purest commercially available HRP preparation from Sigma (Peroxidase from horseradish Type VI/Sigma-Aldrich Handels GmbH, Vienna, Austria).

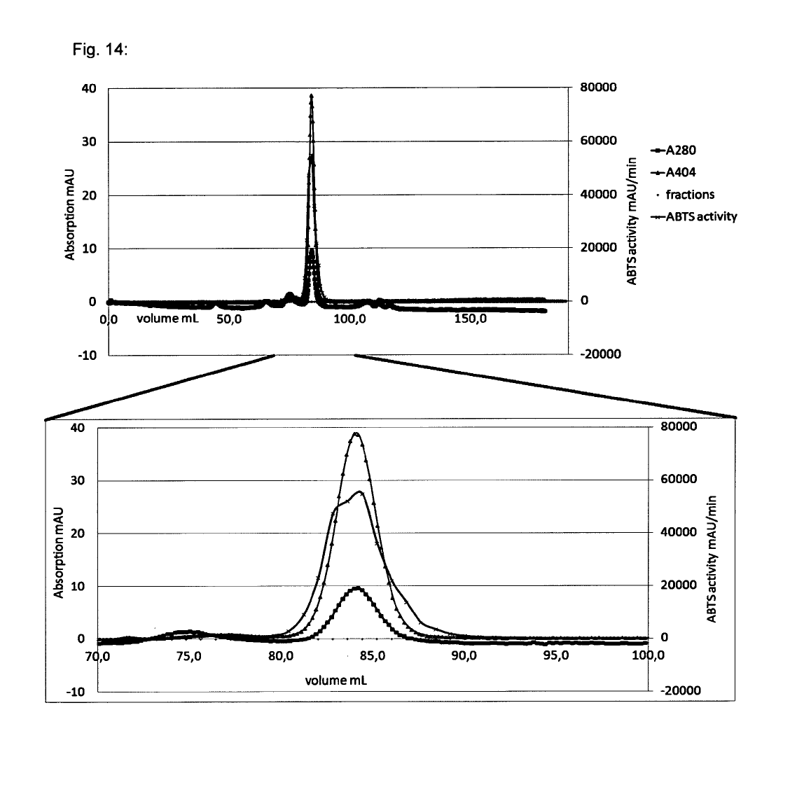

HRP A2A eluted in the fractions from approximately 80-90 mL. The chromatogram of the size exclusion chromatography run and the measured HRP activities are shown in FIG. 14.

The fraction with the highest Rz value and the highest HRP activity (at 84 mL total run volume) was determined to have a protein concentration of 60 ng/μL (see 4.2.3). Considering the fraction volume of 800 μL, the total yield of pure HRP A2A from one 5 L-BIOSTAT reactor was 50 μg.

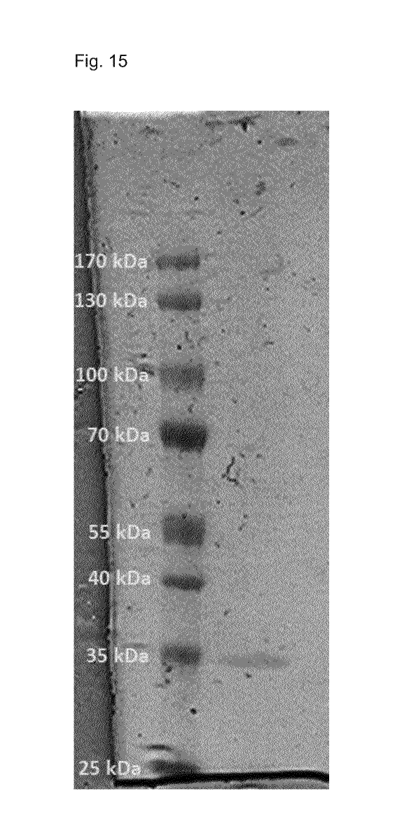

SDS-PAGE of this fraction confirmed the high level of purity and the successful purification of the recombinantly expressed HRP A2A (FIG. 15).

### Example 4

### Characterization of HRP A2A

### 4.1 Experimental

4.1.1 Posttranslational Modifications of HRP A2A

In order to study the purified recombinant HRP A2A for posttranslational modifications, a mass spectrometric analysis at the ZMF Mass Spectrometry—Proteomics Core Facility was conducted. Further, the PredictProtein web tool was used on the mature HRP A2A amino acid sequence in order to compare the predicted results with those from the mass spectrometry.

4.1.2 Isoelectric Focusing of HRP A2A

The binding of HRP A2A to QFF at pH 9.5 happened at a much more basic pH as expected (considering the theoretical IEP of A2A at pH 4.82).

Thus, an isoelectric focusing (see chapter 1.6.3) of HRP A2A was performed and compared to the commercially available HRP preparations Type I, Type II, Type VI, Type VI-A, Type XII from Sigma-Aldrich Handels GmbH and a HRP preparation from Toyobo Co., Ltd.

4.1.3 HRP A2A and ABTS

pH Optimum of HRP A2A Plus ABTS

The interaction of HRP A2A was verified with the HRP standard substrate ABTS. The influence of pH on the catalysis of ABTS was studied. The ABTS assay was performed with the purified HRP A2A in buffers of different pH:

The 50 mM NaOAc, pH 4.5 standard buffer was used (Morawski et al. (2000)), 20 mM citrate-NaOH buffers at pH 3.0, 3.5, 4.0, 4.5, 5.0, 6.0, 7.0 and 20 mM Tris-HCl buffers at pH 7.0, 8.0, 9.0, 10.0. By performing the assay at the same pH with different buffer systems (i.e. 50 mM NaOAc and 20 mM citrate-NaOH at pH 4.5; 20 mM citrate-NaOH and 20 mM Tris-HCl at pH 7.0), any influences of the buffer systems on the reaction was excluded, other than the pH. Changing to the corresponding pH was achieved by 1:200 dilution of the purified A2A in the respective buffer.

Michaelis-Menten Kinetics of HRP A2A Plus ABTS

Since the optimal pH of the reaction of HRP A2A with ABTS was identified. A buffer at this pH was used for determining the kinetic constants Vₘₐₓ and K_{M} for the HRP catalyzed conversion of ABTS. Herefore the assay was performed with varying concentrations of ABTS: 0.1, 0.33, 0.5, 0.75, 1.0, 1.5, 2.0 and 3.0 mM. The measured activities in mAU/min were used to calculate the corresponding ABTS units/mL (1 ABTS unit is defined as the amount of enzyme that converts 1 μmol ABTS per minute) via the equation depicted below.

$$ABTS units \text{/} mL = \frac{\frac{mAU}{min} * f}{ɛ * d} * \frac{V}{v}$$

Equation for the Calculation of ABTS Units Per Milliliter.

f=dilution factor=200.
ε_{ABTS}=absorption coefficient of ABTS=34,700 M-1 cm-1.
d=path length=0.42 cm.
V=total reaction volume=155 μL.
v=enzyme solution volume=15 μL.

In order to be able to measure the increase in the absorption at 404 nm in the ABTS reaction the purified A2A 1:200 was diluted. The dilution factor f is taken into consideration in all calculations. The path length d was verified with the corresponding Path Check function of the SoftMax Pro 4.8 software.

The specific activity of HRP A2A for ABTS was seeked to be verified. Hereto the purified A2A was quantified via the Pierce BCA Protein Assay Kit as described in chapter 1.6.15. Using this data the specific activity of HRP A2A in ABTS units/mg was calculated via dividing the ABTS units/mL by the protein concentration in μg/mL and multiplying with 1000.

4.1.4 HRP A2A and Guaiacol

pH Optimum of HRP A2A Plus Guaiacol

Similarly to the verification of the pH optimum of the HRP-catalyzed ABTS reaction, another assay was performed in buffers of different pH with a second standard substrate, guaiacol. As standard buffer 20 mM phosphate-KOH buffer, pH 7.0 as published by Morawski et al. (2000) was tested. Moreover, further 20 mM phosphate-KOH buffers were tested at pH 5.0, 6.0 and 8.0, 20 mM citrate-NaOH buffers at pH 3.0, 3.5, 4.0, 4.5, 5.0, 6.0, 7.0 and 20 mM Tris-HCl buffers at pH 7.0, 8.0, 9.0, 10.0 and 10.5.

Michaelis-Menten Kinetics of HRP A2A Plus Guaiacol

Using the gathered data on the optimal pH for the conversion of guaiacol by HRP A2A kinetic studies were performed in analogy to those of A2A with ABTS with the following accommodations in the calculations:

f=dilution factor=65 and

ε_{guaiacol}=absorption coefficient of guaiacol=26,000 M-1 cm-1.

Thus guaiacol units were aimed to be obtained corresponding to varying concentrations of the substrate (0.5, 0.75, 1.0, 3.0, 5.0, 7.0, 10.0, 20.0 mM). 1 guaiacol unit is defined as the amount of enzyme that converts 1 μmol guaiacol per minute.

4.1.5 Stability Studies with HRP A2A

Optimal Storage pH of HRP A2A

The influence of pH on the storage stability of HRP A2A was evaluated. Therefore, the purified HRP A2A was diluted 1:20 in buffers of different pH (20 mM citrate-NaOH buffers at pH 3.0, 4.0, 5.0, 6.0 and 7.0 and 20 mM Tris-HCl buffers at pH 8.0, 9.0 and 10.0) and stored the different tubes at 4° C. In order to measure the starting/remaining activities at the time points 0 d, 2 d, 6 d, 10 d, 20 d (for pH 3.0 also measured after 1 d), and at the same assay conditions, aliquots from the tubes were further diluted with the different pHs 1:10 in 50 mM NaOAc buffer, pH 4.5 and the ABTS assay performed.

Temperature Stability of HRP A2A

Pure A2A 1:200 (i.e. 0.3 ng/μL) was diluted in 50 mM NaOAc buffer, pH 4.5 and incubated at different temperatures: 20° C., 37° C., 50° C., 65° C. For each incubation temperature aliquots were taken after several time points and stored on ice until the end of the whole experiment. Finally, the HRP activities from all time points of one incubation temperature were measured at once with the ABTS assay.

Dependence of the Stability of HRP A2A on Protein Concentration

HRP A2A stays stable for long time (>>20 d) as long as it is undiluted. The purified HRP A2A was diluted with the Superdex buffer to the following concentrations: 60.0 ng/μL, 2.0 ng/μL, 1.2 ng/μL, 0.8 ng/μL, 0.6 ng/μL, 0.46 ng/μL and 0.38 ng/μL. The HRP activity was measured after 0 h, 1 h, 5 h, 1 d, 3 d and 10 d. Hereto, aliquots were taken from the various dilutions and separately further diluted to a final total dilution of 1:200 (i.e. 0.3 ng/μL) and the ABTS assay performed.

A 6.7 ng/μL concentrated solution of HRP A2A was examined for the existence of nanoparticles using the ZetaPlus Zeta Potential Analyzer.

### 4.2 Results and Discussion

4.2.1 Posttranslational Modifications of HRP A2A

The acquired MALDI-TOF MS (matrix-assisted laser desorption/ionization—time-of-flight mass spectrometry) data for intact mass determination revealed two main species of A2A.

Top-down sequencing via MALDI-TOF-TOF MS provided data that suggested an unprocessed C-terminus and two varying N-termini: They differed in one amino acid from the C-terminal end of the α-factor signal sequence, which obviously has been heterogeneously cleaved from the mature HRP A2A peptide. One species had a N-terminal AEA upstream the mature HRP A2A sequence, the other species had EAEA. Thus the Step 13 site was not correctly processed in P. pastoris.

Moreover, the gathered data suggested Cys11 to build a disulfide bridge, which also has been predicted by the PredictProtein tool and which is also annotated for the HRP A2 published at UniProt.

PredictProtein further predicted the same seven N-glycosylation sites (N3, N13, N147, N185, N197, N211, N267) that are also annotated at UniProt. The mass spectrometric analysis detected a HexNAc (N-acetylhexose) residue on N3, N13 and N147. N185 did not seem to be modified, the other N-glycosylation sites were not covered by the acquired data.

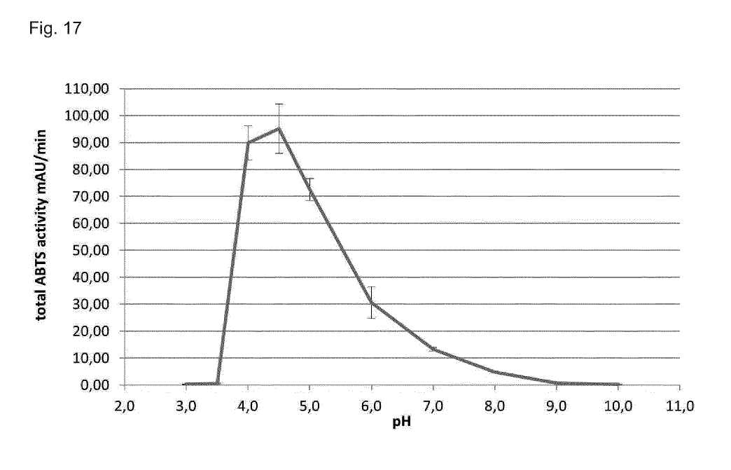

Assuming a total of seven HexNAc residues per molecule, the two species with the differing N-termini fitted perfectly to the determined intact masses. Probably N197, N211 or N267 carries not one, but two HexNAc residues and thus accounts for the seemingly unmodified N185. This small extent of glycosylation was also reflected as a discrete protein band on the SDS gel (FIG. 17).

Having a homogeneously glycosylated HRP A2A might significantly simplify eventual crystallization and structure analysis, which has been hindered so far by the N-glycans' heterogeneity of HRP A2 directly isolated from horseradish.

4.2.2 Isoelectric Focusing of HRP A2A

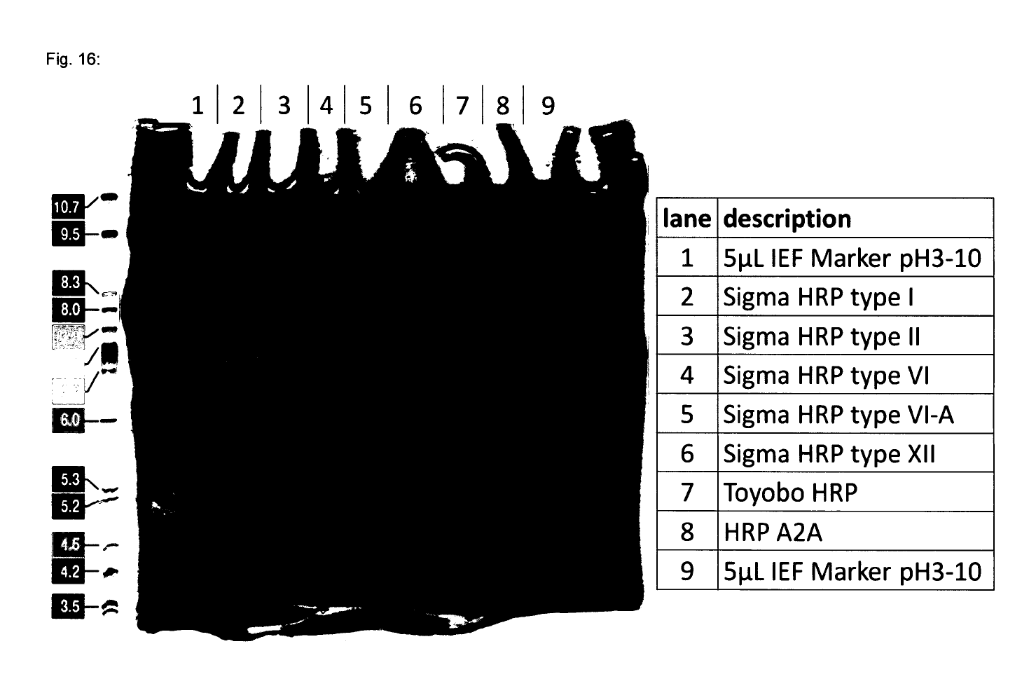

The isoelectric focusing of HRP A2A indicated an IEP at pH 3.5-4.2 (FIG. 16) which was close to the calculated theoretical IEP at pH 4.82. Most probably, the ionizable amino acid groups were predominantly not present on the enzyme's surface, which could explain its unexpected binding behavior towards QFF.

The IEP of the isoenzymes in the Sigma type I preparation was mainly around pH 5.0, the type II preparation showed two predominant isoenzymes at pH 5.3-6.0 and 3.5-4.2. The latter also seemed to be present in type VI, VI-A and XII with additional isoenzymes at 5.3-6.0 in type VI. The Toyobo preparation also showed an isoenzyme at pH 3.5-4.2, but slightly more basic than the A2A and the isoenzyme identified in the Sigma preparations.

4.2.3 HRP A2A and ABTS

pH Optimum of HRP A2A Plus ABTS

The pH optimum for the conversion of ABTS by HRP A2A at pH 4.5 was successfully identified (FIG. 17). Interestingly, Hiner et al. reported in 2001a broad pH optimum of HRP A2 for the conversion of ABTS at pH 5.5-6.5 (Journal of biological inorganic chemistry: JBIC: a publication of the Society of Biological Inorganic Chemistry 6, 504-16). However, the HRP A2 used for that publication was the preparation HRP-5 from Biozyme Labs (Blaenavon, UK) and has just been identified as A2 by isoelectric focusing (but not by sequencing). Most likely, this HRP also differs from the present recombinant A2A in its glycosylation pattern, which might also influence its enzymatic properties. In addition, the verified sequence used for the recombinant production of A2A differed from the published A2, which could also explain the varying results.

No activity could be detected at pH lower than 3.5 or higher than 9.0. Since HRP activity is also absent at pH lower than 3.5 or higher than 9.0 for the conversion of guaiacol (see 4.2.4), it might be that the enzyme itself is impaired in its activity at this pH.

In analogy to current literature on pH profiling using ABTS as substrate (Hiner et al. (2001)), the influence of pH on the absorption spectrum of ABTS was not taken into consideration for these studies. Consequently, in order to be able to evaluate the mere enzymatic activity of HRP A2A in the conversion of ABTS at a certain pH, further experiments need to be performed (e.g. the quantification of formed product by A2A at a certain pH after a given time via HPLC).

Michaelis-Menten Kinetics of HRP A2A Plus ABTS

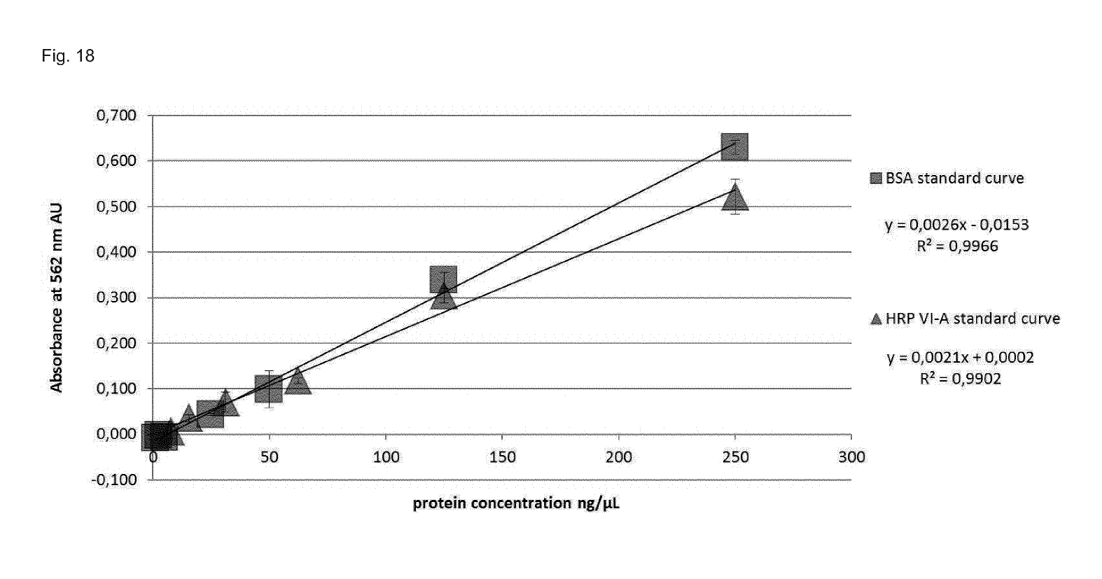

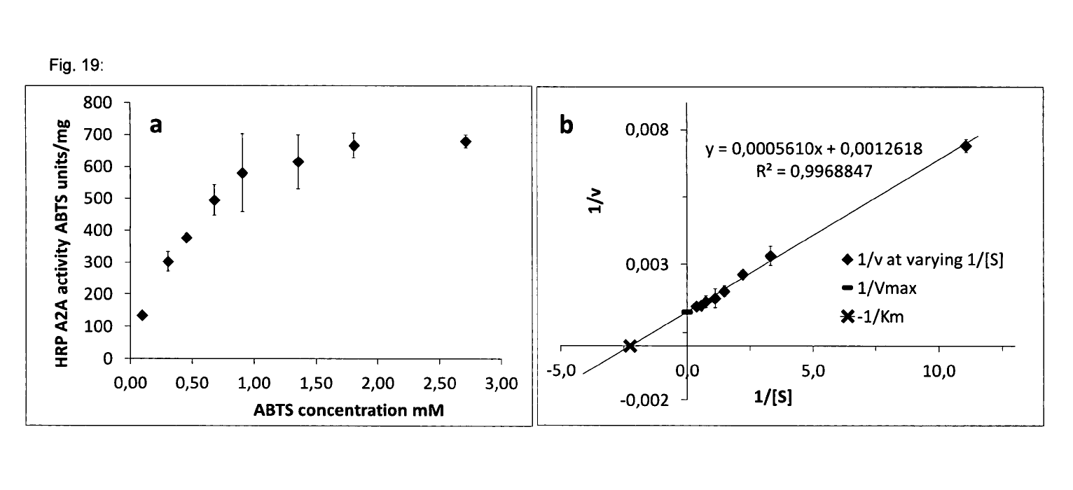

The performed BCA assay for protein quantitation revealed that both, BSA and HRP-VI-A were suitable as standard proteins for calculating the concentration of the purified HRP A2A (see FIG. 18).

HRP A2A quantitation by using the BSA standard curve yielded a concentration of 56.9 ng/μL and 63.1 ng/μL by using the HRP VI-A standard curve, giving an average HRP A2A concentration of 60.0 ng/μL, which was used for all further calculations.

By plotting the calculated ABTS units/mg of the measured activities against the respective ABTS concentrations a saturation curve following Michaelis-Menten kinetics was obtained (FIG. 19a). Plotting of the reciprocal values of the reaction rates against the reciprocal values of the corresponding ABTS concentrations yielded the Lineweaver-Burk plot (FIG. 19b) which allowed the determination of Vₘₐₓ and K_{M} with ABTS as substrate by calculating the reciprocal negative value of the crossing point of the trendline and the x-axis (i.e. K_{M}) and the reciprocal value of the crossing point of the trendline and the y-axis (i.e. Vₘₐₓ):

Vₘₐₓ=793 ABTS units/mg

K_{M}=0.44 mM ABTS.

As for the pH profile of HRP A2A, the values Hiner et al. published for their A2 isoenzyme differed from this one: They reported a Vₘₐₓ=1,432 ABTS units/mg and a K_{M}=4.0 mM at pH 4.5 (34). The HRP A2A was already in saturation at a concentration of 3.0 mM ABTS. Interestingly, this means a two times faster maximum conversion speed, but a ten times lower substrate affinity of their isoenzyme.

4.2.4 HRP A2A and Guaiacol

pH Optimum of HRP A2A Plus Guaiacol

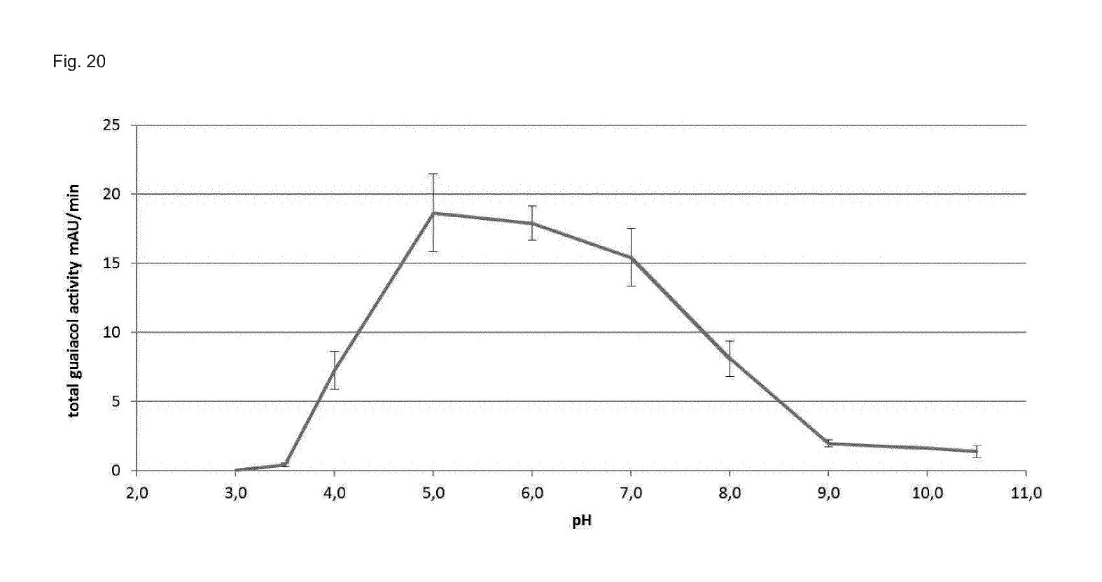

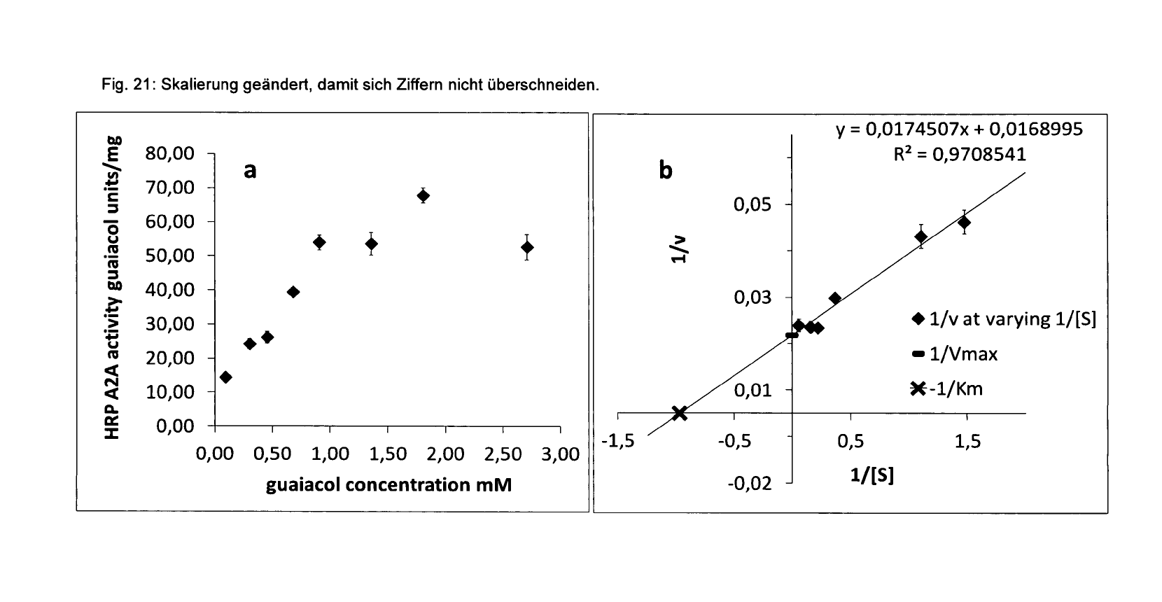

The pH optimum for the conversion of guaiacol by HRP A2A at pH 5.0 was verified, however the measured activities stayed at comparable levels until pH 7.0. In contrast to the corresponding ABTS profile, the enzyme's pH optimum seemed to dominate over the assay's pH optimum, since the profile much more resembled a normal distribution. Similarly to the ABTS profile, no significant activity could be measured below pH 3.5 or above pH 9.0 (FIG. 20).

Contrary to the pH profile with ABTS, the findings for the guaiacol pH profile matched the data published by Hiner et al. (2001). They also reported similar results for the conversion of guaiacol with HRP C1A. Probably, the guaiacol pH profile generally does not show bigger variations among the isoenzymes, which could explain the differences between A2A and their A2 in the ABTS assay, but not in the guaiacol assay. As for the pH profile of ABTS, the dependence of the absorption spectrum of guaiacol on pH was neglected and remains to be verified in further studies.

Michaelis-Menten Kinetics of HRP A2A Plus Guaiacol

As for the graphical representation and determination of the kinetic constants of A2A with ABTS, the calculated guaiacol units/mg of the measured activities were plotted against the corresponding substrate concentrations and thus obtained a curve that goes into saturation at a certain guaiacol concentration. Vₘₐₓ and K_{M} for the conversion of guaiacol by HRP A2A were again determined by creating a Lineweaver-Burk plot (FIG. 21):

Vₘₐₓ=59 guaiacol units/mg,

K_{M}=1.03 mM guaiacol.

Hiner et al. (2001) published a comparable Vₘₐₓ=81 guaiacol units/mg for the conversion of guaiacol by their HRP A2. Unlike for the conversion of ABTS, their isoenzyme seems to show catalytic properties towards guaiacol that are comparable to the obtained data with recombinant A2A.

4.2.5 Stability Studies with HRP A2A

Optimal Storage pH of HRP A2A

The activity of HRP A2A seems to be best preserved in buffers of pH 7.0-10.0 (see Table 9). In pH 3.0 and 4.0, the HRP activity was completely abrogated; in pH 5.0 and 6.0, the activity was significantly decreased; in pH 7.0-10.0, the activity was equivalently well preserved.

TABLE 9

| | residual HRP activity |
|---|---|
| pH | after 20 d % |
| 3.0 | 0.0 |
| 4.0 | 0.0 |
| 5.0 | 10.4 |
| 6.0 | 50.9 |
| 7.0 | 76.7 |
| 8.0 | 77.7 |
| 9.0 | 80.6 |
| 10.0 | 77.3 |

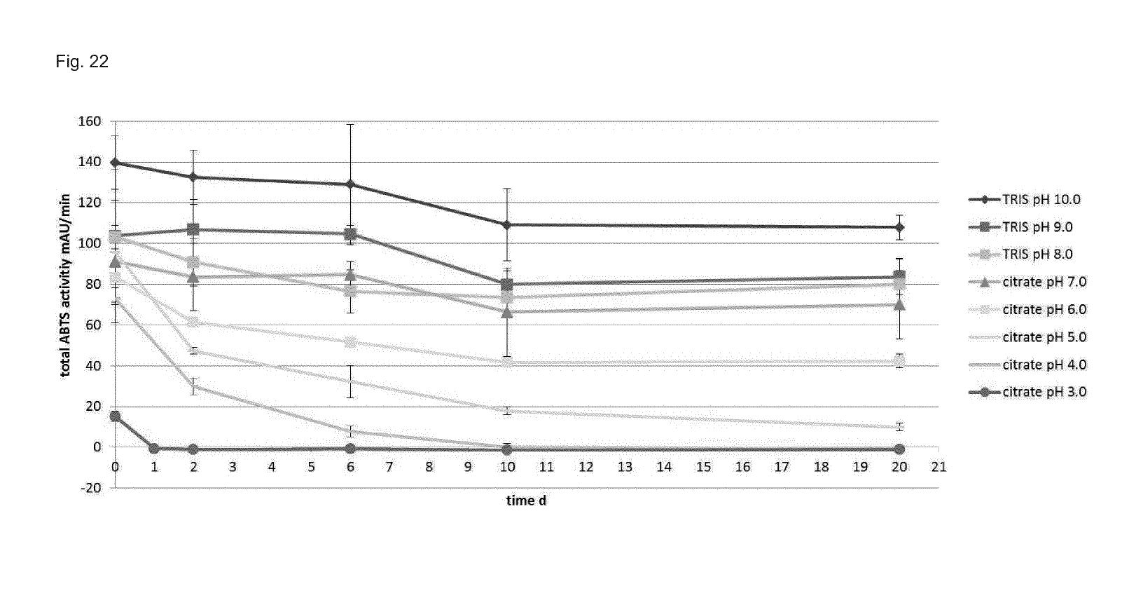

The total measured activities in all pHs over 20 d are depicted in FIG. 22. In pH 3.0, the HRP activity is completely abrogated in short time (no activity measurable after 1 d). In pH 4.0, the activity also continuously decreased and was below detection limits after 10 d. However, even at this relatively low pH still a third of its original activity was detected after two days. HRP A2A still yielded measurable, but significantly decreased activity after 20 d in pH 5.0, and with a further descending trend. In pH 6.0, the activity was decreased to 50% of the starting activity after 20 d, however it seemed to stay stable at this level. The best preserved HRP activity was detected in buffers of pH 7.0-10.0. The residual HRP activity in these four buffers was approximately 78% of the initial activity and stayed stable at this level.

This data extends the findings from the pH profiles: HRP A2A might have shown no activity at pH 3.5, because it was inactivated immediately, when transferred to the buffer of this pH. However, enzyme inactivation at pH higher than 9.0 seems not to be an explanation for the absence of HRP activity at that pH. The decrease in activity at basic pH seems to be rather due to impairments of the assay's reaction itself (e.g. an altered absorption spectrum of the measured product at higher pH).

Temperature Stability of HRP A2A

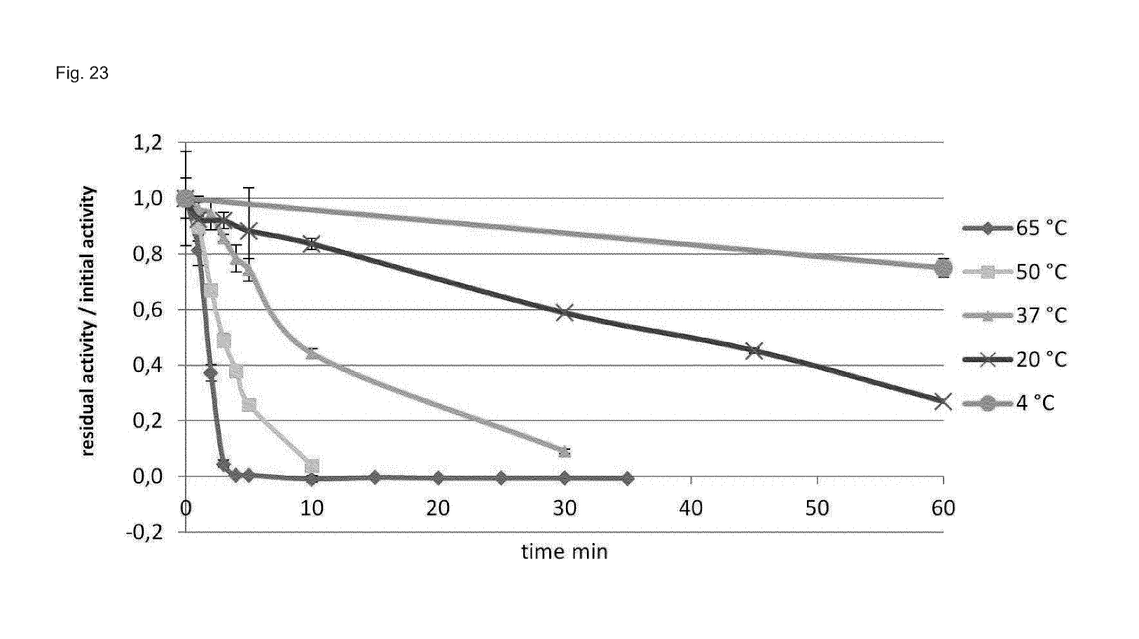

The 0.3 ng/μL diluted HRP A2A met the expectations and lost its activity over time the faster, the higher the incubation temperature was (FIG. 23). Storage for 60 min at 4° C. decreased the activity to 75% of the starting activity (4° C. data was taken from the experiment investigating the influence of protein concentration on stability, see below; data from 0.38 ng/μL HRP A2A). After 60 min, the diluted HRP A2A showed 50% of the initial activity with a descending trend at room temperature (i.e. 20° C.). At 37° C., HRP activity was measurable for 30 min. At 50° C. and 65° C., the activity was abrogated after 10 min and 3 min, respectively.

The finding, that HRP activity decreased relatively fast even at 4° C. did not match the observations on HRP stability made so far: Not any significant loss of activity was seen over months of HRP A2A stored at 4° C. However, contrary to the A2A used for this temperature stability study, the stored A2A was undiluted (i.e. 60 ng/μL), hence it is hypothesized, that protein concentration might play a crucial role in HRP stability (see below).

Dependence of the Stability of HRP A2A on Protein Concentration

It is assumed that the stability of HRP A2A depends on the protein concentration of the solution it is dissolved in. By studying the HRP activities over time at different concentrations, the hypothesis was successfully confirmed and the crucial “point-of-no-return” in protein concentration identified above which HRP stability is guaranteed, and below which HRP activity decreases rather fast.

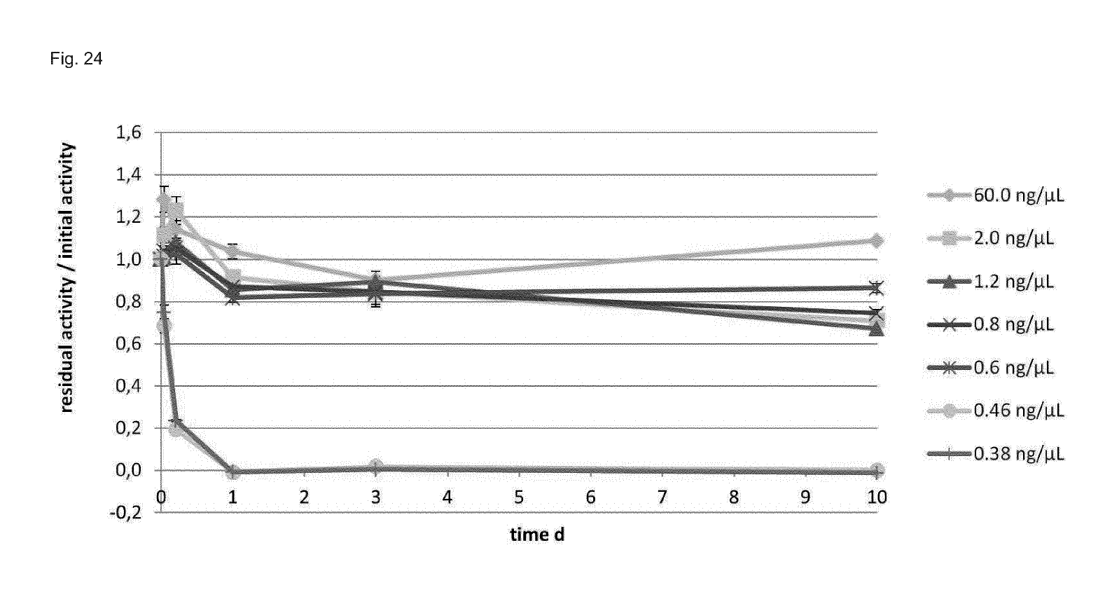

After storing HRP A2A at 4° C. for 10 d in concentrations 0.6 ng/μL no significant loss of HRP activity could be detected. However the two lower concentrated HRPs completely lost activity within one day (FIG. 24).

### Example 5

### Surface Variants for Oriented Enzyme Immobilization

Increased stability and the possibility for oriented immobilization of HRPs are desired features for the diverse uses of HRPs in biotechnological applications. In this study, 2 single and 12 double mutants of solvent exposed lysine residues were created, codon optimized and successfully expressed in Pichia pastoris. The mutations and expression levels of each variant enzyme after standard cultivation and methanol induction are depicted in Table 10. The values from 96-well deep-well cultivations were not normalized and thus not directly comparable between mutants, but demonstrated that substantial amounts (>100 mABS/min using standard ABTS assay) of 12 of the 14 surface variants can be produced in P. pastoris.

The production of three surface variants HRPC1AsynK232N_K241N, HRPC1AsynK174R_K241N and HRPC1AsynK174Q_K241F was up-scaled to 5 L volume (BiostatC fermenta), yielding expression levels of 34.1 U/ml; 30.2 U/ml and 44.3 U/ml, respectively. The yield of codon optimized C1A without modifications was 5.3 U/ml and wt C2 2.2 U/ml. The aforementioned yields are not directly comparable due to varying feeds used. Nevertheless, these data demonstrate the active expression of these surface lysine variants.

TABLE 10

| | SEQ ID | | Activity |
|---|---|---|---|
| Variant # | NO: | AA variation | mABS/min |
| 0 | 50 | HRPC1Asyn | 363 |
| 1 | 35 | HRPC1AsynK232Q_K241N | 236 |
| 2 | 36 | HRPC1AsynK232Q_K241F | 146 |
| 3 | 37 | HRPC1AsynK232N | 1428 |
| 4 | 38 | HRPC1AsynK232N_K241N | 293 |
| 5 | 39 | HRPC1AsynK232N_K241F | 77 |
| 6 | 40 | HRPC1AsynK174R_K241N | 135 |
| 7 | 41 | HRPC1AsynK174R_K241F | 45 |
| 8 | 42 | HRPC1AsynK174R_K232Q | 234 |
| 9 | 43 | HRPC1AsynK174R_K232N | 307 |
| 10 | 44 | HRPC1AsynK174Q_K241N | 157 |
| 11 | 45 | HRPC1AsynK174Q_K241F | 108 |
| 12 | 46 | HRPC1AsynK174Q_K232Q | 196 |
| 13 | 47 | HRPC1AsynK174Q_K232N | 133 |
| 14 | 48 | HRPC1Asyn_T110V | 56 |
| 15 | 49 | HRPC1Asyn_K241F | 134 |

### Example 6

### Production of Isoenzymes in Pichia pastoris

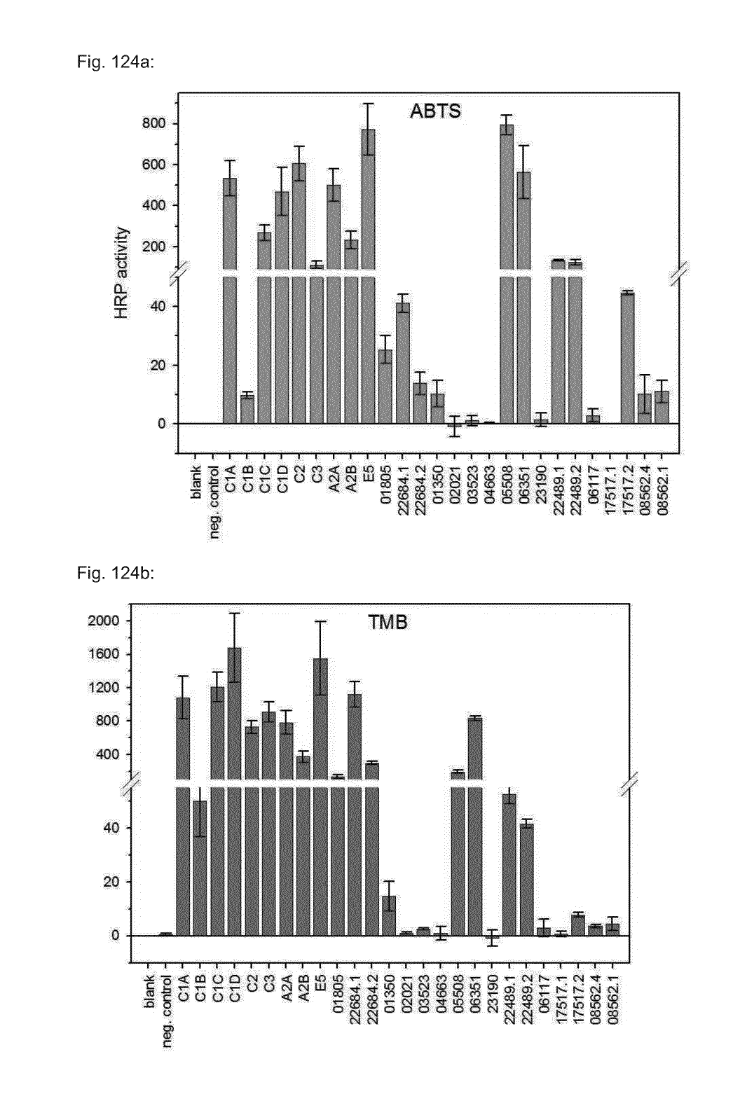

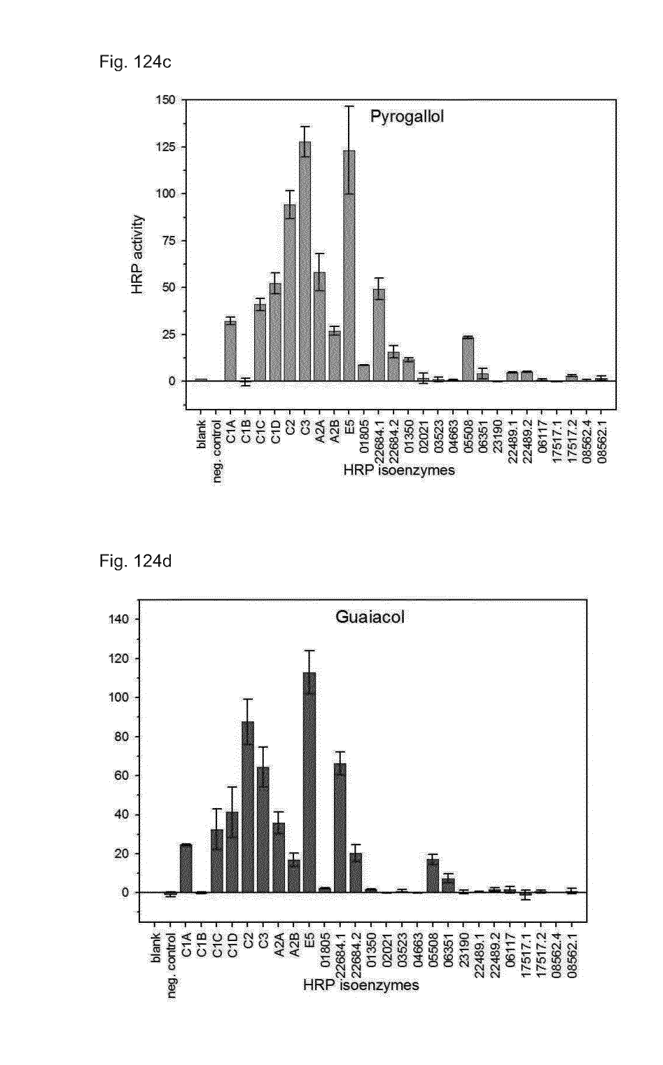

For the heterologous secretory production of single isoenzymes in P. pastoris, the codon usages of the coding sequences were optimized for efficient translation and fragments corresponding to the predicted mature isoenzymes were produced synthetically (SEQ ID NO:66-92). If the signal peptide prediction with SignalP led to two alternative signal peptide junctions, the mature peptides corresponding to the longer signal peptide variants were ordered as synthetic fragments, and the signal peptide variants with shorter mature peptide were successfully amplified via PCR. Correct cloning of all genes into P. pastoris expression vectors was verified by Sanger sequencing. Since the used P. pastoris expression vector pPpT4 already contains the signal sequence of the S. cerevisiae mating factor α, the isoenzymes were produced without the predicted natural signal sequences. From the 26 isoenzymes produced, 22 showed peroxidase activity with at least one of the substrates used. As depicted in Table 11 and FIG. 124, almost all isoenzymes could be produced in an active form in Pichia pastoris. Isoenzymes showing obvious peroxidase activity with the assay used are marked with “+”. Isoenzymes showing very low but detectable peroxidase activity with the assay used are marked with “(+)”. Isoenzymes with no activity detected during an observation period of 2 h are marked with “−”. Allelic variants not produced heterologously are marked with n.d. (no data available). Isoenzymes discovered during this study (previously unknown) are mareked with “*”.

TABLE 11

| | | Activity/ | Activity/ | Activity/ | Activity/ |
|---|---|---|---|---|---|
| Contig number | Name | ABTS | TMB | Guaiacol | Pyrogallol |
| — | C1A | + | + | + | + |
| 15901 | C1B | − | + | − | − |
| 25148 | C1C | + | + | (+) | + |
| 25148_2 | C1D* | + | + | (+) | + |
| 04627 | C2 | + | + | + | + |
| — | C3 | + | + | + | + |
| Manual | A2A* | + | + | + | + |
| assembly | | | | | |
| Manual | A2B* | + | + | (+) | (+) |
| assembly | | | | | |
| 04382 (Contig | E5 | + | + | + | + |
| split manually) | | | | | |
| 01805 | 01805* + | + | + | (+) | + |
| 22684 | 22684.1* + | + | + | + | + |
| 22684_2 | 22684.2* | + | + | + | + |
| | (B2B?) | | | | |
| 01350 (Contig | 01350* | + | + | (+) | + |
| split manually) | | | | | |
| 02021 | 02021* | − | − | − | − |
| 23190.1 | 23190.1* | − | − | − | − |
| 23190 | 23190.2* | n.d. | n.d. | n.d. | n.d. |
| 04663 | 04663.1* | + | (+) | − | − |
| 06351 | 06351* | + | + | + | + |
| 03523 | 03523* | − | − | − | − |
| 05508.1 | 05508.1* | + | + | + | + |
| 05508 | 05508.2* | + | + | n.d. | n.d. |
| 22489_1 | 22489.1* | + | + | (+) | + |
| 22489_2 | 22489.2* | + | + | (+) | + |
| 06117 | 06117* | − | − | − | − |
| 17517_1 | 17517.1* | + | − | − | − |
| 17517_2 | 17517.2* | + | + | (+) | (+) |
| 08562_1 | 08562.1* | + | (+) | − | − |
| 08562_4 (Contig | 08562.2* | + | (+) | − | (+) |
| split manually) | | | | | |

Pichia pastoris tends to hyperglycosylate proteins that pass through the endoplasmatic reticulum and the Golgi apparatus. The activity of an alpha-1,6-mannosyltransferase encoded by the gene OCH1 is believed to initiate the hyperglycosylation by additional mannosyltransferases in the Golgi apparatus. A permanent knock-out of the OCH1 open reading frame from the Pichia pastoris genome results in a Pichia pastoris strain, that allows the production of HRP isoenzymes with an altered glycosylation pattern, compared to HRP isoenzymes that were produced in a Pichia pastoris strain with the wild-type OCH1 gene. We are able to produce HRP isoenzymes with an altered glycosylation pattern in an active form using a Pichia pastoris strain in which the OCH1 gene was permanently knocked-out.
The different properties of the isoenzymes with respect to various substrates are depicted in FIG. 124 a-d. Comparing the measured activities to the four substrates tested, it can be seen that the substrates are differently converted by the isoenzymes under the conditions tested. These experimental data underscore the diversity of the isozymes and consequently the profitability of a kit, which allows a quick and easy identification of an isoenzyme that is best for a specific application.

### Example 7

### Use of the Peroxidase Kit

Use of the presented peroxidase kit is easy and straight-forward. The described horseradish peroxidases are produced in Pichia pastoris and can be tested against any substrate of a desired application under the conditions at which a reaction will be preferably catalyzed. Possible requirements to be met by a peroxidase isoenzyme might be enzymatic activity towards a given substrate at a specific pH or in a specific solvent, maximum stability of the enzyme over time under given conditions (e.g. at a certain temperature or in the presence of hydrogen peroxide at a certain concentration), the possibility for immobilization onto a given carrier material via a specific method etc. An example might be the identification of an isoenzyme that performs best under physiological conditions, since the desired application of the isoenzyme might be in cancer treatment and the enzyme will be administered to humans. In case another reaction needs to be catalyzed at pH 9.0 and 20° C. for several days, the buffer in which a peroxidase isoenzyme is dissolved in can be set to pH 9.0, the temperature can be set to 20° C. and the enzymatic activity of the peroxidase isoenzyme towards the desired substrate can be measured over a time period of one week. Performing an experiment like this with all peroxidase isoenzymes of the kit in parallel allows a rapid and convenient identification of an isoenzyme that either yields the highest activity towards the substrate, or the highest activity after the tested time, i.e. has the highest stability under the tested conditions, or both. The peroxidase kit consists of both, the Pichia pastoris strains producing the peroxidase isoenzymes, as well as the purified isoenzymes themselves. Hereby, a screening can be performed in two ways: either directly from the cultivation supernatant of the production strains, or by using lyophilisates of the isoenzymes and dissolving them directly in a buffer that fits the required conditions of an application.

Due to the vast diversity of applications and the thus versatile requirements to be met by a certain peroxidase isoenzyme, a kit comprising a multitude of isoenzymes to screen in a parallelized way provides a valuable tool for a fast and easy identification of the most favorable peroxidase isoenzyme for any application.

1-15. (canceled)

16. A recombinant heme-containing horseradish peroxidase isoenzyme (HRP) variant displaying a reduced number of lysine residues, wherein at least one solvent exposed lysine is mutated.

17. The HRP variant according to claim 16, wherein at least one lysine at position 174, 232 or 241 derived from SEQ ID NO:50 is mutated.

18. The HRP variant according to claim 17, wherein two lysines at position 174, 232 or 241 derived from SEQ ID NO:50 are mutated.

19. The HRP variant according to claim 16, wherein the lysine is replaced by an amino acid selected from the group consisting of phenylalanine, asparagine, glutamine or arginine.

20. An HRP variant encoded by a nucleic acid sequence of SEQ ID NO: 35-47 or 49.

21. The HRP variant according to claim 20 encoded by a nucleic acid sequence of SEQ ID NO: 38, 40, 45 or 49.

22. The HRP variant according to claim 20, wherein said variant has a biological activity of at least 500 U/mg, preferred of at least 750 U/mg, more preferred of at least 1,000 U/mg and most preferred of at least 1,200 U/mg in the ABTS assay.

23. The HRP variant according to claim 20, wherein said variant has a lower Kₘ when compared to the isoenzyme C1A.

24. An isolated polynucleic acid molecule, characterized in that it codes for a HRP variant according to claim 20.

25. A vector comprising a nucleic acid sequence according to claim 24.

26. A host cell comprising a vector according to claim 25.

27. The host cell according to claim 26, wherein the host cell is a prokaryotic cell, preferably E. coli or B. subtilis or a eukaryotic cell, preferably a yeast cell.

28. The host cell according to claim 26, wherein the host cell is a Pichia cell, preferably a P. pastoris or P. angusta cell.

29. A method for producing a recombinant HRP variant according to claim 16, which comprises
a. providing a recombinant host cell engineered to express a nucleic acid sequence according to claim 24;
b. culturing the host cell under conditions suitable for obtaining the HRP variant, wherein the expression product of the host cell is reacting with heme; and
c. recovering the HRP variant from the culture.

30. A method of using an HRP variant according to claim 16, wherein the HRP variant is used in an assay selected from at least one of a reagent in organic synthesis and biotransformation, a coupled enzyme assay, a chemiluminescent assay, and an immunoassay, in a biosensor, in a diagnostic or in bioremediation.
